SEQUENCE LISTING (126 sequences)

SEQ ID NO: 1 - protein, 29 aa, Artificial Sequence
  Description of Artificial Sequence Synthetic peptide
AALEALAEALEALAEALEALAEAAAAGGC

SEQ ID NO: 2 - protein, 30 aa, Artificial Sequence
  Description of Artificial Sequence Synthetic polypeptide
AALAEALAEALAEALAEALAEALAAAAGGC

SEQ ID NO: 3 - protein, 15 aa, Artificial Sequence
  Description of Artificial Sequence Synthetic peptide
ALEALAEALEALAEA

SEQ ID NO: 4 - protein, 22 aa, Artificial Sequence
  Description of Artificial Sequence Synthetic peptide
GLFEAIEGFIENGWEGMIWDYG

SEQ ID NO: 5 - protein, 23 aa, Artificial Sequence
  Description of Artificial Sequence Synthetic peptide
GLFGAIAGFIENGWEGMIDGWYG

SEQ ID NO: 6 - protein, 48 aa, Artificial Sequence
  Description of Artificial Sequence Synthetic polypeptide
GLFEAIEGFIENGWEGMIDGWYGCGLFEAIEGFIENGWEGMIDGWYGC

SEQ ID NO: 7 - protein, 44 aa, Artificial Sequence
  Description of Artificial Sequence Synthetic polypeptide
GLFEAIEGFIENGWEGMIDGGCGLFEAIEGFIENGWEGMIDGGC

SEQ ID NO: 8 - protein, 35 aa, Artificial Sequence
  Description of Artificial Sequence Synthetic polypeptide
GLFGALAEALAEALAEHLAEALAEALEALAAGGSC

SEQ ID NO: 9 - protein, 34 aa, Artificial Sequence
  Description of Artificial Sequence Synthetic polypeptide
GLFEAIEGFIENGWEGLAEALAEALEALAAGGSC

SEQ ID NO: 10 - protein, 41 aa, Artificial Sequence
  Description of Artificial Sequence Synthetic polypeptide
  MOD_RES (17)..(17): Norleucine
  MOD_RES (38)..(38): Norleucine
GLFEAIEGFIENGWEGLIDGKGLFEAIEGFIENGWEGXLDG

SEQ ID NO: 11 - protein, 19 aa, Artificial Sequence
  Description of Artificial Sequence Synthetic peptide
LFEALLELLESLWELLLEA

SEQ ID NO: 12 - protein, 20 aa, Artificial Sequence
  Description of Artificial Sequence Synthetic peptide
GLFKALLKLLKSLWKLLLKA

SEQ ID NO: 13 - protein, 20 aa, Artificial Sequence
  Description of Artificial Sequence Synthetic peptide
GLFRALLRLLRSLWRLLLRA

SEQ ID NO: 14 - protein, 30 aa, Artificial Sequence
  Description of Artificial Sequence Synthetic polypeptide
WEAKLAKALAKALAKHLAKALAKALKACEA

SEQ ID NO: 15 - protein, 22 aa, Artificial Sequence
  Description of Artificial Sequence Synthetic peptide
GLFFEAIAEFIEGGWEGLIEGC

SEQ ID NO: 16 - protein, 26 aa, Artificial Sequence
  Description of Artificial Sequence Synthetic peptide
GIGAVLKVLTTGLPALISWIKRKRQQ

SEQ ID NO: 17 - protein, 8 aa, Artificial Sequence
  Description of Artificial Sequence Synthetic peptide
HHHHHWYG

SEQ ID NO: 18 - protein, 10 aa, Artificial Sequence
  Description of Artificial Sequence Synthetic peptide
CHKKKKKKHC

SEQ ID NO: 19 - protein, 16 aa, Drosophila sp.
RQIKIWFQNRRMKWKK

SEQ ID NO: 20 - protein, 14 aa, Human immunodeficiency virus 1
GRKKRRQRRRPPQC

SEQ ID NO: 21 - protein, 27 aa, Artificial Sequence
  Description of Artificial Sequence Synthetic peptide
GALFLGWLGAAGSTMGAWSQPKKKRKV

SEQ ID NO: 22 - protein, 18 aa, Artificial Sequence
  Description of Artificial Sequence Synthetic peptide
LLIILRRRIRKQAHAHSK

SEQ ID NO: 23 - protein, 26 aa, Artificial Sequence
  Description of Artificial Sequence Synthetic peptide
GWTLNSAGYLLKINLKALAALAKKIL

SEQ ID NO: 24 - protein, 18 aa, Artificial Sequence
  Description of Artificial Sequence Synthetic peptide
KLALKLALKALKAALKLA

SEQ ID NO: 25 - protein, 9 aa, Artificial Sequence
  Description of Artificial Sequence Synthetic peptide
RRRRRRRRR

SEQ ID NO: 26 - protein, 10 aa, Artificial Sequence
  Description of Artificial Sequence Synthetic peptide
KFFKFFKFFK

SEQ ID NO: 27 - protein, 37 aa, Artificial Sequence
  Description of Artificial Sequence Synthetic polypeptide
LLGDFFRKSKEKIGKEFKRIVQRIKDFLRNLVPRTES

SEQ ID NO: 28 - protein, 31 aa, Artificial Sequence
  Description of Artificial Sequence Synthetic polypeptide
SWLSKTAKKLENSAKKRISEGIAIAIQGGPR

SEQ ID NO: 29 - protein, 30 aa, Artificial Sequence
  Description of Artificial Sequence Synthetic polypeptide
ACYCRIPACIAGERRYGTCIYQGRLWAFCC

SEQ ID NO: 30 - protein, 36 aa, Artificial Sequence
  Description of Artificial Sequence Synthetic polypeptide
DHYNCVSSGGQCLYSACPIFTKIQGTCYRGKAKCCK

SEQ ID NO: 31 - protein, 42 aa, Artificial Sequence
  Description of Artificial Sequence Synthetic polypeptide
RRRPRPPYLPRPRPPPFFPPRLPPRIPPGFPPRFPPRFPGKR

SEQ ID NO: 32 - protein, 13 aa, Artificial Sequence
  Description of Artificial Sequence Synthetic peptide
ILPWKWPWWPWRR

SEQ ID NO: 33 - protein, 16 aa, Unknown
  Description of Unknown RFGF peptide sequence
AAVALLPAVLLALLAP

SEQ ID NO: 34 - protein, 11 aa, Unknown
  Description of Unknown RFGF analogue sequence
AALLPVLLAAP

SEQ ID NO: 35 - protein, 12 aa, Artificial Sequence
  Description of Artificial Sequence Synthetic peptide
RKCRIVVIRVCR

SEQ ID NO: 36 - DNA, 20 bp, Artificial Sequence
  Description of Artificial Sequence Synthetic oligonucleotide
tttttttttttttttttttt

SEQ ID NO: 37 - DNA, 20 bp, Artificial Sequence
  Description of Artificial Sequence Synthetic oligonucleotide
  modified_base (20)..(20): t or a
  See specification as filed for detailed description of substitutions and preferred embodiments
tttttttttttttttttttn

SEQ ID NO: 38 - DNA, 21 bp, Artificial Sequence
  Description of Artificial Sequence Synthetic oligonucleotide
  modified_base (20)..(21): t or a
  See specification as filed for detailed description of substitutions and preferred embodiments
tttttttttttttttttttnn

SEQ ID NO: 39 - DNA, 20 bp, Artificial Sequence
  Description of Artificial Sequence Synthetic oligonucleotide
  modified_base (20)..(20): t or a
  See specification as filed for detailed description of substitutions and preferred embodiments
tttttttttttttttttttn

SEQ ID NO: 40 - DNA, 21 bp, Artificial Sequence
  Description of Artificial Sequence Synthetic oligonucleotide
  modified_base (20)..(21): t or a
  See specification as filed for detailed description of substitutions and preferred embodiments
tttttttttttttttttttnn

SEQ ID NO: 41 - DNA, 20 bp, Artificial Sequence
  Description of Artificial Sequence Synthetic oligonucleotide
tttttttttttttttttttt

SEQ ID NO: 42 - DNA, 20 bp, Artificial Sequence
  Description of Artificial Sequence Synthetic oligonucleotide
attttttttttttttttttt

SEQ ID NO: 43 - DNA, 20 bp, Artificial Sequence
  Description of Artificial Sequence Synthetic oligonucleotide
attttttttttttttttttt

SEQ ID NO: 44 - DNA, 20 bp, Artificial Sequence
  Description of Artificial Sequence Synthetic oligonucleotide
attttttttttttttttttt

SEQ ID NO: 45 - DNA, 20 bp, Artificial Sequence
  Description of Artificial Sequence Synthetic oligonucleotide
attttttttttttttttttt

SEQ ID NO: 46 - RNA, 21 nt, Artificial Sequence
  Description of Artificial Sequence Synthetic oligonucleotide
ugacaaaauaacucacuauaa

SEQ ID NO: 47 - RNA, 23 nt, Artificial Sequence
  Description of Artificial Sequence Synthetic oligonucleotide
uuauagugaguuauuuugucaau

SEQ ID NO: 48 - RNA, 21 nt, Artificial Sequence
  Description of Artificial Sequence Synthetic oligonucleotide
ugacaaaauaacucacuauaa

SEQ ID NO: 49 - RNA, 21 nt, Artificial Sequence
  Description of Artificial Sequence Synthetic oligonucleotide
ugacaaaauaacucacuauaa

SEQ ID NO: 50 - RNA, 21 nt, Artificial Sequence
  Description of Artificial Sequence Synthetic oligonucleotide
ugacaaaauaacucacuauaa

SEQ ID NO: 51 - RNA, 21 nt, Artificial Sequence
  Description of Artificial Sequence Synthetic oligonucleotide
ugacaaaauaacucacuauaa

SEQ ID NO: 52 - RNA, 21 nt, Artificial Sequence
  Description of Artificial Sequence Synthetic oligonucleotide
ugacaaaauaacucacuauaa

SEQ ID NO: 53 - RNA, 21 nt, Artificial Sequence
  Description of Artificial Sequence Synthetic oligonucleotide
ugacaaaauaacucacuauaa

SEQ ID NO: 54 - RNA, 21 nt, Artificial Sequence
  Description of Artificial Sequence Synthetic oligonucleotide
ugacaaaauaacucacuauaa

SEQ ID NO: 55 - RNA, 21 nt, Artificial Sequence
  Description of Artificial Sequence Synthetic oligonucleotide
ugacaaaauaacucacuauaa

SEQ ID NO: 56 - RNA, 21 nt, Artificial Sequence
  Description of Artificial Sequence Synthetic oligonucleotide
ugacaaaauaacucacuauaa

SEQ ID NO: 57 - RNA, 21 nt, Artificial Sequence
  Description of Artificial Sequence Synthetic oligonucleotide
ugacaaaauaacucacuauaa

SEQ ID NO: 58 - RNA, 21 nt, Artificial Sequence
  Description of Artificial Sequence Synthetic oligonucleotide
ugacaaaauaacucacuauaa

SEQ ID NO: 59 - RNA, 23 nt, Artificial Sequence
  Description of Artificial Sequence Synthetic oligonucleotide
uuauagugaguuauuuugucaau

SEQ ID NO: 60 - RNA, 23 nt, Artificial Sequence
  Description of Artificial Sequence Synthetic oligonucleotide
uuauagugaguuauuuugucaau

SEQ ID NO: 61 - RNA, 23 nt, Artificial Sequence
  Description of Artificial Sequence Synthetic oligonucleotide
uuauagugaguuauuuugucaau

SEQ ID NO: 62 - RNA, 23 nt, Artificial Sequence
  Description of Artificial Sequence Synthetic oligonucleotide
uuauagugaguuauuuugucaau

SEQ ID NO: 63 - RNA, 23 nt, Artificial Sequence
  Description of Artificial Sequence Synthetic oligonucleotide
uuauagugaguuauuuugucaau

SEQ ID NO: 64 - RNA, 23 nt, Artificial Sequence
  Description of Artificial Sequence Synthetic oligonucleotide
uuauagugaguuauuuugucaau

SEQ ID NO: 65 - RNA, 21 nt, Artificial Sequence
  Description of Artificial Sequence Synthetic oligonucleotide
uacuguuggauugauucgaaa

SEQ ID NO: 66 - RNA, 23 nt, Artificial Sequence
  Description of Artificial Sequence Synthetic oligonucleotide
uuucgaaucaauccaacaguagc

SEQ ID NO: 67 - RNA, 21 nt, Artificial Sequence
  Description of Artificial Sequence Synthetic oligonucleotide
uacuguuggauugauucgaaa

SEQ ID NO: 68 - RNA, 21 nt, Artificial Sequence
  Description of Artificial Sequence Synthetic oligonucleotide
uacuguuggauugauucgaaa

SEQ ID NO: 69 - RNA, 21 nt, Artificial Sequence
  Description of Artificial Sequence Synthetic oligonucleotide
uacuguuggauugauucgaaa

SEQ ID NO: 70 - RNA, 21 nt, Artificial Sequence
  Description of Artificial Sequence Synthetic oligonucleotide
uacuguuggauugauucgaaa

SEQ ID NO: 71 - RNA, 21 nt, Artificial Sequence
  Description of Artificial Sequence Synthetic oligonucleotide
uacuguuggauugauucgaaa

SEQ ID NO: 72 - RNA, 23 nt, Artificial Sequence
  Description of Artificial Sequence Synthetic oligonucleotide
uuucgaaucaauccaacaguagc

SEQ ID NO: 73 - RNA, 23 nt, Artificial Sequence
  Description of Artificial Sequence Synthetic oligonucleotide
uuucgaaucaauccaacaguagc

SEQ ID NO: 74 - RNA, 23 nt, Artificial Sequence
  Description of Artificial Sequence Synthetic oligonucleotide
uuucgaaucaauccaacaguagc

SEQ ID NO: 75 - RNA, 23 nt, Artificial Sequence
  Description of Artificial Sequence Synthetic oligonucleotide
uuucgaaucaauccaacaguagc

SEQ ID NO: 76 - RNA, 23 nt, Artificial Sequence
  Description of Artificial Sequence Synthetic oligonucleotide
uuucgaaucaauccaacaguagc

SEQ ID NO: 77 - RNA, 23 nt, Artificial Sequence
  Description of Artificial Sequence Synthetic oligonucleotide
uuucgaaucaauccaacaguagc

SEQ ID NO: 78 - RNA, 23 nt, Artificial Sequence
  Description of Artificial Sequence Synthetic oligonucleotide
uuucgaaucaauccaacaguagc

SEQ ID NO: 79 - RNA, 21 nt, Artificial Sequence
  Description of Artificial Sequence Synthetic oligonucleotide
aacaguguucuugcucuauaa

SEQ ID NO: 80 - RNA, 23 nt, Artificial Sequence
  Description of Artificial Sequence Synthetic oligonucleotide
uuauagagcaagaacacuguuuu

SEQ ID NO: 81 - RNA, 21 nt, Artificial Sequence
  Description of Artificial Sequence Synthetic oligonucleotide
aacaguguucuugcucuauaa

SEQ ID NO: 82 - RNA, 21 nt, Artificial Sequence
  Description of Artificial Sequence Synthetic oligonucleotide
aacaguguucuugcucuauaa

SEQ ID NO: 83 - RNA, 21 nt, Artificial Sequence
  Description of Artificial Sequence Synthetic oligonucleotide
aacaguguucuugcucuauaa

SEQ ID NO: 84 - RNA, 21 nt, Artificial Sequence
  Description of Artificial Sequence Synthetic oligonucleotide
aacaguguucuugcucuauaa

SEQ ID NO: 85 - RNA, 21 nt, Artificial Sequence
  Description of Artificial Sequence Synthetic oligonucleotide
aacaguguucuugcucuauaa

SEQ ID NO: 86 - RNA, 21 nt, Artificial Sequence
  Description of Artificial Sequence Synthetic oligonucleotide
aacaguguucuugcucuauaa

SEQ ID NO: 87 - RNA, 23 nt, Artificial Sequence
  Description of Artificial Sequence Synthetic oligonucleotide
uuauagagcaagaacacuguuuu

SEQ ID NO: 88 - RNA, 23 nt, Artificial Sequence
  Description of Artificial Sequence Synthetic oligonucleotide
uuauagagcaagaacacuguuuu

SEQ ID NO: 89 - RNA, 23 nt, Artificial Sequence
  Description of Artificial Sequence Synthetic oligonucleotide
uuauagagcaagaacacuguuuu

SEQ ID NO: 90 - RNA, 23 nt, Artificial Sequence
  Description of Artificial Sequence Synthetic oligonucleotide
uuauagagcaagaacacuguuuu

SEQ ID NO: 91 - RNA, 23 nt, Artificial Sequence
  Description of Artificial Sequence Synthetic oligonucleotide
uuauagagcaagaacacuguuuu

SEQ ID NO: 92 - RNA, 23 nt, Artificial Sequence
  Description of Artificial Sequence Synthetic oligonucleotide
uuauagagcaagaacacuguuuu

SEQ ID NO: 93 - RNA, 23 nt, Artificial Sequence
  Description of Artificial Sequence Synthetic oligonucleotide
uuauagagcaagaacacuguuuu

SEQ ID NO: 94 - RNA, 23 nt, Artificial Sequence
  Description of Artificial Sequence Synthetic oligonucleotide
uuauagagcaagaacacuguuuu

SEQ ID NO: 95 - RNA, 21 nt, Artificial Sequence
  Description of Artificial Sequence Synthetic oligonucleotide
uucuugcucuauaaaccgugu

SEQ ID NO: 96 - RNA, 21 nt, Artificial Sequence
  Description of Artificial Sequence Synthetic oligonucleotide
aacaguguucuugcucuauau

SEQ ID NO: 97 - RNA, 21 nt, Artificial Sequence
  Description of Artificial Sequence Synthetic oligonucleotide
aacaguguucuugcucuauau

SEQ ID NO: 98 - RNA, 21 nt, Artificial Sequence
  Description of Artificial Sequence Synthetic oligonucleotide
aacaguguucuugcucuauau

SEQ ID NO: 99 - RNA, 21 nt, Artificial Sequence
  Description of Artificial Sequence Synthetic oligonucleotide
aacaguguucuugcucuauau

SEQ ID NO: 100 - RNA, 21 nt, Artificial Sequence
  Description of Artificial Sequence Synthetic oligonucleotide
aacaguguucuugcucuauau

SEQ ID NO: 101 - RNA, 21 nt, Artificial Sequence
  Description of Artificial Sequence Synthetic oligonucleotide
aacaguguucuugcucuauau

SEQ ID NO: 102 - RNA, 23 nt, Artificial Sequence
  Description of Artificial Sequence Synthetic oligonucleotide
acacgguuuauagagcaagaaca

SEQ ID NO: 103 - RNA, 23 nt, Artificial Sequence
  Description of Artificial Sequence Synthetic oligonucleotide
acacgguuuauagagcaagaaca

SEQ ID NO: 104 - RNA, 23 nt, Artificial Sequence
  Description of Artificial Sequence Synthetic oligonucleotide
acacgguuuauagagcaagaaca

SEQ ID NO: 105 - RNA, 23 nt, Artificial Sequence
  Description of Artificial Sequence Synthetic oligonucleotide
acacgguuuauagagcaagaaca

SEQ ID NO: 106 - RNA, 23 nt, Artificial Sequence
  Description of Artificial Sequence Synthetic oligonucleotide
acacgguuuauagagcaagaaca

SEQ ID NO: 107 - RNA, 23 nt, Artificial Sequence
  Description of Artificial Sequence Synthetic oligonucleotide
acacgguuuauagagcaagaaca

SEQ ID NO: 108 - RNA, 23 nt, Artificial Sequence
  Description of Artificial Sequence Synthetic oligonucleotide
acacgguuuauagagcaagaaca

SEQ ID NO: 109 - RNA, 23 nt, Artificial Sequence
  Description of Artificial Sequence Synthetic oligonucleotide
acacgguuuauagagcaagaaca

SEQ ID NO: 110 - RNA, 23 nt, Artificial Sequence
  Description of Artificial Sequence Synthetic oligonucleotide
acacgguuuauagagcaagaaca

SEQ ID NO: 111 - RNA, 23 nt, Artificial Sequence
  Description of Artificial Sequence Synthetic oligonucleotide
acacgguuuauagagcaagaaca

SEQ ID NO: 112 - RNA, 23 nt, Artificial Sequence
  Description of Artificial Sequence Synthetic oligonucleotide
auauagagcaagaacacuguuuu

SEQ ID NO: 113 - RNA, 23 nt, Artificial Sequence
  Description of Artificial Sequence Synthetic oligonucleotide
auauagagcaagaacacuguuuu

SEQ ID NO: 114 - RNA, 23 nt, Artificial Sequence
  Description of Artificial Sequence Synthetic oligonucleotide
auauagagcaagaacacuguuuu

SEQ ID NO: 115 - RNA, 23 nt, Artificial Sequence
  Description of Artificial Sequence Synthetic oligonucleotide
auauagagcaagaacacuguuuu

SEQ ID NO: 116 - RNA, 23 nt, Artificial Sequence
  Description of Artificial Sequence Synthetic oligonucleotide
auauagagcaagaacacuguuuu

SEQ ID NO: 117 - RNA, 23 nt, Artificial Sequence
  Description of Artificial Sequence Synthetic oligonucleotide
auauagagcaagaacacuguuuu

SEQ ID NO: 118 - DNA, 12 bp, Artificial Sequence
  Description of Artificial Sequence Synthetic oligonucleotide
tacagtctatgt

SEQ ID NO: 119 - DNA, 12 bp, Artificial Sequence
  Description of Artificial Sequence Synthetic oligonucleotide
  See specification as filed for detailed description of substitutions and preferred embodiments
acatagactgta

SEQ ID NO: 120 - RNA, 12 nt, Artificial Sequence
  Description of Artificial Sequence Synthetic oligonucleotide
uacagucuaugu

SEQ ID NO: 121 - RNA, 12 nt, Artificial Sequence
  Description of Artificial Sequence Synthetic oligonucleotide
  See specification as filed for detailed description of substitutions and preferred embodiments
acauagacugua

SEQ ID NO: 122 - DNA, 20 bp, Artificial Sequence
  Description of Artificial Sequence Synthetic oligonucleotide
tttttttttttttttttttt

SEQ ID NO: 123 - DNA, 20 bp, Artificial Sequence
  Description of Artificial Sequence Synthetic oligonucleotide
ttttttttttttttttttta

SEQ ID NO: 124 - DNA, 20 bp, Artificial Sequence
  Description of Artificial Sequence Synthetic oligonucleotide
ttttttttttttttttttta

SEQ ID NO: 125 - DNA, 20 bp, Artificial Sequence
  Description of Artificial Sequence Synthetic oligonucleotide
ttttttttttttttttttta

SEQ ID NO: 126 - DNA, 20 bp, Artificial Sequence
  Description of Artificial Sequence Synthetic oligonucleotide
ttttttttttttttttttta

(19) United States
(12) Patent Application Publication
(10) Pub. No.: US 2024/0116973 A1
(43) Pub. Date: Apr. 11, 2024

(54) MODIFIED OLIGONUCLEOTIDES

(71) Applicant: ALNYLAM PHARMACEUTICALS, INC., Cambridge, MA (US)

(72) Inventors: Muthiah MANOHARAN, Cambridge, MA (US); Dhrubajyoti DATTA, Cambridge, MA (US)

(73) Assignee: ALNYLAM PHARMACEUTICALS, INC., Cambridge, MA (US)

(21) Appl. No.: 18/272,448

(22) PCT Filed: Jan. 14, 2022
(86) PCT No.: PCT/US22/12435

§ 371 Date: Jul. 14, 2023

Related U.S. Application Data
(60) Provisional application No. 63/138,006, filed on Jan. 15, 2021.

(51) Int. Cl. C07H 19/16; C12N 15/113

(52) U.S. Cl. CPC C07H 19/16; C12N 15/113; C12N 2310/14; C12N 2310/315; C12N 2310/333; C12N 2310/344

(57) ABSTRACT

One aspect of the present invention relates to double-stranded RNA (dsRNA) molecules comprising a 6-methyladenine nucleobase and capable of inhibiting the expression of a target gene. Other aspects of the invention relate to pharmaceutical compositions comprising these dsRNA molecules suitable for therapeutic use, and methods of inhibiting the expression of a target gene by administering these dsRNA molecules, e.g., for the treatment of various disease conditions.

### CROSS-REFERENCE TO RELATED APPLICATIONS

This application is a 35 U.S.C. § 371 National Phase Entry Application of International Application No. PCT/US2022/012435, filed Jan. 14, 2022, which claims benefit under 35 U.S.C. § 119(e) to U.S. Provisional Application No. 63/138,006, filed Jan. 15, 2021, the contents of each of which are incorporated herein by reference in their entireties.

### FIELD OF THE INVENTION

The invention relates to monomers and oligonucleotides, e.g., single-stranded oligonucleotides and dsRNAs comprising such monomers that are advantageous for inhibition of target gene expression, as well oligonucleotide, e.g., single-stranded oligonucleotide compositions and dsRNA compositions, suitable for therapeutic use. Additionally, the invention provides methods of inhibiting the expression of a target gene by administering these oligonucleotides, such as single-stranded oligonucleotides and dsRNAs agents, e.g., for the treatment of various diseases.

### SEQUENCE LISTING

The instant application contains a Sequence Listing which has been submitted electronically in ASCII format and is hereby incorporated by reference in its entirety. Said ASCII copy, created on Jul. 14, 2023, is named 051058-099310WOPT_SL.txt and is 35,073 bytes in size.

### BACKGROUND

RNA interference or “RNAi” is a term initially coined by Fire and co-workers to describe the observation that double-stranded RNAi (dsRNA) can block gene expression (Fire et al. (1998) Nature 391, 806-811; Elbashir et al. (2001) Genes Dev. 15, 188-200). Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, but the protein components of this activity remained unknown.

There remains a need in the art for effective nucleotide or chemical motifs for dsRNA molecules, which are advantageous for inhibition of target gene expression. This invention is directed to that effort.

### SUMMARY

This invention provides effective nucleotide or chemical motifs for oligonucleotides, including dsRNA molecules, which are advantageous for inhibition of target gene expression, as well as RNAi compositions suitable for therapeutic use. The invention further provides the reactive intermediate nucleotides which are useful for preparation of oligonucleotides, including the dsRNA molecules and RNAi compositions provided herein.

In one aspect, provided herein is an oligonucleotide comprising at least one nucleoside of Formula (I):

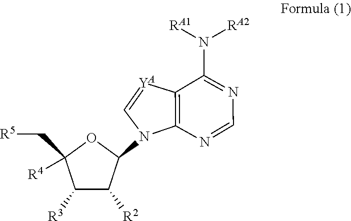

In nucleosides of Formula (I), Y^{A} is N or CH. For example, in some nucleosides of Formula (I), Y^{A} is N.

In nucleosides of Formula (I), R^{A1} is optionally substituted C₁₋₃₀ alkyl, optionally substituted C₂₋₃₀alkenyl, optionally substituted C₂₋₃₀alkynyl, alkylester, optionally substituted aryl, optionally substituted heteroaryl, optionally substituted heterocyclyl, optionally substituted cycloalkyl, or optionally substituted benzyl, a ligand, or a linker covalently bonded to one or more ligands. For example, R^{A1} is optionally substituted C₁₋₃₀ alkyl, optionally substituted C₃-C₈cyclyl, or optionally substituted benzyl. In some nucleosides of Formula (I), R^{A1} is optionally substituted C₁-C₆alkyl (e.g., methyl, ethyl, propyl, isopropyl, butyl, i-butyl, and t-butyl) or optionally substituted C₃-C₈cyclyl (e.g., cyclopropyl). For example, R^{A1} is methyl, ethyl, propyl, isopropyl or cyclopropyl).

In some nucleosides of Formula (I), R^{A1} is an optionally substituted benzyl. For example, in some nucleosides of Formula (I), R^{A1} is

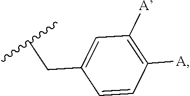

where A and A′ independently are hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl, alkoxyalkylamine, alkoxyoxycarboxylate, amino, alkylamino, dialkylamino, protected amino, a ligand, or a linker covalently bonded to one or more ligands. In some nucleosides of Formula (I), at least one of A and A′ is not H. In some nucleosides of Formula (I), neither one of A and A′ is H. In some nucleosides of Formula (I), only one of A and A′ is H.

In some nucleosides of Formula (I), A and A′ independently are H or C₁₋₃₀ alkyl optionally substituted with one or two substituents independently selected from the group consisting of hydroxyl, C₁-C₆alkoxy, oxo, halogen, caboxy, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxylalkyl, alkanesulfonyl, arenesulfonyl, alkanesulfonamido, arenesulfonamido, aralkylsulfonamido, alkylcarbonyl, acyloxy, cyano and ureido. For example, A and A′ independently are H or C₁₋₆alkyl (e.g., methyl, ethyl or propyl) optionally substituted with a oxo (═O) and C₁-C₆alkoxy (e.g., methoxy). In some nucleosides of Formula (I), A and A′ independently are H, CO₂Me or CH₂CO₂Me. For example, in some nucleosides of Formula (I), R^{A1} is

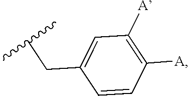

where: (i) A is CH₂CO₂Me and A′ is H; (ii) A is H and A′ is CH₂CO₂Me; (iii) A and A′ each are CH₂CO₂Me; (iv) A is CO₂Me and A′ is H; (v) A is H and A′ is CO₂Me; or (vi) A and A′ each are CO₂Me.

In nucleosides of Formula (I), R^{A2} is H or nitrogen protecting group. In some nucleosides of Formula (I), R^{A2} is H. In some other nucleosides of Formula (I), R^{A2} is a nitrogen protecting group. For example, R^{A} is

In nucleosides of Formula (I), R² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a bond to an internucleotide linkage to a subsequent nucleotide, a 3′-oligonucleotide capping group, a ligand, a linker covalently bonded to one or more ligands, a solid support, a linker or a linker covalently bonded a solid support. In some embodiments, R² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support. For example, R² is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy), alkoxyalkyl (e.g., methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R₈)(CH₂R₉), or —O—C₄₋₃₀alkyl-ON(CH₂R₈)(CH₂R₉). In some embodiments, R² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl) or C₆₋₂₄alkoxy (e.g., n-C₆₋₂₄ alkoxy).

In nucleosides of Formula (I), R³ is a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—N-methylacetamido, —O—C₄₋₃₀alkyl-ON(CH₂R⁸)(CH₂R⁹), —O—C₄₋₃₀alkyl-ON(CH₂R⁸)(CH₂R⁹), a 3′-oligonucleotide capping group, a ligand, a linker covalently bonded to one or more ligands, a solid support, a linker or a linker covalently bonded to a solid support. In some embodiments, R³ is a bond to an internucleotide linkage to a subsequent nucleotide, hydroxyl, optionally substituted C₁₋₃₀ alkoxy, a 3′-oligonucleotide capping group, a solid support, a linker or a linker covalently bonded to a solid support. For example, R³ is a bond to an internucleotide linkage to a subsequent nucleotide, hydroxyl or protected hydroxyl. In some embodiments, R³ is a bond to an internucleotide linkage to a subsequent nucleotide. In some embodiments, R³ is a hydroxyl or protected hydroxyl.

In some nucleosides of Formula (I), R⁴ is hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ in Formula (I) is H.

In some nucleosides of Formula (I), R⁴ and R² taken together are 4′-C(R¹⁰R¹¹)ᵥ—Y-2′ or 4′-Y—C(R¹⁰R¹¹)ᵥ-2′; Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; and v is 1, 2 or 3. For example, R² and R⁴ taken together are 4′-C(R¹⁰R¹¹)ᵥ—Y-2′ or 4′-Y—C(R¹⁰R¹¹)ᵥ-2. In some embodiments, R² and R⁴ taken together are 4′-C(R¹⁰R¹¹)ᵥ—Y-2′, where Y is O, one of R¹⁰ and R¹¹ is H and the other H or C₁-C₆alkyl (e.g., methyl or ethyl), and v is 1. For example, R² and R⁴ taken together are 4′-CH(R¹¹)—O-2′, where R¹¹ is H, methyl or CH₂OCH₃.

In some nucleosides of Formula (I), R⁴ and R³ taken together with the atoms to which they are attached form an optionally substituted C₃₋₈cycloalkyl, optionally substituted C₃₋₈cycloalkenyl, or optionally substituted 3-8 membered heterocyclyl.

In nucleosides of Formula (I), R⁵ represents a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—XP, XP is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R)(OH)(O)P—O-5′, R is optionally substituted C₁₋₃₀ alkyl, e.g., methyl, ethyl, isopropyl, or propyl)], alkyletherphosphonates [(R¹)(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein: X is O or S; a and b are each independently 1-10; and each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some nucleosides of Formula (I), R⁵ is a bond to an internucleotide linkage to a preceding nucleotide, hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate, phosphate mimic, or a bond to an internucleotide linkage to a preceding nucleotide. For example, R⁵ is hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate. In some embodiments, R⁵ is a bond to an internucleotide linkage to a preceding nucleotide. In some embodiments, R⁵ is hydroxyl, protected hydroxyl.

It is noted that in nucleosides of Formula (I) no more than one of R² and R³ is a bond to an internucleotide linkage to a subsequent nucleotide, and when both of R² and R³ are not a bond to an internucleotide linkage, then R⁵ is a bond to an internucleotide linkage to a preceding nucleotide.

Optionally, the nucleoside of Formula (I) is not where Y^{A} is N; R^{A1} is methyl, isopentyl, isopentenyl, propargyl, neopentyl, 1-methylpropyl or 1-methylbutyl; R^{A2} is H or nitrogen protecting group; R² is hydroxyl or protected hydroxyl; R³ is a bond to an internucleotide linkage to a subsequent nucleotide, hydroxyl or protected hydroxyl; R⁴ is H; and R⁵ is a bond to an internucleotide linkage to a preceding nucleotide, hydroxyl or protected hydroxyl, and both of R³ and R⁵ are not hydroxyl or protected hydroxyl at the same time.

It is noted that the nucleoside of Formula (I) can be located anywhere in the oligonucleotide. In some embodiments, the nucleoside of Formula (I) is present at the 5′- or 3′-terminus of the oligonucleotide. In some embodiments, the nucleoside of Formula (I) is present at an internal position of the oligonucleotide.

In another aspect, provided herein is a compound of Formula (II):

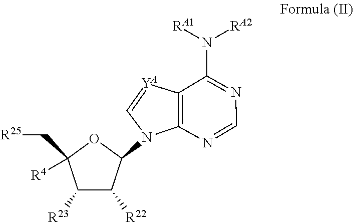

In compounds of Formula (II), Y^{A} is N or CH. For example, in some compounds of Formula (II), Y^{A} is N.

In compounds of Formula (II), R^{A1} is optionally substituted C₁₋₃₀ alkyl, optionally substituted C₂₋₃₀alkenyl, optionally substituted C₂₋₃₀alkynyl, alkylester, optionally substituted aryl, optionally substituted heteroaryl, optionally substituted heterocyclyl, optionally substituted cycloalkyl, or optionally substituted benzyl, a ligand, or a linker covalently bonded to one or more ligands. For example, R^{A1} is optionally substituted C₁₋₃₀ alkyl, optionally substituted C₃-C₈cyclyl, or optionally substituted benzyl. In some compounds of Formula (II), R^{A1} is optionally substituted C₁-C₆alkyl (e.g., methyl, ethyl, propyl, isopropyl, butyl, i-butyl, and t-butyl) or optionally substituted C₃-C₈cyclyl (e.g., cyclopropyl). For example, R^{A1} is methyl, ethyl, propyl, isopropyl or cyclopropyl).

In some compounds of Formula (II), R^{A1} is an optionally substituted benzyl. For example, in some compounds of Formula (II), R^{A1} is

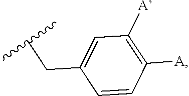

where A and A′ independently are hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl, alkoxyalkylamine, alkoxyoxycarboxylate, amino, alkylamino, dialkylamino, protected amino, a ligand, or a linker covalently bonded to one or more ligands. In some compounds of Formula (II), at least one of A and A′ is not H. In some compounds of Formula (II), neither one of A and A′ is H. In some compounds of Formula (II), only one of A and A′ is H.

In some compounds of Formula (II), A and A′ independently are H or C₁₋₃₀ alkyl optionally substituted with one or two substituents independently selected from the group consisting of hydroxyl, C₁-C₆alkoxy, oxo, halogen, caboxy, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxylalkyl, alkanesulfonyl, arenesulfonyl, alkanesulfonamido, arenesulfonamido, aralkylsulfonamido, alkylcarbonyl, acyloxy, cyano and ureido. For example, A and A′ independently are H or C₁₋₆alkyl (e.g., methyl, ethyl or propyl) optionally substituted with a oxo (═O) and C₁-C₆alkoxy (e.g., methoxy). In some compounds of Formula (II), A and A′ independently are H, CO₂Me or CH₂CO₂Me. For example, in some compounds of Formula (II), R^{A1} is

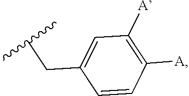

where: (i) A is CH₂CO₂Me and A′ is H; (ii) A is H and A′ is CH₂CO₂Me; (iii) A and A′ each are CH₂CO₂Me; (iv) A is CO₂Me and A′ is H; (v) A is H and A′ is CO₂Me; or (vi) A and A′ each are CO₂Me.

In compounds of Formula (II), R²² is hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a ligand, a linker covalently bonded to one or more ligands, a solid support, a linker or a linker covalently bonded a solid support. In some embodiments, R²² can be hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support. For example, R²² can be hydrogen, hydroxyl, halogen, protected hydroxyl, phosphate group, reactive phosphorous group, optionally substituted C₁₋₃₀ alkyl, optionally substituted C₂₋₃₀ alkenyl, optionally substituted C₂₋₃₀ alkynyl, optionally substituted C₁₋₃₀alkoxy (e.g., methoxy, 2-methoxyethoxy), alkoxyalkyl (e.g., methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, a solid support, a linker or a linker covalently attached to a solid support. In some embodiments, R²² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl), C₆₋₂₄alkoxy (e.g., n-C₆₋₂₄ alkoxy), a reactive phosphorous group, a solid support, a linker or a linker covalently attached to a solid support. For example, R²² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl) or C₆₋₂₄alkoxy (e.g., n-C₆₋₂₄ alkoxy).

In compounds of Formula (II), R²³ hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a ligand, a linker covalently bonded to one or more ligands, a solid support, a linker or a linker covalently bonded to a solid support. For example, R²³ is hydrogen, hydroxyl, protected hydroxyl, a reactive phosphorous group, a solid support, a linker, or a linker covalently attached to a solid support. In some embodiments, R²³ is hydrogen, hydroxyl or protected hydroxyl. In some other embodiments, R²³ is a reactive phosphorous group, a solid support, a linker, or a linker covalently attached to a solid support. For example, R²³ is a reactive phosphorous or a linker covalently attached to a solid support. In some embodiments, R²³ is a reactive phosphorous group. For example, R²³ is phosphoramidite group such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite).

In some compounds of Formula (II), R⁴ is hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ in Formula (II) is H.

In some compounds of Formula (II), R⁴ and R²² taken together are 4′-C(R¹⁰R¹¹)ᵥ—Y-2′ or 4′-Y—C(R¹⁰R¹¹)ᵥ-2′; Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; and v is 1, 2 or 3. For example, R²² and R⁴ taken together are 4′-C(R¹⁰R¹¹)ᵥ—Y-2′ or 4′-Y—C(R¹⁰R¹¹)ᵥ-2. In some embodiments, R²² and R⁴ taken together are 4′-C(R¹⁰R¹¹)ᵥ—Y-2′, where Y is O, one of R¹⁰ and R¹¹ is H and the other H or C₁-C₆alkyl (e.g., methyl or ethyl), and v is 1. For example, R²² and R⁴ taken together are 4′-CH(R¹¹)—O-2′, where R¹¹ is H, methyl or CH₂OCH₃.

In some compounds of Formula (II), R⁴ and R²³ taken together with the atoms to which they are attached form an optionally substituted C₃₋₈cycloalkyl, optionally substituted C₃₋₈cycloalkenyl, or optionally substituted 3-8 membered heterocyclyl.

In compounds of Formula (II), R²⁵ is hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—XP, XP is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R)(OH)(O)P—O-5′, R is optionally substituted C₁₋₃₀ alkyl, e.g., methyl, ethyl, isopropyl, or propyl)], alkyletherphosphonates [(R¹)(OH)(O)P—O-5′, R¹ is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; a and b are each independently 1-10; and each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl. In some embodiments, R²⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic. For example, R²⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic. In some embodiments, R²⁵ is a vinylphosphonategroup, cyclopropylphosphonate. In some other embodiments, R²⁵ is hydroxyl or protected hydroxyl.

Optionally, the compound of Formula (I) is not where Y^{A} is N; R^{A1} is methyl, isopentyl, isopentenyl, propargyl, neopentyl, 1-methylpropyl or 1-methylbutyl; R^{A2} is H or nitrogen protecting group; R²² is hydrogen, hydroxyl, protected hydroxyl, or a reactive phosphorous group; R²³ is hydroxyl, protected hydroxyl or reactive phosphorous group; R⁴ is H; and R²⁵ is hydroxyl or protected hydroxyl, and only one of R²² and R²³ is a reactive phosphorous group.

In some embodiments of any one of the aspects described herein, R²⁵ is a protected hydroxyl (e.g., 4,4′-dimethoxytrityl-protected) or a phosphate group. For example, R²⁵ is a phosphate group.

In some embodiments of any one of the aspects described herein, R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite).

In some embodiments of any one of the aspects described herein, R²² is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy, 2-methoxyethoxy), alkoxyalkyl (e.g., methoxyethyl), alkoxyalkylamine, alkoxyoxycarboxylate, amino, alkylamino, dialkylamino, —O—N-methylacetamido, —O—C₄₋₃₀alkyl-ON(CH₂R₈)(CH₂R₉), or —O—C₄₋₃₀alkyl-ON(CH₂R₈)(CH₂R₉). For example, R²² is hydrogen, hydroxyl, fluoro, chloro, methoxy, ethoxy, 2-methoxyethyl, —O—N-methylacetamido, C₆₋₂₄ alkyl (e.g., n-C₆₋₂₄ alkyl) or C₆₋₂₄alkoxy (e.g., n-C₆₋₂₄ alkoxy).

In some compounds of Formula (II), R²⁵ is a protected hydroxyl (e.g., 4,4′-dimethoxytrityl-protected) or a phosphate group; R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²² is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy, 2-methoxyethoxy), alkoxyalkyl (e.g., methoxyethyl), alkoxyalkylamine, alkoxyoxycarboxylate, amino, alkylamino, dialkylamino, —O—N-methylacetamido, —O—C₄₋₃₀alkyl-ON(CH₂R₈)(CH₂R₉), or —O—C₄₋₃₀alkyl-ON(CH₂R₈)(CH₂R₉); and, optionally R⁴ is H.

In some compounds of Formula (II), R²⁵ is a protected hydroxyl (e.g., 4,4′-dimethoxytrityl-protected) or a phosphate group; R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²² is hydrogen, hydroxyl, fluoro, chloro, methoxy, ethoxy, 2-methoxyethyl, —O—N-methylacetamido, C₆₋₂₄ alkyl (e.g., n-C₆₋₂₄ alkyl), or C₆₋₂₄alkoxy (e.g., n-C₆₋₂₄ alkoxy); and, optionally R⁴ is H.

In some compounds of Formula (II), R²⁵ is a protected hydroxyl (e.g., 4,4′-dimethoxytrityl-protected) or a phosphate group; R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²² is hydrogen, hydroxyl, fluoro, chloro, methoxy, ethoxy, 2-methoxyethyl, —O—N-methylacetamido, C₆₋₂₄ alkyl (e.g., n-C₆₋₂₄ alkyl) or C₆₋₂₄alkoxy (e.g., n-C₆₋₂₄ alkoxy); and, optionally R⁴ is H.

In some compounds of Formula (II), is a protected hydroxyl (e.g., 4,4′-dimethoxytrityl-protected) or a phosphate group; R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R⁴ and R²² taken together are 4′-C(R¹⁰R¹¹)ᵥ—Y-2′.

In some compounds of Formula (II), is a protected hydroxyl (e.g., 4,4′-dimethoxytrityl-protected) or a phosphate group; R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R⁴ and R²² taken together are 4′-C(R¹⁰R¹¹)ᵥ—O-2′.

In some compounds of Formula (II), R²⁵ is a protected hydroxyl (e.g., 4,4′-dimethoxytrityl-protected) or a phosphate group; R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R⁴ and R²² taken together are 4′-C(R¹⁰R¹¹)—O-2′.

In some compounds of Formula (II), R²⁵ is a protected hydroxyl (e.g., 4,4′-dimethoxytrityl-protected) or a phosphate group; R²³ is hydroxyl or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R⁴ and R²² taken together are 4′-CH(R¹¹)—O-2′, where R¹¹ is H or methyl.

In another aspect, provided herein is a compound of Formula (III):

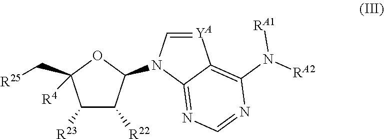

In compounds of Formula (III), Y^{A} is N or CH. For example, Y^{A} is N.

In compounds of Formula (III), R^{A1} is optionally substituted C₁₋₃₀ alkyl, optionally substituted C₂₋₃₀alkenyl, optionally substituted C₂₋₃₀alkynyl, alkylester, optionally substituted aryl, optionally substituted heteroaryl, optionally substituted heterocyclyl, optionally substituted cycloalkyl, or optionally substituted benzyl, a ligand, or a linker covalently bonded to one or more ligands.

In compounds of Formula (III), R^{A2} is hydrogen or a nitrogen protecting group. For example,

In compounds of Formula (III), one of R²² and R²³ is protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a ligand, or a linker covalently bonded to one or more ligands; and the other of R²² and R²³ is a reactive phosphorous group, a protected hydroxyl, or a hydroxyl.

In compounds of Formula (III), R⁴ is hydrogen, optionally substituted C₁₋₆alkyl, optionally substituted C₂₋₆alkenyl, optionally substituted C₂₋₆alkynyl, or optionally substituted C₁₋₆alkoxy.

In some compounds of Formula (III), R⁴ and R²² taken together are 4′-C(R¹⁰R¹¹)ᵥ—Y-2′ or 4′-Y—C(R¹⁰R¹¹)ᵥ-2′; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; and v is 1, 2 or 3.

In compounds of Formula (III), R⁴ and R²³ taken together with the atoms to which they are attached form an optionally substituted C₃₋₈cycloalkyl, optionally substituted C₃₋₈cycloalkenyl, or optionally substituted 3-8 membered heterocyclyl.

In compounds of Formula (III), R²⁵ is protected hydroxyl.

The compounds of Formula (II) and (III) are useful in the synthesis single-stranded and double-stranded oligonucleotides. Accordingly, in another aspect, provided herein is an oligonucleotide prepared using a compound of Formula (II) or (III). For example, an oligonucleotide comprising nucleoside of Formula (I).

Inventors have discovered inter alia that double stranded RNA (dsRNA) molecules comprising a nucleoside of Formula (I) are effective in inducing RNA interference (RNAi) activity. Accordingly, in one aspect provided herein is a double-stranded nucleic acid comprising a first strand and a second strand complementary to the first strand, and wherein at least one of the first and second strand is an oligonucleotide comprising a nucleoside of Formula (I) described herein. It is understood that one strand of the dsRNA (e.g., the antisense strand) has sufficient complementarity to a target sequence to mediate RNA interference. In other words, the dsRNA molecules of the invention are capable of inhibiting the expression of a target gene.

In some embodiments, the dsRNA molecule further comprises a nucleotide comprising a modified sugar. For example, the dsRNA molecule can further comprise a nucleotide with a sugar moiety selected from 2′-F ribose, 2′-OMe ribose, 2′-O,4′-C-methylene ribose, 1,5-anhydrohexitol, cyclohexene, 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose, threose, and 2,3-dihydroxypropyl.

In some embodiments, dsRNA molecule comprises at least one nucleotide with a sugar moiety selected from 2′-F ribose and 2′-OMe ribose. For example, the dsRNA molecule further comprises a 2′-F or 2′-OMe nucleotide. In some embodiments, the dsRNA molecule comprises at least one 2′-F nucleotide and at least one 2′-OMe nucleotide.

In some embodiments, the dsRNA molecule comprises at least one, e.g., 1, 2, 3, 4, 5, 6, 7, 8, 9, 10 or more 2′-fluoro (2′-F) nucleotides. For example, the dsRNA can comprise 1, 2, 3, 4, 5, 6, 7, 8, 9 or 10 2′-F nucleotides. Without limitations, the 2′-fluoro nucleotides all can be present in one strand. The 2′-F nucleotide may occur on any nucleotide of the sense strand or antisense strand or both in any position of the strand. In some embodiments, the sense strand comprises 1, 2, 3, 4, 5, 6, 7, 8, 9, 10 or more 2′-fluoro nucleotides. In some embodiments, the antisense strand comprises 1, 2, 3, 4, 5, 6, 7, 8, 9, 10 or more 2′-fluoro nucleotides. In some embodiments, each of the sense strand and the antisense strand comprises at least one 2′-F nucleotide. In some embodiments, both the sense and the antisense strands comprise at least one 2′-fluoro nucleotide.

In some embodiments, the dsRNA molecule comprises at least one, e.g., 1, 2, 3, 4, 5, 6, 7, 8, 9, 10 or more 2′-deoxy, e.g., 2′-H nucleotides. For example, the dsRNA can comprise 1, 2, 3, 4, 5, 6, 7, 8, 9 or 10 2′-H nucleotides. Without limitations, the 2′-H nucleotides all can be present in one strand. The 2′-H nucleotide may occur on any nucleotide of the sense strand or antisense strand or both in any position of the strand. For example, the antisense strand of the dsRNA molecules described herein can comprise one or more 2′-deoxy, e.g., 2′-H nucleotides. For example, the antisense strand comprises 1, 2, 3, 4, 5, 6 or more 2′-deoxy nucleotides. In some embodiments, the antisense strand comprises 2, 3, 4, 5 or 6 5 2′-deoxy, e.g., 2′-H nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from 5′-end of the antisense strand. In some embodiments, the antisense comprises a 2′-deoxy nucleotide at positions 5 and 7, counting from 5′-end of the antisense strand.

In some embodiments, the sense strand does not comprise a 2′-deoxy, e.g., 2′-H nucleotide.

In some embodiments, the remaining nucleotides in the dsRNA molecule are 2′-OMe nucleotides. For example, the dsRNA molecule comprises, e.g., solely comprises 2′-OMe and 2′-F nucleotides. In another non-limiting example, the dsRNA molecule comprises, e.g., solely comprises 2′-OMe, 2′-F and 2′-deoxy (2′-H) nucleotides. Accordingly, in some embodiments, the sense strand comprises, e.g., solely comprises 2′-OMe and 2′-fluoro nucleotides. In some embodiments, the antisense strand comprises, e.g., solely comprises 2′-OMe and 2′-F nucleotides. In some embodiments, the antisense strand comprises, e.g., solely comprises 2′-OMe, 2′-F and 2′-H nucleotides.

In some embodiments, the remaining nucleotides in the dsRNA are 2′-OMe nucleotides. For example, all of the remaining nucleotides in the sense strand are 2′-OMe nucleotides. In other words, the sense strand solely comprises 2′-fluoro and 2′-OMe nucleotides.

In various embodiments, the dsRNA molecule has a double stranded (duplex) region of between 19 to 25 base pairs. For example, the dsRNA molecule has a duplex region of 20, 21, 22, 23 or 24 basepairs. In some particular embodiments, the dsRNA molecule has a double duplex) region of 20, 21 or 22 base pairs.

In some embodiments, the dsRNA molecule comprises a ligand. For example, the sense strand of the dsRNA molecule comprises a ligand. Exemplary ligands include, but are not limited to, ASGPR ligand ligands.

The dsRNA molecule can comprise one or more, e.g., 1, 2, 3, 4, 5, 6, 7, 8 or more phosphorothioate linkages. The phosphorothioate linkages can be present only in one of the strands or in both strands of the dsRNA. For example, the sense strand can comprise 1, 2, 3 or 4 phosphorothioate linkages. In another non-limiting example, the antisense strand can comprise 1, 2, 3, 4, 5 or 6 phosphorothioate linkages. In some embodiments, the sense strand comprises 1, 2, 3 or 4 phosphorothioate linkages and the antisense independently comprises 1, 2, 3, 4, 5, or 6 phosphorothioate linkages. For example, the sense strand comprises 1 or 2 phosphorothioate linkages and the antisense strand comprises 1, 2, 3 or 4 phosphorothioate linkages.

In some embodiments, the sense strand comprises at least two phosphorothioate internucleotide linkages between the first five nucleotides counting from the 5′ end of the sense strand, the antisense strand comprises at least two phosphorothioate internucleotide linkages between the first five nucleotides counting from the 5′-end of the antisense strand and the antisense further comprises at least two phosphorothioate internucleotide linkages between the first five nucleotides counting from the 3′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages and between nucleotides 1 and 2, and between nucleotides 2 and 3, counting from 5′-end of the antisense strand, and between nucleotides 1 and 2, and between nucleotides 2 and 3, counting from 3′-end of the antisense strand.

In another aspect, provided herein is a method for inhibiting or reducing the expression of a target gene in a subject. The method comprises administering to the subject: (i) a double-stranded RNA described herein, wherein one of the strands of the dsRNA is complementary to a target gene; and/or (ii) an oligonucleotide described herein, wherein the oligonucleotide is complementary to a target gene.

In another aspect, the invention further provides a method for delivering the dsRNA molecule of the invention to a specific target in a subject by subcutaneous or intravenous administration. The invention further provides the dsRNA molecules of the invention for use in a method for delivering said agents to a specific target in a subject by subcutaneous or intravenous administration.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows structures of 6-methyladenosines (m6As) with 2′-modifications.

FIG. 2 is a schematic representation of a polymerase incorporation assay.

FIGS. 3A and 3B are bar graphs showing incorporation of 2′-deoxy, 2′-F, and 2′-F—N6MeA NTP monomers into the primer in the PolGamma primer extension assay (FIG. 3A) and incorporation of ribo, 2′-F, and 2′-F—N6MeA NTP monomers into the primer in the POLRMT primer extension assay (FIG. 3B). Incorporation efficiency was calculated as 100% minus the % area of the remaining primer based on the integration of the fluorescence signal at λₑₓ=436 nm and λₑₘ=485 nm. Average value of two replicate experiments. Note that the values for 2′-F-dNTP were obtained from previously published data (Janas, M. M. et al., Nucleic Acids Research 2019, 47, 3306).

FIGS. 4A and 4B are line graphs showing stability of m6As against 3′-exonuclease (FIG. 8A) and 5′-exonuclease (FIG. 8B) cleavage.

FIGS. 5A-7B show effect of 2′-Flouro and 2′-OMe modified m6A on RNAi activity of siRNAs targeting C5 (FIGS. 5A and 5B), β-catenin (FIGS. 6A and 6B) and mTTR (FIGS. 7A and 7B), and with transfection (FIGS. 5A, 6A and 7A) and free uptake (FIGS. 5B, 6B and 7B).

FIGS. 8A-8D are bar graphs showing thermodynamic stability of m6A when incorporated into the DNA strand of a DNA/DNA (FIG. 8A) or DNA/RNA (FIG. 8B) duplex, and when incorporated into the RNA strand of a RNA/RNA (FIG. 8C) or DNA/RNA (FIG. 8D) duplex.

FIG. 9 shows some exemplary N6-alkyl (methyl and isopropyl) derivatives of adenosine (with ribose, deoxyribose, 2′-fluoro, 2′-OMe, and LNA sugar moieties).

FIGS. 10A-10D show adenine to inosine conversion by adenosine deaminase is mitigated through N6-methyl modification.

FIGS. 11A-11D shows N6-iPr modification also hinders adenosine deaminase activity.

FIGS. 12A and 12B show no deaminated metabolites were observed for an exemplary N6-iPr compound (2′-OMe) in adenosine deaminase assays.

FIGS. 13A-13C show formation of deaminated metabolite from 2′-fluoro-adenosine, used as a positive control in adenosine deaminase assays.

FIG. 14A-14C show formation of demethylated metabolite from verapamil, used as a positive control for CYP activity.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### R²

In some embodiments of any one of the aspects described herein, R² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., methoxyethyl such as 2-methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R⁸)(CH₂R⁹), or —O—C₄₋₃₀alkyl-ON(CH₂R⁸)(CH₂R⁹), a solid support, a linker or a linker covalently attached to a solid support. For example, R² is hydrogen, hydroxyl, protected hydroxyl, halogen, optionally substituted C₁₋₃₀ alkoxy, alkoxyalkyl (e.g., methoxyethyl), alkoxyalkylamine, alkoxyoxycarboxylate, amino, alkylamino, dialkylamino, —O—N-methylacetamido, or C₆₋₂₄ alkyl (e.g., n-C₆₋₂₄ alkyl).

In some embodiments of any one of the aspects, R² is hydrogen, hydroxyl, halogen, protected hydroxyl, optionally substituted C₁₋₃₀ alkyl, optionally substituted C₂₋₃₀ alkenyl, optionally substituted C₂₋₃₀alkynyl, optionally substituted C₁₋₃₀alkoxy (e.g., methoxy, 2-methoxyethoxy), alkoxyalkyl (e.g., methoxyethyl such a 2-methoxyethyl), alkoxyalkylamine, alkoxyoxycarboxylate, amino, alkylamino, dialkylamino, —O—N-methylacetamido, C₆₋₂₄ alkyl (e.g., n-C₆₋₂₄ alkyl), or —O—C₄₋₃₀alkyl-ON(CH₂R₈)(CH₂R₉), or —O—C₄₋₃₀alkyl-ON(CH₂R₈)(CH₂R₉).

In some embodiments of any one of the aspect, R² is hydrogen, hydroxyl, protected hydroxyl, halogen, optionally substituted C₁₋₃₀ alkoxy, optionally substituted C₁₋₃₀ alkyl or alkoxyalkyl (e.g., methoxyethyl). For example, R² is hydrogen, hydroxyl, protected hydroxyl, fluoro, methoxy, ethoxy, 2-methoxyethoxy, —O—N-methylacetamido, C₆₋₂₄ alkoxy (e.g., n-C₆₋₂₄ alkoxy) or C₆₋₂₄ alkyl (e.g., n-C₆₋₂₄ alkyl).

In some embodiments of any one of the aspects, R² is hydrogen, hydroxyl, protected hydroxyl, fluoro, methoxy, 2-methoxyethoxy, —O—N-methylacetamido, or C₆₋₂₄ alkoxy (e.g., n-C₆₋₂₄ alkoxy).

In some embodiments of any one of the aspects R² is halogen. For example, R² can be fluoro, chloro, bromo or iodo. In some embodiments of any one of the aspects described herein, R² is fluoro.

In some embodiments of any one of the aspects described herein, R² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R² is C₁-C₃₀alkoxy optionally substituted with a NH₂, OH, C(O)NH₂, COOH, halo, SH, or C₁-C₆alkoxy. In some embodiments of any one of the aspects described herein, R² is —O(CH₂)ₜCH₃, where t is 1-21. For example, t is 14, 15, 16, 17 or 18. In one non-limiting example, t is 16.

In some embodiments of any one of the aspects, R² is methoxy, 2-methoxyethoxy or C₆₋₂₄ alkoxy such n-C₆₋₂₄alkoxy.

In some embodiments of any one of the aspects, R² is —O(CH₂)ᵤR²⁷, where u is 2-10; R²⁷ is C₁-C₆alkoxy, amino (NH₂), CO₂H, OH or halo. For example, R²⁷ is —CH₃ or NH₂. Accordingly, in some embodiments of any one of the aspects described herein, R² is —O(CH₂)ᵤ—OMe or R² is —O(CH₂)ᵤNH₂. In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R² is a C₁-C₆haloalkyl. For example, R² is a C₁-C₄haloalkyl. In some embodiments of any one of the aspects described herein, R² is —CF₃, —CF₂CF₃, —CF₂CF₂CF₃ or —CF₂(CF₃)₂.

In some embodiments of any one of the aspects described herein, R² is —OCH(CH₂OR²⁸)CH₂OR²⁹, where R²⁸ and R²⁹ independently are H, optionally substituted C₁-C₃₀alkyl, optionally substituted C₂-C₃₀alkenyl or optionally substituted C₂-C₃₀alkynyl. For example, R²⁸ and R²⁹ independently are optionally substituted C₁-C₃₀alkyl.

In some embodiments of any one of the aspects described herein, R² is —CH₂C(O)NHR²¹⁰, where R²¹⁰ is H, optionally substituted C₁-C₃₀alkyl, optionally substituted C₂-C₃₀alkenyl or optionally substituted C₂-C₃₀alkynyl. For example, R²¹⁰ is H or optionally substituted C₁-C₃₀alkyl. In some embodiments, R²¹⁰ is optionally substituted C₁-C₆alkyl.

In some embodiments of any one of the aspects described herein, R² is —O—N-methylacetamido.

In some embodiments of any one of the aspects, R² is optionally substituted C₁₋₃₀ alkyl. For example, R² is C₆₋₂₄ alkyl such n-C₆₋₂₄ alkyl.

In some embodiments of any one of the aspects described herein, R² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, v is 1. In some other embodiments of any one of the aspects, v is 2.

In some embodiments, Y is O. For example, R² and R⁴ taken together are 4′-C(R¹⁰R¹¹)ᵥ—O-2′. In some embodiments, R² and R⁴ taken together are 4′-C(R¹⁰R¹¹)—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃.

In some embodiments of any one of the aspects, R¹⁰ and R¹¹ attached to the same C are the same. For example, R¹⁰ and R¹¹ attached to the same C are H.

In some embodiments of any one of the aspects, R² and R⁴ taken together are 4′-CH₂—O-2′, 4′-CH(CH₃)—O-2′, 4′-CH(CH₂OCH₃)—O-2′, or 4′-CH₂CH₂—O-2′. For example, R² and R⁴ taken together are 4′-CH₂CH₂—O-2′.

In some embodiments of any one of the aspects described herein, R² is a bond to an internucleotide linkage to a subsequent nucleotide. It is noted that only one of R² and R³ can be a bond to an internucleotide linkage to a subsequent nucleotide.

In some embodiments of any one of the aspects, R² is a linker covalently bonded (e.g., —C(O)CH₂CH₂C(O)—) to a solid support. It is noted that only one of R² and R³ can be a linker attached covalently with to a solid support.

### R³

In some embodiments of any one of the aspects described herein, R³ can be a bond to an internucleotide linkage to a subsequent nucleotide, hydroxyl, protected hydroxyl, optionally substituted C₁₋₃₀ alkoxy, halogen, alkoxyalkyl (e.g., methoxyethyl), amino, alkylamino, dialkylamino, a 3′-oligonucleotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

In some embodiments of any one of the aspects described herein, R³ is a bond to an internucleotide linkage to a subsequent nucleotide, hydroxyl, protected hydroxyl, optionally substituted C₁₋₃₀ alkoxy, a 3′-oligonucleotide capping group (e.g., an inverted nucleotide or an inverted abasic nucleotide), a solid support, or a linker covalently bonded (e.g., —C(O)CH₂CH₂C(O)—) to a solid support. For example, R³ is a bond to an internucleotide linkage to a subsequent nucleotide, hydroxyl, a solid support, or a linker covalently bonded (e.g., —C(O)CH₂CH₂C(O)—) to a solid support. In some embodiments of any one of the aspects described herein, R³ is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R³ is a bond to an internucleotide linkage to a subsequent nucleotide.

In some embodiments of any one of the aspects described herein, R³ is a solid support, or a linker (e.g., —C(O)CH₂CH₂C(O)—) covalently bonded to a solid support.

In some embodiments of any one of the aspects described herein, R³ is hydroxyl or protected hydroxyl. For example, R³ is hydroxyl.

In some embodiments of any one of the aspects described herein, R³ and R⁴ taken together with the atoms to which they are attached form an optionally substituted C₃₋₈cycloalkyl, optionally substituted C₃₋₈cycloalkenyl, or optionally substituted 3-8 membered heterocyclyl.

### R⁴

In some embodiments of any one of the aspects described herein, R⁴ can be hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ can be hydrogen, optionally substituted C₁₋₆alkyl or optionally substituted C₁₋₆alkoxy.

In some embodiments of any one of the aspects described herein, R⁴ is H.

### R⁵

In some embodiments of any one of the aspects described herein, R⁵ can be a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10).

In some embodiments of any one of the aspects described herein, R⁵ can be a bond to an internucleotide linkage to a preceding nucleotide, hydroxyl, protected hydroxyl, optionally substituted C₂₋₃₀alkenyl, optionally substituted C₂₋₃₀alkynyl, optionally substituted C₁₋₃₀ alkoxy, vinylphosphonate (VP) group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidates, or alkylphosphonates.

In some embodiments of any one of the aspects described herein, R⁵ is a bond to an internucleotide linkage to a preceding nucleotide, hydroxyl, protected hydroxyl, optionally substituted C₂₋₃₀alkenyl, optionally substituted C₁₋₃₀ alkoxy or a vinylphosphonate (VP) group.

In some embodiments of any one of the aspects described herein, R⁵ is a bond to an internucleotide linkage to a preceding nucleotide.

In some embodiments of any one of the aspects described herein, R⁵ is a hydroxyl or protected hydroxyl.

In some embodiments of any one of the aspects described herein, R⁵ is optionally substituted C₂₋₃₀alkenyl or optionally substituted C₁₋₃₀ alkoxy.

In some embodiments of any one of the aspects described herein, R⁵ is a vinylphosphonate group.

In some embodiments of any one of the aspects described herein, the methylene connecting the R⁵ to the rest of the nucleoside of Formula (I) is absent and R⁵ is connected directly to the rest of the nucleoside of Formula (I).

In some embodiments of any one of the aspects descried herein, R⁵ is —CH(R⁵¹)—X⁵—R⁵², where X⁵ is absent, a bond or O; R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of any one of the aspects described herein, X⁵ is O or a bond. For example, X⁵ is O. In some other embodiments of any one of the aspects described herein, X⁵ is absent, i.e., R⁵ is —CH(R⁵¹)R⁵².

In some embodiments of the various aspects described herein, R⁵ is —CH(R⁵¹)—R⁵² or —C(R⁵¹)═CHR⁵², where R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of the various aspects described herein, R⁵ is —CH(R⁵¹)—X⁵—R⁵². For example, R⁵ is —CH(R⁵¹)—X⁵—R⁵² and where R⁵¹ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of the various aspects described herein, R⁵ is —CH(R⁵¹)—O—R⁵², where R⁵¹ is H or C₁-C₃₀alkyl optionally substituted with 1, 2, 3, 4 or 5 substituents independently selected from 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of any one of the aspects described herein, R⁵ is —C(R⁵¹)═CR⁵². It is noted that the double bond in —C(R⁵¹)═CHR⁵² can be in the cis or trans configuration. Accordingly, in some embodiments of any one of the aspects, R⁵ is —C(R⁵¹)═CHR⁵² and wherein the double bond is in the cis configuration. In some other embodiments of any one of the aspects, R⁵ is —C(R⁵¹)═CHR⁵² and wherein the double bond is in the trans configuration. In some embodiments of any one of the aspects described herein, R⁵ is —CH═CHR⁵².

In some embodiments of any one of the aspects described herein, R⁵² is a bond to an internucleoside linkage to the preceding nucleotide.

In embodiments of the various aspects described herein, R⁵ is optionally substituted C₁₋₆alkyl-R⁵³, optionally substituted —C₂₋₆alkenyl-R⁵³, or optionally substituted —C₂₋₆alkynyl-R⁵³. In embodiments of the various aspects described herein, R⁵³ can be —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; where R⁵⁴ is hydrogen or oxygen protecting group; R⁵⁵ is hydrogen or sulfur protecting group; each R⁵⁶ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁷ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is hydrogen.

In some other embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, or —SP(S)(SR⁵⁷)(OR⁵⁶) is not hydrogen. For example, at least one at least one R⁵⁶ in P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁶ is H and at least one R⁵⁶ is other than H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶).

In some embodiments of any one of the aspects, all R⁵⁶ are H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, all R⁵⁶ are other than H in in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is H.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is other than H. For example, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁷ is H and at least one R⁵⁷ is other than H in —P(S)(SR⁵⁷)₂, —OP(S)(SR⁵⁷)₂ and —SP(S)(SR⁵⁷)₂.

In some embodiments, all R⁵⁷ are H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments, all R⁵⁷ are other than H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments of any one of the aspects described herein, R⁵ is optionally substituted —C₂₋₆alkenyl-R⁵³. For example, R⁵ is —C₂₋₆alkenyl-R⁵³, where C₂₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects, R⁵ is —CH═CHR⁵³. It is noted that a double bond in the optionally substituted —C₂₋₆alkenyl-R⁵³ can be in the cis or trans configuration. Accordingly, in some embodiments of any one of the aspects, R⁵ is —CH═CHR⁵³ and wherein the double bond is in the cis configuration. In some other embodiments of any one of the aspects, R⁵ is —CH═CHR⁵³ and wherein the double bond is in the trans configuration.

In some embodiments of any one of the aspects, R⁵ is —CH═CH—P(O)(OR⁵⁶)₂, —CH═CH—P(S)(OR⁵⁶)₂, —CH═CH—P(S)(SR⁵⁷)(OR⁵⁶), —CH═CH—P(S)(SR⁵⁷)₂, —CH═CH—OP(O)(OR⁵⁶)₂, —CH═CH—OP(S)(OR⁵⁶)₂, —CH═CH—OP(S)(SR⁵⁷)(OR⁵⁶), —CH═CH—OP(S)(SR⁵⁷)₂, —CH═CH—SP(O)(OR⁵⁶)₂, —CH═CH—SP(S)(OR⁵⁶)₂, —CH═CH—SP(S)(SR⁵⁷)(OR⁵⁶), or —CH═CH—SP(S)(SR⁵⁷)₂. For example, R⁵ is —CH═CH—P(O)(OR⁵⁶)₂.

In some embodiments, of any one of the aspects, R⁵⁴ is hydrogen or an oxygen protecting group. For example, R⁵⁴ is hydrogen or 4,4′-dimethoxytrityl (DMT). In some preferred embodiments, R⁵⁴ is H.

In some embodiments of any one of the aspects described herein, R⁵ is optionally substituted —C₁₋₆alkenyl-R⁵³. For example, R⁵ is —C₁₋₆alkenyl-R⁵³, where C₁₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects described herein, R⁵ can be —CH(R⁵⁸)—R⁵³, where R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; and R⁵⁸ is H, optionally substituted C₁₋₃₀alkyl, optionally substituted C₂₋₃₀alkenyl, or optionally substituted C₂₋₃₀alkynyl.

In some embodiments of any one of the aspects described herein, R⁵⁸ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In one non-limiting example, R⁵⁸ is H. In some other non-limiting examples, R⁵⁸ is C₁-C₃₀alkyl optionally substituted with a substituent selected from NH₂, OH, C(O)NH₂, COOH, halo, SH, and C₁-C₆alkoxy.

In some embodiments of any one of the aspects described herein, R⁵ is —CH(R⁵⁸)—O—R⁵⁹, where R⁵⁹ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂. For example, R⁵ is —CH(R⁵⁸)—O—R⁵⁹, where R⁵⁸ is H or optionally substituted C₁-C₃₀alkyl and R⁵⁹ is H or —P(O)(OR⁵⁶)₂.

In some embodiments of any one of the aspects described herein, R⁵ is —CH(R⁵⁸)—S—R⁶⁰, where R⁶⁰ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂.

### R²²

In some embodiments of any one of the aspects described herein, R²² is hydrogen, halogen, —OR²²², —SR²²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O—N-methylacetamido, —O(CH₂CH₂O)ᵣCH₂CH₂OR²²⁴, cyano, alkyl-thio-alkyl, thioalkoxy, cycloalkyl, aryl, heteroaryl, —NH(CH₂CH₂NH)ₛCH₂CH₂—R²²⁵, NHC(O)R²²⁶, a lipid, a linker covalently attached to a lipid, a ligand, a linker covalently attached to a ligand, a solid support, a linker covalently attached to a solid support, or a reactive phosphorus group.

R²²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R²²⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R²² is hydrogen, halogen, —OR²²², —SR²²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O—N-methylacetamido, —O(CH₂CH₂O)ᵣCH₂CH₂OR²²⁴, cyano, alkyl-thio-alkyl, thioalkoxy, cycloalkyl, aryl, heteroaryl, —NH(CH₂CH₂NH)ₛCH₂CH₂—R²²⁵, NHC(O)R²²⁴.

In some embodiments of any one of the aspects described herein, R²² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—N-methylacetamido, —O—C₄₋₃₀alkyl-ON(CH₂R⁸)(CH₂R⁹), or —O—C₄₋₃₀alkyl-ON(CH₂R⁸)(CH₂R⁹). For example, R² is hydrogen, hydroxyl, protected hydroxyl, halogen, optionally substituted C₁₋₃₀ alkoxy, alkoxyalkyl (e.g., methoxyethyl), —O—N-methylacetamido, alkoxyalkylamine, alkoxyoxycarboxylate, amino, alkylamino, or dialkylamino.

In some embodiments of any one of the aspect, R²² is hydrogen, hydroxyl, protected hydroxyl, halogen, optionally substituted C₁₋₃₀ alkoxy, or alkoxyalkyl (e.g., methoxyethyl. In some embodiments of any one of the aspects, R² is hydrogen, hydroxyl, protected hydroxyl, fluoro or methoxy.

In some embodiments of any one of the aspects R²² is halogen. For example, R²² can be fluoro, chloro, bromo or iodo. In some embodiments of any one of the aspects described herein, R²² is fluoro.

In some embodiments of any one of the aspects described herein, R²² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R²² is C₁-C₃₀alkoxy optionally substituted with a NH₂, OH, C(O)NH₂, COOH, halo, SH, or C₁-C₆alkoxy. In some embodiments of any one of the aspects described herein, R²² is —O(CH₂)ₜCH₃, where t is 1-21. For example, t is 14, 15, 16, 17 or 18. In one non-limiting example, t is 16.

In some embodiments of any one of the aspects, R²² is methoxy, 2-methoxyethoxy or C₆₋₂₄ alkoxy such n-C₆₋₂₄alkoxy.

In some embodiments of any one of the aspects, R²² is —O(CH₂)ᵤR²²⁷, where u is 2-10; R²²⁷ is C₁-C₆alkoxy, amino (NH₂), CO₂H, OH or halo. For example, R²²⁷ is —CH₃ or NH₂. Accordingly, in some embodiments of any one of the aspects described herein, R²² is —O(CH₂)ᵤ—OMe or R²² is —O(CH₂)ᵤNH₂. In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R²² is a C₁-C₆haloalkyl. For example, R²² is a C₁-C₄haloalkyl. In some embodiments of any one of the aspects described herein, R²² is —CF₃, —CF₂CF₃, —CF₂CF₂CF₃ or —CF₂(CF₃)₂.

In some embodiments of any one of the aspects described herein, R²² is —OCH(CH₂OR²²⁸)CH₂OR²²⁹, where R²²⁸ and R²²⁹ independently are H, optionally substituted C₁-C₃₀alkyl, optionally substituted C₂-C₃₀alkenyl or optionally substituted C₂-C₃₀alkynyl. For example, R²²⁸ and R²²⁹ independently are optionally substituted C₁-C₃₀alkyl.

In some embodiments of any one of the aspects described herein, R²² is —CH₂C(O)NHR²²¹⁰, where R²²¹⁰ is H, optionally substituted C₁-C₃₀alkyl, optionally substituted C₂-C₃₀alkenyl or optionally substituted C₂-C₃₀alkynyl. For example, R²²¹⁰ is H or optionally substituted C₁-C₃₀alkyl. In some embodiments, R²²¹⁰ is optionally substituted C₁-C₆alkyl.

In some embodiments of any one of the aspects described herein, R²² is —O—N-methylacetamido.

In some embodiments of any one of the aspects, R²² is optionally substituted C₁₋₃₀ alkyl. For example, R² is C₆₋₂₄ alkyl such n-C₆₋₂₄ alkyl.

In some embodiments of any one of the aspects described herein, R²² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group. In some embodiments of any one of the aspects, v is 1. In some other embodiments of any one of the aspects, v is 2. In some embodiments, Y is O. For example, R²² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃. In some embodiments of any one of the aspects, R¹⁰ and R¹¹ attached to the same C are the same. For example, R¹⁰ and R¹¹ attached to the same C are H.

In some embodiments of any one of the aspects, R²² and R⁴ taken together are 4′-CH₂—O-2′, 4′-CH(CH₃)—O-2′, 4′-CH(CH₂OCH₃)—O-2′, or 4′-CH₂CH₂—O-2′. For example, R²² and R⁴ taken together are 4′-CH₂CH₂—O-2′.

In some embodiments of any one of the aspects described herein, R²² is a reactive phosphorus group.

Without wishing to be bound by a theory, reactive phosphorus groups are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. Reactive phosphorous group in the form of phosphoramidites (P^{III} chemistry) as reactive phosphites are a preferred reactive phosphorous group for solid phase oligonucleotide synthesis. The intermediate phosphite compounds are subsequently oxidized to the Pv state using known methods to yield phosphodiester or phosphorothioate internucleoside linkages.

In some embodiments of any one of the aspects described herein, the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}. For example, the reactive phosphorous group is —OP(OR^{P})(N(R^{P2})₂).

In some embodiments of any one of the aspects, R is an optionally substituted C₁₋₆alkyl. For example, R is a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ (CH₂)ₚ—NH₂ or CH₂-aryl-alkoxy, where “m” and “p” are independently 1, 2, 3, 4, 5 or 6. In some embodiments, R^{p} is a C₁₋₆alkyl, optionally substituted with a CN or —SC(O)Ph. For example, R^{p} is cyanoethyl (—CH₂CH₂CN).

In the reactive phosphorous groups, each R^{P2} is independently optionally substituted C₁₋₆alkyl. For example, each R^{P2} can be independently selected from methyl, ethyl, propyl, isopropyl, n-butyl, iso-butyl, pentyl or hexyl. It is noted that when two or more R² groups are present in the reactive phosphorous group, they can be same or different. Thus, in some none-limiting examples, when two or more R^{P}2 groups are present, the R^{P2} groups are different. In some other non-limiting examples, when two or more R^{P}2 groups are present, the R² groups are same. In some embodiments of any one of the aspects, each R^{P2} is isopropyl.

In some embodiments of any one of the aspects, both R^{P2} taken together with the nitrogen atom to which they are attached form an optionally substituted 3-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In some embodiments of any one of the aspects, R and one of R² taken together with the atoms to which they are attached form an optionally substituted 4-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In the reactive phosphorous groups, each R^{P3} is independently optionally substituted C₁₋₆alkyl. For example, R^{P3} can be a C₁₋₆alkyl, optionally substituted with 1, 2, 3, 4 or 5 substituents independently selected from OH, CN, SC(O)Ph, oxo (═O), SH, SO₂NH₂, SO₂(C₁—C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{P3} is methyl, ethyl, propyl, isopropyl, n-butyl, iso-butyl, pentyl or hexyl, each of which can be optionally substituted with a NH₂, OH, C(O)NH₂, COOH, halo, SH, or C₁-C₆alkoxy.

In some embodiments of any one of the aspects, the reactive phosphorous group is —OP(OR)(N(R^{P2})₂). For example, the reactive phosphorous group is —OP(OR)(N(R^{P2})₂), where R is cyanoethyl (—CH₂CH₂CN) and each R^{P2} is isopropyl.

In some embodiments of any one of the aspects described herein, R²² is —OP(OR)(N(R^{P2})₂), —OP(SR)(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR)H, —OP(S)(OR)H, —OP(O)(SR)H, —OP(O)(OR^{P})R^{P3}, —OP(S)(OR^{P})R^{P3}, or —OP(O)(SR^{P})R^{P3}.

In some embodiments of any one of the aspects, R²² is —OP(OR) (N(R²)₂), —OP(SR)(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR) an optionally substituted C₁₋₆alkyl, each R² is independently optionally substituted C₁₋₆alkyl; and each R^{P3} is independently optionally substituted C₁₋₆alkyl.

In some embodiments of any one of the aspects, R²² is —OP(OR^{P})(N(R^{P2})₂). For example, the R²² is —OP(OR)(N(R^{P2})₂), where R is cyanoethyl (—CH₂CH₂CN) and each R² is isopropyl.

In some embodiments of any one of the aspects descried herein, R²² is a solid support or a linker covalently attached to a solid support. For example, R²² is —OC(O)CH₂CH₂C(O)NH—Z, where Z is a solid support. In some embodiments, R²² is —OC(O)CH₂CH₂CO₂H.

In some embodiments of any one of the aspects, when R²² is —OR²²², R²²² can be hydrogen or a hydroxyl protecting group.

When R²² is —SR²²³, R²²³ can be hydrogen or a sulfur protecting group. Accordingly, in some embodiments of any one of the aspects, R²²³ is hydrogen.

When R²² is —O(CH₂CH₂O)ᵣCH₂CH₂OR²²⁴, r can be 1-50; R²²⁴ is independently for each occurrence H, C₁-C₃₀alkyl, cyclyl, heterocyclyl, aryl, heteroaryl, aralkyl, sugar or R²⁵; and R²²⁵ is independently for each occurrence amino (NH₂), alkylamino, dialkylamino, arylamino, diarylamino, heteroarylamino, or diheteroaryl amino.

When R²² is —NH(CH₂CH₂NH)ₛCH₂CH₂—R²²⁵, s can be 1-50 and R²²⁵ can be independently for each occurrence amino (NH₂), alkylamino, dialkylamino, arylamino, diarylamino, heteroarylamino, or diheteroaryl amino.

In some embodiments of any one of the aspects described herein, R²² is hydrogen, halogen, —OR²²², or optionally substituted C₁-C₃₀alkoxy. For example, R²² is halogen, —OR²²², or optionally substituted C₁-C₃₀alkoxy. In some embodiments of any one of the aspects described herein, R²² is F, OH or optionally substituted C₁-C₃₀alkoxy.

In some embodiments of any one of the aspects described herein, R²² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R²² is C₁-C₃₀alkoxy optionally substituted with a NH₂, OH, C(O)NH₂, COOH, halo, SH, or C₁-C₆alkoxy. In some embodiments of any one of the aspects described herein, R²² is —O(CH₂)ₜCH₃, where t is 1-21. For example, t is 14, 15, 16, 17 or 18. In one non-limiting example, t is 16.

In some embodiments of any one of the aspects, R²² is —O(CH₂)ᵤR²²⁷, where u is 2-10; R²²⁷ is C₁-C₆alkoxy, amino (NH₂), CO₂H, OH or halo. For example, R²²⁷ is —CH₃ or NH₂. Accordingly, in some embodiments of any one of the aspects described herein, R²² is —O(CH₂)ᵤ—OMe or R²² is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R²² is a C₁-C₆haloalkyl. For example, R²² is a C₁-C₄haloalkyl. In some embodiments of any one of the aspects described herein, R²² is —CF₃, —CF₂CF₃, —CF₂CF₂CF₃ or —CF₂(CF₃)₂.

In some embodiments of any one of the aspects described herein, R²² is —OCH(CH₂OR²²⁸)CH₂OR²²⁹, where R²²⁸ and R²²⁹ independently are H, optionally substituted C₁-C₃₀alkyl, optionally substituted C₂-C₃₀alkenyl or optionally substituted C₂-C₃₀alkynyl. For example, R²²⁸ and R²²⁹ independently are optionally substituted C₁-C₃₀alkyl.

In some embodiments of any one of the aspects described herein, R²² is —CH₂C(O)NHR²²¹⁰, where R²²¹⁰ is H, optionally substituted C₁-C₃₀alkyl, optionally substituted C₂-C₃₀alkenyl or optionally substituted C₂-C₃₀alkynyl. For example, R²²¹⁰ is H or optionally substituted C₁-C₃₀alkyl. In some embodiments, R²²¹⁰ is optionally substituted C₁-C₆alkyl.

### R²³

In some embodiments of any one of the aspects described herein, R²³ is hydrogen, halogen, —OR²³², —SR²³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O—N-methylacetamido, —O(CH₂CH₂O)ᵣCH₂CH₂OR²³⁴, cyano, alkyl-thio-alkyl, thioalkoxy, cycloalkyl, aryl, heteroaryl, —NH(CH₂CH₂NH)ₛCH₂CH₂—R²³⁵, NHC(O)R²³⁶, a lipid, a linker covalently attached to a lipid, a ligand, a linker covalently attached to a ligand, a solid support, a linker covalently attached to a solid support, or a reactive phosphorus group.

R²³² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R²³⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R²³ is a reactive phosphorus group. For example, R²³ is —OP(OR)(N(R^{P2})₂), —OP(SR)(N(R^{P2})₂), —OP(O)(OR^{P})(N(R^{P2})₂), —OP(S)(OR^{P})(N(R^{P2})₂), —OP(O)(SR)(NR^{P2})₂, —OP(O)(OR)H, —OP(S)(OR^{P})H, —OP(O)(SR)H, —OP(O)(OR^{P})R^{P3}, —OP(S)(OR^{P})R^{P3}, or —OP(O)(SR^{P})R^{P3}.

In some embodiments of any one of the aspects, R²³ is —OP(OR)(N(R^{P2})₂), —OP(SR)(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR) an optionally substituted C₁₋₆alkyl, each R² is independently optionally substituted C₁₋₆alkyl; and each R³ is independently optionally substituted C₁₋₆alkyl.

In some embodiments of any one of the aspects, R²³ is —OP(OR^{P})(N(R^{P2})₂). For example, the R²³ is —OP(OR)(N(R^{P2})₂), where R is cyanoethyl (—CH₂CH₂CN) and each R² is isopropyl.

Optionally, only one of R²² and R²³ is a reactive phosphorous group.

In some embodiments of any one of the aspects descried herein, R²³ is a solid support or a linker covalently attached to a solid support. For example, R²³ is —OC(O)CH₂CH₂C(O)NH—Z, where Z is a solid support.

Optionally, only one of R²² and R²³ is a solid support or a linker covalently attached to a solid support.

In some embodiments of any one of the aspects, when R²³ is —OR²³², R²³² can be hydrogen or a hydroxyl protecting group. For example, R²³² can be hydrogen in some embodiments of any one of the aspects described herein. In some embodiments, R²³ is —OC(O)CH₂CH₂CO₂H.

When R²³ is —SR²³³, R²³³ can be hydrogen or a sulfur protecting group. Accordingly, in some embodiments of any one of the aspects, R²³³ is hydrogen.

When R²³ is —O(CH₂CH₂O)ᵣCH₂CH₂OR²³⁴, r can be 1-50; R²³⁴ is independently for each occurrence H, C₁-C₃₀alkyl, cyclyl, heterocyclyl, aryl, heteroaryl, aralkyl, sugar or R²³⁵; and R²³⁵ is independently for each occurrence amino (NH₂), alkylamino, dialkylamino, arylamino, diarylamino, heteroarylamino, or diheteroaryl amino.

When R²³ is —NH(CH₂CH₂NH)ₛCH₂CH₂—R²³⁵, s can be 1-50 and R²³⁵ can be independently for each occurrence amino (NH₂), alkylamino, dialkylamino, arylamino, diarylamino, heteroarylamino, or diheteroaryl amino.

In some embodiments of any one of the aspects described herein, R²³ is hydrogen, halogen, —OR²³², or optionally substituted C₁-C₃₀alkoxy. For example, R²³ is halogen, —OR²³², or optionally substituted C₁-C₃₀alkoxy. In some embodiments of any one of the aspects described herein, R²³ is F, OH or optionally substituted C₁-C₃₀alkoxy.

In some embodiments of any one of the aspects described herein, R²³ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R²³ is C₁-C₃₀alkoxy optionally substituted with a NH₂, OH, C(O)NH₂, COOH, halo, SH, or C₁-C₆alkoxy. In some embodiments of any one of the aspects described herein, R²³ is —O(CH₂)ₜCH₃, where t is 1-21. For example, t is 14, 15, 16, 17 or 18. In one non-limiting example, t is 16.

In some embodiments of any one of the aspects, R²³ is —O(CH₂)ᵤR²³⁷, where u is 2-10; R²³⁷ is C₁-C₆alkoxy, amino (NH₂), CO₂H, OH or halo. For example, R²³⁷ is —CH₃ or NH₂. Accordingly, in some embodiments of any one of the aspects described herein, R²³ is —O(CH₂)ᵤ—OMe or R²³ is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R²³ is a C₁-C₆haloalkyl. For example, R²³ is a C₁-C₄haloalkyl. In some embodiments of any one of the aspects described herein, R²³ is —CF₃, —CF₂CF₃, —CF₂CF₂CF₃ or —CF₂(CF₃)₂.

In some embodiments of any one of the aspects described herein, R²³ is —OCH(CH₂OR²³⁸)CH₂OR²³⁹, where R²³⁸ and R²³⁹ independently are H, optionally substituted C₁-C₃₀alkyl, optionally substituted C₂-C₃₀alkenyl or optionally substituted C₂-C₃₀alkynyl. For example, R²³⁸ and R²³⁹ independently are optionally substituted C₁-C₃₀alkyl.

In some embodiments of any one of the aspects described herein, R²³ is —CH₂C(O)NHR²³¹⁰, where R²³¹⁰ is H, optionally substituted C₁-C₃₀alkyl, optionally substituted C₂-C₃₀alkenyl or optionally substituted C₂-C₃₀alkynyl. For example, R²³¹⁰ is H or optionally substituted C₁-C₃₀alkyl. In some embodiments, R²³¹⁰ is optionally substituted C₁-C₆alkyl.

In some embodiments of any one of the aspects described herein, R²³ and R⁴ taken together with the atoms to which they are attached form an optionally substituted C₃₋₈cycloalkyl, optionally substituted C₃₋₈cycloalkenyl, or optionally substituted 3-8 membered heterocyclyl.

### R²⁵

In some embodiments of the various aspects described herein, R²⁵ is R⁵⁵¹, optionally substituted C₁₋₆alkyl-R⁵⁵¹, optionally substituted —C₂₋₆alkenyl-R⁵⁵¹, or optionally substituted —C₂₋₆alkynyl-R⁵⁵¹, where R⁵⁵¹ can be —OR⁵⁵², —SR⁵53, hydrogen, a phosphorous group, a solid support or a linker to a solid support. When R⁵⁵¹ is —OR⁵⁵², R⁵⁵² can be H or a hydroxyl protecting group. Similarly, when R⁵⁵¹ is —SR⁵⁵³, R⁵⁵³ can be H or a sulfur protecting group.

In some embodiments of any one of the aspects described herein, R²⁵ is —OR⁵⁵² or —SR⁵⁵³.

In some embodiments of any one of the aspects described herein, R⁵⁵² is a hydroxyl protecting group. Exemplary hydroxyl protecting groups for R⁵⁵² include, but are not limited to, benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In some embodiments of any one of the aspects described herein, R²⁵ is —OR⁵⁵² and R⁵⁵² is 4,4′-dimethoxytrityl (DMT), e.g., R²⁵ is —O-DMT.

In some embodiments of any one of the aspects described herein, the methylene connecting the R²⁵ to the rest of the compound of Formula (II) is absent and R²⁵ is connected directly to the rest of the compound of Formula (II).

In some embodiments of any one of the aspects described herein, R²⁵ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R²⁵ is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R²⁵ is —CH(R⁵⁵⁴)—O—R⁵⁵² where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ (CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R²⁵ is optionally substituted C₁₋₆alkyl-R⁵⁵¹ or optionally substituted —C₂₋₆alkenyl-R⁵⁵¹,

In some embodiments of any one of the aspects described herein, R²⁵ is —C(R⁵⁵⁴)═CHR⁵⁵¹. It is noted that the double bond in —C(R⁵⁵⁴)═CHR⁵⁵¹ can be in the cis or trans configuration. Accordingly, in some embodiments of any one of the aspects, R^{d} is —C(R⁵⁵⁴)═CHR⁵⁵¹ and wherein the double bond is in the cis configuration. In some other embodiments of any one of the aspects, R^{d} is —C(R⁵⁵⁴)═CHR⁵⁵¹ and wherein the double bond is in the trans configuration.

In some embodiments of any one of the aspects described herein, R²⁵ is —CH═CHR⁵⁵¹.

In some embodiments of any one of the aspects, when R²⁵ is —C(R⁵⁵⁴)═CHR⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚNH₂ or CH₂-aryl-alkoxy, where “m” and “p” are independently 1, 2, 3, 4, 5 or 6; and R⁵⁵¹ is a phosphorous group. For example, R²⁵ is —CH═CHR⁵⁵¹.

In some embodiments of any one of the aspects described herein, R⁵⁵¹ is a reactive phosphorous group.

In some embodiments of any one of the aspects, R²⁵ is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵56)(OR⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is hydrogen.

In some other embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, or —SP(S)(SR⁵⁵⁶)(OR⁵⁵⁵) is not hydrogen. For example, at least one at least one R⁵⁵⁵ in P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁵⁵ is H and at least one R⁵⁵⁵ is other than H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵).

In some embodiments of any one of the aspects, all R⁵⁵⁵ are H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, all R⁵⁵⁵ are other than H in in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is H.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is other than H. For example, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁵⁶ is H and at least one R⁵⁵⁶ is other than H in —P(S)(SR⁵⁵⁶)₂, —OP(S)(SR⁵⁵⁶)₂ and —SP(S)(SR⁵⁵⁶)₂.

In some embodiments, all R⁵⁵⁶ are H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments, all R⁵⁵⁶ are other than H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the aspects, R²⁵ is —CH═CH—P(O)(OR⁵⁵⁵)₂, where each R⁵⁵⁵ is H or an oxygen protecting group.

In some embodiments of any one of the aspects, R²³ is a reactive phosphorous group, a solid support, a linker to a solid support, and R²⁵ is a protected hydroxyl.

In some other embodiments of any one of the aspects, R²² is a reactive phosphorous group, a solid support, a linker to a solid support, and R²⁵ is a protected hydroxyl.

### Internucleoside Linkages

As used herein, “internucleoside linkage” refers to a covalent linkage between adjacent nucleosides. The two main classes of internucleoside linkages are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified internucleoside linkages, compared to natural phosphodiester linkages, can be used to alter, typically increase, nuclease resistance of the oligonucleotide compound. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the internucleoside linkage can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphodiester internucleoside linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral. In other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. The non-bridging oxygens can be independently any one of O, S, Se, B, C, H, N, or OR (R is alkyl or aryl).

A phosphodiester internucleoside linkage can also be modified by replacement of bridging oxygen, (i.e. oxygen that links the phosphate to the sugar of the nucleosides), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)—5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C8′), thioethers (C3′-S—C8′), thioacetamido (C3′-N(H)—C(═O)—CH₂—S—C8′, C3′-O—P(O)—O—SS—C8′, C3′-CH₂—NH—NH—C8′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester internucleoside linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Additional exemplary non-phosphorus containing internucleoside linking groups are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, content of each of which is incorporated herein by reference.

In some embodiments of any one of the aspects, the oligonucleotides described herein comprise one or more neutral internucleoside linkages that are non-ionic. Suitable neutral internucleoside linkages include, but are not limited to, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)—5′), formacetal (3′-O—CH₂—O-5′), and thioformacetal (3′-S—CH₂—O-5′); nonionic linkages containing siloxane (dialkylsiloxane), carboxylate ester, carboxamide, sulfide, sulfonate ester and/or amides (See for example: Carbohydrate Modifications in Antisense Research; Y. S. Sanghvi and P. D. Cook Eds. ACS Symposium Series 580; Chapters 3 and 4, (pp. 40-65)); and nonionic linkages containing mixed N, O, S and CH₂ component parts.

In one embodiment, the non-phosphodiester backbone linkage is selected from the group consisting of phosphorothioate, phosphorodithioate, alkyl-phosphonate and phosphoramidate backbone linkages.

In some embodiments of any one of the aspects described herein, the internucleoside linkage is

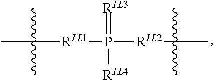

where R^{IL1} and R^{IL2} are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and R^{IL3} and R^{IL4} are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), alkyl or aryl. It is understood that one of R^{IL1} and R^{IL2} is replacing the oxygen linked to 5′ carbon of a first nucleoside sugar and the other of R^{IL1} and R^{IL2} is replacing the oxygen linked to 3′ (or 2′) carbon of a second nucleoside sugar.

In some embodiments of any one of the aspects, R^{IL1}, R^{IL2}, R^{IL3} and R^{IL4} all are O.

In some embodiments, R^{IL1} and R^{IL2} are O and at least one of R^{IL3} and R^{IL4} is other than O. For example, one of R^{IL3} and R^{IL4} is S and the other is O or both of R^{IL3} and R^{IL4} are S.

In some embodiments of any one of the aspects described herein, one of R³ or R⁵ is a bond to a modified internucleoside linkage, e.g., an internucleoside linkage of structure:

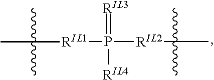

where at least one of R^{IL1}, R^{IL2}, R^{IL3} and R^{IL4} is not O. For example, at least one of R^{IL3} and R^{IL4} is S.

In some embodiments of any one of the aspects described herein, both of R³ and R⁵ are a bond to a modified internucleoside linkage.

In some embodiments of any one of the aspects described herein R³ is a bond to phosphodiester internucleoside linkage.

In some embodiments of any one of the aspects described herein R⁵ is a bond to phosphodiester internucleoside linkage.

In some embodiments of any one of the aspects described herein, R³ is a bond to a modified internucleoside linkage and R⁵ is a bond to phosphodiester internucleoside linkage.

In some embodiments of any one of the aspects described herein, R⁵ is a bond to a modified internucleoside linkage and R³ is a bond to phosphodiester internucleoside linkage.

In some embodiments of any one of the aspects, the oligonucleotide can comprise one or more, e.g., 1, 2, 3, 4, 5, 6, 7, 8 or more modified internucleoside linkages. For example, the oligonucleotide can comprise 1, 2, 3, 4, 5 or 6 (e.g., 1, 2, 3 or 4) modified internucleoside linkages. In some embodiments, the oligonucleotide comprises at least two modified internucleoside linkages between the first five nucleotides counting from the 5′-end of the oligonucleotide and further comprises at least two modified internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects, the modified internucleoside linkage is a phosphorothioate. Accordingly, in some embodiments of any one of the aspects, the oligonucleotide comprises one or more, e.g., 1, 2, 3, 4, 5, 6, 7, 8 or more phosphorothioate internucleoside linkages. For example, the oligonucleotide comprises 1, 2, 3, 4, 5 or 6 (e.g., 1, 2, 3, or 4) phosphorothioate internucleoside linkages. In some embodiments, the oligonucleotide comprises at least two phosphorothioate internucleoside linkages between the first five nucleotides counting from the 5′-end of the oligonucleotide and further comprises at least two phosphorothioate internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments, the oligonucleotide comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages. For example, oligonucleotide comprises 2, 3, 4, 5, 6, 7, 8, or 9 blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages.

In some embodiments of any one of the aspects described herein, the oligonucleotide comprises a pattern of backbone chiral centers. In some embodiments, a common pattern of backbone chiral centers comprises 5, 6, 7, 8, 9, 10, 11, 12, 13, 14, 15, 16, 17, 18 or more internucleotidic linkages in the Sp configuration. In some embodiments, a common pattern of backbone chiral centers comprises no more than 1, 2, 3, 4, 5, 6, 7 or 8 internucleotidic linkages in the Rp configuration. In some embodiments, a common pattern of backbone chiral centers comprises no more than 1, 2, 3, 4, 5, 6, 7 or 8 internucleotidic linkages which are not chiral (as a non-limiting example, a phosphodiester). In some embodiments, a common pattern of backbone chiral centers comprises 10, 11, 12, 13, 14, 15 or more internucleotidic linkages in the Sp configuration, and no more than 8, no more than no more than 7, no more than 6, no more than 5, or no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, the oligonucleotide comprises a block which is a stereochemistry block. For example, the oligonucleotide comprises a block which is an Rp block in that each internucleotidic linkage of the block is Rp. In some embodiments, the oligonucleotide comprises a block which is an Sp block in that each internucleotidic linkage of the block is Sp. In some embodiments, the oligonucleotide comprises a Rp block at the 5′-end. In some embodiments, the oligonucleotide comprises a Rp block at the 3′-end. In some embodiments, the oligonucleotide comprises a Sp block at the 5′-end. In some embodiments, the oligonucleotide comprises a Sp block at the 3′-end. In some embodiments, the oligonucleotide comprises both Rp and Sp blocks. In some embodiments, the oligonucleotide comprises one or more Rp but no Sp blocks. In some embodiments, the oligonucleotide comprises one or more Sp but no Rp blocks. In some embodiments, the oligonucleotide comprises one or more PO blocks wherein each internucleotidic linkage in a natural phosphate linkage.

In some embodiments, an adenosine in the oligonucleotide is followed by Sp. In some embodiments, an adenosine in the oligonucleotide is followed by Rp. In some embodiments, an adenosine in the oligonucleotide is followed by natural phosphate linkage (PO). In some embodiments, a uridine in the oligonucleotide is followed by Sp. In some embodiments, a uridine in the oligonucleotide is followed by Rp. In some embodiments, a uridine in the oligonucleotide is followed by natural phosphate linkage (PO). In some embodiments, a cytidine in the oligonucleotide is followed by Sp. In some embodiments, a cytidine in the oligonucleotide is followed by Rp. In some embodiments, a cytidine in the oligonucleotide is followed by natural phosphate linkage (PO). In some embodiments, a guanosine in the oligonucleotide is followed by Sp. In some embodiments, a guanosine in the oligonucleotide is followed by Rp. In some embodiments, a guanosine in the oligonucleotide is followed by natural phosphate linkage (PO). In some embodiments, cytidine and uridine are followed by Sp. In some embodiments, cytidine and uridine are followed by Rp. In some embodiments, cytidine and uridine are followed by natural phosphate linkage (PO). In some embodiments, adenosine and guanosine are followed by Sp. In some embodiments, adenosine and guanosine are followed by Rp.

### Oligonucleotide Modifications—Sugar

In some embodiments of any one of the aspects described herein, the oligonucleotide further comprises, i.e., in addition to a nucleoside of Formula (I), a nucleoside with a modified sugar. By a “modified sugar” is meant a sugar or moiety other than 2′-deoxy (i.e, 2′-H) or 2′-OH ribose sugar. Some exemplary nucleotides comprising a modified sugar are 2′-F ribose, 2′-OMe ribose, 2′-O,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxylpropyl (glycol nucleic acid, GNA). It is noted that the nucleoside with the modified sugar can be present at any position of the oligonucleotide.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′-F nucleotides. It is noted that the 2′-F nucleotides can be present at any position of the oligonucleotide.

In some embodiments, the oligonucleotide comprises, e.g., solely comprises nucleosides of Formula (I) and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′-OMe nucleotides. It is noted that the 2′-OMe nucleotides can be present at any position of the oligonucleotide.

In some embodiments, the oligonucleotide comprises, e.g., solely comprises solely comprises solely comprises nucleosides of Formula (I) and 2′-OMe nucleosides. In some other embodiments, the oligonucleotide comprises, e.g., solely comprises solely comprises nucleosides of Formula (I), 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises solely comprises nucleosides of Formula (I) and 2′-deoxy (2′-H) nucleotides. In some embodiments, the oligonucleotide comprises, e.g., solely comprises nucleosides of Formula (I), 2′-OMe nucleosides, and 2′-deoxy (2′-H) nucleotides. In some embodiments, the oligonucleotide comprises, e.g., solely comprises nucleosides of Formula (I), 2′-F nucleosides and 2′-deoxy (2′-H) nucleotides. In some embodiments, the oligonucleotide comprises, e.g., solely comprises nucleosides of Formula (I), 2′-OMe nucleosides, 2′-F nucleosides and 2′-deoxy (2′-H) nucleotides.

### Oligonucleotide Modifications—Nucleobase

In some embodiments of any one of the aspects described herein, the oligonucleotide further comprises, i.e., in addition to a nucleoside of Formula (I), a nucleoside with a non-natural nucleobase.

By a “non-natural nucleobase” is meant a nucleobase other than adenine, guanine, cytosine, uracil, or thymine. Exemplary non-natural nucleobases include, but are not limited to, inosine, xanthine, hypoxanthine, nubularine, isoguanisine, tubercidine, and substituted or modified analogs of adenine, guanine, cytosine and uracil, such as 2-aminoadenine and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613, content of all which is incorporated herein by reference.

In some embodiments, the non-natural nucleobase can be selected from the group consisting of inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxyl)-1,3-(diaza)-2-(oxo)-phenoxazin-1-yl, 7-(aminoalkylhydroxyl)-1-(aza)-2-(thio)-3-(aza)-phenoxazin-1-yl, 7-(aminoalkylhydroxyl)-1,3-(diaza)-2-(oxo)-phenthiazin-1-yl, 7-(aminoalkylhydroxyl)-1-(aza)-2-(thio)-3-(aza)-phenthiazin-1-yl, 7-(guanidiniumalkylhydroxyl)-1,3-(diaza)-2-(oxo)-phenoxazin-1-yl, 7-(guanidiniumalkylhydroxyl)-1-(aza)-2-(thio)-3-(aza)-phenoxazin-1-yl, 7-(guanidiniumalkyl-hydroxyl)-1,3-(diaza)-2-(oxo)-phenthiazin-1-yl, 7-(guanidiniumalkylhydroxyl)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, a non-natural nucleobase is a modified nucleobase, i.e., the nucleobase comprises a nucleobase modification described herein, e.g., the nucleobase is a substituted or modified analog of any of the natural nucleobases. Examples of the nucleobase modifications include, but not limited to: C-5 pyrimidine with an alkyl group or aminoalkyls and other cationic groups such as guanidinium and amidine functionalities, N²— and N⁶— with an alkyl group or aminoalkyls and other cationic groups such as guanidinium and amidine functionalities of purines, G-clamps, guanidinium G-clamps, and pseudouridine known in the art.

In some embodiments of any one of the aspects, the non-natural nucleobase is a universal nucleobase. As used herein, a universal nucleobase is any modified or unmodified natural or non-natural nucleobase that can base pair with all of adenine, cytosine, guanine and uracil without substantially affecting the melting behavior, recognition by intracellular enzymes or activity of the oligonucleotide comprising the universal nucleobase. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof.

In some embodiments of any one of the aspects described herein, the non-natural nucleobase is a protected nucleobase. As used herein, a “protected nucleobase” refers to a nucleobase comprising a nitrogen protecting group, and/or an oxygen protecting group, and/or a sulfur protecting group.

In some embodiments of any one of the aspects described herein, the non-natural nucleobase is a modified, protected or substituted analogs of a nucleobase selected from adenine, cytosine, guanine, thymine, and uracil.

In some embodiments, the oligonucleotide can comprise one or more, e.g., 1, 2, 3, 4, 5, 6, 7, 8, 9, 10 or more nucleotides comprising an independently selected non-natural nucleobase. When present, a nucleotide comprising a non-natural nucleobase can be present anywhere in the oligonucleotide.

In some embodiments, the oligonucleotide further comprises a solid support linked thereto.

The oligonucleotides described herein can range from few nucleotides (e.g., 2, 3, 4, 5, 6, 7, 8, 9 or 10 nucleotides) in length to hundreds of nucleotides in length. For example, the oligonucleotide can be from 5 nucleotides to 100 nucleotides in length. In some embodiments, the oligonucleotide is from 10 nucleotides to 50 nucleotides in length. For example, the oligonucleotide is between 15 and 35, more generally between 18 and 25, yet more generally between 19 and 24, and most generally between 19 and 21 base pairs in length. In some embodiments, longer oligonucleotides of between 25 and 30 nucleotides in length are preferred. In some embodiments, shorter oligonucleotides of between 10 and 15 nucleotides in length are preferred. In another embodiment, the oligonucleotide is at least 21 nucleotides in length.

### Oxygen Protecting Groups

Some embodiments of the various aspects described herein include an oxygen protecting group (also referred to as an hydroxyl protecting group herein). Oxygen protecting groups include, but are not limited to, —R^{OP1}, —N(R^{OP2})₂, —C(═O)SR^{OP1}, —C(═O)R^{OP1}, —CO₂R^{OP1}, —C(═O)N(R^{OP2})₂, —C(═NR^{OP2})R^{OP1}, —C(═NR^{OP2})OR^{OP1}, —C(═NR^{OP2})N(R^{OP2})₂, —S(═O)R^{OP1}, —SO⁺₂R^{OP1}, —Si(R^{OP1})₃, —P(R^{OP3})₂, —P(R^{OP3})⁺₃X⁻, —P(OR^{OP3})₂, —P(OR^{OP3})⁺₃X⁻, —P(═O)(R^{OP1})₂, —P(═O)(OR^{OP3})₂, and —P(═O)(N(R^{OP2})₂)₂; wherein each X⁻ is a counterion; each R^{OP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{OP1} groups are joined to form a 3-14 membered heterocyclyl or 5-14 membered heteroaryl ring; each R^{OP2} is hydrogen, —OH, —OR^{OP1}, —N(R^{OP3})₂, —CN, —C(═O)R^{OP1}, —C(═O)N(R^{OP3})₂, —CO₂R^{OP1}, —SO₂R^{OP1}, —C(═NR^{OP3})OR^{OP1}, —C(═NR^{OP3})N(R^{OP3})₂, —SO₂N(R^{OP3})₂, —SO₂R^{OP3}, —SO₂OR^{OP3}, —SOR^{OP1}, —C(═S)N(R^{OP3})₂, —C(═O)SR^{OP3}, —C(═S)SR^{OP3}, —P(═O)(R^{OP1})₂, —P(═O)(OR^{OP3})₂, —P(═O)(N(R^{OP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{OP2} groups are joined to form a 3-14 membered heterocyclyl or 5-14 membered heteroaryl ring; and each R^{OP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{OP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{OP1}, R^{OP2} and R^{OP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Oxygen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl,diphenylmethylsilyl (DPMS), t-butylmethoxyphenylsilyl (TBMPS), formate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuSP3inoate, (E)-2-methyl-2-butenoate, o-(methoxyacyl)benzoate, α-naphthoate, nitrate, alkylN,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In some embodiments of any one of the aspects described herein, oxygen protecting group is acetyl, benzyl, benzoyl, 2,6-dichlorobenzyl, t-butyldimethylsilyl (TBDMS), t-butyldiphenylsilyl, trimethylsilyl (TMS), triisopropylsilyl (TIPS), mesylate, tosylate, 4,4′-dimethoxytrityl (DMT), 9-phenylxanthine-9-yl (Pixyl) and 9-(p-methoxyphenyl)xanthine-9-yl (MOX). In certain embodiments, the hydroxyl protecting group is selected from acetyl, benzyl, t-butyldimethylsilyl, t-butyldiphenylsilyl, trimethylsilyl (TMS), triisopropylsilyl (TIPS), and dimethoxytrityl wherein a more preferred hydroxyl protecting group is 4,4′-dimethoxytrityl.

The terms “protected hydroxyl” and “protected hydroxyl” as used herein mean a group of the formula —OR^{Pro}, wherein R^{Pro} is an oxygen protecting group as defined herein.

### Nitrogen Protecting Groups

Some embodiments of the various aspects described herein include a nitrogen protecting group (also referred to as an amino protecting group herein). Nitrogen protecting groups include, but are not limited to, —OH, —OR^{NP1}, —N(R^{NP2})₂, —C(═O)R^{NP1}, —C(═O)N(R^{NP2})₂, —CO₂R^{NP1}, —SO₂R^{NP1}, —C(═NR^{NP2})R^{NP1}, —C(═NR^{NP2})OR^{NP1}, —C(═NR^{NP2})N(R^{NP2})₂, —SO₂N(R^{NP2})₂, —SO₂R^{NP2}, —SO₂OR^{NP2}, —SOR^{NP1}, —C(═S)N(R^{NP2})₂, —C(═O)SR², —C(═S)SR^{NP2}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, where each R^{NP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{NP1} groups are joined to form a 3-14 membered heterocyclyl or 5-14 membered heteroaryl ring; and each R^{NP2} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring, and wherein each alkyl, alkenyl, alkynyl, carbocyclyl, heterocyclyl, aralkyl, aryl, and heteroaryl of R^{NP1} and R^{NP2} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Nitrogen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary amide (e.g., —C(═O)R^{NP1}) nitrogen protecting groups include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxy acylamino)acetamide, 3-(p-hydroxylphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Exemplary carbamate (e.g., —C(═O)OR^{NP1}) nitrogen protecting groups include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxylpiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxylboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Exemplary sulfonamide (e.g., —S(═O)₂R^{NP1}) nitrogen protecting groups include, but are not limited to, such as p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Additional exemplary nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuNP2inimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl] methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxylphenyl)phenylmethyleneamine, N-cyclohexylideneamine, N-(5,5-dimethyl-3-oxo-1-cyclohexenyl)amine, N-borane and N-diphenylborinic acid derivative, N-[phenyl(pentNPlcylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

### Sulfur Protecting Groups

Some embodiments of the various aspects described herein include sulfur protecting group (also referred to as a thiol protecting group herein). Sulfur protecting groups include, but are not limited to, —R^{SP1}, —N(R^{SP2})₂, —C(═O)SR^{SP1}, —C(═O)R^{SP1}, —CO₂R^{SP1}, —C(═O)N(R^{SP2})₂, —C(═NR^{SP2})R^{SP1}, —C(═NR^{SP2})OR^{SP1}, —C(═NR^{SP2})N(R^{SP2})₂, —S(═O)R^{SP1}, —SO₂R^{SP1}, —Si(R^{SP1})₃, —P(R^{SP3})₂, —P(R^{SP3})⁺₃X⁻, —P(OR^{SP3})₂, —P(OR^{SP3})⁺₃X⁻, —P(═O)(R^{SP1})₂, —P(═O)(OR^{SP3})₂, and —P(═O)(N(R^{SP2})₂)₂, wherein

X⁻ is a counterion; each R^{SP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{SP1} groups are joined to form a 3-14 membered heterocyclyl or 5-14 membered heteroaryl ring; each R^{SP2} is hydrogen, —OH, —OR^{SP1}, —N(R^{SP3})₂, —CN, —C(═O)R^{SP1}, —C(═O)N(R^{SP3})₂, —CO₂R^{SP1}, —SO₂R^{SP1}, —C(═NR^{SP3})OR^{SP1}, —C(═NR^{SP3})N(R^{SP3})₂, —SO₂N(R^{SP3})₂, —SO₂R^{SP3}, —SO₂OR^{SP3}, —SOR^{SP1}, —C(═S)N(R^{SP3})₂, —C(═O)SR^{SP3}, —C(═S)SR^{SP3}, —P(═O)(R^{SP1})₂, —P(═O)(OR^{SP3})₂, —P(═O)(N(R^{SP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{SP2} groups are joined to form a 3-14 membered heterocyclyl or 5-14 membered heteroaryl ring; and each R^{SP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{SP1}, R^{SP2} and R^{SP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ (CH₂)ₚ—NH₂ or CH₂-aryl-alkoxy, where “m” and “p” are independently 1, 2, 3, 4, 5 or 6

Sulfur protecting groups are well known in the art and include those described in detail in Greene's Protecting Groups in Organic Synthesis, P. G. M. Wuts, 5^{th} Edition, John Wiley & Sons, 2014, incorporated herein by reference.

### Double-Stranded RNAs

The skilled person is well aware that double-stranded RNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

Accordingly, in one aspect, provided herein is a double-stranded RNA (dsRNA) comprising a first strand (also referred to as an antisense strand or a guide strand) and a second strand (also referred to as a sense strand or passenger strand, wherein at least one of the first (i.e., the antisense strand) or the second strand (i.e., the sense strand) is an oligonucleotide described herein. In other words, at least one of the first (i.e., the antisense strand) or the second strand (i.e., the sense strand) comprises at least one nucleotide of Formula (I).

In some embodiments of the various aspects described herein, the antisense strand is substantially complementary to a target nucleic acid, e.g., a target gene or mRNA gene and the dsRNA is capable of inducing targeted cleavage of the target nucleic acid. Without limitations, the dsRNAs of the invention can be substituted for the dsRNA molecules and can be used in RNA interference based gene silencing techniques, including, but not limited to, in vitro or in vivo applications.

In some embodiments of any one of the aspects described herein, the sense strand is an oligonucleotide described herein. In other words, the sense strand comprises at least one nucleotide of Formula (I).

In some embodiments of any one of the aspects described herein, the antisense strand is an oligonucleotide described herein. In other words, the antisense strand comprises at least one nucleotide of Formula (I).

As described herein, the dsRNA molecule described herein can comprise at least one, e.g., 1, 2, 3, 4, 5, 6, 7, 8, 9, 10 or more of nucleotide of Formula (I). Without limitations, the nucleotides of Formula (I) all can be present in one strand. The nucleotide of Formula (I) may occur on any nucleotide of the sense strand or antisense strand or both in any position of the strand.

In some embodiments, the sense strand comprises 1, 2, 3, 4, 5, 6, 7, 8, 9, 10 or more nucleotides of Formula (I) described herein. The nucleotide of Formula (I) described herein can be present at any position of the sense strand. For example, the nucleotide of Formula (I) described herein can be present at a terminal region of the sense strand. For example, the nucleotide of Formula (I) described herein can be present at one or more of positions 1, 2, 3 and 4, counting from the 5′-end of the sense strand. In another non-limiting example, the nucleotide of Formula (I) described herein can be present at one or more of positions 1, 2, 3 and 4, counting from the 3′-end of the sense strand. In some embodiments, the nucleotide of Formula (I) can be present at one or more of positions 18, 19, 20 and 21, counting from 5′-end of the sense strand. The nucleotide of Formula (I) described herein can also be located at a central region of sense strand. For example, the nucleotide of Formula (I) described herein can be located at one or more of positions 6, 7, 8, 9, 10, 11, 12 and 13, counting from 5′-end of the sense strand. In some embodiments, the nucleotide of Formula (I) is at the 5-terminus of the sense strand.

In some embodiments, the antisense strand comprises 1, 2, 3, 4, 5, 6, 7, 8, 9, 10 or more of nucleotides of Formula (I) described herein. The nucleotide of Formula (I) described herein can be present at any position of the antisense strand. For example, the nucleotide of Formula (I) described herein can be present at a terminal region of the antisense strand. For example, the nucleotide of Formula (I) described herein can be present at one or more of positions 1, 2, 3 and 4, counting from the 5′-end of the antisense strand. In another non-limiting example, the nucleotide of Formula (I) described herein nucleotide can be present at one or more of positions 1, 2, 3, 4, 5 and 6, counting from the 3′-end of the antisense strand. In some embodiments, the nucleotide of Formula (I) described herein nucleotide can be present at one or more of positions 18, 19, 20, 21, 22 and 23, counting from 5′-end of the antisense strand. The nucleotide of Formula (I) described herein nucleotide can also be located at a central region of the antisense strand. For example, the nucleotide of Formula (I) described herein nucleotide can be located at one or more of positions 6, 7, 8, 9, 10, 11, 12 and 13, counting from 5′-end of the antisense strand. In some embodiments, the nucleotide of Formula (I) is at the 3′-terminus of the antisense strand.

Each strand of the dsRNA molecule can range from 15-35 nucleotides in length. For example, each strand can be between, 17-35 nucleotides in length, 17-30 nucleotides in length, 25-35 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. For example, the sense strand and the antisense strand independently have a length of 18, 19, 20, 21, 22, 23, 24 or 25 nucleotides.

In some embodiments, the antisense strand is of length 15-35 nucleotides. In some embodiments, the antisense strand is 15-35, 17-35, 17-30, 25-35, 27-30, 17-23, 17-21, 17-19, 19-25, 19-23, 19-21, 21-25, 21-25, or 21-23 nucleotides in length. For example, the antisense strand can be 15, 16, 17, 18, 19, 20, 21, 22, 23, 24, 25, 26, 27, 28, 29, 30, 31, 32, 33, 34 or 35 nucleotides in length. In some embodiments, the antisense strand is 19, 20, 21, 22, 23, 24 or 25 nucleotides in length. For example, the antisense strand is 21, 22, 23, 24 or 25 nucleotides in length. In some particular embodiments, the antisense strand is 22, 23 or 24 nucleotides in length. For example, the antisense strand is 23 nucleotides in length.

Similar to the antisense strand, the sense strand can be, in some embodiments, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length. For example, the sense strand can be 15, 16, 17, 18, 19, 20, 21, 22, 23, 24, 25, 26, 27, 28, 29, 30, 31, 32, 33, 34 or 35 nucleotides in length. In some embodiments, the sense strand is 17, 18, 19, 20, 21, 22, 23, 24 or 25 nucleotides in length. For example, the sense strand is 19, 20, 21, 22 or 23 nucleotides in length. In some particular embodiments, the sense strand is 20, 21 or 22 nucleotides in length. For example, the sense strand is 21 nucleotides in length

In some embodiments, the sense strand can be 15-35 nucleotides in length, and the antisense strand can be independent from the sense strand, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length, and the antisense strand is independently 15-35, 17-35, 17-30, 25-35, 27-30, 17-23, 17-21, 17-19, 19-25, 19-23, 19-21, 21-25, 21-25, or 21-23 nucleotides in length. For example, the sense and the antisense strand can be independently 15, 16, 17, 18, 19, 20, 21, 22, 23, 24, 25, 26, 27, 28, 29, 30, 31, 32, 33, 34 or 35 nucleotides in length. In some embodiments, the sense strand and the antisense strand are independently 17, 18, 19, 20, 21, 22, 23, 24 or 25 nucleotides in length. For example, the sense strand is 19, 20, 21, 22 or 23 nucleotides in length and the antisense strand is 21, 22, 23, 24 or 25 nucleotides in length. In some particular embodiments, the sense strand is 20, 21 or 22 nucleotides in length and the antisense strand is 22, 23 or 24 nucleotides in length. For example, the sense strand is 21 nucleotides in length and the antisense strand is 23 nucleotides in length.

The sense strand and antisense strand typically form a double-stranded or duplex region. Without limitations, the duplex region of a dsRNA agent described herein can be 12-35 nucleotide (or base) pairs in length. For example, the duplex region can be between 14-35 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length. In some embodiments, the duplex region is 18, 19, 20, 21, 22, 23, 24 or 25 nucleotide pairs in length. For example, the duplex region is 19, 20, 21, 22 or 23 nucleotide pairs in length. In some embodiments, the duplex region is 20, 21 or 22 nucleotide pairs in length. For example, the dsRNA molecule has a duplex region of 21 base pairs.

### The m6A Modification

Inventors have discovered inter alia that dsRNA molecules having a nucleotide comprising a 6-methyladenine (m6A) nucleobase are effective in inducing RNA interference (RNAi) activity. Accordingly, in one aspect provided herein is a double stranded RNA (dsRNA) comprising at least one nucleotide comprising a 6-methyladenine nucleobase. At least 50%, e.g., 55%, 60%, 65%, 70%, 75%, 80%, 85%, or 90% of the nucleotides in the dsRNA can be 2′-OMe nucleotides. In some embodiments, up to 90% or 95% of the nucleotides in the dsRNA molecule can be 2′-OMe nucleotides. The 2′-OMe nucleotides can be present only in the sense strand, only in the antisense strand or in both the sense stand and the antisense strand. Thus, in some embodiments, at least 50%, e.g., 55%, 60%, 65%, 70%, 75%, 80%, 85%, or 90% of the of the nucleotides in the sense strand can be 2′-OMe nucleotides. For example, up to 90% or 95% of the nucleotides in the sense strand can be 2′-OMe nucleotides. Independently, at least 50%, e.g., 55%, 60%, 65%, 70%, 75%, 80%, 85% or 90% of the nucleotides in the antisense strand can be 2′-OMe nucleotides. For example, up to 90% or 95% of the nucleotides in the antisense strand can be 2′-OMe nucleotides.

Generally, the dsRNA molecule comprises a sense strand and an antisense strand and each strand independently having a length of 15-35 nucleotides. The antisense strand can be substantially complementarity to a target sequence to mediate RNA interference. In other words, the dsRNA molecule is capable of inhibiting the expression of a target gene.

The nucleotide comprising a 6-methyladenine (m6A) nucleobase is also referred to as m6A nucleotide herein. The m6A nucleotide can be present anywhere in the dsRNA molecule. For example, the m6A nucleotide is present in the antisense strand. In some embodiments, the m6A nucleotide is present in the sense strand. In some embodiments, both the sense strand and the antisense strand independently comprise at least one m6A nucleotide.

When the m6A nucleotide is present in the antisense strand, it can be located anywhere in the antisense strand. In some embodiments, the m6A nucleotide is present at a terminal region of the antisense strand. For example, the m6A nucleotide can be present at one or more of positions 1, 2, 3 and 4, counting from the 5′-end of the antisense strand. In another non-limiting example, the m6A nucleotide can be present at one or more of positions 1, 2, 3, 4, 5 and 6, counting from the 3′-end of the antisense strand. In some embodiments, the m6A nucleotide can be present at one or more of positions 18, 19, 20, 21, 22 and 23, counting from 5′-end of the antisense strand.

The m6A nucleotide can also be located at a central region of the antisense strand. For example, the m6A nucleotide can be located at one or more of positions 6, 7, 8, 9, 10, 11, 12 and 13, counting from 5′-end of the antisense strand. In some embodiments, the antisense strand does not comprise a m6A nucleotide in a central region of the antisense strand.

Similar to the antisense strand, the m6A nucleotide can be located anywhere in the sense strand. In some embodiments, the m6A nucleotide is present at a terminal region of the sense strand. For example, the m6A nucleotide can be present at one or more of positions 1, 2, 3 and 4, counting from the 5′-end of the sense strand. In another non-limiting example, the m6A nucleotide can be present at one or more of positions 1, 2, 3 and 4, counting from the 3′-end of the sense strand. In some embodiments, the m6A nucleotide can be present at one or more of positions 18, 19, 20 and 21, counting from 5′-end of the sense strand. The m6A nucleotide can also be located at a central region of the sense strand. For example, the m6A nucleotide can be located at one or more of positions 6, 7, 8, 9, 10, 11, 12 and 13, counting from 5′-end of the sense strand.

The nucleotide comprising the 6-methyladenine nucleobase can comprise a modified sugar. In some embodiments, the nucleotide comprising the 6-methyladenine nucleobase is a 2′-F nucleotide. In some other embodiments, the nucleotide comprising the 6-methyladenine nucleobase is a 2′-OMe nucleotide.

As described herein, the dsRNA molecule of the invention can comprise at least one, e.g., 1, 2, 3, 4, 5, 6, 7, 8, 9, 10 or more m6A nucleotides. Without limitations, the m6A nucleotides all can be present in one strand. The m6A nucleotide may occur on any nucleotide of the sense strand or antisense strand or both in any position of the strand.

In some embodiments, the sense strand comprises 1, 2, 3, 4, 5, 6, 7, 8, 9, 10 or more m6A nucleotides. The m6A nucleotide can be present at any position of the sense strand. For example, the m6A nucleotide can be present at a terminal region of the sense strand. For example, the m6A nucleotide can be present at one or more of positions 1, 2, 3 and 4, counting from the 5′-end of the sense strand. In another non-limiting example, the m6A nucleotide can be present at one or more of positions 1, 2, 3 and 4, counting from the 3′-end of the sense strand. In some embodiments, the m6A nucleotide can be present at one or more of positions 18, 19, 20 and 21, counting from 5′-end of the sense strand. The m6A nucleotide can also be located at a central region of the sense strand. For example, the m6A nucleotide can be located at one or more of positions 6, 7, 8, 9, 10, 11, 12 and 13, counting from 5′-end of the sense strand.

In some embodiments, the antisense strand comprises 1, 2, 3, 4, 5, 6, 7, 8, 9, 10 or more m6A nucleotides. The m6A nucleotide can be present at any position of the antisense strand. For example, the m6A nucleotide can be present at a terminal region of the antisense strand. For example, the m6A nucleotide can be present at one or more of positions 1, 2, 3 and 4, counting from the 5′-end of the antisense strand. In another non-limiting example, the m6A nucleotide can be present at one or more of positions 1, 2, 3, 4, 5 and 6, counting from the 3′-end of the antisense strand. In some embodiments, the m6A nucleotide can be present at one or more of positions 18, 19, 20, 21, 22 and 23, counting from 5′-end of the antisense strand. The m6A nucleotide can also be located at a central region of the antisense strand. For example, the m6A nucleotide can be located at one or more of positions 6, 7, 8, 9, 10, 11, 12 and 13, counting from 5′-end of the antisense strand. In some embodiments, the antisense strand does not comprise a m6A nucleotide in a central region of the antisense strand.

### Additional Modifications

As described herein, the oligonucleotides, e.g. dsRNAs described herein can comprise one or more, e.g., 1, 2, 3, 4, 5, 6, 7, 8, 9, 10 or more nucleotides comprising a modified sugar. Accordingly, in some embodiments, the oligonucleotides, e.g. dsRNAs can comprise one or more, e.g., 1, 2, 3, 4, 5, 6, 7, 8, 9, 10 or more nucleotides independently selected from the group consisting of 2′-F, 2-OMe, acyclic nucleotides, locked nucleic acid (LNA), HNA, CeNA, 2′-methoxyethyl, 2′-O-allyl, 2′-C-allyl, 2′-O—N-methylacetamido (2′-O-NMA), a 2′-O-dimethylaminoethoxyethyl (2′-O-DMAEOE), 2′-O-aminopropyl (2′-O-AP), and 2′-ara-F. A nucleotide comprising modified sugar can be present anywhere in the dsRNA molecule. For example, a nucleotide comprising a modified sugar can be present in the sense strand or a nucleotide comprising a modified sugar can be present in the antisense strand. When two or more nucleotides comprising a modified sugar are present in the dsRNA molecule, they can all be in the sense strand, antisense strand or both in the sense and antisense strands.

### 2′-Fluoro Modifications

As described herein, the dsRNA molecule of the invention can comprise at least one, e.g., 1, 2, 3, 4, 5, 6, 7, 8, 9, 10 or more 2′-fluoro (2′-F) nucleotides.

In some embodiments, the sense strand comprises 1, 2, 3, 4, 5, 6, 7, 8, 9, 10 or more 2′-fluoro nucleotides. The 2′-fluoro nucleotides can be located anywhere in the sense strand. For example, the sense strand comprises a 2′-fluoro nucleotide at position 10, counting from 5′-end of the sense strand. In some embodiments, the sense strand comprises a 2′-fluoro nucleotide at position 10, counting from 5′-end of the sense strand and the sense strand further comprises a 2′-fluoro nucleotide at position 8, 9, 11 or 12, counting from 5′-end of the sense strand. For example, the sense strand comprises a 2′-fluoro nucleotide at positions 9 10, counting from 5′-end of the sense strand. In another example, the sense strand comprises a 2′-fluoro nucleotide at positions 10 and 11, counting from 5′-end of the sense strand. In some embodiments, the sense strand comprises a 2′-fluoro nucleotide at positions 9, 10 and 11, counting from 5′-end of the sense strand. In some other embodiments, the sense strand comprises a 2′-fluoro nucleotide at positions 8, 9 and 10, counting from 5′-end of the sense strand. In yet some other embodiments, the sense strand comprises a 2′-fluoro nucleotide at positions 10, 11 and 12, counting from 5′-end of the sense strand.

In some embodiments, the antisense comprises 2′-fluoro nucleotides at positions 7, 10 and 11 from the 5′-end. In some other embodiments, the sense strand comprises 2′-fluoro nucleotides at positions 7, 9, 10 and 11 from the 5′-end. In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13 and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to a thermally destabilizing modification of the duplex in the antisense strand.

In some embodiments, the antisense strand comprises 1, 2, 3, 4, 5, 6, 7, 8, 9, 10 or more 2′-fluoro nucleotides. The 2′-fluoro nucleotides can be located anywhere in the antisense strand. For example, the antisense strand can comprise a 2′-fluoro nucleotide at position 14, counting from 5′-end of the antisense strand. In some embodiments, the antisense comprises 2′-fluoro nucleotides at positions 2, 14 and 16, counting from the 5′-end of the antisense strand. In some other embodiments, the antisense comprises 2′-fluoro nucleotides at positions 2, 6, 14 and 16 from the 5′-end. In still some embodiments, the antisense comprises 2′-fluoro nucleotides at positions 2, 6, 8, 9, 14 and 16 from the 5′-end.

In some embodiments, the antisense strand comprises at least one 2′-fluoro nucleotide adjacent to a destabilizing modification. For example, the 2′-fluoro nucleotide can be the nucleotide at the 5′-end or the 3′-end of a destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification. In some embodiments, the antisense strand comprises at least two 2′-fluoro nucleotides at the 3′-end of the destabilizing modification, i.e., at positions +1 and +2 from the position of the destabilizing modification.

In some embodiments, both the sense and the antisense strands comprise at least one 2′-fluoro nucleotide. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

### 2′-OMe Modifications

As described herein, the dsRNA molecule of the invention can comprise at least one, e.g., 1, 2, 3, 4, 5, 6, 7, 8, 9, 10 or more 2′-OMe nucleotides. Without limitations, the 2′-OMe nucleotides all can be present in one strand. The 2′-OMe nucleotide may occur on any nucleotide of the sense strand or antisense strand or both in any position of the strand.

In some embodiments, the sense strand comprises 1, 2, 3, 4, 5, 6, 7, 8, 9, 10 or more 2′-OMe nucleotides. The 2′-OMe nucleotides can be located anywhere in the sense strand. In some embodiments, the antisense strand comprises 1, 2, 3, 4, 5, 6, 7, 8, 9, 10 or more 2′-OMe nucleotides. The 2′-OMe nucleotides can be located anywhere in the antisense strand.

### 2′-Deoxy Modifications

As described herein, the dsRNA molecule of the invention can comprise at least one, e.g., 1, 2, 3, 4, 5, 6, 7, 8, 9, 10 or more 2′-deoxy, e.g., 2′-H ribose nucleotides. For example, the dsRNA can comprise 1, 2, 3, 4, 5, 6, 7, 8, 9 or 10 2′-deoxy, e.g., 2′-H nucleotides. The 2′-deoxy nucleotide may occur on any nucleotide of the sense strand or antisense strand or both in any position of the strand.

As described herein, the dsRNA can comprise at least one, e.g., at least two, at least three, at least four, at least five, at least six, at least seven or more, 2′-deoxy modifications in a central region of the sense strand and/or the antisense strand. For example, at least one of the sense stand and the antisense can comprise at least one, e.g., at least two, at least three, at least four, at least five, at least six, at least seven or more, 2′-deoxy modification in positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the sense strand or the antisense strand.

In some embodiments, the antisense strand comprises 1, 2, 3, 4, 5 or 6 of 2′-deoxy nucleotides. For example, antisense strand can comprise 2, 3, 4, 5 or 6 of 2′-deoxy nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from 5′-end of the antisense strand. In one non-limiting example, the antisense strand comprises a 2′-deoxy nucleotide at 1, 2, 3 or 4 of positions 2, 5, 7, and 12, counting from 5′-end of the antisense strand.

In some embodiments, the antisense comprises a 2′-deoxy nucleotide at positions 5 and 7, counting from 5′-end of the antisense strand. For example, the antisense strand comprises a 2′-deoxy nucleotide at positions 5, 7 and 12, counting from 5′-end of the antisense strand. In some embodiments, the antisense strand comprises a 2′-deoxy nucleotide at positions 2, 5 and 7, counting from 5′-end of the antisense strand. For example, the antisense strand comprises a 2′-deoxy nucleotide at positions 2, 5, 7 and 12, counting from 5′-end of the antisense strand. In some embodiments, the antisense strand comprises a 2′-deoxy nucleotide at positions 2, 5, 7, 12 and 14, counting, from 5′-end of the antisense strand. For example, the antisense strand comprises a 2′-deoxy nucleotide at positions 2, 5, 7, 12, 14 and 16, counting from 5′-end of the antisense strand

In some embodiments, the antisense comprises a 2′-deoxy nucleotide at position 2 or 12, counting from 5′-end of the antisense strand. For example, the antisense comprises a 2′-deoxy nucleotide at position 12, counting from 5′-end of the antisense strand.

In some embodiments, the dsRNA comprises at least three 2′-deoxy modifications, wherein the 2′-deoxy modifications are at positions 2 and 14 of the antisense strand, counting from 5′-end of the antisense strand, and at position 11 of the sense strand, counting from 5′-end of the sense strand.

In some embodiments, the dsRNA comprises at least five 2′-deoxy modifications, wherein the 2′-deoxy modifications are at positions 2, 12 and 14 of the antisense strand, counting from 5′-end of the antisense strand, and at positions 9 and 11 of the sense strand, counting from 5′-end of the sense strand.

In some embodiments, the dsRNA comprises at least seven 2′-deoxy modifications, wherein the 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In some embodiments, the antisense strand comprises at least five 2′-deoxy modifications at positions 2, 5, 7, 12 and 14, counting from 5′-end of the antisense strand.

In one non-limiting example, the sense strand does not comprise a 2′-deoxy nucleotide at position 11, counting from 5′-end of the sense strand.

### Non-Natural Nucleobases

In some embodiments, the dsRNA can comprise one or more, e.g., 1, 2, 3, 4, 5, 6, 7, 8, 9, 10 or more nucleotides comprising a non-natural nucleobase. A nucleotide comprising a non-natural nucleobase can be present anywhere in the dsRNA molecule. For example, a nucleotide comprising a non-natural nucleobase can be present in the sense strand or a nucleotide comprising a non-natural nucleobase can be present in the antisense strand. When two or more nucleotides comprising a non-natural nucleobase are present in the dsRNA molecule, they can all be in the sense strand, antisense strand or both in the sense and antisense strands.

### Internucleoside Linkages

In some embodiments of any one of the aspects, the dsRNA can comprise one or more, e.g., 1, 2, 3, 4, 5, 6, 7, 8 or more modified internucleoside linkages. For example, the dsRNA can comprise 1, 2, 3, 4, 5 or 6 modified internucleoside linkages. For example, the dsRNA comprises 1, 2, 3 or 4 modified internucleoside linkages. In some embodiments, the dsRNA comprises at least two modified internucleoside linkages between the first five nucleotides counting from the 5′-end of one strand and further comprises at least two modified internucleoside linkages between the first five nucleotides counting from the 3′-end of the said strand. For example, the dsRNA comprises modified internucleoside linkages between nucleotides 1 and 2, and between nucleotides 2 and 3, counting from 5′-end of one strand, and between nucleotides 1 and 2, and between nucleotides 2 and 3, counting from 3′-end of said strand.

In some embodiments of any one of the aspects, the dsRNA comprises one or more, e.g., 1, 2, 3, 4, 5, 6, 7, 8 or more phosphorothioate internucleoside linkages. For example, the dsRNA comprises 1, 2, 3, 4, 5 or 6 phosphorothioate internucleoside linkages. For example, the dsRNA comprises 1, 2, 3 or 4 phosphorothioate internucleoside linkages. In some embodiments, the dsRNA comprises at least two phosphorothioate internucleoside linkages between the first five nucleotides counting from the 5′-end of a strand and further comprises at least two phosphorothioate internucleoside linkages between the first five nucleotides counting from the 3′-end of said strand. For example, the dsRNA comprises modified internucleoside linkages between nucleotides 1 and 2, and between nucleotides 2 and 3, counting from 5′-end of a strand, and between nucleotides 1 and 2, and between nucleotides 2 and 3, counting from 3′-end of said strand.

The dsRNA molecule of the invention can further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule of the invention further comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the dsRNA molecule of the invention comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule of the invention further comprises one to five phosphorothioate or methylphosphonate internucleotide linkage modification(s) within position 1-5 and one to five phosphorothioate or methylphosphonate internucleotide linkage modification(s) within the last 3 positions of the sense strand (counting from the 5′-end), and one to five phosphorothioate or methylphosphonate internucleotide linkage modification at positions 1 and 2 and one to five phosphorothioate or methylphosphonate internucleotide linkage modification within the last six positions of the antisense strand (counting from the 5′-end).

In some embodiments, the dsRNA molecule of the invention further comprises one phosphorothioate internucleotide linkage modification within position 1-5 and one phosphorothioate or methylphosphonate internucleotide linkage modification within the last six positions of the sense strand (counting from the 5′-end), and one phosphorothioate internucleotide linkage modification at positions 1 and 2 and two phosphorothioate or methylphosphonate internucleotide linkage modifications within the last six the last six positions of the antisense strand (counting from the 5′-end).

In some embodiments, the dsRNA molecule of the invention further comprises two phosphorothioate internucleotide linkage modifications within position 1-5 and one phosphorothioate internucleotide linkage modification within the last six positions of the sense strand (counting from the 5′-end), and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and two phosphorothioate internucleotide linkage modifications within the last four positions of the sense strand (counting from the 5′-end), and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and two phosphorothioate internucleotide linkage modifications within the last four positions of the sense strand (counting from the 5′-end), and one phosphorothioate internucleotide linkage modification at positions 1 and 2 and one phosphorothioate internucleotide linkage modification within the last six positions of the antisense strand (counting from the 5′-end).

In some embodiments, the dsRNA molecule of the invention further comprises one phosphorothioate internucleotide linkage modification within position 1-5 and one phosphorothioate internucleotide linkage modification within the last four positions of the sense strand (counting from the 5′-end), and two phosphorothioate internucleotide linkage modifications at positions 1 and 2 and two phosphorothioate internucleotide linkage modifications within the last six positions of the antisense strand (counting from the 5′-end).

In some embodiments, the dsRNA molecule of the invention further comprises one phosphorothioate internucleotide linkage modification within position 1-5 and one within the last six positions of the sense strand (counting from the 5′-end), and two phosphorothioate internucleotide linkage modification at positions 1 and 2 and one phosphorothioate internucleotide linkage modification within the last six positions of the antisense strand (counting from the 5′-end).

In some embodiments, the dsRNA molecule of the invention further comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within the last six positions of the antisense strand (counting from the 5′-end).

In some embodiments, the dsRNA molecule of the invention further comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and one within the last six positions of the sense strand (counting from the 5′-end), and two phosphorothioate internucleotide linkage modifications at positions 1 and 2 and one phosphorothioate internucleotide linkage modification within the last six positions of the antisense strand (counting from the 5′-end).

In some embodiments, the dsRNA molecule of the invention further comprises two phosphorothioate internucleotide linkage modifications within position 1-5 and one phosphorothioate internucleotide linkage modification within the last six positions of the sense strand (counting from the 5′-end), and two phosphorothioate internucleotide linkage modifications at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and one phosphorothioate internucleotide linkage modification within the last six positions of the sense strand (counting from the 5′-end), and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule of the invention further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule of the invention further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 22 and 23 the antisense strand (counting from the 5′-end).

In some embodiments, the sense strand comprises at least two phosphorothioate internucleotide linkages between the first five nucleotides counting from the 5′ end of the sense strand. For example, the sense strand comprises phosphorothioate linkages between nucleotides 1 and 2, and between nucleotides 2 and 3, counting from 5′-end of the sense strand.

In some embodiments, the antisense strand comprises at least two phosphorothioate internucleotide linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand.

In some embodiments, the antisense strand comprises at least two phosphorothioate internucleotide linkages between the first five nucleotides counting from the 3′ end of the antisense strand. For example, the antisense strand comprises phosphorothioate linkages between nucleotides n and n−1, and between nucleotides n−1 and n−2, where n is length of the antisense strand, i.e, number of nucleotides in the antisense strand. In other words, the antisense strand comprises phosphorothioate linkages between nucleotides 1 and 2, and between nucleotides 2 and 3, counting from 3′-end of the antisense strand.

In some embodiments, the antisense strand comprises at least two phosphorothioate internucleotide linkages between the first five nucleotides counting from the 5′-end of the antisense strand and at least two phosphorothioate internucleotide linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand and between nucleotides 1 and 2, and between nucleotides 2 and 3, counting from 3′-end of the antisense strand.

In some embodiments, the sense strand comprises at least two phosphorothioate internucleotide linkages between the first five nucleotides counting from the 5′ end of the sense strand and the antisense strand comprises at least two phosphorothioate internucleotide linkages between the first five nucleotides counting from the 5′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 5′-end of the antisense strand.

In some embodiments, the sense strand comprises at least two phosphorothioate internucleotide linkages between the first five nucleotides counting from the 5′ end of the sense strand and the antisense strand comprises at least two phosphorothioate internucleotide linkages between the first five nucleotides counting from the 3′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 3′-end of the antisense strand.

In some embodiments, oligonucleotide of the invention comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, compound of the invention comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, compound of the invention comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleotidic linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, compound of the invention comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

Various publications describe multimeric siRNA which can all be used with the dsRNA of the invention. Such publications include WO2007/091269, U.S. Pat. No. 7,858,769, WO2010/141511, WO2007/117686, WO2009/014887 and WO2011/031520 which are hereby incorporated by their entirely.

### Ligands

Without wishing to be bound by a theory, ligands modify one or more properties of the attached molecule (e.g., the oligonucleotide described herein) including but not limited to pharmacodynamic, pharmacokinetic, binding, absorption, cellular distribution, cellular uptake, charge and clearance. Ligands are routinely used in the chemical arts and are linked directly or via an optional linking moiety or linking group to a parent compound. A preferred list of ligands includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred ligands amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxylpropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, bomeol, menthol, 1,3-propanediol, heptadecyl group, palmitic acid, myristic acid,O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 IAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a. helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA); AALAEALAEALAEALAEALAEALAAAAGGC (EALA); ALEALAEALEALAEA; GLFEAIEGFIENGWEGMIWDYG (INF-7); GLFGAIAGFIENGWEGMIDGWYG (Inf HA-2); GLFEAIEGFIENGWEGMIDGWYGCGLFEAIEGFIENGWEGMID GWYGC (diINF-7); GLFEAIEGFIENGWEGMIDGGCGLFEAIEGFIENGWEGMIDGGC (diINF-3); GLFGALAEALAEALAEHLAEALAEALEALAAGGSC (GLF); GLFEAIEGFIENGWEGLAEALAEALEALAAGGSC (GALA-INF3); GLF EAI EGFI ENGW EGnI DG K GLF EAI EGFI ENGW EGnI DG (INF-5, n is norleucine); LFEALLELLESLWELLLEA (JTS-1); GLFKALLKLLKSLWKLLLKA (ppTG1); GLFRALLRLLRSLWRLLLRA (ppTG20); WEAKLAKALAKALAKHLAKALAKALKACEA (KALA); GLFFEAIAEFIEGGWEGLIEGC (HA); GIGAVLKVLTTGLPALISWIKRKRQQ (Melittin); H₅WYG; and CHK6HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin); GRKKRRQRRRPPQC (Tat fragment 48-60); GALFLGWLGAAGSTMGAWSQPKKKRKV (signal sequence based peptide); LLIILRRRIRKQAHAHSK (PVEC); GWTLNSAGYLLKINLKALAALAKKIL (transportan); KLALKLALKALKAALKLA (amphiphilic model peptide); RRRRRRRRR (Arg9); KFFFKFFKFFK (Bacterial cell wall permeating peptide); LLGDFFRKSKEKIGKEFKRIVQRIKDFLRNLVPRTES (LL-37); SWLSKTAKKLENSAKKRISEGIAIAIQGGPR (cecropin P1); ACYCRIPACIAGERRYGTCIYQGRLWAFCC (α-defensin); DHYNCVSSGGQCLYSACPIFTKIQGTCYRGKAKCCK (β-defensin); RRRPRPPYLPRPRPPPFFPPRLPPRIPPGFPPRFPPRFPGKR-NH2 (PR-39); ILPWKWPWWPWRR-NH2 (indolicidin); AAVALLPAVLLALLAP (RFGF); AALLPVLLAAP (RFGF analogue); and RKCRIVVIRVCR (bactenecin).

Exemplary cationic groups include, but are not limited to, protonated amino groups, derived from e.g., O-AMINE (AINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc2 and GalNAc3; D-mannose, multivalent mannose, multivalent lactose, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of oligonucleotides described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleoside linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments of any one of the aspects, the ligand has a structure shown in any of Formula (IV)-(VII):

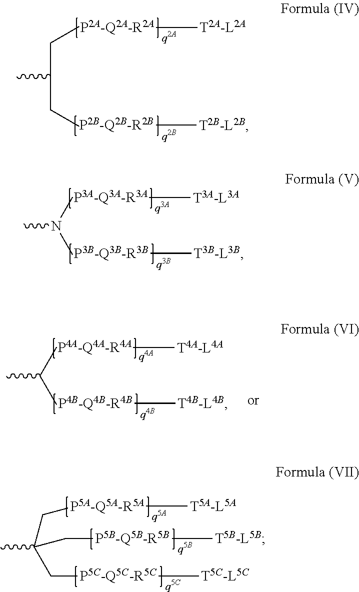

wherein:
q^{2A}, q^{2B}, q^{3A}, q^{3B} q^{4A}, q^{4B}, q^{5A}, q^{5B} and q^{5C} represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R¹¹), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

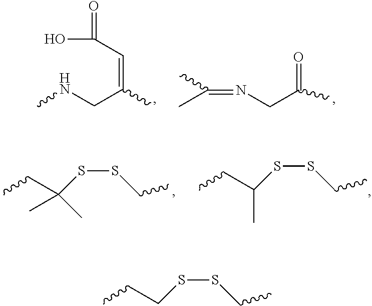

or heterocyclyl;
L^{2A}, L^{2B}, L^{3A}, L^{3B} L^{4A}, L^{4B} L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

In some embodiments of any one of the aspects, the ligand is of Formula (VII):

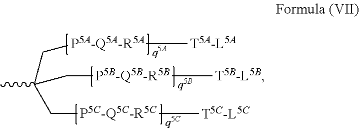

wherein L^{5A}, L^{5B} and L^{5C} represent a monosaccharide, such as GalNAc derivative.

Exemplary ligands include, but are not limited to, the following:

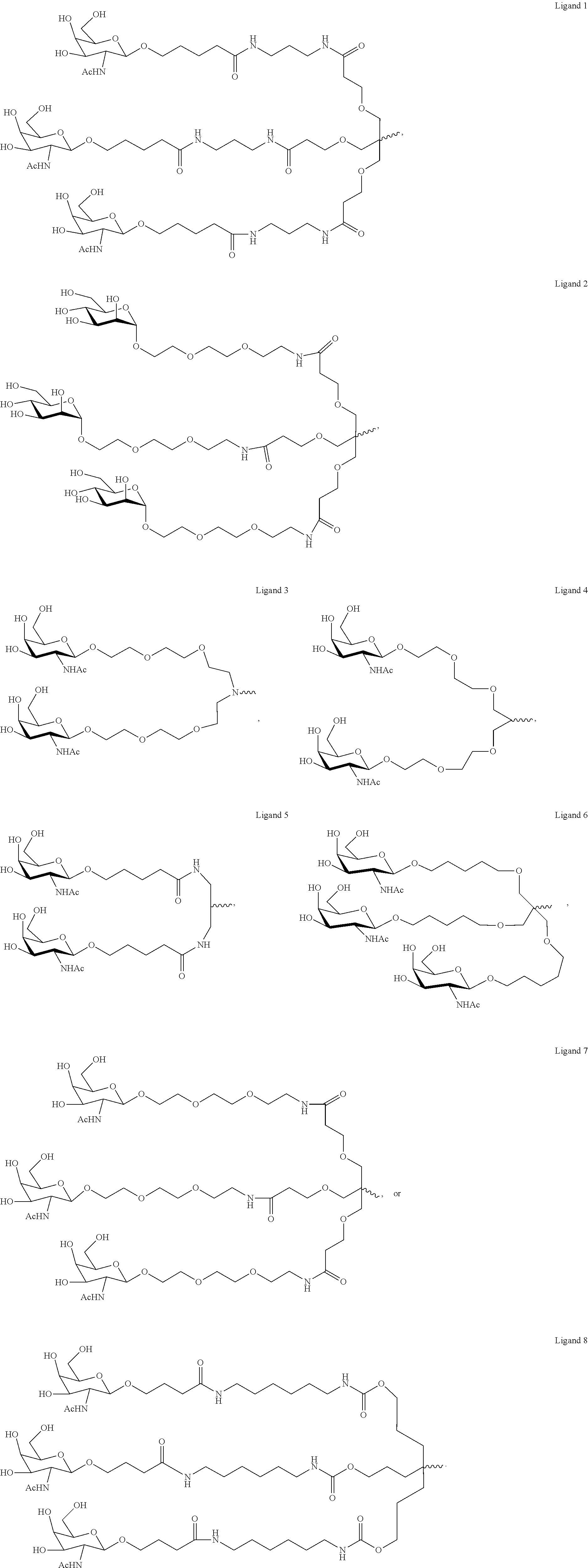

In some embodiments of any one of the aspects described herein, the ligand is a ligand described in U.S. Pat. No. 5,994,517 or 6,906,182, content of each of which is incorporated herein by reference in its entirety.

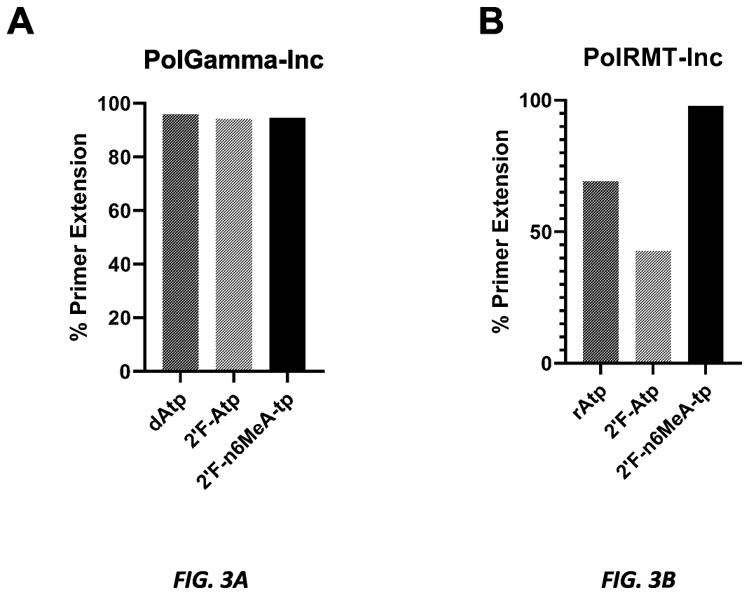

In some embodiments, the ligand can be a tri-antennary ligand described in FIG. 3 of U.S. Pat. No. 6,906,182. For example, the ligand is selected from the following tri-antennary ligands:

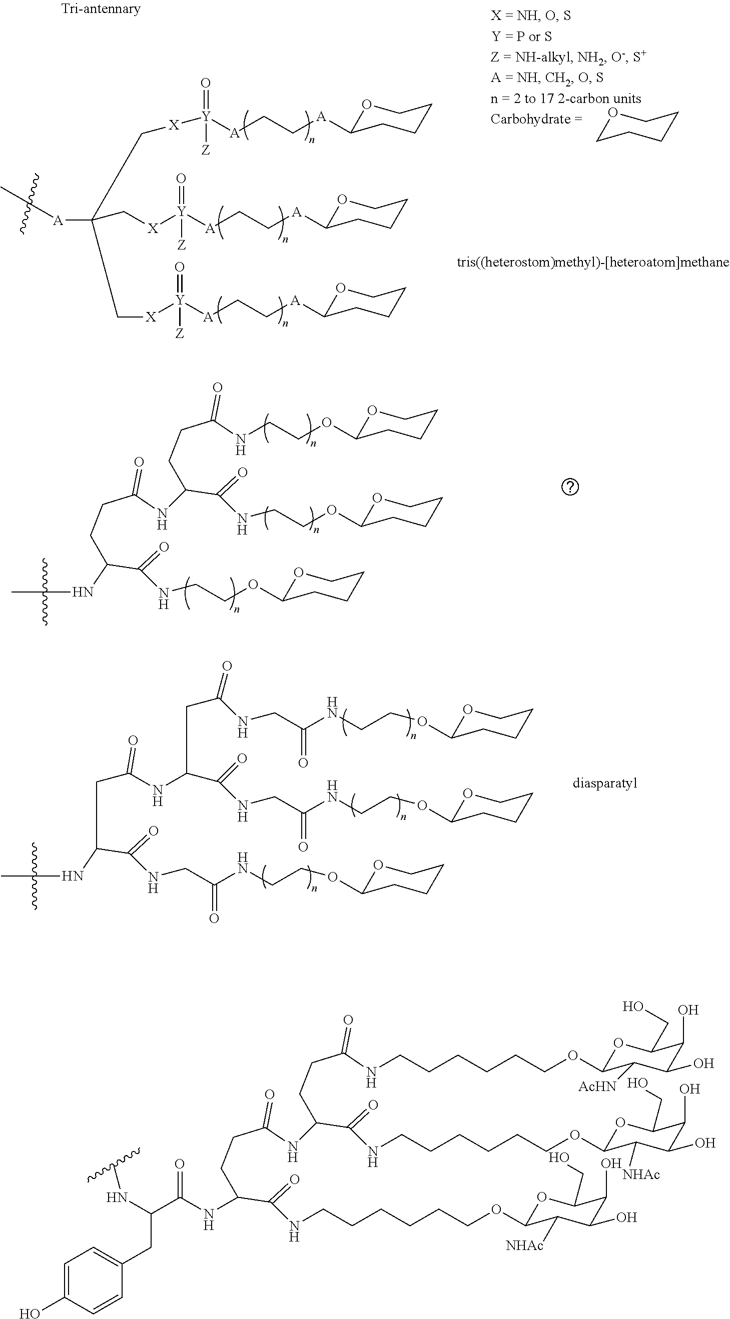

It is noted that when more than one ligand are present, they can be same or different. Accordingly, in some embodiments of any one of the aspects described herein, all ligands are same. In some other embodiments of any one of the aspects described herein, ligands are different.

The ligand can be attached to the sense strand, antisense strand or both strands, at the 3′-end, 5′-end or both ends. For instance, the ligand can be conjugated to the sense strand, in particular, the 3′-end of the sense strand.

### Linkers

Embodiments of the various aspects described herein include a linker. As used herein, the term “linker” means an organic moiety that connects two parts of a compound. Linkers typically comprise a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a cleavable linker. Cleavable linkers are those that rely on processes inside a target cell to liberate the two parts the linker is holding together, as reduction in the cytoplasm, exposure to acidic conditions in a lysosome or endosome, or cleavage by specific enzymes (e.g. proteases) within the cell. As such, cleavable linkers allow the two parts to be released in their original form after internalization and processing inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

Generally, the cleavable linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used according to the present invention that are cleaved upon reduction or oxidation. An example of reductively cleavable linking group is a disulfide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups, which may be used in the compounds, oligonucleotides and dsRNA molecules according to the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, hydrogen, C1-C20 alkyl, C1-C20 haloalkyl, C6-C10 aryl, C7-C12 aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used in the dsRNA molecule according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the compounds, oligonucleotides and dsRNA molecules according to the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used in the compounds, oligonucleotides and dsRNA molecules according to the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCR^{B}C(O)—, where R^{A} and R^{B} are the R groups of the two adjacent amino acids.

In some embodiments of any one of the aspects described herein, the linker is —C(O)CH₂CH₂C(O)—, —OC(O)CH₂CH₂C(O)—, —OC(O)CH₂CH₂C(O)O—, —C(O)CH₂CH₂C(O)NH—, or —OC(O)CH₂CH₂C(O)NH—. For example, the linker is —OC(O)CH₂CH₂C(O)NH—.

In some embodiments, the dsRNA molecule of the invention comprises one or more overhang regions and/or capping groups of dsRNA molecule at the 3′-end, or 5′-end or both ends of a strand. The overhang can be 1-10 nucleotides in length. For example, the overhang can be 1, 2, 3, 4, 5, 6, 7, 8, 9 or 10 nucleotides in length. In some embodiments, the overhang is 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target sequence or it can be complementary to the gene sequences being targeted or it can be the other sequence. The first and second strands can also be joined, e.g., by additional bases to form a hairpin, or by other non-base linkers.

In some embodiments, the nucleotides in the overhang region of the dsRNA molecule of the invention can each independently be a modified or unmodified nucleotide including, but not limited to 2′-sugar modified, such as, 2′-Fluoro 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h′GNA, and any combinations thereof. For example, dTdT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA molecule of the invention may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

The dsRNA molecule of the invention may comprise only a single overhang, which can strengthen the interference activity of the dsRNA, without affecting its overall stability. For example, the single-stranded overhang is located at the 3-terminal end of the sense strand or, alternatively, at the 3-terminal end of the antisense strand. The dsRNA can also have a blunt end, located at the 5′-end of the antisense strand (or the 3′-end of the sense strand) or vice versa.

Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. For example, the single overhang is at least one, two, three, four, five, six, seven, eight, nine, or ten nucleotides in length. In some embodiments, the dsRNA has a 2 nucleotide overhang on the 3′-end of the antisense strand and a blunt end at the 5′-end of the antisense strand.

The dsRNA of the invention can comprise one or more modified nucleotides. For example, every nucleotide in the sense strand and antisense strand of the dsRNA molecule can be modified. Each nucleotide can be modified with the same or different modification which can include one or more alteration of one or both of the non-linking phosphate oxygens and/or of one or more of the linking phosphate oxygens; alteration of a constituent of the ribose sugar; replacement of the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a central region, may only occur at a non-terminal region, or may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, the dsRNA molecule of the invention comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

### 5′-Modifications

In some embodiments dsRNA molecules of the invention are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-). Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se. In one example, the 5′-modification can in placed in the antisense strand of a dsRNA molecule.

In some embodiments of any one of the aspects described herein, the oligonucleotide or at least one (e.g., both) strand of a dsRNA described herein comprises a 5′-vinylphosphonate group. For example, the oligonucleotide or at least one (e.g., both) strand of a dsRNA described herein comprises a 5′-E-vinyl or at least one (e.g., both) strand of a dsRNA described herein phosphonate group. In some other non-limiting example, the oligonucleotide comprises a 5′-Z-vinylphosphonate group.

In one example, the 5′-modification can be placed in the antisense strand of a double-stranded nucleic acid, e.g., dsRNA molecule. For example, the antisense comprises a 5′-E-vinylphosphonate. In some other non-limiting example, the antisense strand comprises a 5′-Z-vinylphosphonate group.

In some embodiments, the sense strand comprises a 5′-morpholino, a 5′-dimethylamino, a 5′-deoxy, an inverted abasic, or an inverted abasic locked nucleic acid modification at the 5′-end.

In some embodiments of any one of the aspects, the oligonucleotide described herein can comprise a thermally destabilizing modification. For example, the oligonucleotide can comprise at least one thermally destabilizing modification of the duplex within the first 9 nucleotide positions, counting from the 5′-end of the oligonucleotide. In some embodiments, the thermally destabilizing modification is located at position 2, 3, 4, 5, 6, 7, 8 or 9, counting from the 5′-end of the antisense strand. In some embodiments, thermally destabilizing modification is located in positions 2-9, or preferably positions 4-8, counting from the 5′-end of the oligonucleotide. In some further embodiments, the thermally destabilizing modification is located at position 5, 6, 7 or 8, counting from the 5′-end of the oligonucleotide. In still some further embodiments, the thermally destabilizing modification is located at position 7, counting from the 5′-end of the oligonucleotide.

Similarly, the dsRNAs of the invention can comprise thermally destabilizing modifications in the seed region of the antisense strand (i.e., at positions 2-9 of the 5′-end of the antisense strand) to reduce or inhibit off-target gene silencing. Without wishing to be bound by a theory, dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, thermally destabilizing modification of the duplex is located in positions 2-9, or preferably positions 4-8, from the 5′-end of the antisense strand. In some embodiments, the thermally destabilizing modification of the duplex is located at position 2, 3, 4, 5, 6, 7, 8 or 9 from the 5′-end of the antisense strand. In some further embodiments, the thermally destabilizing modification of the duplex is located at position 5, 6, 7 or 8 from the 5′-end of the antisense strand. In still some further embodiments, the thermally destabilizing modification of the duplex is located at position 7 from the 5′-end of the antisense strand.

The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s).

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA). For example, the thermally destabilizing modifications can include, but are not limited to, mUNA and GNA building blocks as follows:

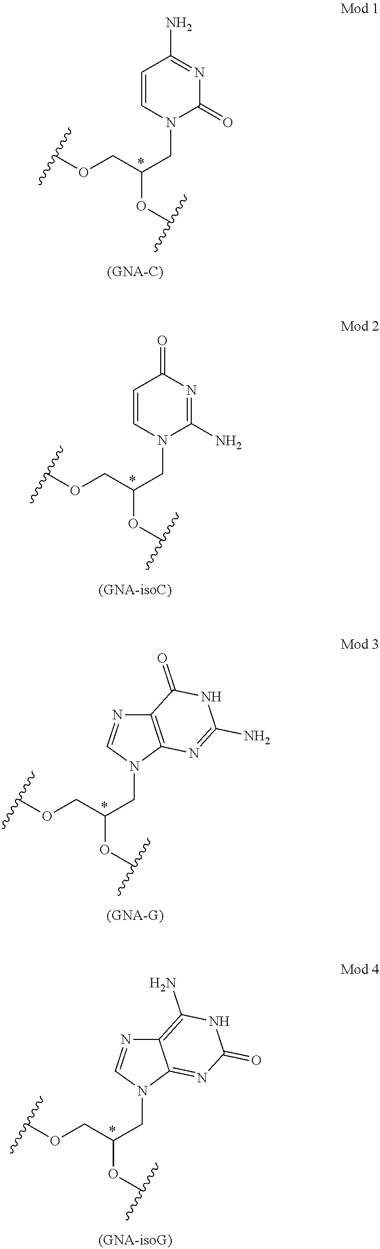 [SMILES: *OCC(Cn1ccc(N)nc1=O)O* ; *OCC(Cn1ccc(=O)nc1N)O* ; *OCC(Cn1cnc2c(=O)[nH]c(N)nc21)O* ; *OCC(Cn1cnc2c(N)[nH]c(=O)nc21)O*]

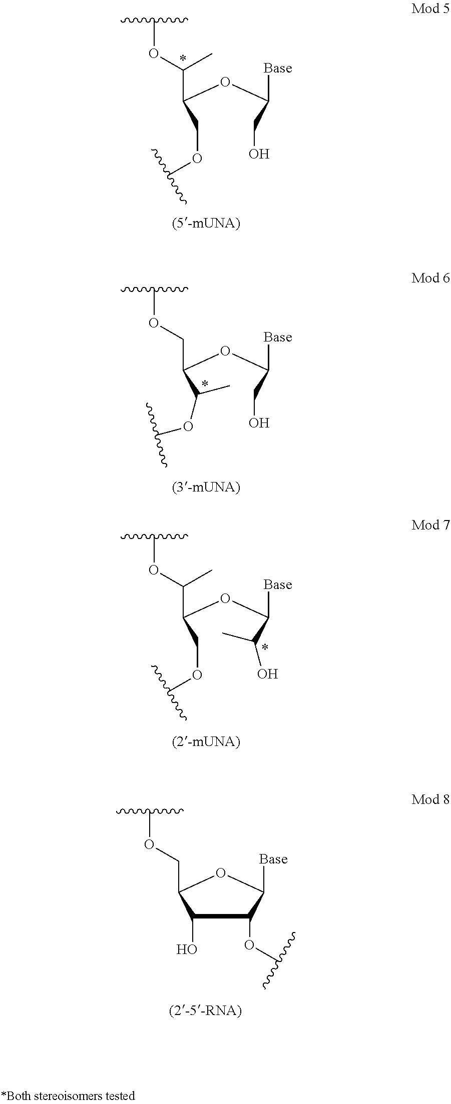

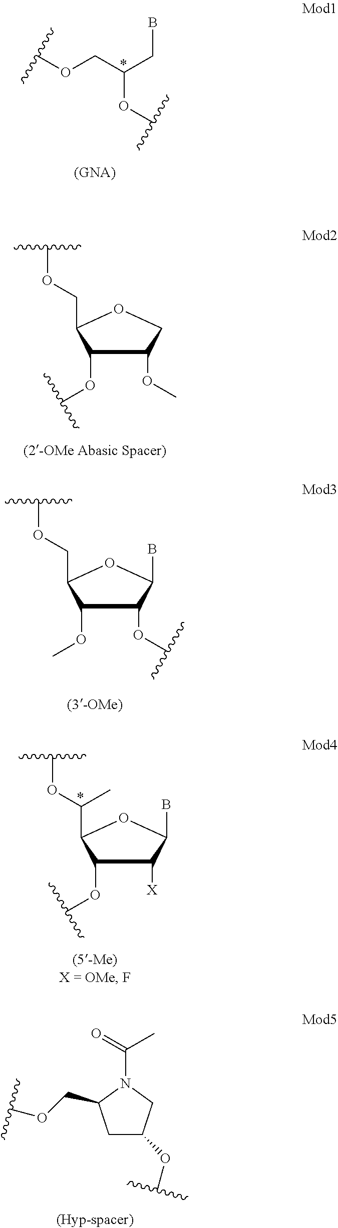

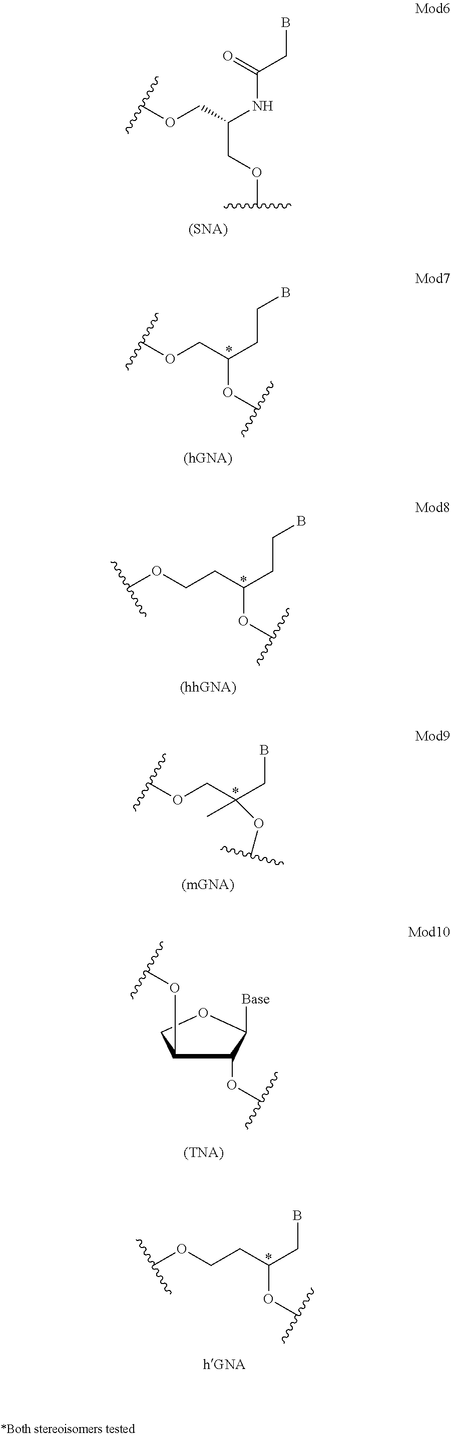

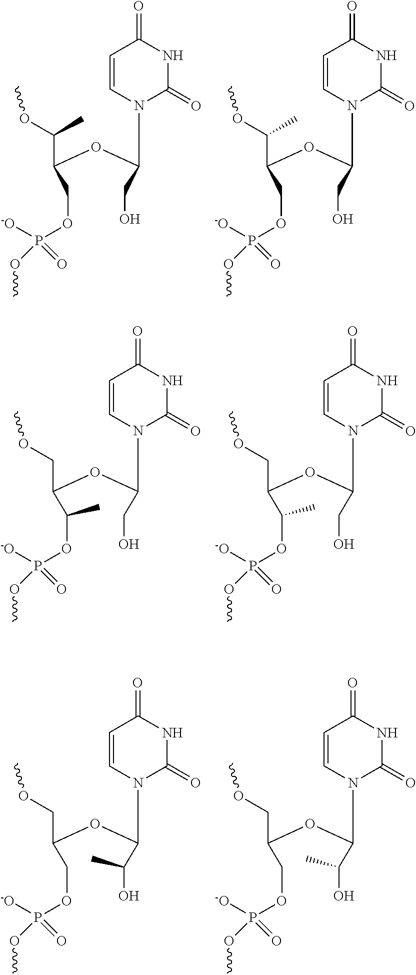

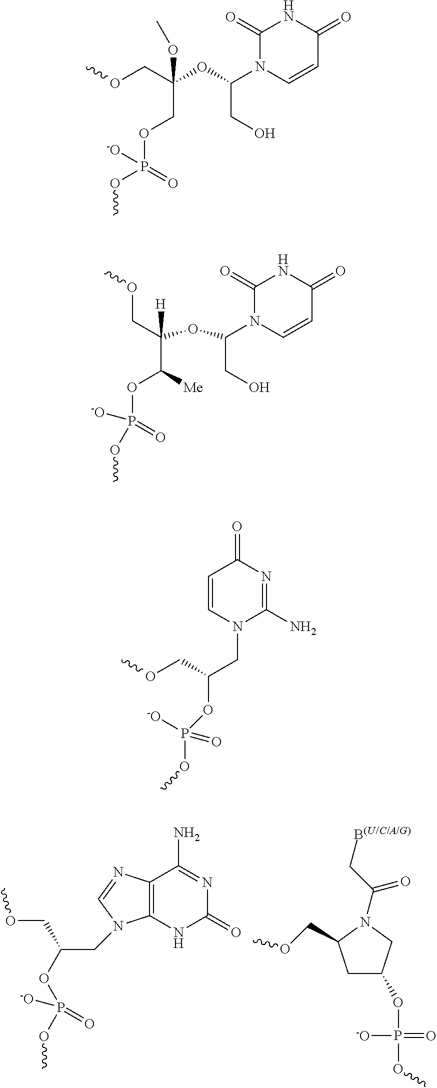

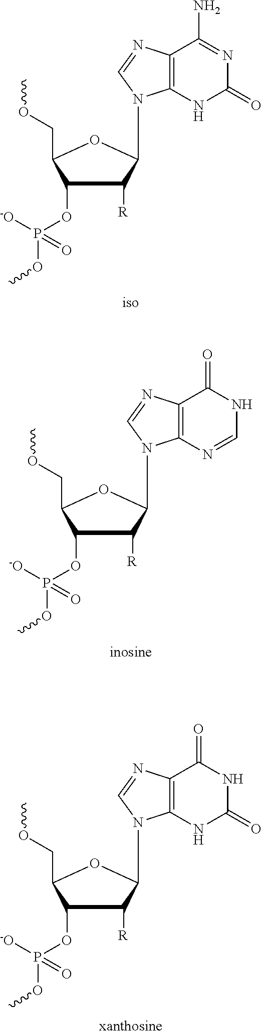

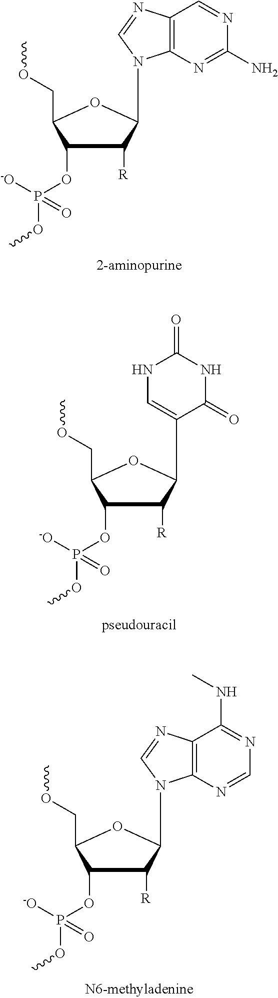

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

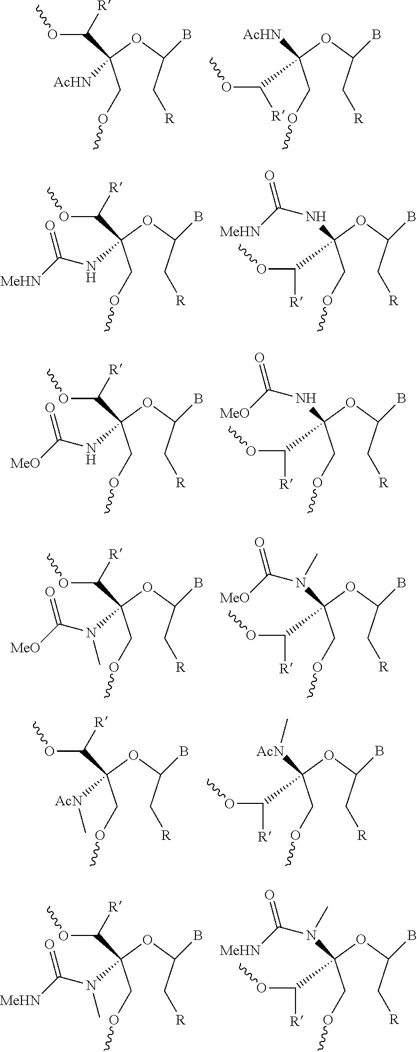

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

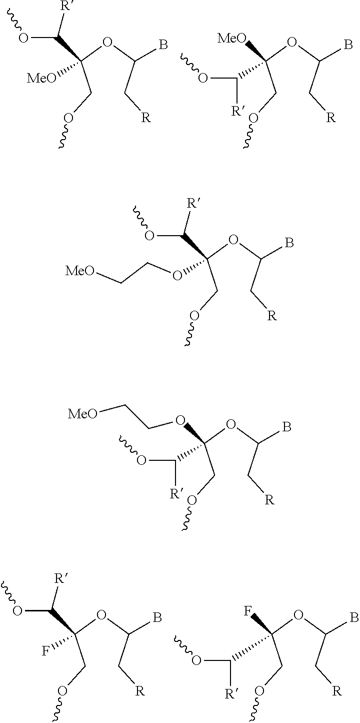

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and Stereochemistry is R or S and combination of R and S for the unspecified chiral centers.

In some embodiments, the destabilizing modification mUNA is selected from the group consisting of

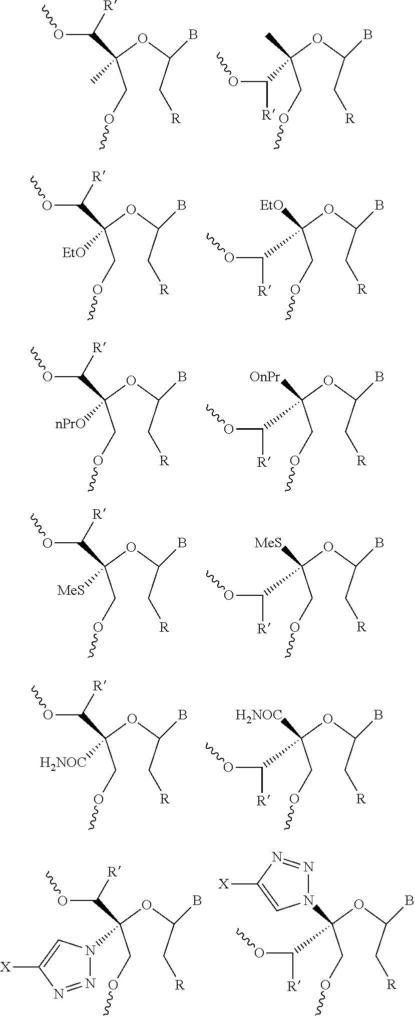

R=H, OMe; F; OH; O—(CH₂)₂₀Me; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 7-deazapurines; and Stereochemistry is R or S and combination of R and S for the unspecified chiral centers.

In some embodiments, the destabilizing modification mUNA is selected from the group consisting of

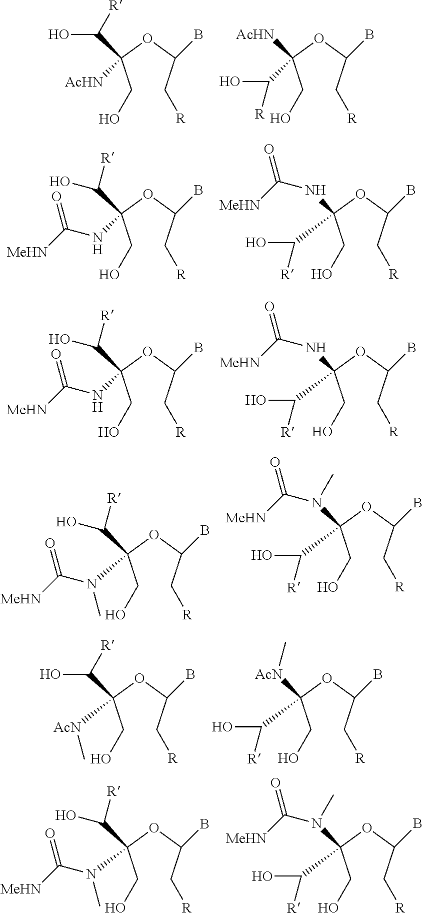

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the destabilizing modification mUNA is selected from the group consisting of

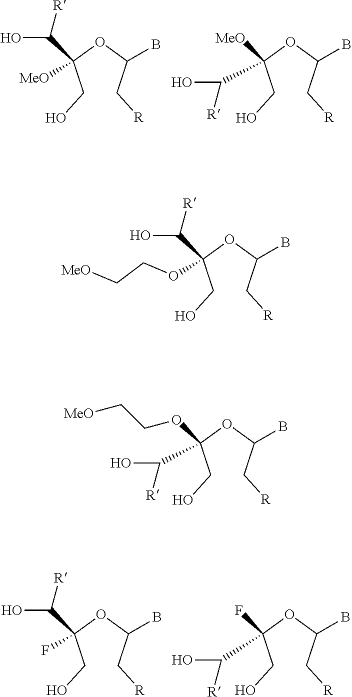

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

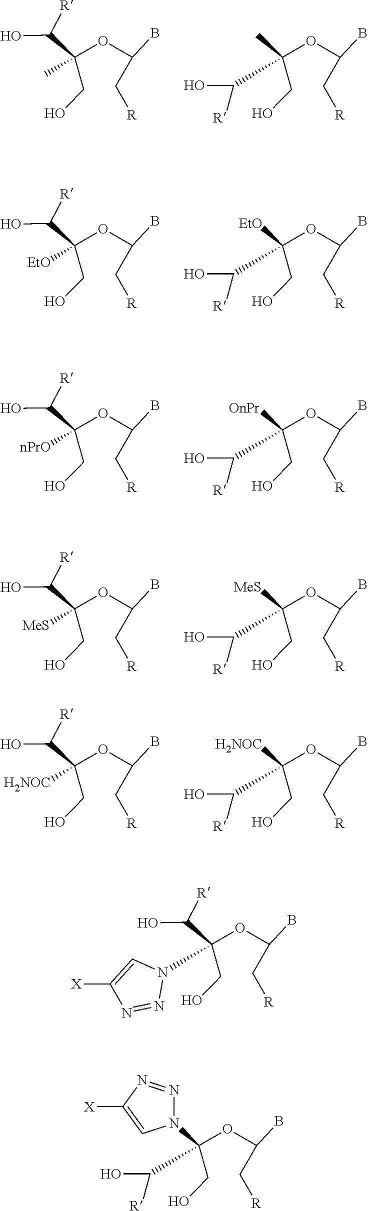

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 7-deazapurines; and
Stereochemistry is R or S and combination of R and S for the unspecified chiral centers

Exemplary abasic modifications include, but are not limited to the following:

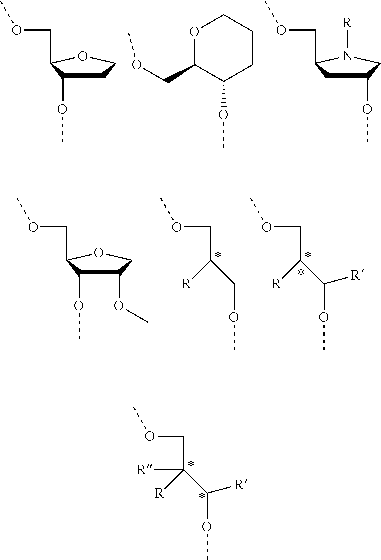

Wherein R=H, Me, Et or OMe; R′=H, Me, Et or OMe; R″=H, Me, Et or OMe

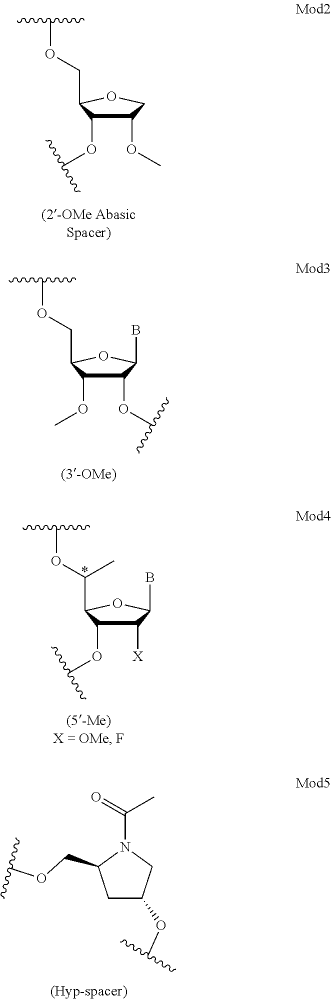

wherein B is a modified or unmodified nucleobase and the asterisk on each structure represents either R, S or racemic.

Exemplified sugar modifications include, but are not limited to the following.

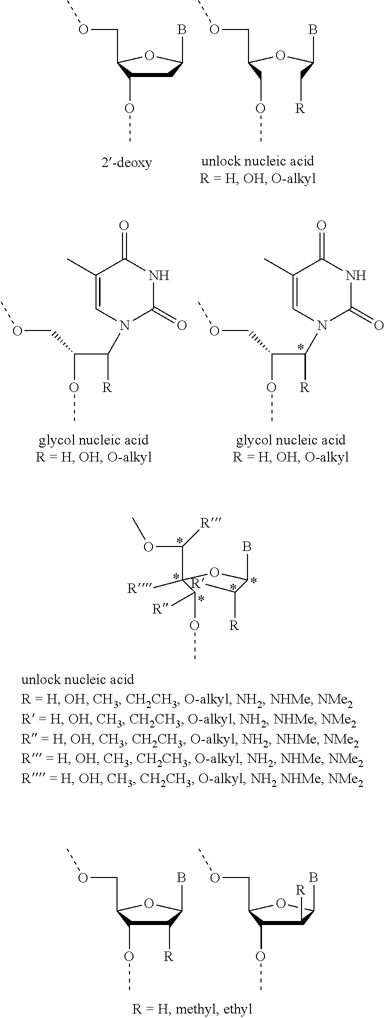

wherein B is a modified or unmodified nucleobase and the asterisk on each structure represents either R, S or racemic.

In some embodiments the thermally destabilizing modification is selected from the mUNA and GNA building blocks described in Examples 1-3 herein. In some embodiments, the destabilizing modification is selected from the group consisting of GNA-isoC, GNA-isoG, 5′-mUNA, 4′-mUNA, 3′-mUNA, and 2′-mUNA. In some further embodiments of this, the dsRNA molecule further comprises at least one thermally destabilizing modification selected from the group consisting of GNA, 2′-OMe, 3′-OMe, 5′-Me, Hy p-spacer, SNA, hGNA, hhGNA, mGNA, TNA and h′GNA (Mod A-Mod K).

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

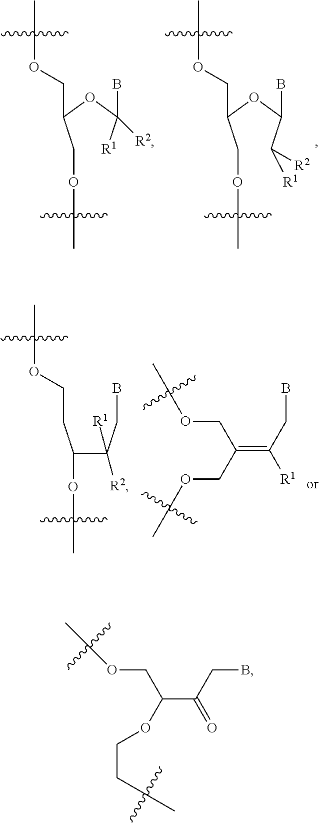

wherein B is a modified or unmodified nucleobase, R1 and R2 independently are H, halogen, OR3, or alkyl; and R3 is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

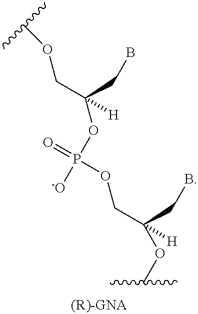

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA, such as:

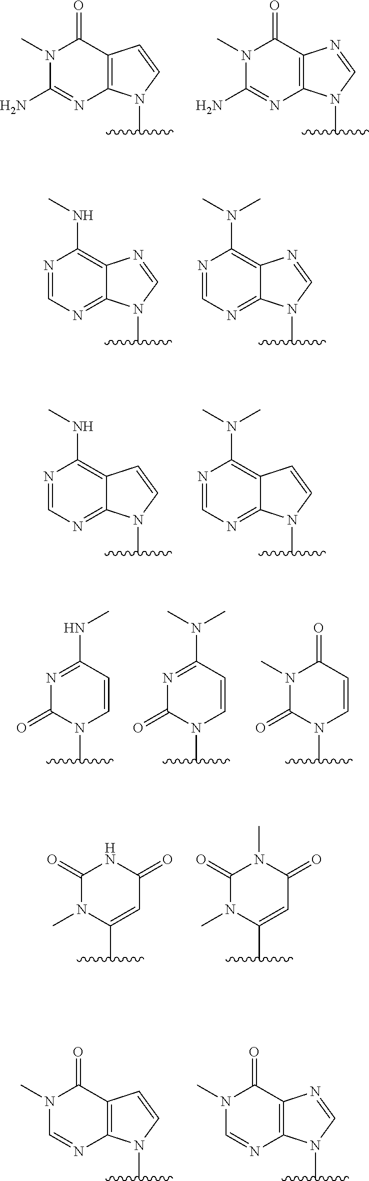

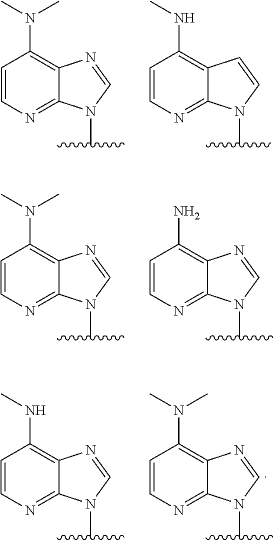

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

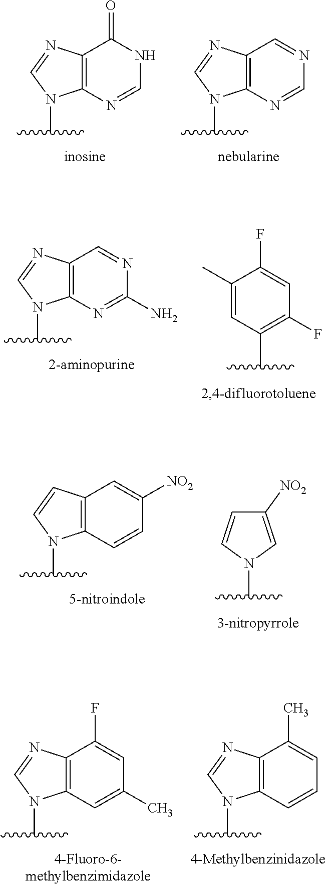

In some embodiments, the thermally destabilizing modification of the duplex in the seed region of the antisense strand includes one or more □-nucleotide complementary to the base on the target mRNA, such as:

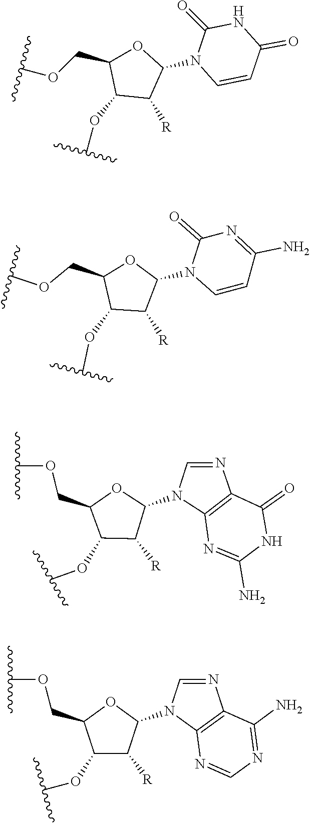

Wherein R is H, OH, OCH₃, F, NH₂, NHMe, NMe₂ or O-alkyl

Exemplary phosphate modifications known to decrease the thermal stability of dsRNA duplexes compared to natural phosphodiester linkages are:

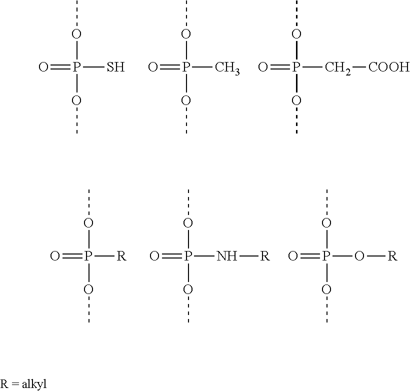

The alkyl for the R group can be a C₁-C₆alkyl. Specific alkyls for the R group include, but are not limited to methyl, ethyl, propyl, isopropyl, butyl, pentyl and hexyl.

It is noted a thermally destabilizing modification can replace a 2′-doexy nucleotide in the antisense strand. For example, a 2′-deoxy nucleotide at positions 2, 5, 7, 12, 14 and/or 16, counting from 5′-end, of the antisense strand can be replaced with a thermally destabilizing modification described herein. Thus, in some embodiments, the antisense strand comprises a thermally destabilizing modification at 1, 2, 3, 4, 5 and/or 6 of positions 2, 5, 7, 12, 14 and/or 16, counting from 5′-end of the antisense strand. For example, the antisense strand comprises a thermally destabilizing modification at positions 5 and 7, counting from 5′-end of the antisense strand.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand and/or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14 and 16 from the 5′-end. In some other embodiments, the antisense comprises stabilizing modifications at positions 2, 6, 14 and 16 from the 5′-end. In still some other embodiments, the antisense comprises stabilizing modifications at positions 2, 14 and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification. In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10 and 11 from the 5′-end. In some other embodiments, the sense strand comprises stabilizing modifications at positions 7, 9, 10 and 11 from the 5′-end. In some embodiments, the sense strand comprises stabilizing modifications at positions opposite or complimentary to positions 11, 12 and 15 of the antisense strand, counting from the 5′-end of the antisense strand. In some other embodiments, the sense strand comprises stabilizing modifications at positions opposite or complimentary to positions 11, 12, 13 and 15 of the antisense strand, counting from the 5′-end of the antisense strand. In some embodiments, the sense strand comprises a block of two, three or four stabilizing modifications.

In some embodiments, the sense strand does not comprise a stabilizing modification in position opposite or complimentary to the thermally destabilizing modification of the duplex in the antisense strand.

Exemplary thermally stabilizing modifications include, but are not limited to 2′-fluoro modifications. Other thermally stabilizing modifications include, but are not limited to LNA.

It is noted a thermally stabilizing modification can replace a 2′-fluoro nucleotide in the sense and/or antisense strand. For example, a 2′-fluoro nucleotide at positions 8, 9, 10, 11 and/or 12, counting from 5′-end, of the sense strand, can be replaced with a thermally stabilizing modification. Similarly, a 2′-fluoro nucleotide at position 14, counting from 5′-end, of the antisense strand, can be replaced with a thermally stabilizing modification.

For the dsRNA molecules to be more effective in vivo, the antisense strand must have some metabolic stability. In other words, for the dsRNA molecules to be more effective in vivo, some amount of the antisense stand may need to be present in vivo after a period time after administration. Accordingly, in some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 5 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 6 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 7 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 8 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 9 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 10 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 11 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 12 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 13 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 14 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 15 after in vivo administration.

Uses of dsRNA

The present invention further relates to a use of a dsRNA molecule as defined herein for inhibiting expression of a target gene. In some embodiments, the present invention further relates to a use of a dsRNA molecule for inhibiting expression of a target gene in vitro.

The present invention further relates to a dsRNA molecule as defined herein for use in inhibiting expression of a target gene in a subject. The subject may be any animal, such as a mammal, e.g., a mouse, a rat, a sheep, a cattle, a dog, a cat, or a human

In some embodiments, the dsRNA molecule of the invention is administered in buffer.

In some embodiments, siRNA compounds described herein can be formulated for administration to a subject. A formulated siRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A dsRNA preparation can be formulated in combination with another agent, e.g., another therapeutic agent or an agent that stabilizes a dsRNA, e.g., a protein that complexes with dsRNA to form an iRNP. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

In some embodiments, the dsRNA preparation includes another dsRNA compound, e.g., a second dsRNA that can mediate RNAi with respect to a second gene, or with respect to the same gene. Still other preparation can include at least 3, 5, ten, twenty, fifty, or a hundred or more different siRNA species. Such dsRNAs can mediate RNAi with respect to a similar number of different genes.

In some embodiments, the dsRNA preparation includes at least a second therapeutic agent (e.g., an agent other than a RNA or a DNA). For example, a dsRNA composition for the treatment of a viral disease, e.g., HIV, might include a known antiviral agent (e.g., a protease inhibitor or reverse transcriptase inhibitor). In another example, a dsRNA composition for the treatment of a cancer might further comprise a chemotherapeutic agent.

Exemplary formulations which can be used for administering the dsRNA molecule according to the present invention are discussed below.

Liposomes. A dsRNA preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the dsRNA are delivered into the cell where the dsRNA can specifically bind to a target RNA and can mediate RNAi. In some cases the liposomes are also specifically targeted, e.g., to direct the dsRNA to particular cell types.

A liposome containing a dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of dsRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging siRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with dsRNA described herein are useful for treating a dermatological disorder.

Liposomes that include dsRNA described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include dsRNA described herein can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. The dsRNA compositions can include a surfactant. In some embodiments, the dsRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. For ease of exposition the micelles and other formulations, compositions and methods in this section are discussed largely with regard to unmodified siRNA compounds. It may be understood, however, that these micelles and other formulations, compositions and methods can be practiced with other siRNA compounds, e.g., modified siRNA compounds, and such practice is within the invention. The siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof)) composition can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the dsRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method, a first micellar composition is prepared which contains the dsRNA composition and at least the alkali metal alkyl sulphate. The first micellar composition is then mixed with at least three micelle forming compounds to form a mixed micellar composition. In another method, the micellar composition is prepared by mixing the dsRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, dsRNA preparations can be incorporated into a particle, e.g., a microparticle. Microparticles can be produced by spray-drying, but may also be produced by other methods including lyophilization, evaporation, fluid bed drying, vacuum drying, or a combination of these techniques.

### Pharmaceutical Compositions

The oligonucleotides, e.g. dsRNAs of the invention can be formulated for pharmaceutical use. The present invention further relates to a pharmaceutical composition comprising the dsRNA molecule as defined herein. Pharmaceutically acceptable compositions comprise a therapeutically-effective amount of one or more of the dsRNA molecules in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a compound of the invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

The oligonucleotide, e.g. dsRNA preparation can be formulated in combination with another agent, e.g., another therapeutic agent or an agent that stabilizes a dsRNA, e.g., a protein that complexes with the dsRNA to form an iRNP. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The compounds according to the invention may be formulated for administration in any convenient way for use in human or veterinary medicine, by analogy with other pharmaceuticals.

The term “treatment” is intended to encompass therapy and cure. The patient receiving this treatment is any animal in need, including primates, in particular humans, and other mammals such as equines, cattle, swine and sheep; and poultry and pets in general.

Double-stranded RNA agents are produced in a cell in vivo, e.g., from exogenous DNA templates that are delivered into the cell. For example, the DNA templates can be inserted into vectors and used as gene therapy vectors. Gene therapy vectors can be delivered to a subject by, for example, intravenous injection, local administration (U.S. Pat. No. 5,328,470, which is incorporated by reference in its entirety), or by stereotactic injection (see, e.g., Chen et al. (1994) Proc. Natl. Acad. Sci. USA 91:3054-3057, which is incorporated by reference in its entirety). The pharmaceutical preparation of the gene therapy vector can include the gene therapy vector in an acceptable diluent, or can comprise a slow release matrix in which the gene delivery vehicle is imbedded. The DNA templates, for example, can include two transcription units, one that produces a transcript that includes the top strand of a dsRNA molecule and one that produces a transcript that includes the bottom strand of a dsRNA molecule. When the templates are transcribed, the dsRNA molecule is produced, and processed into siRNA agent fragments that mediate gene silencing.

The dsRNA molecule as defined herein or a pharmaceutical composition comprising a dsRNA molecule as defined herein can be administered to a subject using different routes of delivery. A composition that includes a dsRNA described herein can be delivered to a subject by a variety of routes. Exemplary routes include: intravenous, subcutaneous, topical, rectal, anal, vaginal, nasal, pulmonary, ocular.

The dsRNA molecule of the invention can be incorporated into pharmaceutical compositions suitable for administration. Such compositions typically include one or more species of dsRNAs and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the dsRNA in aerosol form. The vascular endothelial cells could be targeted by coating a balloon catheter with the dsRNA and mechanically introducing the dsRNA.

In one aspect, the invention features a method of administering an oligonucleotide, e.g., a dsRNA described herein, to a subject (e.g., a human subject). In another aspect, the present invention relates to a dsRNA molecule as defined herein for use in inhibiting expression of a target gene in a subject. The method or the medical use includes administering a unit dose of the oligonucleotide, e.g., a dsRNA described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA agent per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities. In some embodiments, the subject has a viral infection and the modality is an antiviral agent other than a dsRNA molecule, e.g., other than a siRNA agent. In another embodiment, the subject has atherosclerosis and the effective dose of a dsRNA molecule, e.g., a siRNA agent, is administered in combination with, e.g., after surgical intervention, e.g., angioplasty.

In some embodiments, a subject is administered an initial dose and one or more maintenance doses of a dsRNA molecule, e.g., a siRNA agent, (e.g., a precursor, e.g., a larger dsRNA molecule which can be processed into a siRNA agent, or a DNA which encodes a dsRNA molecule, e.g., a siRNA agent, or precursor thereof). The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The dsRNA molecules of the invention described herein can be administered to mammals, particularly large mammals such as nonhuman primates or humans in a number of ways.

In some embodiments, the administration of the dsRNA molecule, e.g., a siRNA agent, composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of dsRNA molecules described herein

In particular embodiments, the present invention relates to the dsRNA molecules of the present invention for use in the methods described above.

### Methods of Inhibiting Expression of the Target Gene

Embodiments of the invention also relate to methods for inhibiting the expression of a target gene. The method comprises the step of administering the dsRNA molecules in any of the preceding embodiments, in an amount sufficient to inhibit expression of the target gene. The present invention further relates to a use of a dsRNA molecule as defined herein for inhibiting expression of a target gene in a target cell. In a preferred embodiment, the present invention further relates to a use of a dsRNA molecule for inhibiting expression of a target gene in a target cell in vitro.

Another aspect the invention relates to a method of modulating the expression of a target gene in a cell, comprising providing to said cell a dsRNA molecule of this invention. In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, IEKK gene, INK gene, RAF gene, Erk1/2 gene, PCNA(p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

In particular embodiments, the present invention relates to the dsRNA molecules of the present invention for use in the methods described above.

Exemplary embodiments of the disclosure can be described by the following numbered embodiments:

Embodiment 1: A double-stranded RNA (dsRNA) molecule capable of inhibiting the expression of a target gene, comprising a sense strand and an antisense strand, independently having a length of 15-35 nucleotides, wherein the antisense strand is substantially complementarity to the target sequence to mediate RNA interference, wherein the dsRNA molecule comprises at least one nucleotide comprising a 6-methyladenine nucleobase and wherein at least 50% of the nucleotides in the dsRNA are 2′-OMe nucleotides.

Embodiment 2: The dsRNA molecule of claim 1, wherein said nucleotide comprising the 6-methyladenine nucleobase further comprises a modified sugar.

Embodiment 3: The dsRNA molecule of claim 2, wherein said modified sugar comprises a 2′-modified ribose.

Embodiment 4: The dsRNA molecule of claim 2 or 3, wherein the modified sugar is selected from the group consisting of 2′-F ribose, 2′-OMe ribose, 2′-O,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxypropyl (glycol nucleic acid, GNA).

Embodiment 5: The dsRNA molecule of claim 4, wherein the modified sugar is 2′-F ribose or 2′-OMe ribose.

Embodiment 6: The dsRNA molecule of any one of claim 1-5, wherein the antisense strand comprises the nucleotide comprising the 6-methyladenine nucleobase.

Embodiment 7: The dsRNA molecule of claim 6, wherein the nucleotide comprising the 6-methyladenine nucleobase is present in a terminal region of the antisense strand.

Embodiment 8: The dsRNA molecule of claim 7, wherein the nucleotide comprising the 6-methyladenine nucleobase is present in a 5′-terminal region of the antisense strand.

Embodiment 9: The dsRNA molecule of claim 7, wherein the nucleotide comprising the 6-methyladenine nucleobase is present in a 3′-terminal region of the antisense strand.

Embodiment 10: The dsRNA molecule of claim 7, wherein the nucleotide comprising the 6-methyladenine nucleobase is not in a central region of the antisense strand.

Embodiment 11: The dsRNA molecule of any one of claim 1-10, wherein the sense strand comprises the nucleotide comprising the 6-methyladenine nucleobase.

Embodiment 12: The dsRNA molecule of claim 11, wherein the nucleotide comprising the 6-methyladenine nucleobase is present in a terminal region of the sense strand.

Embodiment 13: The dsRNA molecule of claim 11, wherein the nucleotide comprising the 6-methyladenine nucleobase is present in a 5′-terminal region of the sense strand.

Embodiment 14: The dsRNA molecule of claim 11, wherein the nucleotide comprising the 6-methyladenine nucleobase is present in a 3′-terminal region of the sense strand.

Embodiment 15: The dsRNA molecule of claim 11, the nucleotide comprising the 6-methyladenine nucleobase is present in a central region of the sense strand.

Embodiment 16: The dsRNA molecule of claim 11, wherein the nucleotide comprising the 6-methyladenine nucleobase is not in a central region of the sense strand.

Embodiment 17: The dsRNA molecule of any one of claims 1-16, wherein the dsRNA molecule further comprises a nucleotide comprising a modified sugar and a nucleobase other than 6-methyladenine.

Embodiment 18: The dsRNA molecule of 17, wherein the nucleotide comprising a modified sugar and a nucleobase other than 6-methyladenine is selected from the group consisting of 2′-F nucleotides, 2′-OMe nucleotides, locked nucleic acid (LNA) nucleotides, HNA nucleotides, CeNA nucleotides, 2′-methoxyethyl nucleotides, 2′-O-allyl nucleotides, 2′-C-allyl nucleotides, 2′-O—N-methylacetamido (2′-O-NMA) nucleotides, 2′-O-dimethylaminoethoxyethyl (2′-O-DMAEOE) nucleotides, 2′-O-aminopropyl (2′-O-AP) nucleotides, 2′-ara-F nucleotides, TNA nucleotides, and GNA nucleotides.

Embodiment 19: The dsRNA molecule of any one of claims 1-18, wherein the dsRNA molecule comprises a 2′-F nucleotide comprising a nucleobase other than 6-methyladenine.

Embodiment 20: The dsRNA molecule of claim 19, wherein said 2′-F nucleotide is present in a central region of the sense strand.

Embodiment 21: The dsRNA molecule of any one of claims 1-20, wherein the dsRNA molecule comprises a 2′-OMe nucleotide comprising a nucleobase other than 6-methyladenine.

Embodiment 22: The dsRNA molecule of any one claims 1-21, wherein the dsRNA molecule further comprises a 2′-doexy (2′-H) nucleotide comprising a nucleobase other than 6-methyladenine.

Embodiment 23: The dsRNA molecule of any one of claims 1-22, wherein the dsRNA molecule comprises a ligand.

Embodiment 24: The dsRNA molecule of any one of claims 1-23, wherein the sense strand comprises a ligand.

Embodiment 25: The dsRNA molecule of claim 23 or 24, wherein the ligand is an ASGPR ligand.

Embodiment 26: The dsRNA molecule of any one of claims 1-25, wherein the dsRNA molecule comprises at least two phosphorothioate internucleotide linkages.

Embodiment 27: The dsRNA molecule of any one of claims 1-26, wherein the sense strand comprises at least two phosphorothioate internucleotide linkages between the first five nucleotides counting from the 5′ end of the sense strand.

Embodiment 28: The dsRNA molecule of any one of claims 1-27, wherein the antisense strand comprises at least two phosphorothioate internucleotide linkages between the first five nucleotides counting from the 5′ end of the antisense strand and at least two phosphorothioate internucleotide linkages between the first five nucleotides counting from the 3′ end of the antisense strand.

Embodiment 29: The dsRNA molecule of any one of claims 1-28, wherein the dsRNA has a duplex region of from 18 to about 25 basepairs.

Embodiment 30: The dsRNA molecule of any one of claims 1-29, wherein the sense strand is 18-23 nucleotides in length.

Embodiment 31: The dsRNA molecule of any one of claims 1-30, wherein the antisense strand is 18-25 nucleotides in length.

Embodiment 32: The dsRNA molecule of any one of claims 1-31, wherein the dsRNA molecule comprises a single stranded overhang at 3′-end of the antisense strand.

Embodiment 33: The dsRNA molecule of any one of claims 1-32, wherein the dsRNA molecule comprises a blunt end at the 5′-end of the antisense strand.

Embodiment 34: The dsRNA molecule of any one of claims 1-33, wherein at least 50% of the nucleotides in the sense strand are 2′-OMe nucleotides.

Embodiment 35: The dsRNA molecule of any one of claims 1-34, wherein at least 50% of the nucleotides in the antisense strand are 2′-OMe nucleotides.

Embodiment 36: A pharmaceutical composition comprising the dsRNA molecule of any one of claims 1-35 alone or in combination with a pharmaceutically acceptable carrier or excipient.

Embodiment 37: A gene silencing kit containing the dsRNA molecule of any one of any one claims 1-35.

Embodiment 38: A method for silencing a target gene in a cell, the method comprising a step of introducing the dsRNA molecule of any one of claims 1-35 into the cell.

Embodiment 39: A method for silencing a target gene in a subject, the method comprising administering a dsRNA molecule of any one of claims 1-35 to the subject.

Embodiment 40: The method of claim 39, wherein said administering the dsRNA molecule is subcutaneous or intravenous administration.

Some additional exemplary embodiments of the various aspects described herein can be described by one or more of the following numbered embodiments:

An oligonucleotide comprising at least one nucleoside of Formula (I), optionally provided that the nucleoside of Formula (I) is not where Y^{A} is N; R^{A1} is methyl; R^{A2} is H or nitrogen protecting group; R²² is hydroxyl or protected hydroxyl; R²³ is a bond to an internucleotide linkage to a subsequent nucleotide, hydroxyl or protected hydroxyl; R⁴ is H; and R²⁵ is a bond to an internucleotide linkage to a preceding nucleotide, hydroxyl or protected hydroxyl, and both of R²³ and R²⁵ are not hydroxyl or protected hydroxyl at the same time.

The oligonucleotide of Embodiment 1, wherein Y^{A} is N.

The oligonucleotide of Embodiment 1, wherein Y^{A} is CH.

The oligonucleotide of any one of Embodiments 1-3, wherein R^{A1} is optionally substituted C₁₋₃₀ alkyl, optionally substituted C₂₋₃₀alkenyl, optionally substituted C₂₋₃₀alkynyl, optionally substituted aryl, optionally substituted cycloalkyl, or optionally substituted benzyl group.

The oligonucleotide of any one of Embodiments 1-4, wherein R^{A1} is optionally substituted C₁₋₃₀ alkyl, or

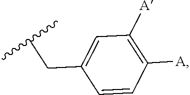

where A and A′ independently are hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl, alkoxyalkylamine, alkoxyoxycarboxylate, amino, alkylamino, dialkylamino, protected amino, a ligand, or a linker covalently bonded to one or more ligands.

The oligonucleotide of any one of Embodiments 1-5, wherein R^{A1} is methyl, isopropyl, or cyclopropyl.

The oligonucleotide of any one of Embodiments 1-5, wherein R^{A1} is

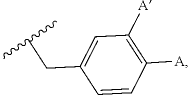

where: (i) A is CH₂CO₂Me and A′ is H; (ii) A is H and A′ is CH₂CO₂Me; (iii) A and A′ each are CH₂CO₂Me; (iv) A is CO₂Me and A′ is H; (v) A is H and A′ is CO₂Me; or (vi) A and A′ each are CO₂Me.

The oligonucleotide of any one of Embodiments 1-7, wherein R^{A2} is hydrogen.

The oligonucleotide of any one of Embodiments 1-7, wherein R^{A2} is a nitrogen protecting group.

The oligonucleotide of any one of Embodiments 1-9, wherein R² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support; or R² and R⁴ taken together are 4′-C(R¹⁰R¹¹)ᵥ—Y-2′ or 4′-Y—C(R¹⁰R¹¹)ᵥ-2′.

The oligonucleotide of any one of Embodiments 1-10, wherein R² is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy, 2-methoxyethoxy), alkoxyalkyl (e.g., methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R₈)(CH₂R₉), —O—C₄₋₃₀alkyl-ON(CH₂R₈)(CH₂R₉); or R² and R⁴ taken together are 4′-C(R¹⁰R¹¹)ᵥ—Y-2′ or 4′-Y—C(R¹⁰R¹¹)ᵥ-2′.

The oligonucleotide of any one of Embodiments 1-11, wherein R² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl) or C₆₋₂₄alkoxy (e.g., n-C₆₋₂₄ alkoxy); or R² and R⁴ taken together are 4′-C(R¹⁰R¹¹)ᵥ—Y-2′ or 4′-Y—C(R¹⁰R¹¹)ᵥ-2′.

The oligonucleotide of any one of Embodiments 1-12, wherein R² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl) or C₆₋₂₄alkoxy (e.g., n-C₆₋₂₄ alkoxy).

The oligonucleotide of any one of Embodiments 1-13, wherein R⁴ is H.

The oligonucleotide of any one of Embodiments 1-14, wherein R³ is a bond to an internucleotide linkage to a subsequent nucleotide, hydroxyl, optionally substituted C₁₋₃₀ alkoxy, a 3′-oligonucleotide capping group, a solid support, a linker or a linker covalently bonded to a solid support.

The oligonucleotide of any one of Embodiments 1-15, wherein R³ is a bond to an internucleotide linkage to a subsequent nucleotide.

The oligonucleotide of any one of Embodiments 1-15, wherein R³ is hydroxyl or protected hydroxyl.

The oligonucleotide of any one of Embodiments 1-17, wherein R⁵ is a bond to an internucleotide linkage to a preceding nucleotide, hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate, phosphate mimic, or a bond to an internucleotide linkage to a preceding nucleotide.

The oligonucleotide of any one of Embodiments 1-18, wherein R⁵ is hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate.

The oligonucleotide of any one of Embodiments 1-18, wherein R⁵ is a bond to an internucleotide linkage to a preceding nucleotide.

The oligonucleotide of any one of Embodiments 1-20, wherein the oligonucleotide comprises from 3 to 50 nucleotides.

The oligonucleotide of any one of Embodiments 1-21, wherein the oligonucleotide comprises at least one ribonucleotide.

The oligonucleotide of any one of Embodiments 1-22, wherein the oligonucleotide comprises at least one 2′-deoxyribonucleotide.

The oligonucleotide of any one of Embodiments 1-23, wherein the oligonucleotide comprises at least one nucleotide with a modified or non-natural nucleobase in addition to the nucleotide of Formula (IA) or (IB).

The oligonucleotide of any one of Embodiments 1-24, wherein the oligonucleotide comprises at least one nucleotide with a modified ribose sugar in addition to the nucleotide of Formula (IA) or (IB).

The oligonucleotide of any one of Embodiments 1-25, wherein the oligonucleotide comprises at least one nucleotide comprising a group other than H or OH at the 2′-position of the ribose sugar in addition to the nucleotide of Formula (I).

The oligonucleotide of any one of Embodiments 1-26, wherein the oligonucleotide comprises at least one nucleotide with a 2′-F ribose in addition to the nucleotide of Formula (I).

The oligonucleotide of any one of Embodiments 1-27, wherein the oligonucleotide comprises at least one nucleotide with a 2′-OMe ribose in addition to the nucleotide of Formula (I).

The oligonucleotide of any one of Embodiments 1-28, wherein the oligonucleotide comprises at least one nucleotide comprising a moiety other than a ribose sugar in addition to the nucleotide of Formula (I).

The oligonucleotide of any one of Embodiments 1-29, wherein the oligonucleotide comprises at least one modified internucleotide linkage.

The oligonucleotide of any one of Embodiments 1-30, wherein the oligonucleotide is attached to a solid support.

The oligonucleotide of any one of Embodiments 1-31, wherein oligonucleotide comprises at least one ligand.

The oligonucleotide of any one of Embodiments 1-32, wherein the oligonucleotide comprises at least one hydroxyl, phosphate or amino protecting group.

A double-stranded nucleic acid comprising a first oligonucleotide strand and a second oligonucleotide strand substantially complementary to the first strand, wherein the first or second strand is an oligonucleotide of any one of Embodiments 1-33.

The double-stranded nucleic acid of Embodiment 34, wherein the first and second strand are independently 15 to 25 nucleotides in length.

The double-stranded nucleic acid any one of Embodiments 34-35, wherein double-stranded nucleic acid is capable of inducing RNA interference.

The double-stranded nucleic acid of any one of Embodiments 34-36, wherein one or both strands have a 1-5 nucleotide overhang on its respective 5′-end or 3′-end.

The double-stranded nucleic acid of any one of Embodiments 34-37, wherein only one strand has a 2 nucleotide overhang on its 5′-end or 3′-end.

The double-stranded nucleic acid of any one of Embodiments 34-38, wherein only one strand has a 2 nucleotide overhand on its 3′-end.

A method of reducing the expression of a target gene in a subject, comprising administering to the subject either: (i) a double-stranded RNA according to any one of Embodiments 34-39, wherein the first strand or the second strand is complementary to a target gene; or (ii) an oligonucleotide according to any one of Embodiments 1-33, wherein the oligonucleotide is complementary to a target gene.

A compound of Formula (II), optionally provided that the compound is not where Y^{A} is N; R^{A1} is methyl; R^{A2} is H or nitrogen protecting group; R²² is hydroxyl, protected hydroxyl, reactive phosphorous, a linker or a linker attached to a solid-support; R²³ is hydroxyl, protected hydroxyl, reactive phosphorous, a linker or a linker attached to a solid-support; R⁴ is H; and R²⁵ is hydroxyl or protected hydroxyl.

The compound of Embodiment 41, wherein Y^{A} is N.

The compound of Embodiment 41, wherein Y^{A} is CH.

The compound of any one of Embodiments 41-43, wherein R^{A1} is optionally substituted C₁₋₃₀ alkyl, optionally substituted C₂₋₃₀alkenyl, optionally substituted C₂₋₃₀alkynyl, optionally substituted aryl, optionally substituted cycloalkyl, or optionally substituted benzyl group.

The compound of any one of Embodiments 41-44, wherein R^{A1} is optionally substituted C₁₋₃₀ alkyl, or

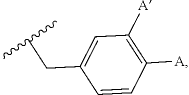

where A and A′ independently are hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl, alkoxyalkylamine, alkoxyoxycarboxylate, amino, alkylamino, dialkylamino, protected amino, a ligand, or a linker covalently bonded to one or more ligands.

The compound of any one of Embodiments 41-45, wherein R^{A1} is methyl, isopropyl, or cyclopropyl.

The compound of any one of Embodiments 41-45, wherein R^{A1} is

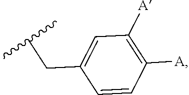

where: (i) A is CH₂CO₂Me and A′ is H; (ii) A is H and A′ is CH₂CO₂Me; (iii) A and A′ each are CH₂CO₂Me; (iv) A is CO₂Me and A′ is H; (v) A is H and A′ is CO₂Me; or (vi) A and A′ each are CO₂Me.

The compound of any one of Embodiments 41-47, wherein R^{A2} is hydrogen.

The compound of any one of Embodiments 41-47, wherein R^{A2} is a nitrogen protecting group.

The compound of any one of Embodiments 41-49, wherein R²² is hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support;
or R²² and R⁴ taken together are 4′-C(R¹⁰R¹¹)ᵥ—Y-2′ or 4′-Y—C(R¹⁰R¹¹)ᵥ-2′.

The compound of any one of Embodiments 41-50, wherein R²² is hydrogen, hydroxyl, halogen, protected hydroxyl, phosphate group, reactive phosphorous group, optionally substituted C₁₋₃₀ alkyl, optionally substituted C₂₋₃₀ alkenyl, optionally substituted C₂₋₃₀ alkynyl, optionally substituted C₁₋₃₀alkoxy (e.g., methoxy, 2-methoxyethoxy), alkoxyalkyl (e.g., methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, a solid support, a linker or a linker covalently attached to a solid support; or R²² and R⁴ taken together are 4′-C(R¹⁰R¹¹)ᵥ—Y-2′ or 4′-Y—C(R¹⁰R¹¹)ᵥ-2′.

The compound of any one of Embodiments 41-51, wherein R²² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl), C₆₋₂₄alkoxy (e.g., n-C₆₋₂₄ alkoxy), a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite), a solid support, a linker or a linker covalently attached to a solid support;
or R²² and R⁴ taken together are 4′-C(R¹⁰R¹¹)ᵥ—Y-2′ or 4′-Y—C(R¹⁰R¹¹)ᵥ-2′.

The compound of any one of Embodiments 41-52, wherein, R²² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl), C₆₋₂₄alkoxy (e.g., n-C₆₋₂₄ alkoxy), or a linker covalently attached to a solid support; or R²² and R⁴ taken together are 4′-C(R¹⁰R¹¹)ᵥ—Y-2′ or 4′-Y—C(R¹⁰R¹¹)ᵥ-2′.

The compound of any one of Embodiments 41-53, wherein R²² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl), or C₆₋₂₄alkoxy (e.g., n-C₆₋₂₄ alkoxy); or R²² and R⁴ taken together are 4′-C(R¹⁰R¹¹)ᵥ—Y-2′ or 4′-Y—C(R¹⁰R¹¹)ᵥ-2′.

The compound of any one of Embodiments 41-54, wherein R²² is R²² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl), or C₆₋₂₄alkoxy (e.g., n-C₆₋₂₄ alkoxy).

The compound of any one of Embodiments 41-55, wherein R⁴ is H.

The compound of any one of Embodiments 41-56, wherein R²³ is hydrogen, hydroxyl, protected hydroxyl, a reactive phosphorous group, a solid support, a linker, or a linker covalently attached to a solid support.

The compound of any one of Embodiments 41-57, wherein R²³ is hydrogen, hydroxyl or protected hydroxyl.

The compound of any one of Embodiments 41-58, wherein R²³ is a reactive phosphorous group, a solid support, a linker, or a linker covalently attached to a solid support.

The compound of any one of Embodiments 41-59, wherein R²³ is a reactive phosphorous or a linker covalently attached to a solid support.

The compound of any one of Embodiments 41-60, wherein R²³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite).

The compound of any one of Embodiments 41-61, wherein R²⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic.

The compound of any one of Embodiments 41-62, wherein R²⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

The compound of any one of Embodiments 41-63, wherein R²⁵ is hydroxyl or protected hydroxyl.

The compound of any one of Embodiments 41-64, wherein: R²² is hydrogen, hydroxyl, protected hydroxyl (e.g., tert-butyldimethylsilyl protected), fluoro, methoxy, 2-methoxyethoxy, —O—N-methylacetamido or C₆₋₂₄alkoxy; R²³ is hydrogen, hydroxyl, protected hydroxyl (e.g., tert-butyldimethylsilyl protected), a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite), a solid support, a linker, or a linker covalently attached to a solid support; R⁴ is H; and R²⁵ is hydroxyl or protected hydroxyl (e.g., 4,4′-dimethoxytrityl-protected).

The compound of any one of Embodiments 41-64, wherein: R²² is hydrogen, hydroxyl, protected hydroxyl (e.g., tert-butyldimethylsilyl protected), a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite), a solid support, a linker, or a linker covalently attached to a solid support; R²³ is hydrogen, hydroxyl, protected hydroxyl (e.g., tert-butyldimethylsilyl protected), fluoro, methoxy, 2-methoxyethoxy, —O—N-methylacetamido or C₆₋₂₄alkoxy; R⁴ is H; and R²⁵ is hydroxyl or protected hydroxyl (e.g., 4,4′-dimethoxytrityl-protected protected).

The compound of any one of Embodiments 41-63, wherein: R²³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R²⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates, or a phosphate mimic.

The compound of any one of Embodiments 41-63, wherein: R²³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R²⁵ is vinylphosphonate (VP) group, cyclopropylphosphonate, or a phosphate mimic.

The compound of any one of Embodiments 41-63, wherein: R²³ is a phosphoramidite group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R²⁵ is vinylphosphonate (VP) group.

The compound of any one of Embodiments 67-68, wherein R²² is hydrogen, hydroxyl, protected hydroxyl, fluoro, chloro, methoxy, ethoxy, 2-methoxyethyl, —O—N-methylacetamido, C₆₋₂₄alkoxy or C₆₋₂₄ alkyl (e.g., n-C₆₋₂₄ alkyl).

The compound of any one of Embodiments 41-63, wherein: R²² is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R²⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates, or a phosphate mimic.

The compound of any one of Embodiments 41-63, wherein: R²² is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R²⁵ is vinylphosphonate (VP) group, cyclopropylphosphonate, or a phosphate mimic.

The compound of any one of Embodiments 41-63, wherein: R²² is a phosphoramidite group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); and R²⁵ is vinylphosphonate (VP) group.

The compound of any one of Embodiments 71-73, wherein R²³ is hydrogen, hydroxyl, protected fluoro, chloro, methoxy, ethoxy, 2-methoxyethyl, —O—N-methylacetamido, C₆₋₂₄alkoxy or C₆₋₂₄ alkyl (e.g., n-C₆₋₂₄ alkyl).

The compound of any one of Embodiments 67-74, wherein R⁴ is H.

The compound of any one of Embodiments 41-63, wherein: R²³ is hydrogen, hydroxyl, protected hydroxyl (e.g., tert-butyldimethylsilyl protected), a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite), a solid support, a linker, or a linker covalently attached to a solid support; R²⁵ is hydroxyl or protected hydroxyl (e.g., 4,4′-dimethoxytrityl-protected); and R⁴ and R²² taken together are 4′-C(R¹⁰R¹¹)ᵥ—Y-2′.

The compound of any one of Embodiments 41-63, wherein: R²³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²⁵ is hydroxyl or protected hydroxyl (e.g., 4,4′-dimethoxytrityl-protected); and R⁴ and R²² taken together are 4′-C(R¹⁰R¹¹)ᵥ—Y-2′.

The compound of any one of Embodiments 41-63, wherein: R²³ is a phosphoramidite group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²⁵ is hydroxyl or protected hydroxyl (e.g., 4,4′-dimethoxytrityl-protected); and R⁴ and R²² taken together are 4′-C(R¹⁰R¹¹)ᵥ—Y-2′.

The compound of any one of Embodiments 41-63, wherein: R²³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates, or a phosphate mimic; and R⁴ and R²² taken together are 4′-C(R¹⁰R¹¹)ᵥ—Y-2′

The compound of any one of Embodiments 41-63, wherein: R²³ is hydrogen, hydroxyl, protected hydroxyl (e.g., tert-butyldimethylsilyl protected), a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates, or a phosphate mimic; and R⁴ and R²² taken together are 4′-C(R¹⁰R¹¹)ᵥ—Y-2′.

The compound of any one of Embodiments 41-63, wherein: R²³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²⁵ is vinylphosphonate (VP) group, cyclopropylphosphonate, or a phosphate mimic; and R⁴ and R²² taken together are 4′-C(R¹⁰R¹¹)ᵥ—Y-2′.

The compound of any one of Embodiments 41-63, wherein: R²³ is a phosphoramidite group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R²⁵ is vinylphosphonate (VP) group; and R⁴ and R²² taken together are 4′-C(R¹⁰R¹¹)ᵥ—Y-2′.

The compound of any one of Embodiments 76-82, wherein Y is O.

The compound of any one of Embodiments 76-83, wherein v is 1 or 2.

The compound of any one of Embodiments 76-84, wherein one of R¹⁰ and R¹¹ is H and the other is H or C₁-C₆alkyl (e.g., methyl, ethyl, propyl or isopropyl).

An oligonucleotide prepared using a compound of any one of Embodiments 41-85.

A compound of Formula (III).

The compound of Embodiment 87, wherein R^{A2} is hydrogen

The compound of Embodiment 87, wherein R^{A2} is a nitrogen protecting group.

The compound of Embodiment 89, wherein the nitrogen protecting group is-C(═O)R^{NP1}, wherein R^{NP1} is C1-10 alkyl, C1-10 perhaloalkyl, C2-10 alkenyl, C2-10 alkynyl, heteroC1-10 alkyl, heteroC2-10alkenyl, heteroC2-10alkynyl, C3-10 carbocyclyl, 3-14 membered heterocyclyl, C6-14 aryl, or 5-14 membered heteroaryl, wherein each alkyl, alkenyl, alkynyl, carbocyclyl, heterocyclyl, aralkyl, aryl, and heteroaryl of R^{P1} is optionally substituted with 1, 2, 3, 4 or 5 substituents independently selected from OH, CN, SC(O)Ph, oxo (═O), SH, SO2NH2, SO2(C1-C4)alkyl, SO2NH(C1-C4)alkyl, halogen, carbonyl, thiol, cyano, NH2, NH(C1-C4)alkyl, N[(C1-C4)alkyl]2, C(O)NH2, COOH, COOMe, acetyl, (C1-C8)alkyl, O(C1-C8)alkyl (i.e., C1-C8alkoxy), O(C1-C8)haloalkyl, (C2 C8)alkenyl, (C2-C8)alkynyl, haloalkyl, thioalkyl, cyanomethylene, alkylaminyl, aryl, heteroaryl, substituted aryl, NH2-C(O)-alkylene, NH(Me)-C(O)-alkylene, CH2-C(O)-alkyl, C(O)-alkyl, alkylcarbonylaminyl, CH2-[CH(OH)]m (CH2)p-OH, CH₂—[CH(OH)]ₘ—(CH2)p-NH2 or CH2-aryl-alkoxy, where “m” and “p” are independently 1, 2, 3, 4, 5 or 6.

Embodiment 91: The compound of Embodiment 89, wherein the nitrogen protecting group is formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxy acylamino)acetamide, 3-(p-hydroxylphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Embodiment 92: The compound of Embodiment 89, wherein the nitrogen protecting group is benzoyl.

Embodiment 93: The compound of any one of Embodiments 87-92, wherein R²⁵ is —OR^{Pro}, wherein R^{Pro} is an oxygen protecting group.

Embodiment 94: The compound of Embodiment 93, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

Embodiment 95: The compound of Embodiment 93, wherein R^{Pro} is 4,4′-dimethoxytrityl.

Embodiment 96: The compound of any one of Embodiments 87-95, wherein Y^{A} is CH.

Embodiment 97: The compound of any one of Embodiments 87-95, wherein Y^{A} is N.

Embodiment 98: The compound of any one of Embodiments 87-97, wherein one of R²² and R²³ is protected hydroxyl, halogen, optionally substituted C₁₋₃₀ alkyl, or optionally substituted C₁₋₃₀ alkoxy (e.g., methoxy, 2-methoxyethoxy); and the other of R²² and R²³ is a reactive phosphorous group.

Embodiment 99: The compound of any one of Embodiments 87-97, wherein one of R²² and R²³ is halogen or optionally substituted C₁₋₃₀ alkoxy; and the other of R²² and R²³ is a reactive phosphorous group.

Embodiment 100: The compound of any one of Embodiments 87-97, wherein one of R²² and R²³ is fluoro, methoxy, or 2-methoxyethoxy; and the other of R²² and R²³ is a reactive phosphorous group.

Embodiment 101: The compound of any one of Embodiments 98-100, wherein the reactive phosphorous group is —OP(OR)(N(R^{P2})₂), —OP(SR)(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR)H, —OP(S)(OR^{P})H, —OP(O)(SR)H, —OP(O)(OR^{P})R^{P3}, —OP(S)(OR^{P})R^{P3}, or —OP(O)(SR^{P})R^{P3}.

Embodiment 102: The compound of any one of Embodiments 98-100, wherein the reactive phosphorous group is —OP(OR^{P})(N(R^{P2})₂).

Embodiment 103: The compound of any one of Embodiments 98-100, wherein the reactive phosphorous group is OP(OR)(N(R^{P2})₂), wherein R^{p} is cyanoethyl (—CH₂CH₂CN) and each R^{P2} is isopropyl or both R^{P2} taken together with the nitrogen atom to which they are attached form an optionally substituted 3-8 membered heterocyclyl.

Embodiment 104: The compound of any one of Embodiments 98-103, wherein R²² is the reactive phosphorous group.

Embodiment 105: The compound of any one of Embodiments 98-103, wherein R²³ is the reactive phosphorous group.

Embodiment 106: The compound of any one of Embodiments 87-97, wherein one of R²² and R²³ is protected hydroxyl, halogen, optionally substituted C₁₋₃₀ alkyl, or optionally substituted C₁₋₃₀ alkoxy (e.g., methoxy, 2-methoxyethoxy); and the other of R²² and R²³ is a protected hydroxyl.

Embodiment 107: The compound of any one of Embodiments 87-97, wherein one of R²² and R²³ is halogen or optionally substituted C₁₋₃₀ alkoxy; and the other of R²² and R²³ is a protected hydroxyl.

Embodiment 108: The compound of any one of Embodiments 87-97, wherein one of R²² and R²³ is fluoro, methoxy, or 2-methoxyethoxy; and the other of R²² and R²³ is a protected hydroxyl.

Embodiment 109: The compound of any one of Embodiments 106-108, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

Embodiment 110: The compound of any one of Embodiments 106-108, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

Embodiment 111: The compound of any one of Embodiments 106-108, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

Embodiment 112: The compound of any one of Embodiments 106-111, wherein R²² is the protected hydroxyl group.

Embodiment 113: The compound of any one of Embodiments 106-111, wherein R²³ is the protected hydroxyl group.

Embodiment 114: The compound of any one of Embodiments 87-97, wherein one of R²² and R²³ is protected hydroxyl, halogen, optionally substituted C₁₋₃₀ alkyl, or optionally substituted C₁₋₃₀ alkoxy (e.g., methoxy, 2-methoxyethoxy); and the other of R²² and R²³ is hydroxyl.

Embodiment 115: The compound of any one of Embodiments 87-97, wherein one of R²² and R²³ is halogen or optionally substituted C₁₋₃₀ alkoxy; and the other of R²² and R²³ is a hydroxyl.

Embodiment 116: The compound of any one of Embodiments 87-97, wherein one of R²² and R²³ is fluoro, methoxy, or 2-methoxyethoxy; and the other of R²² and R²³ is a hydroxyl.

Embodiment 117: The compound of any one of Embodiments 114-116, wherein R²² is the hydroxyl group.

Embodiment 118: The compound of any one of Embodiments 114-116, wherein R²³ is the hydroxyl group.

Embodiment 119: The compound of any one of Embodiments 87-97, wherein R⁴ and R²² taken together are 4′-C(R¹⁰R¹¹)ᵥ—Y-2′ or 4′-Y—C(R¹⁰R¹¹)ᵥ-2′; and R²³ is a reactive phosphorous group, protected hydroxyl or hydroxyl group.

Embodiment 120: The compound of Embodiment 119, wherein in R⁴ and R²² taken together are —CH₂—Y-2′ or 4′-Y—CH²-2′.

Embodiment 121: The compound of Embodiment 119, wherein in R⁴ and R²² taken together are —CH₂—O-2′ or 4′-O—CH²-2′.

Embodiment 122: The compound of any one of Embodiments 119-121, wherein R²³ is a hydroxyl group.

Embodiment 123: The compound of any one of Embodiments 119-121, wherein R²³ is a protected hydroxyl.

Embodiment 124: The compound of Embodiment 123, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

Embodiment 125: The compound of Embodiment 123, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

Embodiment 126: The compound of Embodiment 123, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

Embodiment 127: The compound of any one of Embodiments 119-121, wherein R²³ is a R²³ is a reactive phosphorous group.

Embodiment 128: The compound of Embodiment 127, wherein the reactive phosphorous group is —OP(OR)(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR)H, —OP(S)(OR^{P})H, —OP(O)(SR)H, —OP(O)(OR^{P})R^{P3}, —OP(S)(OR^{P})R^{P3}, or —OP(O)(SR^{P})R^{P3}.

Embodiment 129: The compound of Embodiment 127, wherein the reactive phosphorous group is —OP(OR^{P})(N(R^{P2})₂).

Embodiment 130: The compound of Embodiment 127, wherein the reactive phosphorous group is OP(OR)(N(R^{P2})₂), wherein R^{p} is cyanoethyl (—CH₂CH₂CN) and each R² is isopropyl or both R^{P2} taken together with the nitrogen atom to which they are attached form an optionally substituted 3-8 membered heterocyclyl.

### Some Selected Definitions

For convenience, certain terms employed herein, in the specification, examples and appended claims are collected herein. Unless stated otherwise, or implicit from context, the following terms and phrases include the meanings provided below Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxyl, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxyl, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and N(aryl)₂. The term “heteroarylamino” means a nitrogen moiety having at least one heteroaryl radical attached to the nitrogen. For example —NHheteroaryl, and N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, NHCH₂CH₃, —NHCH₂CH₂CH₃, and —NHCH(CH₃)₂. Exemplary dialkylamino includes, but is not limited to, N(C₁-C₁₀alkyl)₂, such as N(CH₃)₂, N(CH₂CH₃)₂, N(CH₂CH₂CH₃)₂, and N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxyl” and “hydroxyl” mean the radical OH.

The terms “alkoxyl” or “alkoxy” as used herein refers to an alkyl group, as defined above, having an oxygen radical attached thereto, and can be represented by one of —O-alkyl, —O— alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical C(O)—. It is noted that the carbonyl radical can be further substituted with a variety of substituents to form different carbonyl groups including acids, acid halides, amides, esters, ketones, and the like.

As used herein, the term “oxo” means double bonded oxygen, i.e., ═O.

The term “carboxy” means the radical C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO3H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′R^{N}—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxyl, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxylalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚNH₂ or CH₂-aryl-alkoxy; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of assays or therapeutic treatment, or in the case of in vitro assays, under conditions in which the assays are performed. The non-target sequences typically differ by at least 5 nucleotides.

In some embodiments, a dsRNA molecule of the invention is “sufficiently complementary” to a target RNA, e.g., a target mRNA, such that the dsRNA molecule silences production of protein encoded by the target mRNA. In another embodiment, the dsRNA molecule of the invention is “exactly complementary” to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA molecule of the invention specifically discriminates a single-nucleotide difference. In this case, the dsRNA molecule only mediates RNAi if exact complementary is found in the region (e.g., within 7 nucleotides of) the single-nucleotide difference.

The term ‘BNA’ refers to bridged nucleic acid, and is often referred as constrained or inaccessible RNA. BNA can contain a 5-, 6-membered, or even a 7-membered bridged structure with a “fixed” C3′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

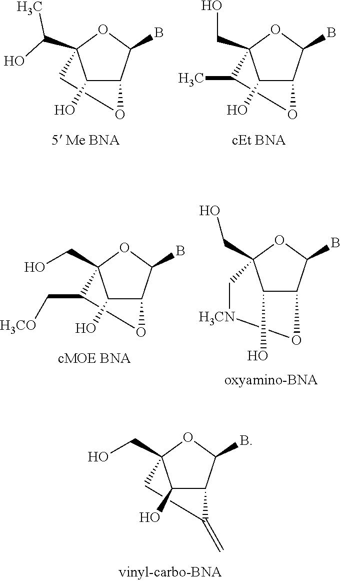

The term ‘LNA’ refers to locked nucleic acid, and is often referred as constrained or inaccessible RNA. LNA is a modified RNA nucleotide. The ribose moiety of an LNA nucleotide is modified with an extra bridge (e.g., a methylene bridge or an ethylene bridge) connecting the 2′ hydroxyl to the 4′ carbon of the same ribose sugar. For instance, the bridge can “lock” the ribose in the 3′-endo North) conformation:

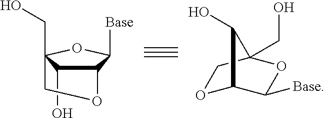

The term ‘ENA’ refers to ethylene-bridged nucleic acid, and is often referred as constrained or inaccessible RNA.

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, a “terminal region” of a strand refers to positions 1-4, e.g., positions 1, 2, 3, and 4, counting from the nearest end of the strand. For example, a 5′-terminal region refers to positions 1-4, e.g., positions 1, 2, 3 and 4 counting from the 5′-end of the strand. Similarly, a 3′-terminal region refers to positions 1-4, e.g., positions 1, 2, 3 and 4 counting from the 3′-end of the strand.

For example, a 5′-terminal region for the antisense strand is positions 1, 2, 3 and 4 counting from the 5′-end of the antisense strand. A preferred 5′-terminal region for the antisense strand is positions 1, 2 and 3 counting from the 5′-end of the antisense strand. A 3′-terminal region for the antisense strand can be positions 1, 2, 3, and 4 counting from the 3′-end of the strand. A preferred 3′-terminal region for the antisense strand is positions 1, 2 and 3 counting from the 3′-end of the antisense strand.

Similarly, a 5′-terminal region for the sense strand is positions 1, 2, 3 and 4 counting from the 5′-end of the sense strand. A preferred 5′-terminal region for the sense strand is positions 1, 2 and 3 counting from the 5′-end of the sense strand. A 3′-terminal region for the sense strand can be positions 1, 2, 3, and 4 counting from the 3′-end of the strand. A preferred 3′-terminal region for the sense strand is positions 1, 2 and 3 counting from the 3′-end of the sense strand.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

### EXAMPLES

The invention is further illustrated by the following examples, which should not be construed as further limiting. The contents of all references, pending patent applications and published patents, cited throughout this application are hereby expressly incorporated by reference.

### Example 1: siRNAs Modified at the 2′ Position and with N6-Methyl Adenosine (m6A)

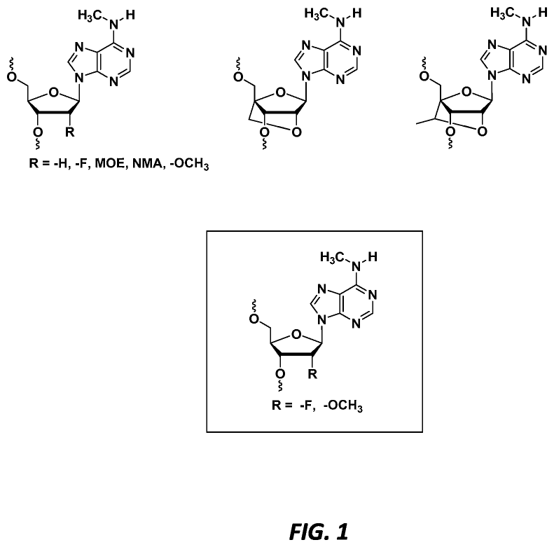

N(6)-methyladenosine (m(6)A) is a known DNA and RNA natural modification and has important links to epigenetic and epitranscriptomic regulation¹⁻². Such natural modifications will reduce immune response and toxicity if used in synthetic nucleic acid drugs. Inventors have studied the effects of this natural base modification along with common sugar modifications 2′-O-methyl (2′-OMe) and 2′-fluoro (2′-F) (FIG. 1) in therapeutically relevant siRNA duplexes. Described herein are the synthesis, biophysical properties, and RNAi activity of siRNAs modified 2′-OMe m6A and 2′-F m6A nucleotides.

The 2′-F- and 2′-OMe-modified m6A residues are thermodynamically less destabilizing in RNA:RNA duplexes than the DNA:DNA duplexes, compared to the non-methylated analogue as predicted from their sugar puckers³. When incorporated at the terminus of an oligonucleotide, 2′-F m6A had comparable 5′-exonuclease stability to 2′-F adenosine, whereas 2′-F m6A and 2′-OMe m6A were less resistant to 3′-exonuclease than the parent 2′-modified adenosine analogs. Thus, phosphorothioate linkages were needed to maintain nuclease stability when these m6As were incorporated at a terminal residue. Interestingly, 2′-modified m6A could be incorporated at any of the adenosine residues in the guide and passenger strands of a N-acetylgalactosamine-conjugated siRNA duplex targeting three different gene targets (mouse TTR, C5, and β-Catenin) without loss of RNAi activity. Thus, 2′-F- and 2′-OMe-modified m6A does not interfere with catalytic activity of the RNAi machinery⁴⁻⁵.

General synthetic routes are shown in the following schemes:

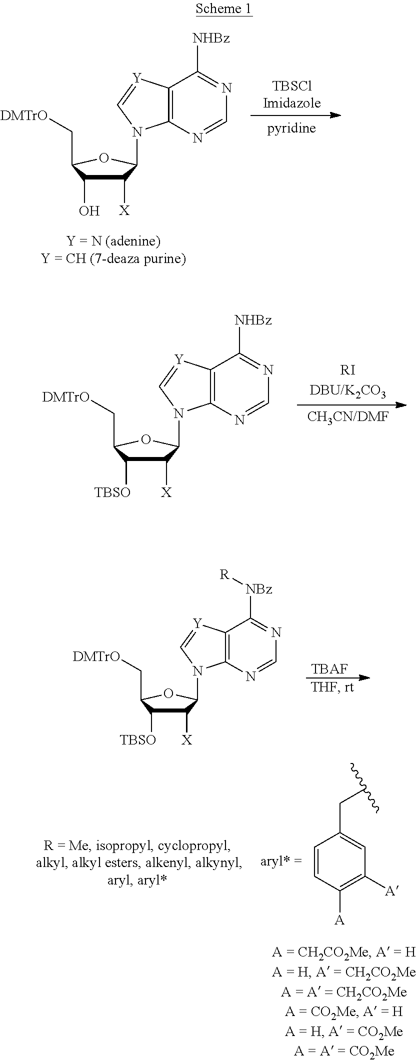

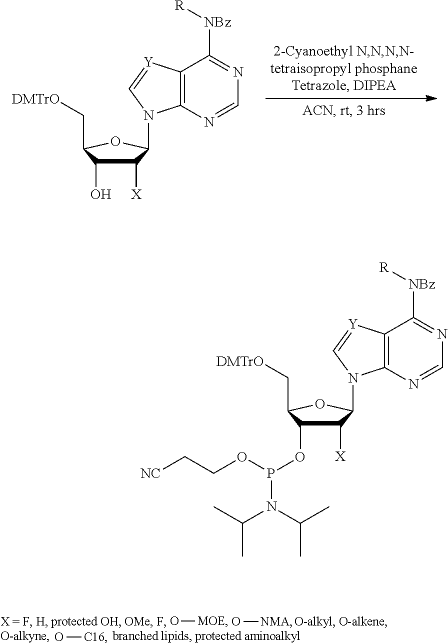

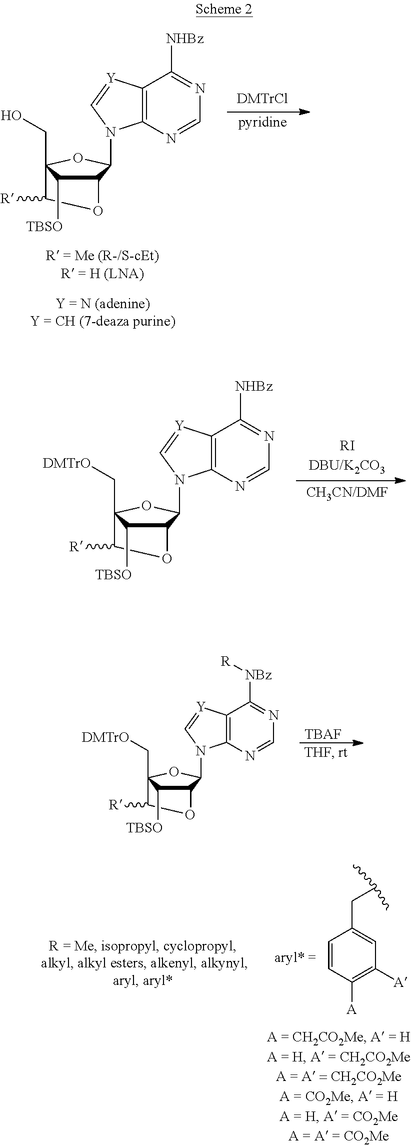

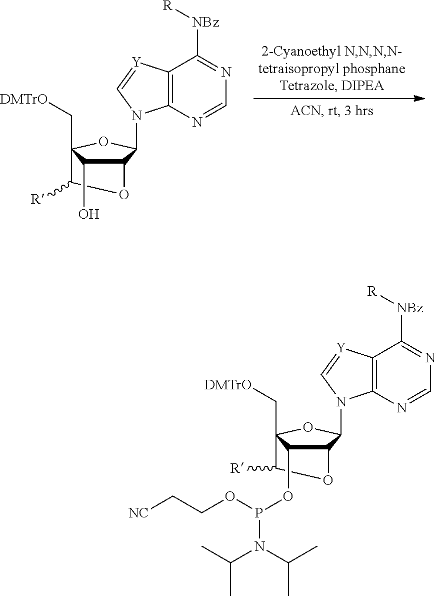

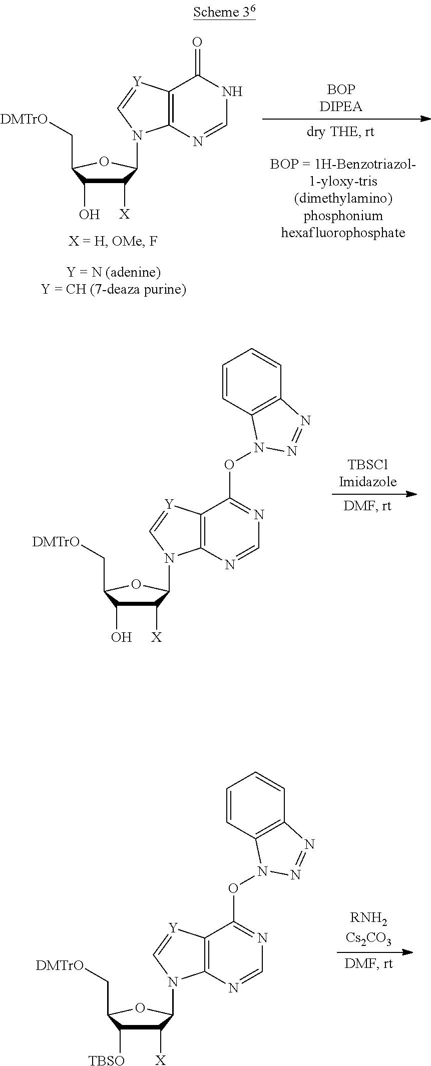

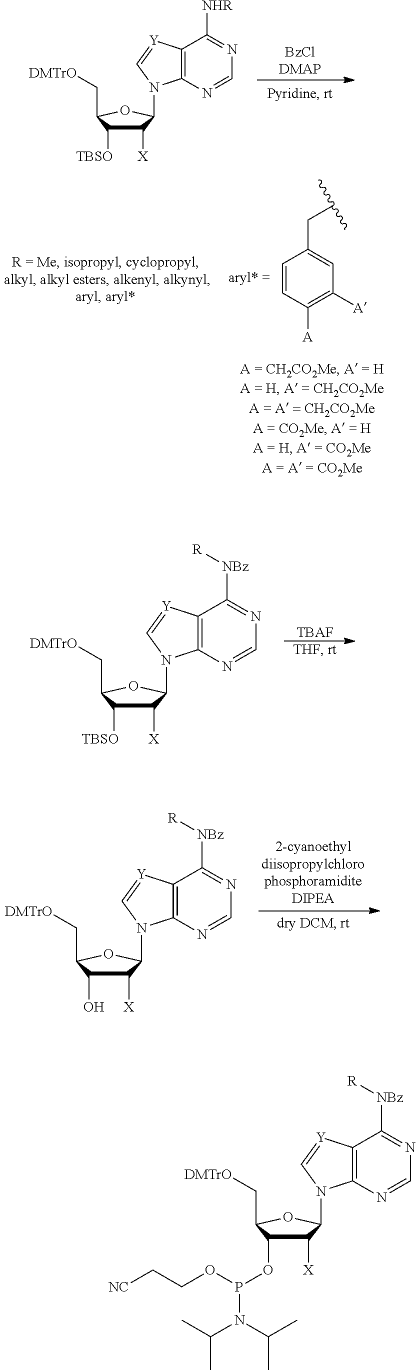

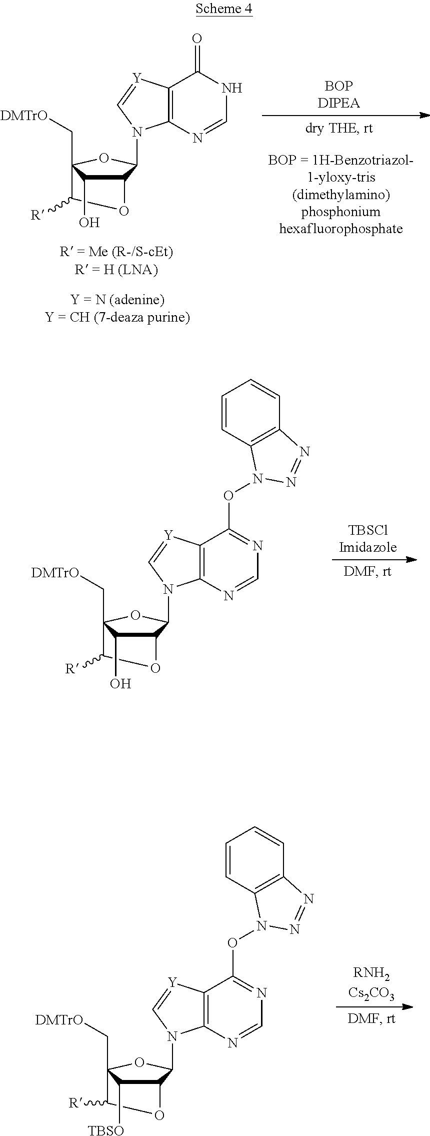

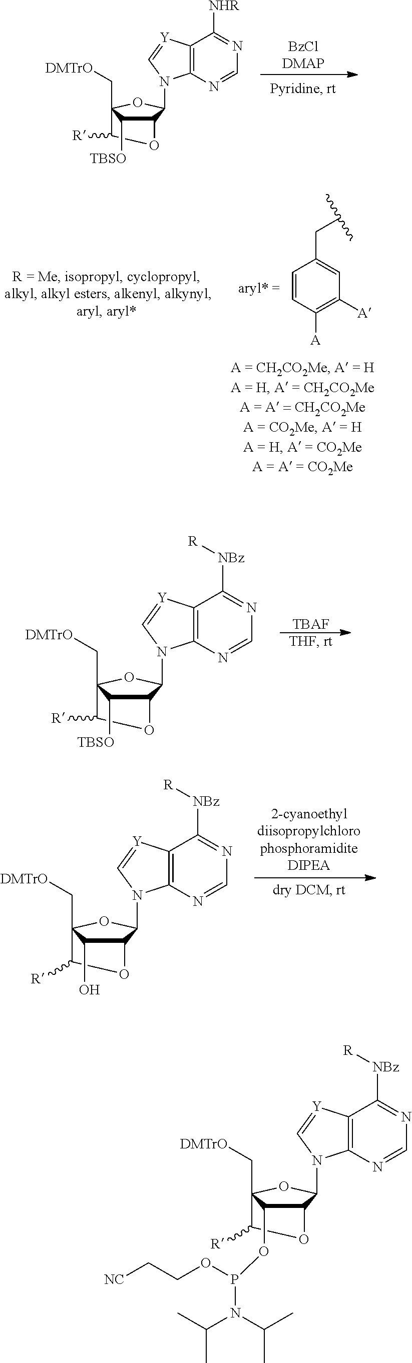

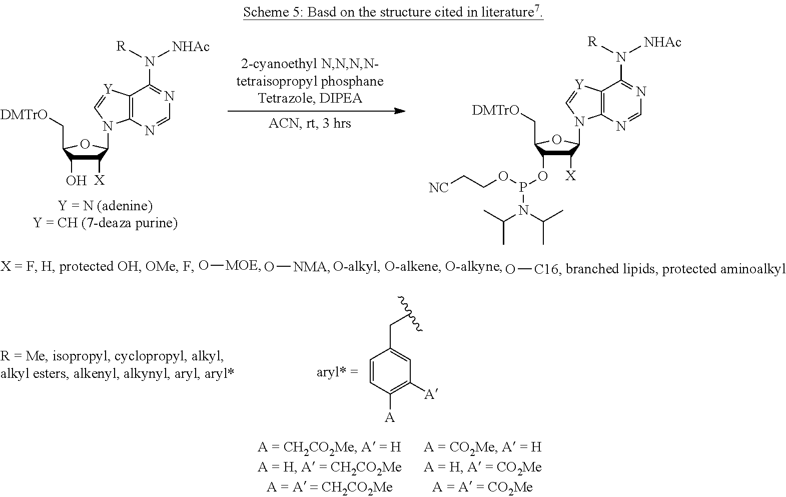

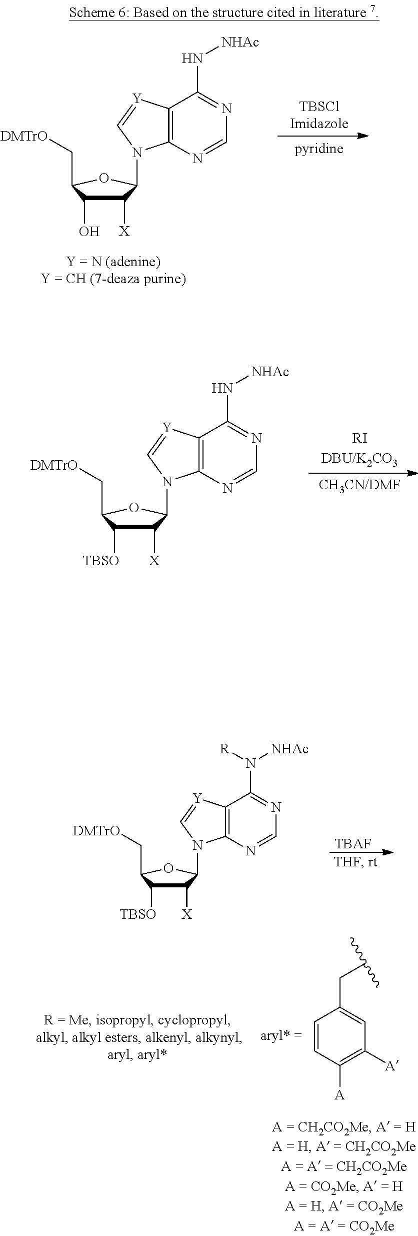

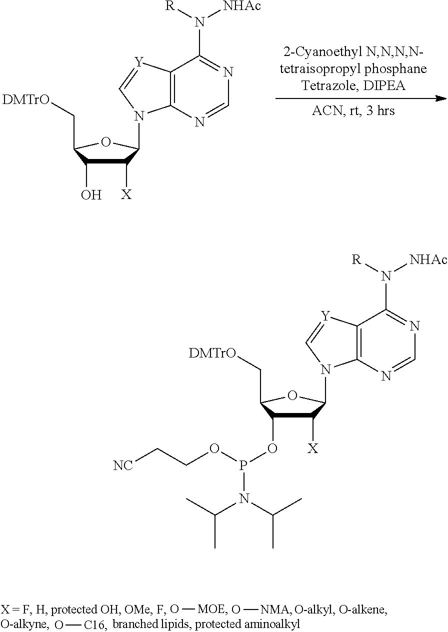

### Monomer Synthesis:

3′-Hydroxy group of commercially available nucleoside 1⁸ was protected by tert-butyldimethylsilyl (TBS) group to afford 2 in good yield. Compound 2 was then reacted with iodomethane (MeI) under basic condition to obtain 3 in moderate yield. Deprotection of TBS groups from compound 3 with tetra-butylammonium fluoride (TBAF) afforded compound 4 in quantitative yield. Finally, phosphitylation reaction of compound 4 produced the desired amidite 5 in moderate yield (Scheme 7).

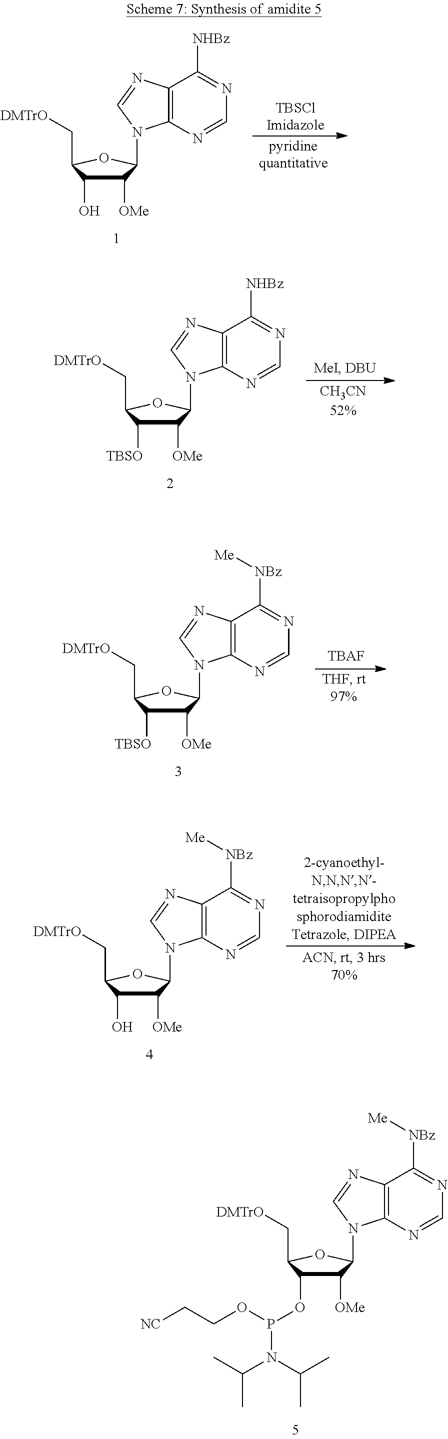

3′-hydroxy group of commercially available nucleoside 6 was protected by TBS groups to afford 7 in quantitative yield. N6 methylation of 7 was successfully achieved after treating with MeI under basic condition to obtain compound 8. Removal of TBS group from compound 8 with TBAF afforded 9 in quantitative yield. Phosphitylation reaction of compound 9 produced the desired amidite 10 in good yield (Scheme 8).

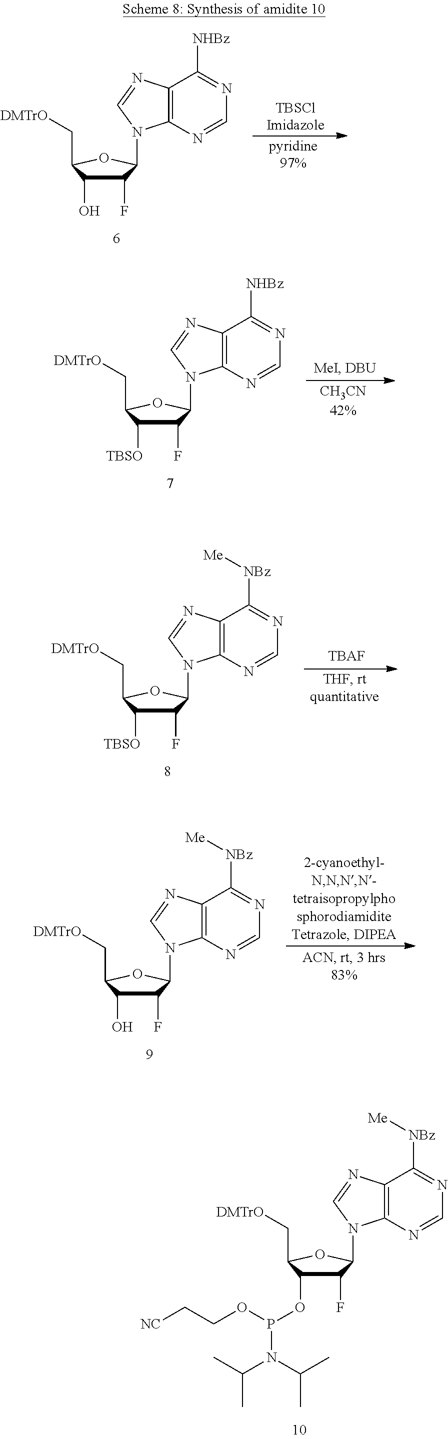

To check the effect of sterically hindered alkyl group at N6 position on thermodynamic destabilization, the N6-ⁱPr analogue of amidite 5 was synthesized. N6-position of compound 2 was alkylated with 2-iodopropane under basic condition. Here, poor yield in alkylation step prompted us to change the condition from DBU to inorganic base potassium carbonate to afford 11 in moderate yield. TBS group of 11 was then removed under desilylation condition with TBAF to obtain 12 in good yield. Phosphitylation reaction of 12 with 2-cyanoethyl-N,N-diisopropylchlorophosphoramidite, afforded the amidite 13 (Scheme 9).

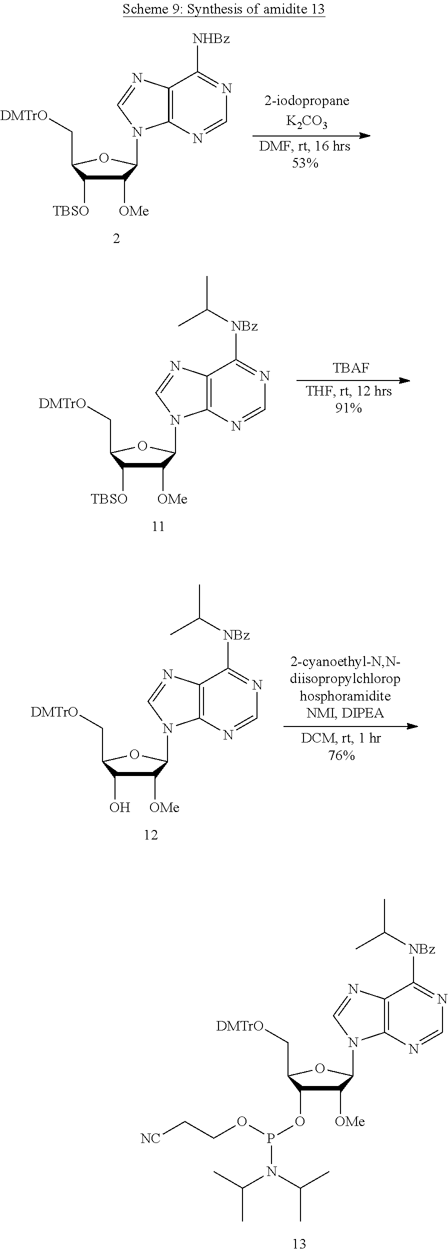

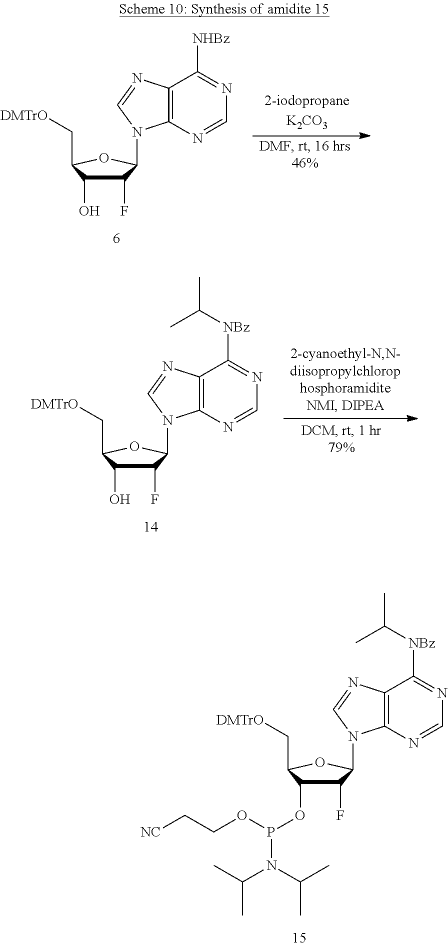

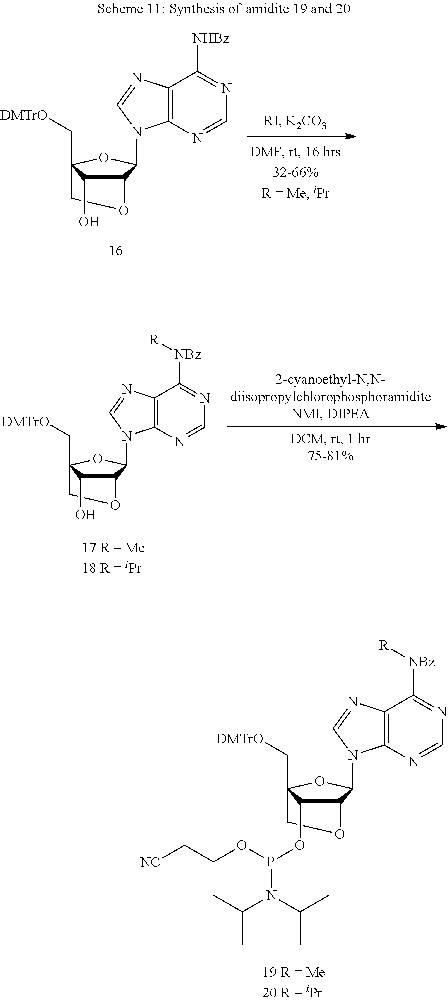

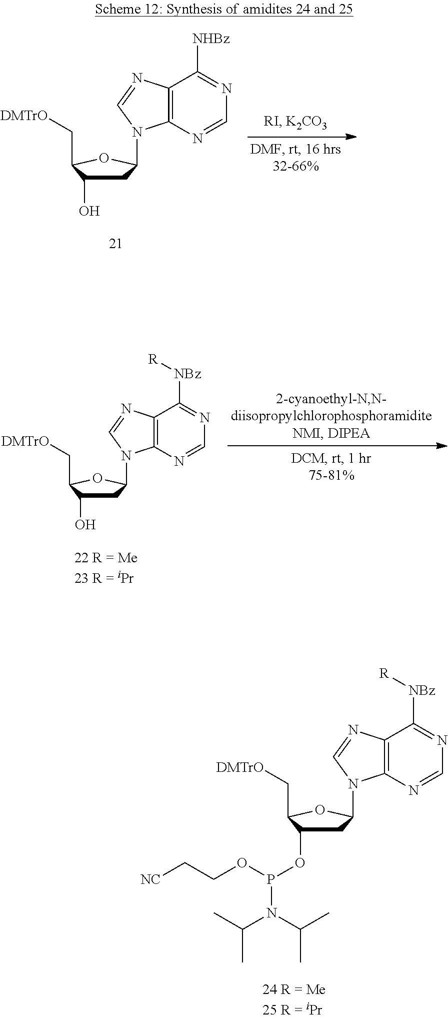

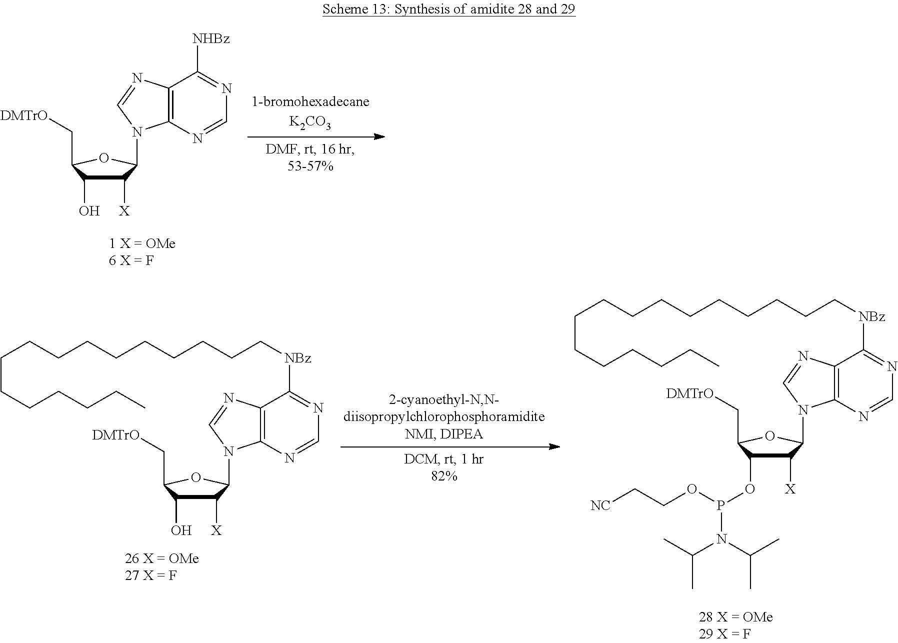

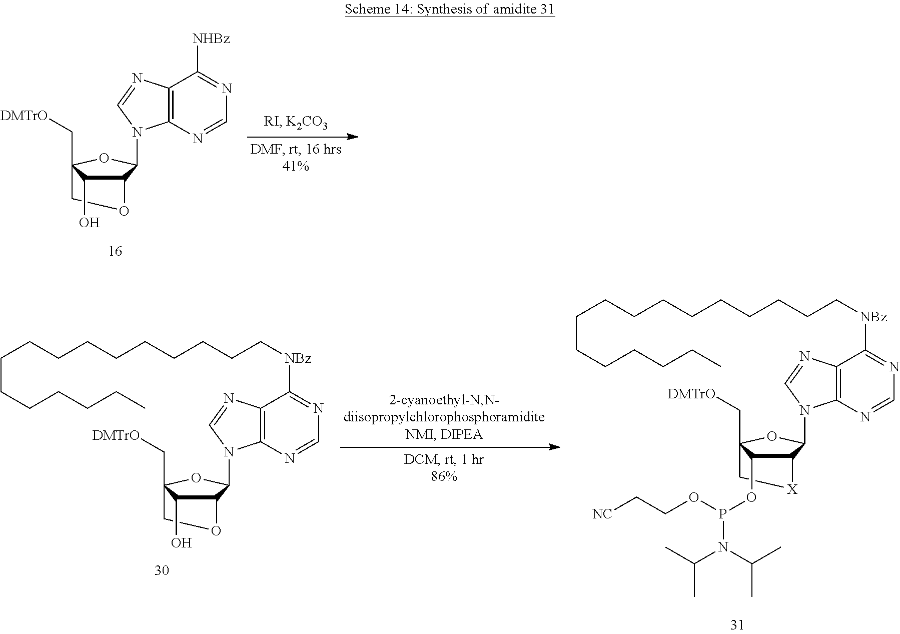

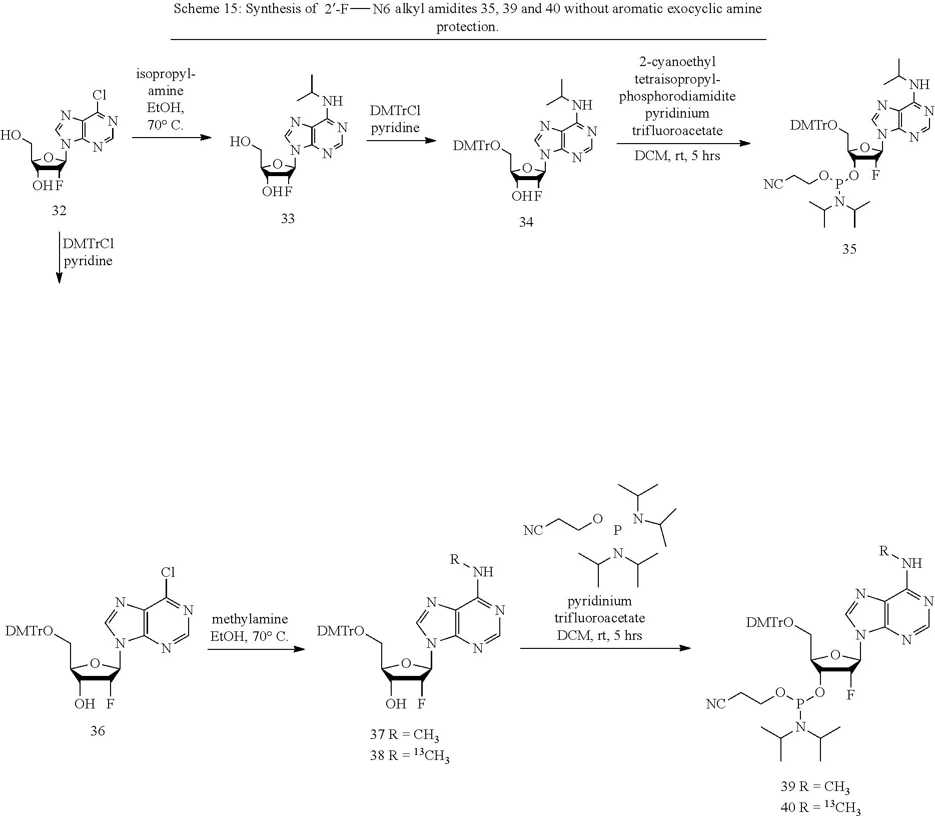

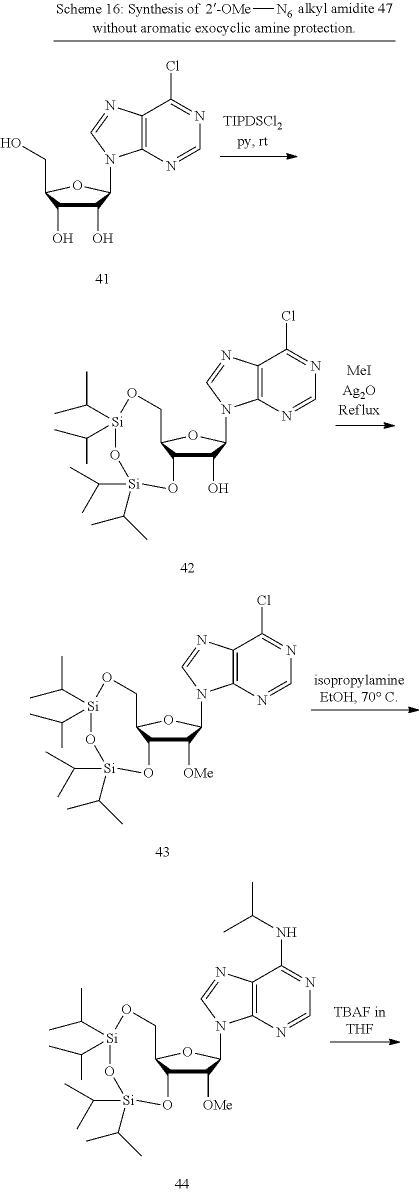

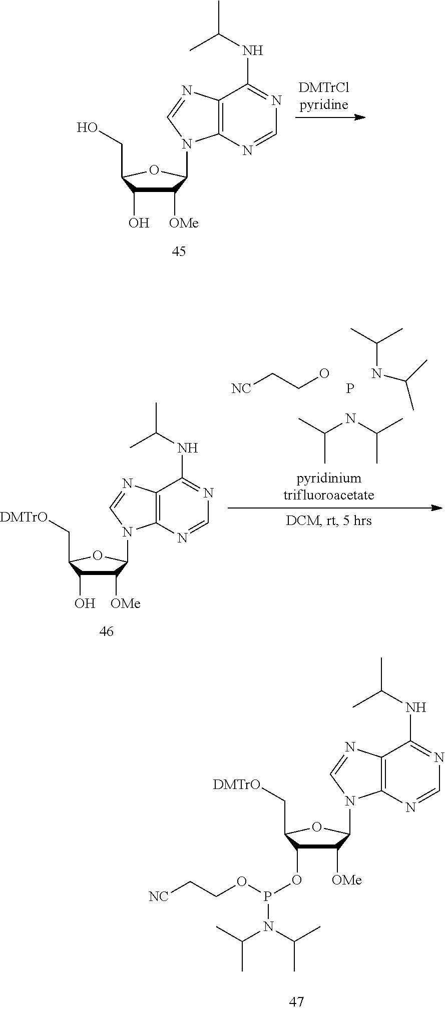

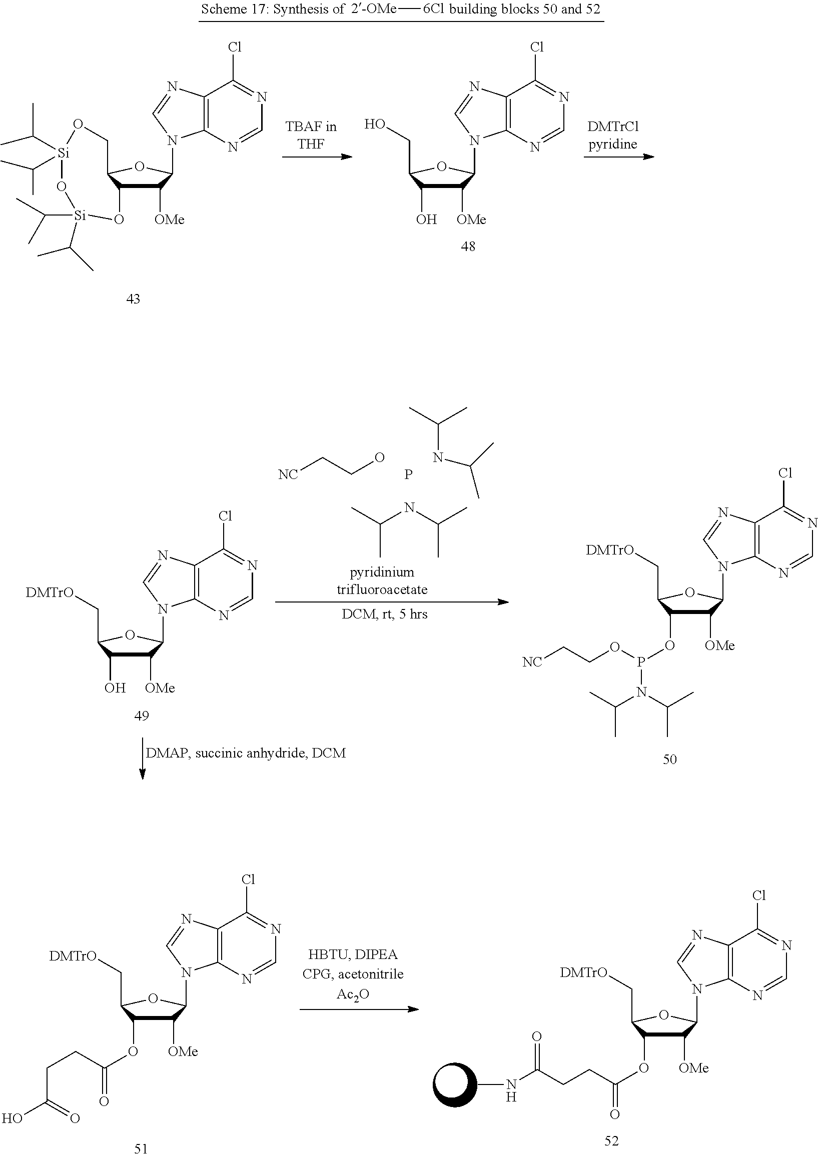

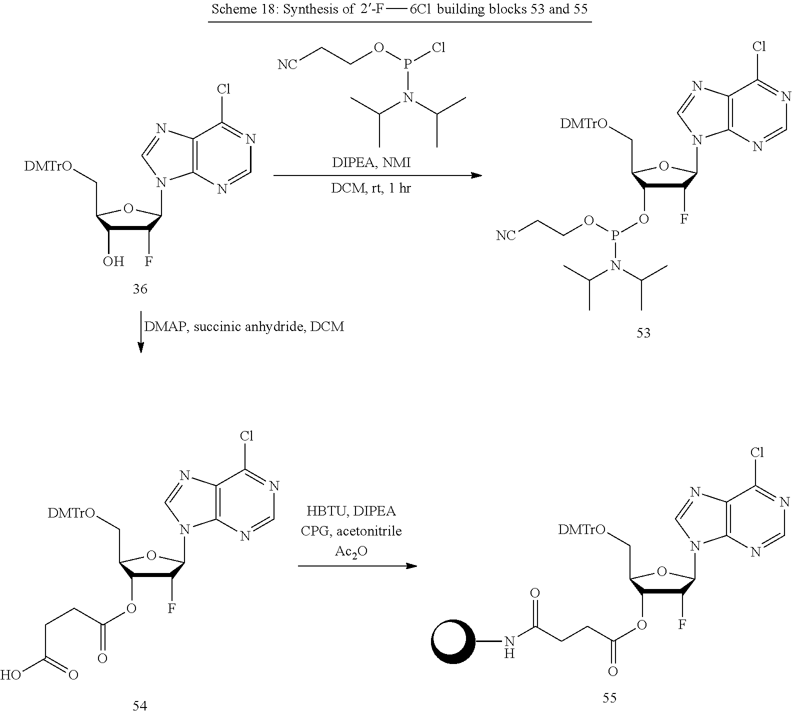

### Synthetic Procedures and Compound Characterization

General conditions: TLC was performed on Merck silica gel 60 plates coated with F254. Compounds were visualized under UV light (254 nm) or after spraying with the p-anisaldehyde staining solution followed by heating. Flash column chromatography was performed using a Teledyne ISCO Combi Flash system with pre-packed RediSep Teledyne ISCO silica gel cartridges. All moisture-sensitive reactions were carried out under anhydrous conditions using dry glassware, anhydrous solvents, and argon atmosphere. All commercially available reagents and solvents were purchased from Sigma-Aldrich unless otherwise stated and were used as received. ESI-MS spectra were recorded on a Waters Qtof Premier instrument using the direct flow injection mode. ¹H NMR spectra were recorded at 400 or 500 MHz. ¹³C NMR spectra were recorded at 101 or 126 MHz. ¹⁹F NMR spectra were recorded at 470 MHz. ³¹P NMR spectra were recorded at 202 MHz. Chemical shifts are given in ppm, coupling constants are given in Hertz and signal splitting patterns are described as singlet (s), doublet (d), triplet (t), septet (sept), broad signal (brs), or multiplet (m).

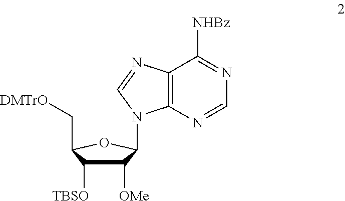

N-(9-((2R,3R,4R,5R)-5-((bis(4-methoxyphenyl)(phenyl)methoxy)methyl)-4-((tert-butyldimethylsilyl)oxy)-3-methoxytetrahydrofuran-2-yl)-9H-purin-6-yl)benzamide [Compound 2]: To a clear solution of compound 1 (10.0 g, 14.6 mmol) in dry pyridine (100 mL) was added imidazole (4.03 g, 59.2 mmol) in single portion. To the resulting mixture was added tert-butyldimethylsilyl chloride (TBSCl) (4.46 g, 29.6 mmol) at room temperature under argon atmosphere. The resulting mixture was stirred for 16 hr at room temperature after which it was poured into a mixture of hexane (300 mL), ether (100 mL) and water (600 mL) and stirred vigorously. White precipitate was collected through filtration and dissolved in dichloromethane (DCM) (500 mL). Organic layer was dried over anhydrous Na₂SO₄, filtered and filtrate was evaporated under high vacuum pump to afford compound 2 as white foam (11.6 g, quantitative). ¹H NMR (500 MHz, CDCl₃) δ 8.70 (s, 1H), 8.27 (s, 1H), 8.00 (d, J=7.5 Hz, 2H), 7.59-7.17 (m, 13H), 7.01 (d, J=3.1 Hz, OH), 6.86-6.65 (m, 4H), 6.16 (d, J=4.3 Hz, 1H), 4.52 (t, J=4.6 Hz, 1H), 4.43 (t, J=4.6 Hz, 1H), 4.24 (q, J=4.2 Hz, 1H), 3.74 (s, 6H), 3.58 (dd, J=10.9, 4.2 Hz, 1H), 3.45 (s, 3H), 3.33 (dd, J=10.8, 4.0 Hz, 1H), 0.87 (s, 9H), 0.05 (d, J=31.1 Hz, 6H) ppm. ¹³C NMR (126 MHz, CDCl₃) δ 165.16, 158.54, 152.40, 151.47, 149.71, 149.62, 144.37, 142.07, 135.99, 135.60, 135.26, 133.62, 132.62, 130.03, 128.66, 128.17, 127.95, 127.83, 126.95, 123.72, 123.71, 113.14, 87.21, 86.56, 84.42, 82.64, 70.51, 62.52, 58.48, 55.17, 25.68, 18.05, −4.68, −4.80 ppm. HRMS calc. for C₄₅H₅₂N₅O₇Si [M+H]⁺ 802.3636, found 802.3629.

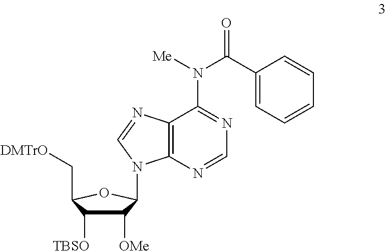

N-(9-((2R,3R,4R,5R)-5-((bis(4-methoxyphenyl)(phenyl)methoxy)methyl)-4-((tert-butyldimethylsilyl)oxy)-3-methoxytetrahydrofuran-2-yl)-9H-purin-6-yl)-N-methylbenzamide [Compound 3]: To a clear solution of 3 (11.6 g, 14.5 mmol) in dry acetonitrile (ACN) (100 mL) was added 1,8-diazabicyclo[5.4.0]undec-7-ene (DBU) (6.72 g, 44.1 mmol). To this resulting mixture was added methyl iodide (MeI) (5.22 g, 36.8 mmol) dropwise at 0° C. Ice bath was removed after completion of addition and reaction mixture was stirred for 16 hrs at room temperature. All the volatile matters were removed under high vacuum pump and the crude residue thus obtained was purified by column chromatography (Gradient: 0-50% EtOAc in hexane) to afford compound 3 as white foam (6.13 g, 52% yield). ¹H NMR (400 MHz, CDCl₃) δ 8.52 (s, 1H), 8.14 (s, 1H), 7.47-7.36 (m, 6H), 7.34-7.16 (m, 10H), 7.09 (t, J=7.8 Hz, 3H), 6.89-6.66 (m, 6H), 6.10 (d, J=4.7 Hz, 1H), 4.52 (t, J=4.6 Hz, 1H), 4.37 (t, J=4.7 Hz, 1H), 4.21 (q, J=4.2 Hz, 1H), 3.79 (s, 3H), 3.76 (s, 6H), 3.52 (dd, J=10.7, 4.1 Hz, 1H), 3.40 (s, 3H), 3.28 (dd, J=10.7, 4.0 Hz, 1H), 0.87 (s, 9H), 0.05 (d, J=23.7 Hz, 6H) ppm. ¹³C NMR (126 MHz, CDCl₃) δ 172.16, 158.58, 154.89, 152.34, 151.81, 144.47, 142.48, 136.05, 135.63, 135.61, 130.58, 130.06, 128.63, 128.14, 127.85, 127.81, 126.93, 126.74, 113.16, 113.15, 86.95, 86.56, 84.42, 82.56, 70.60, 62.57, 58.46, 55.20, 55.19, 35.81, 25.72, 18.10, −4.64, −4.65, −4.75 ppm. HRMS calc. for C₄₆H₅₄N₅O₇Si [M+H]⁺ 816.3793, found 816.3785.

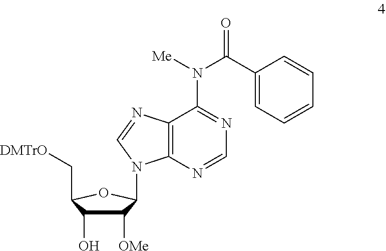

N-(9-((2R,3R,4R,5R)-5-((bis(4-methoxyphenyl)(phenyl)methoxy)methyl)-4-hydroxy-3-methoxy tetrahydrofuran-2-yl)-9H-purin-6-yl)-N-methylbenzamide [Compound 4]: Compound 3 (6.1 g, 7.48 mmol) was dissolved in tetrahydrofuran (THF) (30 mL) and to this resulting clear solution, was added tetrabutylammonium fluoride solution (TBAF) (15.2 mL, 1.0 M in THF, 15.2 mmol) at room temperature. Reaction mixture was stirred for 2 hrs and TLC showed completion of reaction. All the volatile matters were removed under high vacuum pump and the crude compound was purified by column chromatography (Gradient: 30-100% EtOAc in hexane) to afford compound 4 as white solid (5.1 g, 97.2% yield). ¹H NMR (500 MHz, CDCl₃) δ 8.50 (d, J=3.0 Hz, 1H), 8.13 (d, J=3.0 Hz, 1H), 7.58-7.00 (m, 16H), 6.81 (d, J=8.2 Hz, 4H), 6.15 (d, J=3.9 Hz, 1H), 4.48 (q, J=5.4 Hz, 1H), 4.35 (t, J=4.4 Hz, 1H), 4.20 (q, J=4.1 Hz, 1H), 4.15-4.06 (m, 1H), 3.79 (d, J=5.3 Hz, 9H), 3.56-3.46 (m, 4H), 3.40 (dd, J=10.7, 4.3 Hz, 1H), 2.74 (d, J=6.1 Hz, 1H) ppm. ¹³C NMR (126 MHz, CDCl₃) δ 172.34, 158.69, 154.95, 152.28, 151.96, 144.54, 141.92, 136.16, 135.70, 135.63, 130.73, 130.17, 130.15, 128.73, 128.21, 128.00, 127.96, 127.06, 126.63, 113.31, 86.76, 86.63, 84.05, 83.35, 69.81, 63.02, 58.92, 55.32, 35.93 ppm. HRMS calc. for C₄₀H₄₀N₅O₇ [M+H]⁺ 702.2928, found 702.2926.

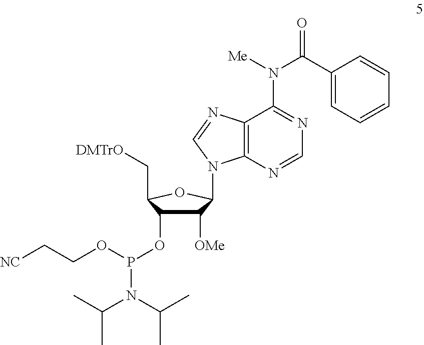

(2R,3R,4R,5R)-2-((bis(4-methoxyphenyl)(phenyl)methoxy)methyl)-4-methoxy-5-(6-(N-methylbenzamido)-9H-purin-9-yl)tetrahydrofuran-3-yl(2-cyanoethyl)diisopropyl phosphoramidite [Compound 5]: To a clear solution of compound 4 (2.0 g, 2.85 mmol) in dry ACN (30 mL) was added N,N-diisopropylethylamine (DIPEA) (0.45 g, 3.44 mmol). To the resulting mixture were added 2-cyanoethyl-N,N,N′,N′-tetraisopropylphosphorodiamidite (1.27 g, 4.28 mmol) and tetrazole (7.0 mL, 0.45 M in ACN, 3.15 mmol) successively in single portions and reaction mixture was stirred for 3 hrs at room temperature. After consumption of starting material, volatile matters were removed under high vacuum pump at low temperature. Residue was dissolved in DCM (30 mL) and organic layer was washed with saturated bicarbonate solution (3×50 mL). Organic layer was separated, dried over anhydrous Na₂SO₄, filtered and filtrate was evaporated to dryness. Residue was triturated with minimum amount of hexane a obtain white solid which was further purified by column chromatography (Gradient: 20-60% EtOAc in hexane) to afford amidite 5 (1.8 g, 70% yield). ¹H NMR (500 MHz, Acetone-d₆) δ 8.32 (d, J=4.3 Hz, 1H), 8.29 (d, J=6.3 Hz, 1H), 7.43-7.31 (m, 2H), 7.29-7.08 (m, 9H), 7.01 (q, J=7.8 Hz, 2H), 6.86-6.60 (m, 4H), 6.05 (dd, J=10.8, 4.8 Hz, 1H), 4.78-4.55 (m, 2H), 4.24 (dq, J=26.2, 4.2 Hz, 1H), 3.93-3.75 (m, 1H), 3.67 (d, J=5.1 Hz, 6H), 3.66-3.52 (m, 4H), 3.46-3.33 (m, 3H), 3.34-3.21 (m, 3H), 2.76-2.61 (m, 2H), 2.50 (t, J=6.0 Hz, 1H), 1.25-1.06 (m, 11H), 1.01 (d, J=6.8 Hz, 3H). ³¹P NMR (202 MHz, CDCl₃) δ 151.07, 150.31 ppm. HRMS calc. for C₄₉H₅₇N₇O₈P [M+H]⁺ 902.4006, found 902.4008.

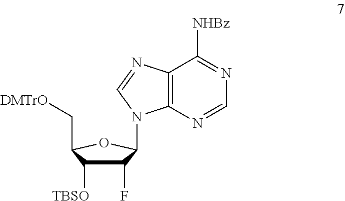

N-(9-((2R,3R,4R,5R)-5-((bis(4-methoxyphenyl)(phenyl)methoxy)methyl)-4-((tert-butyldimethyl silyl)oxy)-3-fluorotetrahydrofuran-2-yl)-9H-purin-6-yl)benzamide [Compound 7]: Commercially available compound 6 (10.0 g, 14.8 mmol) was converted to 7 as white foam following the same method described for compound 2 (11.3 g, 97% yield). ¹H NMR (400 MHz, Chloroform-d) δ 9.59 (s, 1H), 8.73 (s, 1H), 8.27 (s, 1H), 7.99 (dt, J=7.2, 1.3 Hz, 2H), 7.56-7.47 (m, 1H), 7.45-7.34 (m, 5H), 7.30-7.11 (m, 9H), 6.78 (d, J=8.9 Hz, 4H), 6.28 (dd, J=16.4, 2.7 Hz, 1H), 5.61 (ddd, J=52.8, 4.5, 2.7 Hz, 1H), 4.81 (ddd, J=16.2, 6.6, 4.4 Hz, 1H), 4.40-4.14 (m, 1H), 3.74 (d, J=1.1 Hz, 7H), 3.59 (dd, J=10.9, 3.0 Hz, 1H), 3.26 (dd, J=10.9, 3.9 Hz, 1H), 0.86 (s, 10H), 0.11 (s, 3H), 0.02 (s, 4H) ppm. ¹³C NMR (126 MHz, CDCl₃) δ 158.54, 152.67, 151.26, 149.92, 149.71, 144.36, 142.13, 135.96, 135.58, 135.52, 133.58, 132.70, 129.99, 129.98, 128.72, 128.09, 127.96, 127.84, 126.92, 123.71, 123.67, 113.16, 113.14, 92.80, 91.26, 87.42, 87.16, 86.45, 83.33, 83.32, 70.28, 70.16, 61.88, 55.19, 25.63, 18.06, −4.71, −5.04 ppm. ¹⁹F NMR (376 MHz, CDCl₃) δ −204.93 (dt, J=52.9, 16.3 Hz). HRMS calc. for C₄₄H₄₉FN₅O₆Si [M+H]⁺ 790.3436, found 790.3440.

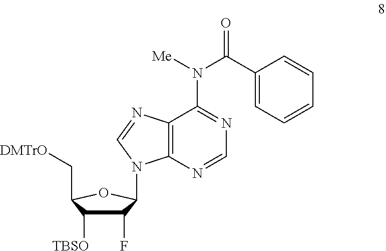

N-(9-((2R,3R,4R,5R)-5-((bis(4-methoxyphenyl)(phenyl)methoxy)methyl)-4-((tert-butyldimethylsilyl)oxy)-3-fluorotetrahydrofuran-2-yl)-9H-purin-6-yl)-N-methylbenzamide [Compound 8]: Compound 7 (11.2 g, 14.2 mmol) was converted to 8 as white solid following the procedure described for compound 3 (4.8 g, 42% yield). ¹H NMR (400 MHz, CDCl₃) δ 8.57 (s, 1H), 8.13 (s, 1H), 7.52-7.09 (m, 16H), 7.02 (t, J=7.8 Hz, 3H), 6.88-6.43 (m, 5H), 6.20 (dd, J=17.0, 2.8 Hz, 1H), 5.57 (ddd, J=53.0, 4.5, 2.8 Hz, 1H), 4.85 (ddd, J=16.2, 6.6, 4.5 Hz, 1H), 4.21-4.15 (m, 1H), 3.78 (d, J=7.5 Hz, 11H), 3.54 (dd, J=11.0, 3.0 Hz, 1H), 3.19 (dd, J=11.0, 3.7 Hz, 1H), 0.85 (s, 9H), 0.10 (s, 3H), 0.01 (s, 3H) ppm. ¹³C NMR (126 MHz, CDCl₃) δ 172.30, 158.71, 158.65, 158.64, 155.30, 152.08, 151.35, 144.55, 143.23, 142.61, 139.62, 135.97, 135.73, 135.61, 131.09, 130.83, 130.11, 130.09, 129.24, 128.83, 128.74, 128.15, 127.93, 127.91, 127.88, 127.14, 126.98, 126.72, 113.25, 113.24, 113.21, 92.87, 91.33, 87.37, 87.10, 86.47, 83.31, 83.30, 70.27, 70.15, 61.82, 55.31, 35.95, 25.73, 18.17, −4.64, −4.93 ppm. ¹⁹F NMR (376 MHz, CDCl₃) δ −204.76 (dt, J=53.0, 16.6 Hz). HRMS calc. for C₄₅H₅₁FN₅O₆Si [M+H]⁺ 804.3593, found 804.3581.

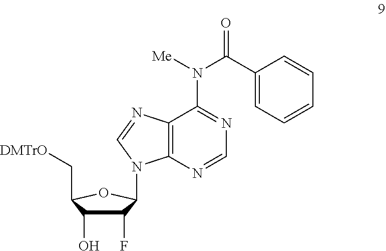

N-(9-((2R,3R,4R,5R)-5-((bis(4-methoxyphenyl)(phenyl)methoxy)methyl)-3-fluoro-4-hydroxy tetrahydrofuran-2-yl)-9H-purin-6-yl)-N-methylbenzamide [Compound 9]: Compound 8 (4.8 g, 4.98 mmol) was converted to 9 as white solid following the procedure described for compound 4 (4.1 g, quantitative yield). ¹H NMR (400 MHz, CDCl₃) δ 8.50 (s, 1H), 8.10 (s, 1H), 7.45-7.33 (m, 5H), 7.29-7.17 (m, 10H), 7.11 (t, J=7.7 Hz, 3H), 6.82-6.61 (m, 5H), 6.23 (dd, J=17.1, 2.5 Hz, 1H), 5.56 (ddd, J=52.9, 4.6, 2.5 Hz, 1H), 4.78 (dq, J=17.5, 6.3 Hz, 1H), 4.19 (dt, J=7.2, 3.6 Hz, 1H), 3.77 (d, J=3.6 Hz, 11H), 3.53 (dd, J=10.9, 3.0 Hz, 1H), 3.39 (dd, J=10.9, 4.1 Hz, 1H), 2.67 (d, J=6.7 Hz, 1H) ppm. ¹³C NMR (126 MHz, Chloroform-d) δ 172.42, 158.70, 155.17, 152.15, 152.06, 144.51, 142.17, 136.02, 135.61, 135.60, 130.90, 130.14, 128.76, 128.16, 128.04, 128.02, 127.08, 126.60, 113.33, 94.01, 92.52, 87.03, 86.80, 86.77, 82.74, 70.21, 70.08, 62.42, 60.54, 55.34, 35.99 ppm. ¹⁹F NMR (376 MHz, CDCl₃) δ −204.77 (dt, J=52.8, 17.0 Hz). HRMS calc. for C₃₉H₃₇FN₅O₆ [M+H]⁺ 690.2728, found 690.2742.

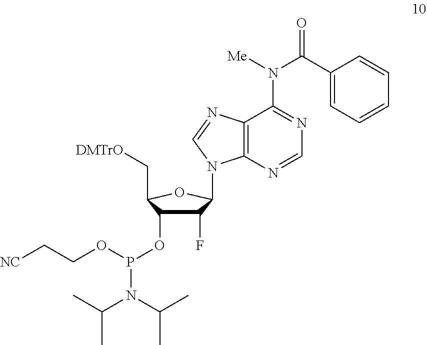

(2R,3R,4R,5R)-2-((bis(4-methoxyphenyl)(phenyl)methoxy)methyl)-4-fluoro-5-(6-(N-methylbenzamido)-9H-purin-9-yl)tetrahydrofuran-3-yl(2-cyanoethyl)diisopropyl phosphoramidite [Compound 10]: Compound 9 (2.26 g, 3.28 mmol) was converted to amidite 10 as white solid following the procedure described for compound 5 (2.42 g, 83% yield). ¹H NMR (400 MHz, CDCl₃) δ 8.51 (d, J=9.6 Hz, 1H), 8.13 (s, 1H), 7.47-7.11 (m, 14H), 7.06 (td, J=7.8, 2.0 Hz, 2H), 6.87-6.66 (m, 5H), 6.24 (ddd, J=17.9, 16.1, 2.2 Hz, 1H), 5.67 (dddd, J=52.8, 19.8, 4.5, 2.2 Hz, 1H), 5.20-4.80 (m, 1H), 4.31 (dt, J=7.1, 3.2 Hz, 1H), 3.84-3.70 (m, 11H), 3.73-3.43 (m, 5H), 3.28 (dt, J=11.1, 2.6 Hz, 1H), 2.50 (dt, J=76.5, 6.3 Hz, 2H), 1.32-1.23 (m, 2H), 1.20-1.13 (m, 11H), 1.04 (d, J=6.8 Hz, 3H) ppm. ¹⁹F NMR (376 MHz, CDCl₃) δ −201.50-−201.96 (m), −201.98-−202.43 (m) ppm. ³¹P NMR (202 MHz, Chloroform-d) δ 151.32 (d, J=8.1 Hz), 150.55 (d, J=12.2 Hz) ppm. HRMS calc. for C₄₄H₄₉FN₅O₆P [M+H]⁺ 890.3806, found 890.3804.

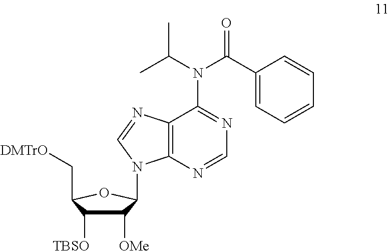

N-[9-[(2R,5R)-5-[[bis(4-methoxyphenyl)-phenyl-methoxy]methyl]-4-[tert-butyl(dimethyl)silyl]oxy-3-methoxy-tetrahydrofuran-2-yl]purin-6-yl]-N-isopropyl-benzamide [Compound 11]: To a clear solution of 2 (3.0 g, 3.74 mmol) in dry dimethylformamide (20 mL) was added potassium carbonate (2.09 g, 14.96 mmol) and 2-iodopropane (1.93 g, 11.22 mmol, 1.13 mL) in single portion and stirred for 16 hrs at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 10-40% EtOAc in hexane) to afford 11 (1.67 g mmol, 53% yield) as white foam. ¹H NMR (500 MHz, DMSO-d₆) δ 8.71 (s, 1H), 8.62 (s, 1H), 7.39-7.05 (m, 12H), 7.01-6.63 (m, 6H), 6.03 (d, J=4.3 Hz, 1H), 5.09-4.90 (m, 1H), 4.64 (t, J=5.0 Hz, 1H), 4.44 (t, J=4.5 Hz, 1H), 4.01-3.89 (m, 1H), 3.72 (d, J=1.8 Hz, 6H), 3.31-3.27 (m, 1H), 3.23 (s, 3H), 3.11-3.04 (m, 1H), 1.35 (dd, J=6.8, 2.2 Hz, 6H), 0.81 (s, 9H), 0.06 (s, 3H), 0.00 (s, 3H) ppm. ¹³C NMR (101 MHz, CDCl₃) δ 171.58, 158.71, 158.70, 154.53, 152.44, 152.11, 144.56, 142.96, 137.40, 135.77, 135.74, 130.20, 130.19, 130.16, 129.65, 128.57, 128.26, 127.99, 127.64, 127.06, 113.28, 86.85, 86.67, 84.62, 82.62, 70.78, 62.76, 58.55, 55.37, 51.63, 25.83, 21.05, 20.96, 18.24, −4.56, −4.66 ppm. HRMS calc. for C₄₈H₅₈N₅O₇Si [M+H]⁺ 844.4106, found 844.4105.

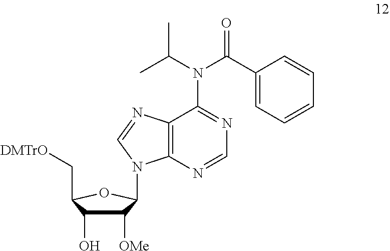

N-[9-[(2R,5R)-5-[[bis(4-methoxyphenyl)-phenyl-methoxy]methyl]-4-hydroxy-3-methoxy-tetrahydrofuran-2-yl]purin-6-yl]-N-isopropyl-benzamide [Compound 12]: To a clear solution of 11 (1.17 g, 1.39 mmol) in dry THF (20 mL) at 22° C., was added tetrabutylammonium fluoride (0.48 g, 1.80 mmol) slowly in single portion and then stirred for 12 hrs. All the volatile matters were removed under high vacuum pump and the residue thus obtained was purified by column chromatography (gradient: 0-5% Methanol in DCM) to afford 12 (0.92 g, 91% yield) as white solid. ¹H NMR (500 MHz, DMSO-d₆) δ 8.67 (s, 1H), 8.57 (s, 1H), 7.36-7.28 (m, 2H), 7.26-7.12 (m, 10H), 7.04 (t, J=7.8 Hz, 2H), 6.88-6.76 (m, 4H), 6.06 (d, J=4.3 Hz, 1H), 5.30 (s, 1H), 5.13-4.89 (m, 1H), 4.40 (q, J=5.4 Hz, 2H), 4.07-4.04 (m, 1H), 3.73 (d, J=2.8 Hz, 6H), 3.30 (s, 3H), 3.27-3.09 (m, 2H), 1.38 (dd, J=6.7, 1.2 Hz, 6H) ppm. ¹³C NMR (126 MHz, DMSO-d₆) δ 170.42, 158.03, 158.02, 153.32, 152.18, 151.67, 144.69, 144.62, 136.95, 135.56, 135.40, 130.20, 129.68, 129.61, 128.69, 127.86, 127.70, 127.62, 127.60, 126.59, 113.11, 85.83, 85.44, 83.61, 81.47, 68.86, 63.36, 59.72, 57.66, 55.00, 50.52, 20.73, 20.50, 14.06 ppm. HRMS calc. for C₄₂H₄₄N₅O₇ [M+H]⁺ 730.3241, found 730.3245.

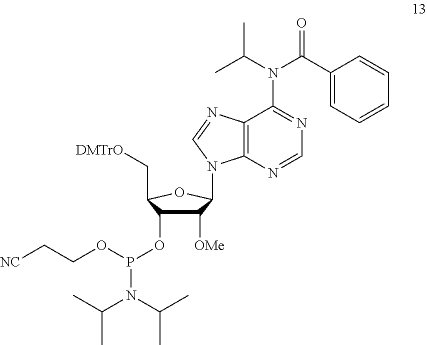

N-[9-[(2R,5R)-5-[[bis(4-methoxyphenyl)-phenyl-methoxy]methyl]-4-[2-cyanoethoxy-(diisopropylamino)phosphanyl]oxy-3-methoxy-tetrahydrofuran-2-yl]purin-6-yl]-N-isopropyl-benzamide [Compound 13]: To a clear solution of 12 (1.02 g, 1.40 mmol) in dry dichloromethane (20 mL) at room temperature (rt, 22° C.) was added N-methyl imidazole (0.23 g, 2.80 mmol, 0.23 mL) and diisopropylethylamine (0.91 g, 6.99 mmol, 1.23 mL). The reaction mixture was stirred for 5 minutes at rt and 2-cyanoethyl-N,N-diisopropylchlorophosphoramidite (0.7 g, 2.80 mmol, 0.66 mL) was added slowly into it. Reaction was kept for stirring at rt and TLC was checked after 1 hr. Reaction mixture was diluted with dichloromethane (20 mL) and washed with 10% NaHCO₃ solution (2×20 mL). Organic layer separated, dried over anhydrous Na₂SO₄, filtered and the filtrate was evaporated to dryness. The crude mass obtained was purified by column chromatography (gradient: 20-60% EtOAc in hexane) to afford 13 (0.99 g, 76% yield) as white foam. ¹H NMR (500 MHz, CD₃CN) δ 8.58 (d, J=9.9 Hz, 1H), 8.15 (d, J=14.1 Hz, 1H), 7.54-7.09 (m, 12H), 7.09-6.92 (m, 2H), 6.82 (td, J=9.1, 1.8 Hz, 4H), 6.00 (d, J=4.8 Hz, 1H), 5.18-4.92 (m, 1H), 4.79-4.60 (m, 1H), 4.52 (dt, J=28.4, 4.9 Hz, 1H), 4.29-4.17 (m, 1H), 4.06 (q, J=7.1 Hz, 1H), 3.90-3.54 (m, 11H), 3.44-3.32 (m, 4H), 3.27 (ddd, J=17.3, 10.8, 4.8 Hz, 1H), 2.71-2.58 (m, 1H), 2.49 (t, J=6.0 Hz, 1H), 1.50-1.30 (m, 6H), 1.29-0.80 (m, 14H) ppm. ¹³C NMR (126 MHz, CD₃CN) δ 172.02, 171.64, 159.70, 155.08, 155.05, 153.48, 153.46, 152.71, 152.68, 145.90, 145.88, 144.81, 144.60, 138.49, 136.75, 136.73, 136.67, 131.15, 131.08, 131.05, 131.02, 130.59, 130.57, 129.22, 129.04, 128.98, 128.83, 128.81, 128.57, 127.87, 127.85, 119.56, 119.34, 114.06, 87.97, 87.74, 87.24, 84.35, 84.32, 84.30, 84.26, 82.73, 82.71, 82.58, 82.54, 71.97, 71.85, 71.75, 71.62, 63.96, 63.57, 60.96, 59.96, 59.82, 59.40, 59.24, 59.10, 59.07, 58.81, 58.79, 55.94, 55.93, 52.00, 51.98, 44.17, 44.08, 43.98, 25.05, 25.03, 24.99, 24.97, 24.93, 24.87, 21.15, 21.08, 21.04, 21.01, 20.96, 14.53 ppm. ³¹P NMR (202 MHz, CD₃CN) δ 151.46, 151.06 ppm. HRMS calc. for C₅₁H₆₁N₇O₈P [M+H]⁺ 930.4319, found 930.4309.

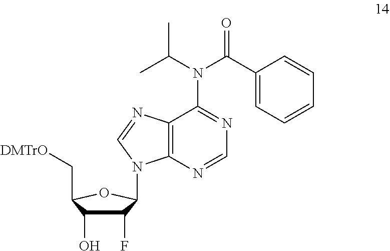

N-[9-[5-[[bis(4-methoxyphenyl)-phenyl-methoxy]methyl]-3-fluoro-4-hydroxy-tetrahydrofuran-2-yl]purin-6-yl]-N-isopropyl-benzamide [Compound 14]: To a clear solution of 6 (1.5 g, 2.18 mmol) in dry dimethylformamide (20 mL) was added potassium carbonate (1.21 g, 8.70 mmol) and 2-iodopropane (1.12 g, 6.53 mmol, 0.66 mL) in single portion and stirred for 12 hrs at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 10-40% EtOAc in hexane) to afford 14 (0.72 g, 46% yield) as white foam. ¹H NMR (400 MHz, CDCl₃) δ 8.65 (s, 1H), 8.03 (s, 1H), 7.45-6.94 (m, 14H), 6.87-6.74 (m, 4H), 6.18 (dd, J=17.3, 2.5 Hz, 1H), 5.62-5.39 (m, 1H), 5.16 (p, J=6.8 Hz, 1H), 4.75 (dtd, J=17.0, 7.1, 4.6 Hz, 1H), 4.19-4.13 (m, 1H), 3.78 (d, J=0.8 Hz, 6H), 3.52 (dd, J=10.9, 3.1 Hz, 1H), 3.37 (dd, J=10.9, 4.1 Hz, 1H), 2.45 (dd, J=7.3, 2.5 Hz, 1H), 1.48 (dd, J=6.8, 2.5 Hz, 6H) ppm. ¹³C NMR (126 MHz, CDCl₃) δ 171.73, 158.76, 158.75, 154.81, 152.36, 152.02, 144.50, 142.60, 137.30, 135.65, 135.63, 130.46, 130.16, 130.14, 129.34, 128.63, 128.19, 128.03, 127.80, 127.09, 113.35, 93.94, 92.45, 86.97, 86.81, 86.70, 82.64, 70.22, 70.09, 62.41, 60.53, 55.36, 51.82 ppm. HRMS calc. for C₄₁H₄₁FN₅O₆ [M+H]⁺ 718.3041, found 718.3038.

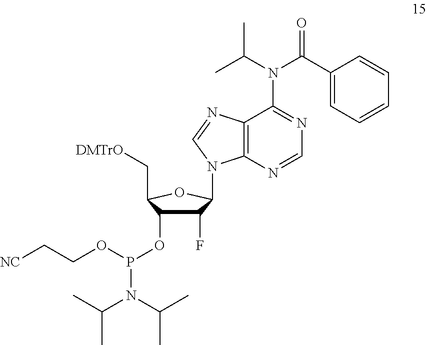

N-[9-[5-[[bis(4-methoxyphenyl)-phenyl-methoxy]methyl]-4-[2-cyanoethoxy-(diisopropylamino)phosphanyl]oxy-3-fluoro-tetrahydrofuran-2-yl]purin-6-yl]-N-isopropyl-benzamide [Compound 15]: To a clear solution of 14 (0.78 g, 1.09 mmol) in DCM (20 mL) at 22° C. was added N-methyl imidazole (0.14 g, 1.63 mmol, 0.13 mL) and diisopropylethylamine (0.71 g, 5.43 mmol, 0.96 mL). The reaction mixture was stirred for 5 minutes at 22° C. and 2-cyanoethyl-N,N-diisopropylchlorophosphoramidite (0.54 g, 2.17 mmol, 0.51 mL) was added slowly into it. Reaction was kept for stirring at 22° C. and TLC was checked after 1 hr. Reaction mixture was diluted with DCM (20 mL) and washed with 10% NaHCO₃ solution (2×30 mL). Organic layer separated, dried over anhydrous Na₂SO₄, filtered and the filtrate was evaporated to dryness. The crude mass obtained was purified by column chromatography (gradient: 20-60% EtOAc in hexane) to afford 15 (0.79 g, 79% yield) as white foam. ¹H NMR (400 MHz, CD₃CN) δ 8.63 (d, J=7.0 Hz, 1H), 8.19 (d, J=0.7 Hz, 1H), 7.50-7.01 (m, 13H), 6.95 (ddd, J=8.1, 7.3, 6.2 Hz, 2H), 6.85-6.66 (m, 4H), 6.27-6.14 (m, 1H), 5.73-5.57 (m, 1H), 5.36-4.84 (m, 2H), 4.20 (dd, J=8.2, 3.4 Hz, 1H), 3.92-3.71 (m, 8H), 3.69-3.51 (m, 3H), 3.44 (ddd, J=13.1, 11.1, 2.2 Hz, 1H), 3.21 (ddd, J=11.2, 4.3, 3.2 Hz, 1H), 2.66-2.58 (m, 1H), 2.49 (t, J=6.0 Hz, 1H), 1.42-1.34 (m, 6H), 1.30-1.12 (m, 12H), 1.05 (d, J=6.8 Hz, 3H) ppm. ¹³C NMR (101 MHz, CD₃CN) δ 172.04, 171.66, 159.65, 155.22, 155.20, 153.01, 152.78, 152.76, 145.85, 145.17, 145.05, 138.32, 136.77, 136.68, 131.25, 131.03, 131.01, 130.97, 130.49, 130.45, 129.22, 129.01, 128.96, 128.75, 128.61, 128.59, 127.82, 127.79, 119.48, 119.30, 114.00, 113.98, 113.96, 94.66, 94.21, 92.79, 92.31, 88.88, 88.53, 87.04, 86.95, 82.30, 82.24, 71.21, 71.06, 70.91, 70.50, 70.34, 70.19, 62.75, 62.16, 60.96, 60.05, 59.85, 59.79, 59.59, 55.92, 55.90, 52.13, 52.10, 44.21, 44.18, 44.09, 44.06, 25.09, 25.02, 24.99, 24.93, 24.86, 24.79, 21.15, 21.10, 21.08, 21.03, 20.99, 20.95, 20.93 ppm. ³¹P NMR (162 MHz, CD₃CN) δ 150.81 (d, J=6.8 Hz), 150.61 (d, J=10.7 Hz) ppm. HRMS calc. for C₅₀H₅₈FN₇O₇P [M+H]⁺ 918.4119, found 918.4124.

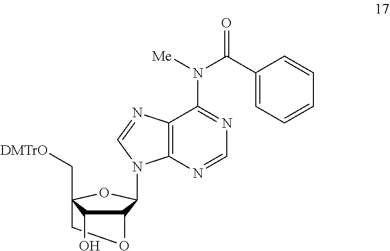

N-[9-[(4R,6R)-4-[[bis(4-methoxyphenyl)-phenyl-methoxy]methyl]-7-hydroxy-2,5-dioxabicyclo[2.2.1]heptan-6-yl]purin-6-yl]-N-methyl-benzamide [Compound 17]: To a clear solution of 169 (1.02 g, 1.46 mmol) in dry dimethylformamide (20 mL) was added potassium carbonate (0.81 g, 5.83 mmol) and iodomethane (0.62 g, 4.37 mmol, 0.27 mL) in single portions and stirred for 16 hrs at 22° C. Reaction mixture was diluted with EtOAc (30 mL) and organic layer was washed with water (30 mL) and brine (2×30 mL). Organic layer was separated, dried over anhydrous Na₂SO₄, filtered and filtrate was evaporated to dryness. Crude compound was purified by column chromatography (gradient: 25-75% EtOAc in hexane) to afford 17 (0.67 g, 66% yield) as white foam. ¹H NMR (400 MHz, CDCl₃) δ 8.52 (s, 1H), 8.17 (s, 1H), 7.53-7.10 (m, 14H), 6.85 (d, J=9.0 Hz, 4H), 6.05 (d, J=0.6 Hz, 1H), 4.63 (s, 1H), 4.33 (d, J=6.0 Hz, 1H), 4.01 (d, J=1.7 Hz, 2H), 3.79 (d, J=3.6 Hz, 9H), 3.61-3.40 (m, 2H), 2.43 (d, J=6.0 Hz, 1H) ppm. ¹³C NMR (101 MHz, CDCl₃) δ 172.49, 158.86, 154.97, 152.08, 151.57, 144.38, 140.82, 136.14, 135.48, 135.42, 130.89, 130.13, 128.78, 128.20, 128.15, 128.09, 127.27, 126.67, 113.49, 87.61, 86.88, 86.45, 79.49, 72.28, 71.97, 60.54, 59.25, 55.39, 36.03 ppm. HRMS calc. for C₄₀H₃₈N₅O₇ [M+H]⁺ 700.2771, found 700.2780

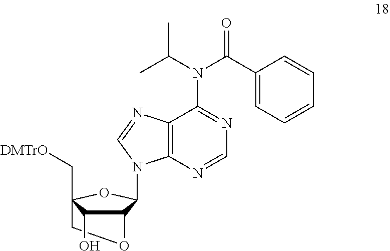

N-[9-[(4R,6R)-4-[[bis(4-methoxyphenyl)-phenyl-methoxy]methyl]-7-hydroxy-2,5-dioxabicyclo[2.2.1]heptan-6-yl]purin-6-yl]-N-isopropyl-benzamide [Compound 18]: To a clear solution of 16 (3.0 g, 4.29 mmol) in dry dimethylformamide (20 mL) was added potassium carbonate (2.39 g, 17.15 mmol) and 2-iodopropane (2.19 g, 12.86 mmol, 1.29 mL) in single portion and stirred for 16 hrs at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 25-60% EtOAc in hexane) to afford 18 (1.01 g, 32% yield) as white foam. ¹H NMR (400 MHz, CDCl₃) δ 8.67 (s, 1H), 8.10 (s, 1H), 7.47-7.24 (m, 12H), 7.17-6.99 (m, 3H), 6.88-6.80 (m, 4H), 5.99 (d, J=0.7 Hz, 1H), 5.15 (p, J=6.8 Hz, 1H), 4.60 (s, 1H), 4.26 (d, J=6.0 Hz, 1H), 4.04-3.96 (m, 2H), 3.80 (s, 6H), 3.63-3.46 (m, 2H), 2.42 (d, J=5.9 Hz, 1H), 1.49 (dd, J=6.8, 2.5 Hz, 6H) ppm. ¹³C NMR (126 MHz, CDCl₃) δ 171.83, 158.87, 154.51, 152.22, 151.51, 144.38, 141.33, 137.38, 135.47, 135.40, 130.38, 130.13, 130.11, 129.42, 129.27, 128.59, 128.19, 128.14, 127.98, 127.91, 127.82, 127.26, 113.48, 113.31, 79.43, 72.27, 71.99, 60.54, 59.30, 55.39, 51.81, 21.04, 20.99 ppm. HRMS calc. for C₄₂H₄₂N₅O₇ [M+H]⁺ 728.3084, found 728.3088.

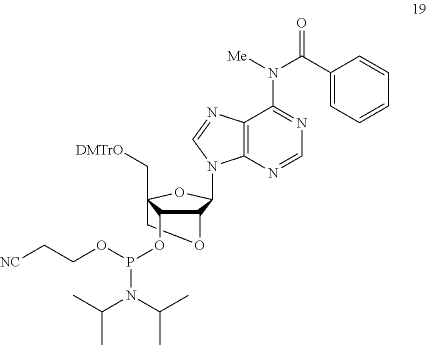

N-[9-[(4R,6R)-4-[[bis(4-methoxyphenyl)-phenyl-methoxy]methyl]-7-[2-cyanoethoxy-(diisopropylamino)phosphanyl]oxy-2,5-dioxabicyclo[2.2.1]heptan-6-yl]purin-6-yl]-N-methyl-benzamide [Compound 19]: To a clear solution of 17 (1.25 g, 1.79 mmol) in dichloromethane (20 mL) at 22° C. was added N-methyl imidazole (0.22 g, 2.68 mmol, 0.22 mL) and diisopropylethylamine (1.17 g, 8.93 mmol, 1.57 mL). The reaction mixture was stirred for 5 minutes at 22° C. and 2-cyanoethyl-N,N-diisopropylchlorophosphoramidite (0.89 g, 3.57 mmol, 0.84 mL) was added slowly into it. Reaction was kept for stirring at 22° C. and TLC was checked after 1 hr. Reaction mixture was diluted with dichloromethane (20 mL) and washed with 10% NaHCO₃ solution (2×30 mL). Organic layer separated, dried over anhydrous Na₂SO₄, filtered and the filtrate was evaporated to dryness. The crude mass obtained was purified by column chromatography (gradient: 20-60% EtOAc in hexane) to afford 19 (1.2 g, 75% yield) as white foam. ¹H NMR (400 MHz, CD₃CN) δ 8.46 (d, J=2.0 Hz, 1H), 8.22 (d, J=10.1 Hz, 1H), 7.53-7.07 (m, 14H), 7.01-6.57 (m, 4H), 6.05 (dd, J=2.9, 0.6 Hz, 1H), 4.76 (d, J=14.0 Hz, 1H), 4.51 (dd, J=17.9, 8.0 Hz, 1H), 4.10-3.88 (m, 2H), 3.82-3.62 (m, 10H), 3.63-3.36 (m, 5H), 2.55-2.38 (m, 2H), 1.15-0.69 (m, 12H) ppm. ¹³C NMR (101 MHz, CD₃CN) δ 172.85, 171.65, 159.79, 155.71, 155.68, 152.87, 152.78, 152.44, 145.92, 145.85, 142.48, 137.41, 136.64, 136.61, 136.47, 136.38, 131.63, 131.60, 131.11, 131.07, 131.00, 129.51, 129.48, 129.01, 128.92, 128.90, 128.86, 127.99, 127.95, 127.68, 119.29, 119.25, 114.14, 88.67, 88.63, 88.61, 88.55, 87.70, 87.14, 79.53, 79.21, 79.18, 73.67, 73.54, 73.02, 72.88, 60.96, 60.46, 60.18, 59.67, 59.48, 59.39, 59.20, 55.95, 55.93, 44.14, 44.08, 44.01, 43.95, 36.13, 24.93, 24.86, 24.83, 24.76, 24.68, 21.16, 20.98, 20.94, 20.91, 20.87, 14.53 ppm. ³¹P NMR (162 MHz, CD₃CN) δ 149.39, 149.15 ppm. HRMS calc. for C₄₉H₅₅N₇O₈P [M+H]⁺ 900.3850, found 900.3856.

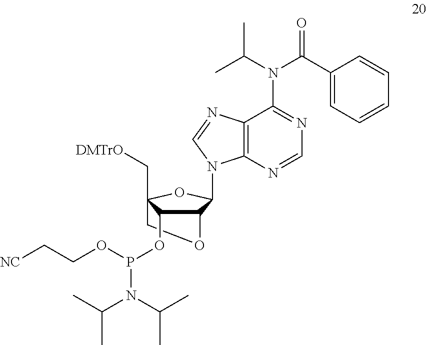

N-[9-[(4R,6R)-4-[[bis(4-methoxyphenyl)-phenyl-methoxy]methyl]-7-[2-cyanoethoxy-(diisopropylamino)phosphanyl]oxy-2,5-dioxabicyclo[2.2.1]heptan-6-yl]purin-6-yl]-N-isopropyl-benzamide [Compound 20]: To a clear solution of 18 (0.78 g, 1.07 mmol) in DCM (20 mL) at 22° C. was added N-methyl imidazole (0.133 g, 1.61 mmol, 0.13 mL) and diisopropylethylamine (0.699 g, 5.36 mmol, 0.94 mL). The reaction mixture was stirred for 5 minutes at 22° C. and 2-cyanoethyl-N,N-diisopropylchlorophosphoramidite (0.534 g, 2.14 mmol, 0.50 mL) was added slowly into it. Reaction was kept for stirring at 22° C. and TLC was checked after 1 hr. Reaction mixture was diluted with DCM (20 mL) and washed with 10% NaHCO₃ solution (2×30 mL). Organic layer separated, dried over anhydrous Na₂SO₄, filtered and the filtrate was evaporated to dryness. The crude mass obtained was purified by column chromatography (gradient: 20-60% EtOAc in hexane) to afford 20 (0.81 g, 81% yield) as white foam. ¹H NMR (400 MHz, CD₃CN) δ 8.62 (d, J=1.3 Hz, 1H), 8.19 (d, J=10.5 Hz, 1H), 7.45 (ddt, J=6.2, 4.9, 1.4 Hz, 2H), 7.36-7.18 (m, 10H), 7.14-7.07 (m, 2H), 6.93-6.82 (m, 4H), 6.00 (dd, J=2.8, 0.6 Hz, 1H), 5.16-5.03 (m, 1H), 4.81-4.68 (m, 1H), 4.51-4.39 (m, 1H), 4.14-3.87 (m, 3H), 3.78 (dd, J=2.5, 0.7 Hz, 6H), 3.72-3.60 (m, 1H), 3.55-3.40 (m, 5H), 2.55-2.37 (m, 2H), 1.42 (dd, J=6.8, 2.1 Hz, 6H), 1.21 (t, J=7.1 Hz, 2H), 1.08 (dd, J=6.8, 1.3 Hz, 6H), 0.92-0.80 (m, 6H) ppm. ¹³C NMR (126 MHz, CD₃CN) δ 172.10, 172.08, 159.81, 154.99, 154.97, 152.75, 152.68, 145.92, 145.84, 143.17, 143.13, 138.56, 138.54, 136.65, 136.60, 136.48, 136.40, 131.25, 131.21, 131.13, 131.09, 131.07, 131.01, 130.65, 129.25, 129.02, 128.92, 128.91, 128.69, 128.67, 128.01, 127.97, 119.28, 119.24, 114.14, 114.13, 88.66, 88.63, 88.59, 87.68, 87.16, 79.40, 79.20, 79.17, 73.68, 73.57, 73.52, 73.46, 72.91, 72.79, 60.97, 60.39, 60.11, 59.73, 59.57, 59.46, 59.31, 55.97, 55.94, 52.04, 52.01, 44.12, 44.05, 44.02, 43.95, 24.93, 24.87, 24.83, 24.77, 24.75, 24.69, 21.10, 21.07, 21.05 ppm. ³¹P NMR (162 MHz, CD₃CN) δ 150.08, 149.97 ppm. HRMS calc. for C₅₁H₅₉N₇O₈P [M+H]⁺ 928.4163, found 928.4166.

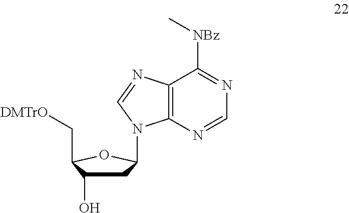

N-[9-[5-[[bis(4-methoxyphenyl)-phenyl-methoxy]methyl]-4-hydroxy-tetrahydrofuran-2-yl]purin-6-yl]-N-methyl-benzamide [Compound 22]: To a clear solution of 21¹⁰ (0.6 g, 894.01 μmol) in dry DMF (20 mL) was added potassium carbonate (499.24 mg, 3.58 mmol) and iodomethane (384.53 mg, 2.68 mmol, 168.65 μL) in single portion and stirred for 10 hrs at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 10-40% EtOAc in hexane) to afford 22 (0.28 g, 47% yield) as white foam. ¹H NMR (400 MHz, CDCl₃) δ 8.45 (s, 1H), 8.05 (s, 1H), 7.47-7.33 (m, 4H), 7.30-7.09 (m, 11H), 6.86-6.73 (m, 5H), 6.43 (dd, J=8.5, 5.6 Hz, 1H), 5.41 (dt, J=6.0, 2.0 Hz, 1H), 4.35 (td, J=4.1, 2.0 Hz, 1H), 3.82 (s, 3H), 3.78 (s, 10H), 3.47-3.38 (m, 2H), 3.29 (d, J=4.1 Hz, OH), 2.99 (ddd, J=14.3, 8.5, 6.0 Hz, 1H), 2.68 (ddd, J=14.1, 5.7, 1.9 Hz, 1H) ppm. ¹³C NMR (101 MHz, CDCl₃) δ 172.35, 158.74, 155.01, 152.46, 151.92, 144.48, 141.71, 136.22, 135.58, 130.77, 130.15, 128.75, 128.22, 128.17, 128.05, 128.03, 128.00, 127.11, 126.54, 113.36, 113.29, 86.87, 84.53, 84.19, 78.68, 63.57, 55.35, 55.26, 37.92, 35.98 ppm.

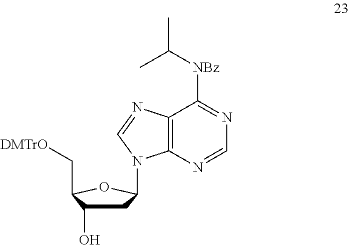

N-[9-[5-[[bis(4-methoxyphenyl)-phenyl-methoxy]methyl]-4-hydroxy-tetrahydrofuran-2-yl]purin-6-yl]-N-isopropyl-benzamide [Compound 23]: To a clear solution of 21¹⁰ (1.0 g, 1.49 mmol) in dry DMF (20 mL) was added potassium carbonate (832.06 mg, 5.96 mmol) and 2-iodopropane (3.07 g, 17.88 mmol, 1.81 mL) in single portion and stirred for 16 hrs at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 10-40% EtOAc in hexane) to afford 23 (0.30 g, 29% yield) as white foam. ¹H NMR (500 MHz, CDCl₃) δ 8.62 (s, 1H), 7.98 (s, 1H), 7.37 (tt, J=6.3, 1.3 Hz, 5H), 7.30-7.21 (m, 7H), 7.15-7.08 (m, 1H), 7.06-6.99 (m, 2H), 6.84-6.75 (m, 5H), 6.37 (t, J=6.5 Hz, 1H), 5.20-5.08 (m, J=6.6 Hz, 1H), 4.62 (dq, J=6.3, 3.8 Hz, 1H), 4.16-4.06 (m, 2H), 3.79 (d, J=1.2 Hz, 8H), 3.35 (h, J=5.4 Hz, 2H), 2.71 (dt, J=13.2, 6.4 Hz, 1H), 2.47 (ddd, J=13.5, 6.3, 4.1 Hz, 1H), 1.47 (dd, J=6.9, 3.8 Hz, 8H) ppm. ¹³C NMR (101 MHz, CDCl₃) δ 171.70, 158.75, 158.74, 155.41, 154.39, 152.36, 152.06, 151.43, 147.47, 144.55, 143.48, 142.41, 139.61, 137.38, 137.29, 135.72, 135.70, 130.63, 130.30, 130.26, 130.14, 130.10, 129.51, 129.27, 128.73, 128.56, 128.17, 128.05, 127.97, 127.93, 127.90, 127.78, 127.20, 127.10, 113.36, 113.30, 113.25, 89.61, 87.73, 86.76, 86.07, 84.31, 73.33, 72.53, 63.68, 60.54, 55.37, 51.72, 40.08 ppm.

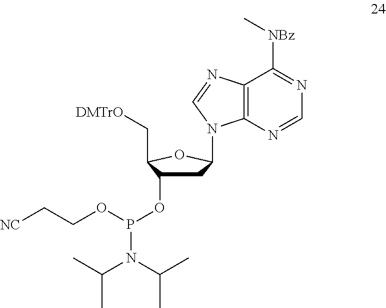

N-[9-[5-[[bis(4-methoxyphenyl)-phenyl-methoxy]methyl]-4-[2-cyanoethoxy-(diisopropylamino)phosphanyl]oxy-tetrahydrofuran-2-yl]purin-6-yl]-N-methyl-benzamide [Compound 24]: To a clear solution of 22 (1.41 g, 2.10 mmol) in DCM (20 mL) at room temperature was added N-methyl imidazole (344.66 mg, 4.20 mmol, 334.62 μL) and DIPEA (1.36 g, 10.50 mmol, 1.83 mL). The reaction mixture was stirred for 5 minutes at 22° C. and 2-cyanoethyl-N,N-diisopropylchlorophosphoramidite (993.60 mg, 4.20 mmol, 937.35 μL) was added slowly into it. Reaction was kept for stirring at 22° C. and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 24 (1.49 g, 81% yield) as white foam. ¹H NMR (500 MHz, CD₃CN) δ 8.39 (d, J=3.1 Hz, 1H), 8.17 (d, J=3.1 Hz, 1H), 7.54-6.92 (m, 15H), 6.78 (ddd, J=8.8, 6.3, 3.4 Hz, 4H), 6.37 (td, J=6.9, 5.7 Hz, 1H), 4.91-4.78 (m, 1H), 4.18 (dq, J=13.1, 4.5 Hz, 1H), 3.88-3.66 (m, 11H), 3.62-3.53 (m, 1H), 3.32 (ddd, J=14.8, 10.5, 3.8 Hz, 1H), 3.23 (ddd, J=10.4, 8.2, 5.3 Hz, 1H), 2.99 (dtd, J=14.0, 6.3, 2.0 Hz, 1H), 2.66-2.50 (m, 3H), 1.25-1.11 (m, 11H), 1.08 (d, J=6.7 Hz, 3H) ppm. ¹³C NMR (126 MHz, CD₃CN) δ 172.80, 171.63, 159.63, 155.65, 153.42, 153.37, 152.30, 146.01, 144.26, 144.22, 137.39, 136.84, 136.81, 136.76, 131.52, 130.99, 130.96, 129.42, 128.97, 128.92, 128.77, 128.75, 127.79, 127.77, 127.71, 119.55, 119.39, 114.00, 87.04, 86.43, 86.40, 86.24, 86.20, 85.47, 85.43, 74.29, 74.16, 73.75, 73.62, 64.32, 64.18, 60.96, 59.64, 59.54, 59.49, 59.38, 55.90, 55.89, 44.11, 44.07, 44.01, 43.97, 39.02, 38.99, 38.90, 38.87, 36.09, 24.97, 24.94, 24.91, 24.89, 24.84, 21.16, 21.09, 21.04, 21.01, 20.96, 14.53 ppm. ³¹P NMR (202 MHz, CD₃CN) δ 149.29, 149.15 ppm.

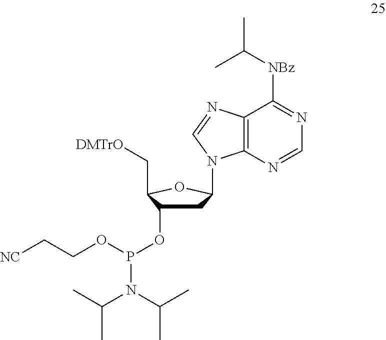

N-[9-[5-[[bis(4-methoxyphenyl)-phenyl-methoxy]methyl]-4-[2-cyanoethoxy-(diisopropylamino)phosphanyl]oxy-tetrahydrofuran-2-yl]purin-6-yl]-N-isopropyl-benzamide [Compound 25]: To a clear solution of 23 (0.58 g, 828.82 μmol) in DCM (20 mL) at 22° C. was added N-methyl imidazole (136.09 mg, 1.66 mmol, 132.13 μL) and DIPEA (535.58 mg, 4.14 mmol, 721.81 μL). The reaction mixture was stirred for 5 minutes at 22° C. and 2-cyanoethyl-N,N-diisopropylchlorophosphoramidite (392.33 mg, 1.66 mmol, 370.12 μL) was added slowly into it. Reaction was kept for stirring at 22° C. and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 25 (0.58 g, 78% yield) as white foam. ¹H NMR (500 MHz, CD₃CN) δ 8.55 (d, J=3.8 Hz, 1H), 8.15 (d, J=1.8 Hz, 1H), 7.68-6.93 (m, 13H), 6.80 (tt, J=6.8, 1.7 Hz, 4H), 6.33 (q, J=6.5 Hz, 1H), 5.05 (pd, J=6.8, 1.3 Hz, 1H), 4.89-4.75 (m, 1H), 4.19-4.11 (m, 1H), 3.90-3.66 (m, 8H), 3.66-3.52 (m, 2H), 3.31 (ddd, J=14.6, 10.5, 3.9 Hz, 1H), 3.20 (ddd, J=10.5, 8.4, 5.2 Hz, 1H), 2.93 (dtd, J=13.6, 6.3, 4.3 Hz, 1H), 2.72-2.39 (m, 3H), 1.39 (ddd, J=6.8, 2.2, 1.1 Hz, 6H), 1.21-1.13 (m, 10H), 1.08 (d, J=6.8 Hz, 3H) ppm. ¹³C NMR (126 MHz, CD₃CN) δ 172.03, 171.65, 159.66, 154.92, 153.45, 153.41, 152.57, 145.98, 144.81, 144.78, 138.53, 136.88, 136.85, 136.82, 136.75, 131.13, 131.02, 131.00, 130.99, 130.95, 130.61, 130.58, 129.18, 128.99, 128.94, 128.78, 128.59, 127.81, 127.79, 119.55, 119.40, 114.02, 87.06, 86.33, 86.29, 86.16, 86.11, 85.32, 85.29, 74.19, 74.06, 73.61, 73.48, 64.20, 64.03, 60.96, 59.65, 59.54, 59.50, 59.39, 55.93, 55.91, 52.02, 52.00, 44.11, 44.08, 44.01, 43.98, 38.97, 38.86, 38.83, 24.97, 24.94, 24.91, 24.89, 24.83, 21.16, 21.10, 21.05, 21.02, 20.96, 14.53 ppm. ³¹P NMR (202 MHz, CD₃CN) δ 149.27, 149.18 ppm.

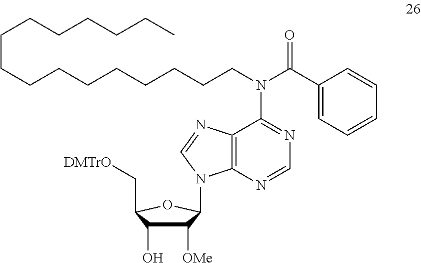

N-[9-[(2R,5R)-5-[[bis(4-methoxyphenyl)-phenyl-methoxy]methyl]-4-hydroxy-3-methoxy-tetrahydrofuran-2-yl]purin-6-yl]-N-hexadecyl-benzamide [compound 26]: To a clear solution of 1 (3.05 g, 4.35 mmol) in dry dimethylformamide (20 mL) was added potassium carbonate (2.43 g, 17.40 mmol, 1.06 mL), 1-bromohexadecane (6.85 g, 21.76 mmol, 6.85 mL) in single portion and stirred for 16 hr at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 10-50% EtOAc in hexane) to afford 26 (2.1 g, 53% yield) as white foam. ¹H NMR (400 MHz, CDCl₃) δ 8.52 (s, 1H), 8.08 (s, 1H), 7.41 (dt, J=8.1, 1.3 Hz, 4H), 7.35-7.17 (m, 8H), 7.16-7.07 (m, 2H), 6.85-6.75 (m, 4H), 6.13 (d, J=3.9 Hz, 1H), 4.46 (dt, J=6.4, 5.3 Hz, 1H), 4.36-4.28 (m, 3H), 4.18 (ddd, J=5.5, 4.2, 3.1 Hz, 1H), 3.79 (d, J=0.8 Hz, 6H), 3.52 (s, 4H), 3.38 (dd, J=10.7, 4.3 Hz, 1H), 2.60 (d, J=6.4 Hz, 1H), 1.80-1.67 (m, 2H), 1.35-1.20 (m, 27H), 0.91-0.83 (m, 3H) ppm. ¹³C NMR (101 MHz, CDCl₃) δ 172.13, 158.75, 154.78, 152.16, 144.61, 141.77, 136.73, 135.73, 135.69, 130.58, 130.21, 128.75, 128.25, 128.05, 127.89, 127.10, 113.36, 86.80, 86.61, 84.00, 83.36, 69.88, 63.06, 58.96, 55.36, 48.86, 32.06, 29.83, 29.81, 29.79, 29.74, 29.71, 29.49, 29.47, 28.91, 27.07, 22.82, 14.26 ppm.

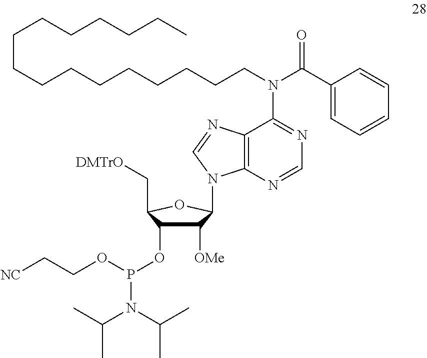

N-[9-[(2R,5R)-5-[[bis(4-methoxyphenyl)-phenyl-methoxy]methyl]-4-[2-cyanoethoxy-(diisopropylamino)phosphanyl]oxy-3-methoxy-tetrahydrofuran-2-yl]purin-6-yl]-N-hexadecyl-benzamide [compound 28]: To a clear solution of 26 (1.9 g, 2.08 mmol) in DCM (20 mL) at room temperature was added N-methyl imidazole (342.02 mg, 4.17 mmol, 332.06 μL) and diisopropylethylamine (1.35 g, 10.41 mmol, 1.81 mL). The reaction mixture was stirred for 5 minutes at rt and 2-cyanoethyl-N,N-diisopropylchlorophosphoramidite (985.99 mg, 4.17 mmol, 930.18 μL) was added slowly into it. Reaction was kept for stirring at rt and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 28 (1.89 g, 82% yield) as white foam. ¹H NMR (500 MHz, CD₃CN) δ 8.43 (d, J=8.7 Hz, 1H), 8.15 (d, J=12.6 Hz, 1H), 7.38 (tt, J=7.8, 1.6 Hz, 2H), 7.33-7.19 (m, 11H), 7.08 (tdd, J=7.4, 4.6, 1.7 Hz, 2H), 6.85-6.76 (m, 4H), 6.04 (d, J=4.5 Hz, 1H), 4.79-4.67 (m, 1H), 4.63-4.52 (m, 1H), 4.31-4.21 (m, 3H), 3.92-3.78 (m, 1H), 3.75 (dd, J=5.0, 1.2 Hz, 6H), 3.72 (s, 2H), 3.45-3.35 (m, 4H), 3.27 (ddd, J=17.8, 10.8, 4.7 Hz, 1H), 2.69-2.61 (m, 1H), 2.48 (t, J=6.0 Hz, 1H), 1.69-1.58 (m, 2H), 1.26-1.13 (m, 29H), 1.07 (d, J=6.7 Hz, 4H), 0.90-0.83 (m, 3H) ppm. ¹³C NMR (101 MHz, CD₃CN) δ 172.58, 159.67, 155.43, 155.41, 153.34, 152.61, 152.59, 145.91, 145.89, 144.23, 144.03, 137.75, 137.73, 136.73, 136.71, 136.67, 131.46, 131.06, 131.03, 131.01, 129.43, 129.03, 128.97, 128.81, 128.79, 128.72, 128.17, 128.13, 127.84, 127.81, 119.54, 119.32, 114.03, 88.06, 87.82, 87.21, 84.27, 84.24, 84.22, 84.17, 82.78, 82.74, 82.62, 82.57, 71.94, 71.76, 71.60, 63.89, 63.50, 60.95, 59.97, 59.79, 59.43, 59.23, 59.13, 59.10, 58.84, 58.82, 55.92, 55.90, 49.10, 44.17, 44.09, 44.05, 43.96, 32.65, 30.40, 30.38, 30.36, 30.34, 30.30, 30.21, 30.16, 30.08, 29.87, 29.33, 27.50, 25.08, 25.05, 25.00, 24.98, 24.94, 24.87, 23.40, 21.06, 21.00, 20.94, 14.53, 14.42 ppm. ³¹P NMR (202 MHz, CD₃CN) δ 151.38, 150.99 ppm.

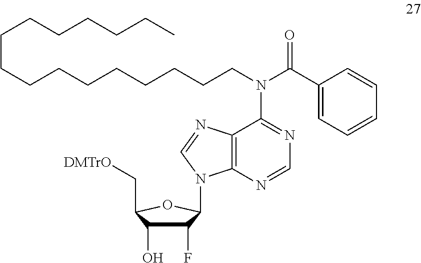

N-[9-[(2R,5R)-5-[[bis(4-methoxyphenyl)-phenyl-methoxy]methyl]-3-fluoro-4-hydroxy-tetrahydrofuran-2-yl]purin-6-yl]-N-hexadecyl-benzamide [compound 27]: To a clear solution of 6 (3.00 g, 4.35 mmol) in dry dimethylformamide (20 mL) was added potassium carbonate (2.43 g, 17.40 mmol, 1.06 mL), 1-bromohexadecane (6.85 g, 21.76 mmol, 6.85 mL) in single portion and stirred for 16 hr at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 10-50% EtOAc in hexane) to afford 27 (2.25 g, 57% yield) as white foam. ¹H NMR (400 MHz, CDCl₃) δ 8.53 (s, 1H), 8.04 (s, 1H), 7.44-7.31 (m, 4H), 7.30-7.15 (m, 9H), 7.12-7.03 (m, 2H), 6.83-6.74 (m, 4H), 6.21 (dd, J=17.4, 2.5 Hz, 1H), 5.68-5.29 (m, 1H), 4.78 (dtd, J=17.1, 7.1, 4.6 Hz, 1H), 4.36-4.27 (m, 2H), 4.21-4.12 (m, 1H), 3.78 (s, 6H), 3.54 (dd, J=10.9, 3.1 Hz, 1H), 3.38 (dd, J=10.9, 4.1 Hz, 1H), 2.34 (dd, J=7.4, 2.6 Hz, 1H), 1.78-1.65 (m, 2H), 1.31-1.19 (m, 28H), 0.91-0.83 (m, 3H) ppm. ¹³C NMR (101 MHz, CDCl₃) δ 172.19, 158.75, 154.99, 152.34, 151.93, 144.54, 142.05, 136.56, 135.64, 135.60, 130.75, 130.15, 128.75, 128.17, 128.04, 127.95, 127.08, 127.03, 113.35, 94.20, 92.34, 87.03, 86.82, 86.70, 82.66, 70.28, 70.11, 62.41, 55.35, 48.91, 32.06, 29.83, 29.79, 29.74, 29.70, 29.49, 29.45, 28.89, 27.05, 22.82, 14.26 ppm. ¹⁹F NMR (471 MHz, CDCl₃) δ −204.93 (dt, J=52.8, 17.2 Hz).

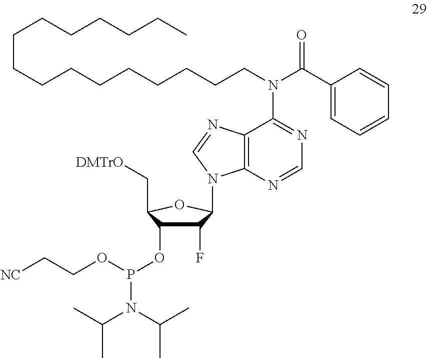

N-[9-[(2R,5R)-5-[[bis(4-methoxyphenyl)-phenyl-methoxy]methyl]-4-[2-cyanoethoxy-(diisopropylamino)phosphanyl]oxy-3-fluoro-tetrahydrofuran-2-yl]purin-6-yl]-N-hexadecyl-benzamide [compound 29]: To a clear solution of 27 (2.1 g, 2.33 mmol) in DCM (20 mL) at room temperature was added N-methyl imidazole (383.08 mg, 4.67 mmol, 371.92 μL) and diisopropylethylamine (1.51 g, 11.67 mmol, 2.03 mL). The reaction mixture was stirred for 5 minutes at rt and 2-cyanoethyl-N,N-diisopropylchlorophosphoramidite (1.10 g, 4.67 mmol, 1.04 mL) was added slowly into it. Reaction was kept for stirring at rt and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 29 (2.1 g, 82% yield) as white foam. ¹H NMR (500 MHz, CD₃CN) δ 8.49 (d, J=9.3 Hz, 1H), 8.19 (d, J=4.0 Hz, 1H), 7.35-7.26 (m, 4H), 7.18 (dddd, J=7.6, 5.8, 4.2, 1.9 Hz, 8H), 7.00 (dt, J=8.4, 7.2 Hz, 2H), 6.81-6.72 (m, 4H), 6.28-6.17 (m, 1H), 5.89-5.60 (m, 1H), 5.36-4.97 (m, 1H), 4.31-4.17 (m, 3H), 3.92-3.70 (m, 7H), 3.68-3.53 (m, 3H), 3.45 (ddd, J=13.9, 11.1, 2.2 Hz, 1H), 3.21 (dt, J=11.2, 4.0 Hz, 1H), 2.65-2.59 (m, 1H), 2.49 (t, J=6.0 Hz, 1H), 1.59 (pd, J=6.9, 2.2 Hz, 2H), 1.44-1.12 (m, 28H), 1.05 (d, J=6.8 Hz, 3H), 0.87 (t, J=6.9 Hz, 2H) ppm. ¹³C NMR (101 MHz, CD₃CN) δ 172.57, 159.61, 155.56, 155.51, 152.89, 152.86, 152.70, 152.66, 145.84, 144.63, 144.49, 137.57, 136.75, 136.67, 136.66, 131.54, 130.99, 130.95, 129.42, 128.99, 128.94, 128.72, 128.05, 128.01, 127.78, 127.75, 119.45, 119.26, 113.98, 113.96, 113.94, 94.68, 94.23, 92.81, 92.36, 88.94, 88.92, 88.59, 88.57, 87.02, 86.93, 82.33, 82.27, 71.19, 71.04, 70.89, 70.51, 70.35, 70.19, 62.69, 62.11, 60.96, 60.05, 59.86, 59.80, 59.60, 55.91, 55.88, 49.15, 44.22, 44.19, 44.09, 44.07, 32.65, 30.40, 30.39, 30.36, 30.31, 30.22, 30.16, 30.08, 29.86, 29.33, 29.32, 27.50, 25.12, 25.03, 24.95, 24.88, 24.81, 23.40, 21.08, 21.01, 20.99, 20.92, 14.53, 14.41 ppm. ¹⁹F NMR (471 MHz, CD₃CN) δ −196.49-−209.61 (m) ppm. ³¹P NMR (202 MHz, CD₃CN) δ 151.59 (d, J=6.8 Hz), 151.33 (d, J=10.9 Hz) ppm.

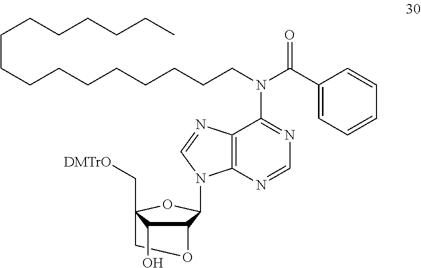

N-[9-[(4R,6R)-4-[[bis(4-methoxyphenyl)-phenyl-methoxy]methyl]-7-hydroxy-2,5-dioxabicyclo[2.2.1]heptan-6-yl]purin-6-yl]-N-hexadecyl-benzamide [compound 30]: To a clear solution of 16 (3.0 g, 4.29 mmol) in dry dimethylformamide (20 mL) was added potassium carbonate (2.39 g, 17.15 mmol, 1.05 mL), 1-bromohexadecane (6.75 g, 21.44 mmol, 6.75 mL) in single portion and stirred for 16 hr at 22° C. Reaction mixture was diluted with EtOAc (30 mL) and organic layer was washed with water (30 mL) and brine (2×30 mL). Organic layer was dried over anhydrous Na₂SO₄, filtered and filtrate was evaporated to dryness. Crude compound was purified by column chromatography (gradient: 10-50% EtOAc in hexane) to afford 30 (1.62 g, 41% yield) as white foam. ¹H NMR (400 MHz, CDCl₃) δ 8.54 (s, 1H), 8.13 (s, 1H), 7.43 (tt, J=7.1, 1.5 Hz, 4H), 7.37-7.20 (m, 9H), 7.18-7.09 (m, 2H), 6.89-6.79 (m, 4H), 6.03 (s, 1H), 4.60 (s, 1H), 4.36-4.28 (m, 3H), 4.01 (d, J=2.3 Hz, 2H), 3.80 (s, 6H), 2.32 (d, J=6.0 Hz, 1H), 1.81-1.66 (m, 2H), 1.23 (d, J=11.1 Hz, 24H), 0.91-0.83 (m, 3H) ppm. ¹³C NMR (101 MHz, CDCl₃) δ 172.27, 158.87, 154.71, 152.19, 151.43, 144.41, 140.72, 136.66, 135.45, 135.38, 130.69, 130.14, 130.12, 128.71, 128.20, 128.13, 127.99, 127.26, 127.08, 113.49, 87.54, 86.88, 86.42, 79.49, 72.26, 72.02, 59.27, 55.38, 48.91, 32.06, 29.83, 29.79, 29.74, 29.70, 29.49, 29.47, 28.92, 27.06, 22.83, 14.26 ppm.

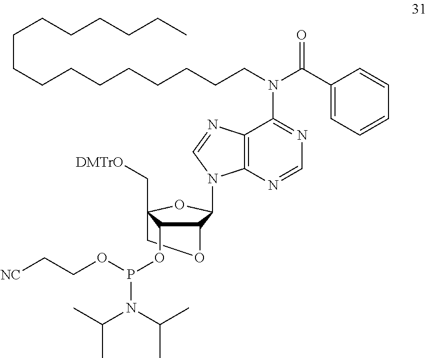

N-[9-[(4R,6R)-4-[[bis(4-methoxyphenyl)-phenyl-methoxy]methyl]-7-[2-cyanoethoxy-(diisopropylamino)phosphanyl]oxy-2,5-dioxabicyclo[2.2.1]heptan-6-yl]purin-6-yl]-N-hexadecyl-benzamide [compound 31]: To a clear solution of 30 (1.6 g, 1.76 mmol) in DCM (20 mL) at room temperature was added N-methyl imidazole (288.66 mg, 3.52 mmol, 280.25 μL) and diisopropylethylamine (1.14 g, 8.79 mmol, 1.53 mL). The reaction mixture was stirred for 5 minutes at rt and 2-cyanoethyl-N,N-diisopropylchlorophosphoramidite (832.15 mg, 3.52 mmol, 785.04 μL) was added slowly into it. Reaction was kept for stirring at rt and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 31 (1.68 g, 86% yield) as white foam. ¹H NMR (500 MHz, CD₃CN) δ 8.46 (d, J=3.0 Hz, 1H), 8.21 (d, J=14.2 Hz, 1H), 7.46 (ft, J=6.7, 1.3 Hz, 2H), 7.38-7.20 (m, 11H), 7.15 (ddd, J=9.0, 7.5, 1.7 Hz, 2H), 6.91-6.82 (m, 5H), 6.03 (d, J=4.3 Hz, 1H), 4.94-4.59 (m, 1H), 4.55-4.37 (m, 1H), 4.29 (tq, J=8.5, 3.0 Hz, 2H), 4.07-3.90 (m, 2H), 3.81-3.65 (m, 7H), 3.59-3.40 (m, 5H), 2.65-2.35 (m, 2H), 1.67 (p, J=7.5 Hz, 2H), 1.39-1.14 (m, 32H), 1.09 (dd, J=6.8, 2.4 Hz, 6H), 0.93-0.84 (m, 10H) ppm. ¹³C NMR (101 MHz, CD₃CN) δ 172.64, 159.78, 155.27, 155.24, 152.69, 152.59, 152.53, 145.92, 145.85, 142.40, 142.37, 137.81, 136.61, 136.55, 136.42, 136.35, 131.52, 131.49, 131.13, 131.07, 130.99, 129.45, 129.42, 129.00, 128.90, 128.80, 128.08, 128.06, 127.97, 127.93, 119.23, 119.18, 114.13, 114.11, 88.65, 88.61, 88.58, 87.71, 87.16, 79.42, 79.39, 79.19, 79.16, 73.68, 73.50, 73.36, 72.81, 72.66, 60.31, 60.00, 59.74, 59.55, 59.45, 59.26, 55.95, 55.92, 49.10, 44.11, 44.04, 43.98, 43.92, 32.65, 30.40, 30.38, 30.36, 30.32, 30.25, 30.19, 30.08, 29.95, 29.35, 29.33, 27.54, 24.94, 24.86, 24.78, 24.76, 24.71, 24.68, 23.40, 20.98, 20.95, 20.91, 20.88, 14.42 ppm. ³¹P NMR (202 MHz, CD₃CN) δ 149.96, 149.80 ppm.

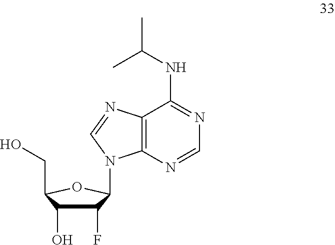

(2R,5R)-4-fluoro-2-(hydroxymethyl)-5-[6-(isopropylamino)purin-9-yl]tetrahydrofuran-3-ol [Compound 33]: To a suspension of commercially available 32 (1.5 g, 5.20 mmol) in ethanol (10 mL) was added triethylamine (1.58 g, 15.59 mmol, 2.17 mL) and isopropylamine (921.47 mg, 15.59 mmol, 1.33 mL). The resulting mixture was heated at 80° C. for 10 hr and TLC was checked. All the volatile matters were removed under high vacuum pump and crude compound was purified by flash column chromatography (gradient: 0-7% MeOH in DCM) to afford 33 (1.51 g, 93% yield) as white foam. ¹H NMR (600 MHz, DMSO) δ 8.36 (s, 1H), 8.21 (s, 1H), 7.66 (d, J=8.1 Hz, 1H), 6.23 (dd, J=16.9, 3.0 Hz, 1H), 5.71 (d, J=6.0 Hz, 1H), 5.51-5.35 (m, 1H), 5.26 (dd, J=6.3, 4.9 Hz, 1H), 4.49 (dtd, J=17.2, 6.2, 4.5 Hz, 2H), 3.98 (qd, J=3.8, 1.4 Hz, 1H), 3.75 (ddd, J=12.3, 5.0, 2.8 Hz, 1H), 3.58 (ddd, J=12.4, 6.3, 4.0 Hz, 1H), 1.21 (dd, J=6.5, 2.1 Hz, 6H). ¹⁹F NMR (565 MHz, DMSO) δ −204.44. ¹³C NMR (151 MHz, DMSO) δ 153.90, 152.67, 138.99, 119.42, 93.98, 92.74, 85.92, 85.70, 84.17, 68.40, 68.29, 60.47, 54.91, 41.32, 22.23 ppm.

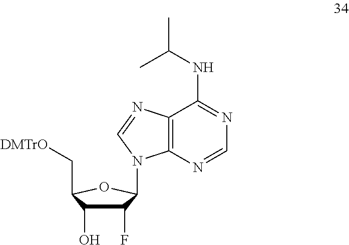

(2R,5R)-2-[[bis(4-methoxyphenyl)-phenyl-methoxy]methyl]-4-fluoro-5-[6-(isopropylamino) purin-9-yl]tetrahydrofuran-3-ol [Compound 34]: To a clear solution of 33 (1.0 g, 3.21 mmol) in dry pyridine (30 mL) was added 4,4′-dimethoxytrityl chloride (1.31 g, 3.85 mmol) in three portions. Reaction mixture was stirred for 16 hr at 22° C. and then quenched with saturated NaHCO₃ solution (30 mL). Resultant mixture was extracted with DCM (2×40 mL). The combined organic layer was separated, washed with brine (40 mL), dried over anhydrous Na2SO4, filtered and the filtrate was evaporated to dryness. Crude compound was purified by combiflash chromatography (gradient: 10-50% EtOAc in hexane) to afford white foam of 34 (1.60 g, 81% yield) as white foam. ¹H NMR (600 MHz, DMSO) δ 8.29 (s, 1H), 8.23-8.20 (m, 1H), 7.66 (d, J=8.1 Hz, 1H), 7.31 (dt, J=6.5, 1.4 Hz, 2H), 7.25-7.14 (m, 7H), 6.83-6.76 (m, 4H), 6.33-6.25 (m, 1H), 5.70 (d, J=6.8 Hz, 1H), 5.67-5.51 (m, 1H), 4.89-4.77 (m, 1H), 4.45 (s, 1H), 4.09 (ddd, J=8.1, 5.3, 2.5 Hz, 1H), 3.71 (d, J=2.1 Hz, 6H), 3.27 (dd, J=10.7, 2.5 Hz, 2H), 3.20 (dd, J=10.7, 5.3 Hz, 1H). ¹³C NMR (151 MHz, DMSO) δ 158.02, 158.00, 144.80, 139.47, 135.47, 135.45, 129.67, 127.71, 127.65, 126.57, 113.08, 113.06, 93.99, 92.77, 86.53, 86.30, 85.34, 81.26, 68.72, 68.61, 62.64, 59.75, 54.98, 22.23 ppm.

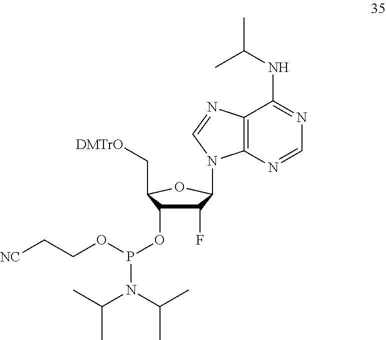

3-[[(2R,5R)-2-[[bis(4-methoxyphenyl)-phenyl-methoxy]methyl]-4-fluoro-5-[6-(isopropylamino)purin-9-yl]tetrahydrofuran-3-yl]oxy-(diisopropylamino)phosphanyl]oxypropanenitrile [Compound 35]: Compound 34 was converted to 35 following the literature procedure¹¹. To a clear solution of 34 (0.49 g, 798.47 μmol) in dry dichloromethane (30 mL) was added 2-cyanoethyl tetraisopropylphosphorodiamidite (361.00 mg, 1.20 mmol, 380.40 μL) followed by pyridinium trifluoroacetate (231.30 mg, 1.20 mmol) in single portions. After stirring the reaction mixture for 5.0 hr at 22° C., volatile matters were removed under high vacuum pump at 36° C. and the residue thus obtained, was purified by flash chromatography (30-60% EtOAc in hexane) to afford 35 (0.54 g, 663.48 μmol, 83% yield) as white foam. ¹H NMR (600 MHz, CD₃CN) δ 8.26 (s, 1H), 8.06 (d, J=3.0 Hz, 1H), 7.37-7.30 (m, 2H), 7.23-7.16 (m, 4H), 6.83-6.74 (m, 4H), 6.39-6.18 (m, 1H), 6.13 (s, 1H), 5.98-5.71 (m, 1H), 5.55-5.17 (m, 1H), 4.50 (s, 1H), 4.28-4.22 (m, 1H), 3.96-3.78 (m, 1H), 3.78-3.74 (m, 6H), 3.69-3.55 (m, 2H), 3.53-3.41 (m, 1H), 3.24-3.16 (m, 1H), 2.72-2.65 (m, 1H), 1.31-1.26 (m, 6H), 1.21-1.15 (m, 10H), 1.06 (d, J=6.8 Hz, 3H). ³¹P NMR (243 MHz, CD₃CN) δ 150.05, 150.03, 149.83, 149.79. ¹⁹F NMR (565 MHz, CD₃CN) δ −200.19, −200.20, −200.39, −200.40. ¹³C NMR (151 MHz, CD₃CN) δ 159.54, 159.52, 155.32, 153.89, 150.70, 145.96, 145.93, 140.84, 140.77, 136.88, 136.77, 136.69, 136.61, 136.57, 130.96, 130.94, 130.91, 128.92, 128.85, 128.67, 127.71, 127.68, 124.72, 119.52, 119.29, 113.88, 113.86, 113.84, 94.58, 94.20, 94.18, 93.35, 92.95, 88.88, 88.86, 88.63, 86.81, 86.71, 82.12, 82.07, 82.03, 71.33, 71.23, 71.13, 70.53, 70.43, 70.32, 62.99, 62.30, 60.94, 60.03, 59.90, 59.80, 59.67, 55.82, 55.80, 44.10, 44.08, 44.02, 44.00, 25.01, 24.99, 24.96, 24.94, 24.91, 24.87, 24.82, 24.78, 22.82, 21.05, 21.01, 20.93, 20.88 ppm.

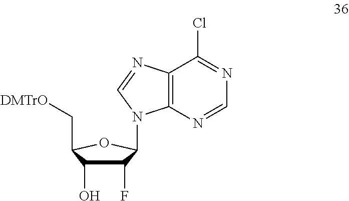

(2R,5R)-2-[[bis(4-methoxyphenyl)-phenyl-methoxy]methyl]-S-(6-chloropurin-9-yl)-4-fluoro-tetrahydrofuran-3-ol [Compound 36]: To a clear solution of commercially available 32 (1.5 g, 5.20 mmol) in dry pyridine (30 mL) was added 4,4′-dimethoxytrityl chloride (2.11 g, 6.24 mmol) in three portions. Reaction mixture was stirred for 16 hr at 22° C. and then quenched with saturated NaHCO₃ solution (30 mL). Resultant mixture was extracted with DCM (2×40 mL). The combined organic layer was separated, washed with brine (40 mL), dried over anhydrous Na₂SO₄, filtered and the filtrate was evaporated to dryness. Crude compound was purified by flash column chromatography (gradient: 10-50% EtOAc in hexane) to afford 36 (2.48 g, 81% yield) as yellowish white foam. ¹H NMR (600 MHz, DMSO-d₆) δ 8.83 (s, 1H), 8.80 (s, 1H), 7.25-7.12 (m, 9H), 6.85-6.72 (m, 6H), 6.49-6.38 (m, 1H), 5.79 (d, J=7.0 Hz, 1H), 5.70-5.52 (m, 1H), 4.84-4.74 (m, 1H), 4.15 (ddd, J=8.3, 5.2, 2.5 Hz, 1H), 3.71 (d, J=3.4 Hz, 9H), 3.31-3.23 (m, 3H). ¹⁹F NMR (565 MHz, DMSO-d₆) δ −201.27. ¹³C NMR (151 MHz, DMSO-d₆) δ 158.05, 158.02, 151.84, 151.03, 149.55, 149.49, 146.27, 144.77, 136.35, 135.39, 135.37, 131.53, 129.70, 129.66, 127.76, 127.61, 126.68, 124.02, 113.10, 113.06, 93.93, 92.71, 87.24, 87.01, 85.42, 81.58, 68.72, 68.61, 62.60, 59.82, 55.03, 55.01 ppm.

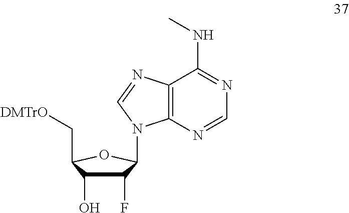

(2R,5R)-2-[[bis(4-methoxyphenyl)-phenyl-methoxy]methyl]-4-fluoro-5-[6-(methylamino) purin-9-yl]tetrahydrofuran-3-ol [Compound 37]: To a clear solution of 36 (0.6 g, 1.02 mmol) in ethanol (15 mL) was added triethylamine (410.90 mg, 4.06 mmol, 565.98 μL) and methylamine hydrochloride (137.09 mg, 2.03 mmol). The resulting mixture was heated at 70° C. for 10 hr and TLC was checked. All the volatile matters were removed under high vacuum pump and crude compound was purified by flash column chromatography (gradient: 0-5% MeOH in DCM) to afford 37 (0.5 g, 84% yield) as white foam. ¹H NMR (600 MHz, DMSO-d₆) δ 8.30 (d, J=2.7 Hz, 1H), 8.25 (s, 1H), 7.88 (s, 1H), 7.33-7.28 (m, 3H), 7.25-7.14 (m, 9H), 6.79 (ddd, J=12.2, 9.0, 2.9 Hz, 5H), 6.34-6.27 (m, 1H), 5.76 (d, J=0.9 Hz, 1H), 5.74-5.70 (m, 1H), 5.69-5.54 (m, 1H), 4.91-4.74 (m, 1H), 4.08 (ddd, J=8.2, 5.3, 2.5 Hz, 1H), 3.73-3.69 (m, 7H), 3.26 (dt, J=10.7, 2.8 Hz, 1H), 3.17 (dd, J=10.7, 5.1 Hz, 1H), 2.94 (d, J=4.5 Hz, 3H) ppm. ¹³C NMR (151 MHz, DMSO-d₆) δ 158.03, 158.00, 155.06, 152.90, 147.79, 144.83, 139.68, 135.51, 135.47, 129.72, 129.67, 127.78, 127.68, 126.64, 119.70, 113.11, 113.08, 94.04, 92.82, 86.56, 86.33, 85.35, 81.26, 68.73, 68.62, 62.65, 55.01, 55.00, 54.98, 27.01. ¹⁹F NMR (565 MHz, DMSO-d₆) δ −201.15 ppm.

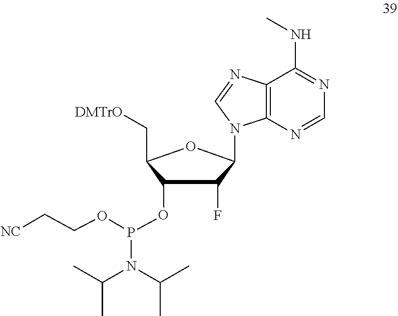

3-[[(2R,5R)-2-[[bis(4-methoxyphenyl)-phenyl-methoxy]methyl]-4-fluoro-5-[6-(methylamino)purin-9-yl]tetrahydrofuran-3-yl]oxy-(diisopropylamino)phosphanyl]oxypropanenitrile [Compound 39]: Compound 37 was converted to 39 following the literature procedure¹¹. To a clear solution of 37 (0.49 g, 836.71 μmol) in dry dichloromethane (20 mL) was added 2-cyanoethyl tetraisopropylphosphorodiamidite (378.29 mg, 1.26 mmol, 398.62 μL) followed by pyridinium trifluoroacetate (242.38 mg, 1.26 mmol) in single portions. After stirring the reaction mixture for 5 hr at 22° C., volatile matters were removed under high vacuum pump at 36° C. and the residue thus obtained, was purified by flash chromatography (30-60% EtOAc in hexane) to afford 39 (0.50 g, 76% yield) as white foam. ¹H NMR (600 MHz, CD₃CN) δ 8.26 (s, 1H), 8.02 (d, J=3.4 Hz, 1H), 6.79-6.71 (m, 4H), 6.30 (s, 1H), 6.27-6.14 (m, 1H), 5.94-5.69 (m, 1H), 5.41-5.13 (m, 1H), 4.22 (dtd, J=8.9, 4.4, 2.0 Hz, 1H), 3.94-3.76 (m, 1H), 3.74 (dd, J=4.4, 1.3 Hz, 6H), 3.67-3.52 (m, 2H), 3.51-3.36 (m, 1H), 3.17 (td, J=11.2, 4.4 Hz, 1H), 3.05 (s, 3H), 2.69-2.62 (m, 1H), 2.48 (t, J=6.0 Hz, 1H), 1.21-1.10 (m, 9H), 1.03 (d, J=6.8 Hz, 3H). ¹³C NMR (151 MHz, CD₃CN) δ 159.55, 159.52, 156.54, 153.94, 150.71, 145.93, 145.87, 140.87, 140.80, 136.84, 136.76, 136.63, 136.58, 130.96, 130.95, 130.89, 130.87, 128.96, 128.88, 128.68, 127.74, 127.70, 119.53, 119.30, 113.88, 113.86, 113.84, 94.58, 94.18, 93.34, 92.96, 88.88, 88.86, 88.65, 88.63, 86.82, 86.73, 82.14, 82.09, 82.06, 71.36, 71.26, 71.16, 70.57, 70.46, 70.36, 63.05, 62.37, 60.94, 60.04, 59.91, 59.82, 59.68, 55.82, 55.80, 55.32, 44.11, 44.09, 44.03, 44.01, 27.40, 25.01, 24.99, 24.96, 24.93, 24.91, 24.86, 24.82, 24.78, 21.06, 21.01, 20.93, 20.88. ³¹P NMR (243 MHz, CD₃CN) δ 150.05 (d, J=5.8 Hz), 149.81 (d, J=10.1 Hz). ¹⁹F NMR (565 MHz, CD₃CN) δ −200.31, −200.52 ppm.

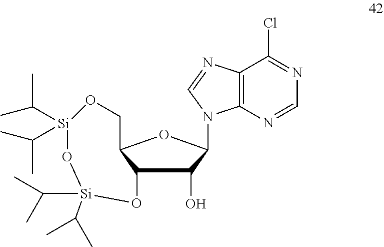

(6aR,8R)-8-(6-chloropurin-9-yl)-2,2,4,4-tetraisopropyl-6a,8,9,9a-tetrahydro-6H-furo[3,2-f][1,3,5,2,4]trioxadisilocin-9-ol [Compound 42⁸]: To a clear solution of commercially available 41 (3.8 g, 13.26 mmol) in pyridine (40 mL) was added 1,3-dichloro-1,1,3,3-tetraisopropyldisiloxane (5.02 g, 15.91 mmol) in single portion. Reaction mixture was stirred at 22° C. for 16 hr. All volatile matters were removed under high vacuum pump and residue was dissolved in EtOAc (100 mL). Organic layer was washed with 10% NaHCO₃ solution (50 mL) and brine (2×40 mL). EtOAc layer was separated, dried over anhydrous Na₂SO₄, filtered and filtrate was evaporated to dryness. crude compound was purified by flash column chromatography (gradient: 10-40% EtOAc in hexane) to afford 42 (4.93 g, 9.32 mmol, 70.28% yield) as white foam. ¹H NMR (600 MHz, DMSO-d₆) δ 8.72 (s, 1H), 8.72 (s, 2H), 6.02 (d, J=1.2 Hz, 1H), 5.74 (d, J=4.6 Hz, 1H), 4.70 (dd, J=8.6, 5.0 Hz, 1H), 4.61 (td, J=4.9, 1.2 Hz, 1H), 4.12-4.03 (m, 2H), 3.94 (dd, J=12.8, 2.7 Hz, 1H), 1.08-0.91 (m, 32H). ¹³C NMR (151 MHz, DMSO-d₆) δ 151.43, 150.85, 149.33, 145.47, 131.67, 89.93, 81.03, 73.40, 69.59, 60.55, 17.29, 17.12, 17.11, 17.06, 16.97, 16.87, 16.84, 16.78, 12.69, 12.40, 12.24, 12.03. ppm.

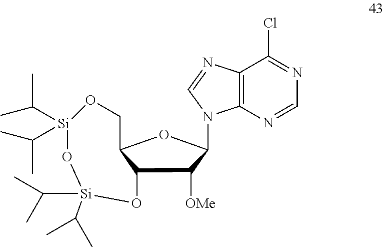

9-[(6aR,8R)-2,2,4,4-tetraisopropyl-9-methoxy-6a,8,9,9a-tetrahydro-6H-furo[3,2-f][1,3,5,2,4]trioxadisilocin-8-yl]-6-chloro-purine [Compound 43⁸]: A mixture of 42 (2.0 g, 3.78 mmol) silver (I) oxide (4.40 g, 18.99 mmol) and iodomethane (45.60 g, 321.26 mmol, 20.00 mL) was stirred under heating condition at 40° C. for 1.0 hr. Reaction mixture was filtered and solid residue was washed with CHCl₃ (30 mL). All the volatile matters from the filtrate were removed under high vacuum pump and residue was dissolved in CHCl₃ (30 mL). Organic layer was washed with water (30 mL), brine (2×20 mL) and then separated, dried over anhydrous Na₂SO₄, filtered and the filtrate was evaporated to dryness. Crude residue was purified by flash column chromatography (gradient: 20-50% EtOAc in hexane) to afford 43 (1.2 g, 58% yield) as white solid. ¹H NMR (600 MHz, CDCl₃) δ 8.74 (s, 1H), 8.47 (s, 1H), 6.10 (s, 1H), 4.68 (dd, J=9.4, 4.6 Hz, 1H), 4.28-4.23 (m, 1H), 4.18 (ddd, J=9.4, 2.6, 1.4 Hz, 1H), 4.08-4.00 (m, 2H), 3.72 (s, 3H), 1.15-0.95 (m, 29H). ¹³C NMR (151 MHz, CDCl₃) δ 152.14, 151.27, 150.72, 143.66, 132.65, 88.65, 83.80, 81.72, 69.41, 59.78, 59.68, 17.59, 17.51, 17.46, 17.43, 17.28, 17.17, 17.00, 13.59, 13.08, 13.01, 12.64 ppm.

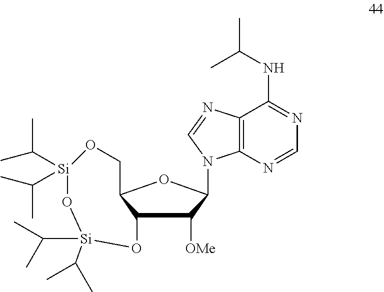

9-[(6aR,8R)-2,2,4,4-tetraisopropyl-9-methoxy-6a,8,9,9a-tetrahydro-6H-furo[3,2-f][1,3,5,2,4]trioxadisilocin-8-yl]-N-isopropyl-purin-6-amine [Compound 44]: To a clear solution of 43 (1.3 g, 2.39 mmol) in ethanol (30 mL) was added isopropyl amine (565.85 mg, 9.57 mmol, 818.89 μL) and isopropyl amine (565.85 mg, 9.57 mmol, 818.89 μL) in single portions. Reaction mixture was stirred at 70° C. for 10 hr. All the volatile matters were removed under high vacuum pump and the residue thus obtained was purified by flash column chromatography (gradient: 10-40% EtOAc in hexane) to afford 44 (1.31 g, 97% yield) as white solid. ¹H NMR (600 MHz, CDCl₃) δ 8.35 (s, 1H), 8.05 (s, 1H), 6.01 (s, 1H), 5.65 (s, 1H), 4.75 (dd, J=9.4, 4.7 Hz, 1H), 4.50 (s, 1H), 4.23 (dd, J=13.3, 1.7 Hz, 1H), 4.14 (dt, J=9.4, 2.0 Hz, 1H), 4.05 (d, J=4.7 Hz, 1H), 4.02 (dd, J=13.3, 2.6 Hz, 1H), 3.70 (s, 3H), 1.32 (dd, J=6.5, 3.0 Hz, 6H), 1.14-0.96 (m, 29H). ¹³C NMR (151 MHz, CDCl₃) δ 154.26, 153.39, 138.01, 120.39, 88.35, 83.93, 81.39, 69.64, 59.99, 59.70, 42.61, 23.13, 17.61, 17.51, 17.46, 17.30, 17.21, 17.19, 17.03, 13.57, 13.10, 13.01, 12.64 ppm.

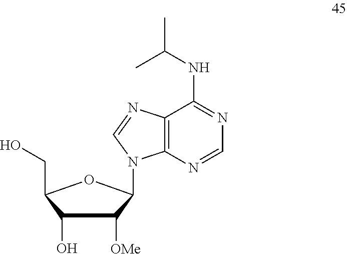

(2R,5R)-2-(hydroxymethyl)-5-[6-(isopropylamino)purin-9-yl]-4-methoxy-tetrahydrofuran-3-ol [Compound 45]: To a clear solution of 44 (1.11 g, 1.96 mmol) in THF (30 mL) was added tetrabutylammonium fluoride (1.23 g, 4.71 mmol, 1.36 mL) in single portion. Reaction mixture was stirred for 2 hr at 22° C. and TLC was checked which showed complete consumption of starting material. All the volatile matters were removed under high vacuum pump and the crude residue was purified by flash column chromatography (gradient: 0-5% MeOH in DCM) to afford 45 (0.62 g, 98% yield) as white foam. ¹H NMR (600 MHz, DMSO) δ 8.38 (s, 1H), 8.21 (s, 1H), 7.67 (s, 1H), 6.00 (d, J=6.1 Hz, 1H), 5.75 (s, 1H), 5.44 (d, J=5.9 Hz, 1H), 5.27 (d, J=5.1 Hz, 1H), 4.44 (s, 1H), 4.39-4.31 (m, 2H), 3.98 (q, J=3.4 Hz, 1H), 3.67 (dt, J=12.1, 3.9 Hz, 1H), 3.56 (ddd, J=12.1, 6.9, 3.6 Hz, 1H), 3.30 (s, 3H), 1.21 (dd, J=6.5, 2.0 Hz, 6H) ppm. ¹³C NMR (151 MHz, DMSO) δ 153.93, 152.51, 148.23, 139.36, 119.58, 86.47, 85.88, 82.47, 68.80, 61.50, 57.47, 54.93, 41.33, 22.24, 17.32, 12.63 ppm.

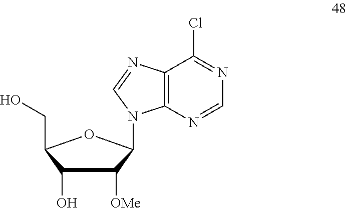

(2R,5R)-5-(6-chloropurin-9-yl)-2-(hydroxymethyl)-4-methoxy-tetrahydrofuran-3-ol [Compound 48]: To a clear solution of 43 (1.8 g, 3.31 mmol) in THF (30 mL) was added tetrabutylammonium fluoride (2.25 g, 8.62 mmol, 2.49 mL) in single portion and stirred for 5 hr at 22° C. All the volatile matters were evaporated under high vacuum pump and the crude residue thus obtained, was purified by flash column chromatography (gradient: 0-5% MeOH in DCM) to afford 48 (0.95 g, 95% yield) as white foam. ¹H NMR (600 MHz, DMSO) δ 8.98 (s, 1H), 8.83 (s, 1H), 6.21-6.14 (m, 1H), 5.35 (s, 1H), 5.16 (s, 1H), 4.36 (d, J=3.5 Hz, 3H), 4.00 (q, J=3.7 Hz, 1H), 3.70 (dd, J=12.1, 3.8 Hz, 1H), 3.59 (dd, J=12.0, 3.8 Hz, 1H), 3.37 (s, 3H). ¹³C NMR (151 MHz, DMSO) δ 151.91, 151.47, 149.40, 145.62, 131.41, 86.17, 82.87, 79.23, 68.51, 60.80, 57.69 ppm.

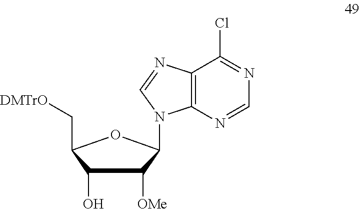

(2R,5R)-2-[[bis(4-methoxyphenyl)-phenyl-methoxy]methyl]-5-(6-chloropurin-9-yl)-4-methoxy-tetrahydrofuran-3-ol [Compound 49]: To a clear solution of 48 (0.85 g, 2.83 mmol) in dry pyridine (20 mL) was added 4,4′-dimethoxytrityl chloride (1.15 g, 3.39 mmol) in three portions. Reaction mixture was stirred for 16 hr at 22° C. and then quenched with saturated NaHCO₃ solution (30 mL). Resultant mixture was extracted with DCM (2×40 mL). The combined organic layer was separated, washed with brine (40 mL), dried over anhydrous Na₂SO₄, filtered and the filtrate was evaporated to dryness. Crude compound was purified by flash column chromatography (gradient: 10-50% EtOAc in hexane) to afford 49 (1.25 g, 73% yield) as white foam. ¹H NMR (600 MHz, DMSO) δ 8.83 (s, 1H), 8.74 (s, 1H), 7.35-7.30 (m, 2H), 7.26-7.16 (m, 7H), 6.85-6.76 (m, 4H), 6.20 (d, J=3.9 Hz, 1H), 5.36 (d, J=6.2 Hz, 1H), 4.53-4.45 (m, 2H), 4.12 (td, J=5.6, 3.3 Hz, 1H), 3.39 (s, 3H), 3.31-3.20 (m, 2H). ¹³C NMR (151 MHz, DMSO) δ 158.05, 158.01, 151.73, 151.28, 149.52, 149.47, 146.15, 144.80, 136.27, 135.45, 135.43, 131.56, 129.70, 129.65, 127.75, 127.63, 126.65, 123.96, 113.10, 113.07, 86.69, 85.47, 83.68, 81.78, 69.03, 63.40, 59.76, 57.88, 55.00, 54.99 ppm.

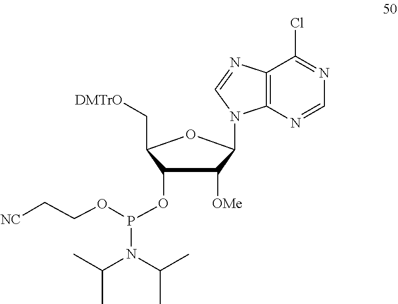

3-[[(2R,5R)-2-[[bis(4-methoxyphenyl)-phenyl-methoxy]methyl]-5-(6-chloropurin-9-yl)-4-methoxy-tetrahydrofuran-3-yl]oxy-(diisopropylamino)phosphanyl]oxypropanenitrile [Compound 50]: To a clear solution of 49 (1.0 g, 1.66 mmol) in dichloromethane (30 mL) was added N-methylimidazole (204.21 mg, 2.49 mmol, 198.26 μL) and diisopropylethylamine (1.07 g, 8.29 mmol, 1.44 mL) in single portions. After stirring the reaction mixture for 5 minutes at 22° C., 2-cyanoethyl N,N-diisopropylchlorophosphoramidite (784.93 mg, 3.32 mmol, 740.50 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (30-55% EtOAc in hexane) to afford 50 (1.10 g, 83% yield) as white foam. ¹H NMR (600 MHz, CD₃CN) δ 8.65-8.59 (m, 1H), 8.45-8.39 (m, 1H), 7.58-7.06 (m, 13H), 6.80 (dddd, J=12.8, 9.7, 5.8, 2.3 Hz, 8H), 6.14 (dq, J=5.3, 2.6 Hz, 2H), 4.80-4.69 (m, 2H), 4.63 (dt, J=9.4, 4.6 Hz, 2H), 4.36-4.28 (m, 2H), 3.94-3.73 (m, 12H), 3.68-3.56 (m, 4H), 3.51-3.39 (m, 7H), 3.35 (ddd, J=15.9, 10.8, 4.9 Hz, 2H), 2.70-2.60 (m, 2H), 2.50 (t, J=6.0 Hz, 2H), 1.24-1.14 (m, 30H), 1.08 (t, J=6.2 Hz, 8H). ³¹P NMR (243 MHz, CD₃CN) δ 149.97, 149.74. ¹³C NMR (151 MHz, CD₃CN) δ 159.67, 159.65, 152.61, 152.57, 152.50, 151.17, 151.15, 146.23, 146.04, 145.92, 145.90, 136.68, 136.65, 136.61, 133.30, 133.25, 131.08, 131.03, 131.00, 129.01, 128.95, 128.85, 128.80, 128.78, 127.87, 127.85, 119.59, 119.37, 113.99, 88.65, 88.41, 87.24, 84.44, 84.43, 84.36, 84.32, 82.96, 82.95, 82.72, 82.69, 71.91, 71.81, 71.70, 63.92, 63.49, 60.95, 59.87, 59.75, 59.39, 59.25, 59.19, 59.17, 58.90, 55.88, 55.87, 44.13, 44.05, 43.97, 25.02, 24.97, 24.90, 24.86, 21.14, 21.04, 21.00, 20.95 ppm.

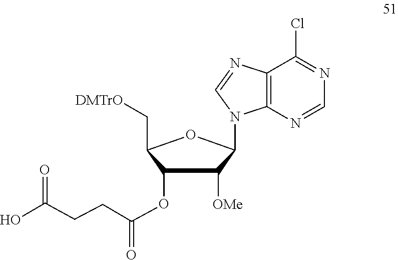

4-[(2R,5R)-2-[[bis(4-methoxyphenyl)-phenyl-methoxy]methyl]-5-(6-chloropurin-9-yl)-4-methoxy-tetrahydrofuran-3-yl]oxy-4-oxo-butanoic acid [Compound 51]: To a clear solution of 49 (0.45 g, 746.19 μmol) in dichloromethane (20 mL) was added DMAP (364.65 mg, 2.98 mmol) and succinic anhydride (224.01 mg, 2.24 mmol) sequentially. The resulting mixture was stirred for 4.0 hr at 22° C. Reaction mixture was diluted with DCM (15 mL) and organic layer was washed with 10% NH₄Cl solution (2×20 mL). Organic layer was separated, dried over anhydrous Na2SO4, filtered and the filtrate was evaporated to dryness. The crude mass thus obtained was purified by flash column chromatography (gradient: 0-5% MeOH in DCM) for afford 51 (0.43 g, 82% yield) as white foam. ¹H NMR (600 MHz, DMSO) δ 12.27 (s, 1H), 8.88 (s, 1H), 8.68 (s, 1H), 7.35-7.30 (m, 2H), 7.22-7.16 (m, 6H), 6.86-6.76 (m, 5H), 6.20 (d, J=5.8 Hz, 1H), 5.51 (dd, J=5.3, 3.8 Hz, 1H), 4.96 (t, J=5.6 Hz, 1H), 4.30 (dt, J=5.9, 3.8 Hz, 1H), 3.72 (d, J=3.0 Hz, 7H), 3.39 (dd, J=10.6, 6.0 Hz, 1H), 3.28 (dd, J=10.6, 3.7 Hz, 1H), 3.25 (s, 3H), 2.61 (dd, J=7.7, 5.4 Hz, 2H), 2.55-2.52 (m, 2H). ¹³C NMR (151 MHz, DMSO) δ 173.26, 171.50, 158.09, 158.04, 151.72, 151.31, 149.65, 146.56, 144.70, 135.33, 135.26, 131.72, 129.73, 129.61, 127.76, 127.60, 126.70, 113.12, 113.08, 86.63, 85.69, 81.91, 79.35, 70.84, 63.26, 58.22, 55.01, 54.98, 28.70, 28.66 ppm.

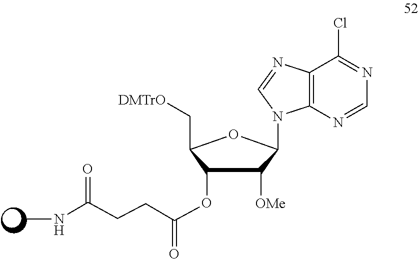

CPG 52: Added 51 (0.43 g, 611.55 μmol) and diisopropylethylamine (316.14 mg, 2.45 mmol, 426.07 μL) into rb flask. Then added dry acetonitrile (50 mL). Stirred well to dissolve and then HBTU (243.52 mg, 642.12 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H2O/THF, then MeOH, then 10% H2O/THF, then MeOH, then ACN, the ether (˜250 mL each solvent for washing). Transferred to rb flask and dried CPG in high vacuum overnight.

Checking the Loading: Weighted out 44.8 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 4.1 g, Loading: 102 μmol/g.

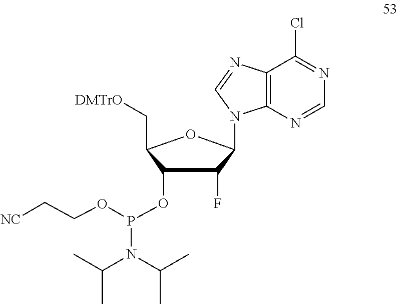

3-[[(2R,5R)-2-[[bis(4-methoxyphenyl)-phenyl-methoxy]methyl]-5-(6-chloropurin-9-yl)-4-fluoro-tetrahydrofuran-3-yl]oxy-(diisopropylamino)phosphanyl]oxypropanenitrile [Compound 53]: To a clear solution of 36 (1.65 g, 2.79 mmol) in dichloromethane was added N-methylimidazole (229.20 mg, 2.79 mmol, 222.53 μL) and diisopropylethylamine (360.81 mg, 2.79 mmol, 486.26 μL) in single portions. After stirring the reaction mixture for 5 minutes at 22° C., 2-cyanoethyl-N,N-diisopropylchlorophosphoramidite (660.75 mg, 2.79 mmol) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (30-55% EtOAc in hexane) to afford 53 (1.77 g, 80% yield) as white foam. ¹H NMR (600 MHz, CD₃CN) δ 8.68 (d, J=4.5 Hz, 1H), 8.46 (d, J=3.5 Hz, 1H), 7.36-7.30 (m, 2H), 7.26-7.15 (m, 8H), 6.81-6.73 (m, 4H), 6.46-6.23 (m, 1H), 5.85-5.56 (m, 1H), 5.30-4.97 (m, 1H), 4.33-4.27 (m, 1H), 3.89-3.71 (m, 8H), 3.68-3.54 (m, 3H), 3.53-3.44 (m, 1H), 3.34-3.27 (m, 1H), 2.67-2.60 (m, 1H), 2.50 (t, J=6.0 Hz, 1H), 1.20-1.14 (m, 10H), 1.05 (d, J=6.8 Hz, 3H). ³¹P NMR (243 MHz, CD₃CN) δ 150.15 (d, J=7.2 Hz), 150.05 (d, J=9.5 Hz). ¹⁹F NMR (565 MHz, CD₃CN) δ −200.86 (d, J=9.4 Hz), −201.08 (d, J=6.8 Hz). ¹³C NMR (151 MHz, CD₃CN) δ 159.58, 159.56, 152.67, 152.06, 151.29, 151.23, 146.49, 146.36, 145.80, 145.78, 136.57, 136.50, 136.49, 136.44, 133.16, 130.96, 130.94, 130.92, 128.90, 128.84, 128.72, 127.81, 127.79, 119.51, 119.31, 113.89, 113.87, 94.45, 93.90, 93.88, 93.20, 92.65, 92.63, 89.19, 89.17, 88.95, 87.06, 86.97, 82.56, 82.54, 82.51, 82.47, 71.26, 71.17, 71.06, 70.56, 70.46, 70.35, 62.85, 62.28, 60.94, 59.93, 59.80, 59.62, 59.49, 55.84, 55.81, 44.10, 44.09, 44.02, 44.01, 25.02, 24.97, 24.95, 24.92, 24.90, 24.88, 24.80, 24.76, 21.13, 21.02, 20.97, 20.95, 20.90 ppm.

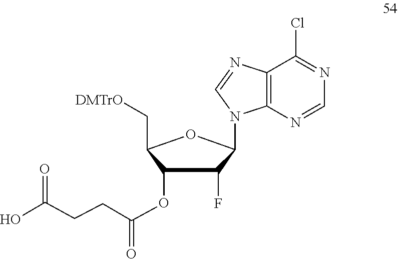

4-[(2R,5R)-2-[[bis(4-methoxyphenyl)-phenyl-methoxy]methyl]-5-(6-chloropurin-9-yl)-4-fluoro-tetrahydrofuran-3-yl]oxy-4-oxo-butanoic acid [Compound 54]: To a clear solution of 36 (0.75 g, 1.27 mmol) in dichloromethane (20 mL) was added DMAP (620.12 mg, 5.08 mmol) and succinic anhydride (380.96 mg, 3.81 mmol) sequentially. The resulting mixture was stirred for 4 hr at 22° C. Reaction mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-5% MeOH in DCM) for afford 54 (0.60 g, 68% yield) as white foam. ¹H NMR (600 MHz, DMSO-d₆) δ 12.31 (s, 1H), 8.86 (s, 1H), 8.76 (d, J=0.8 Hz, 1H), 7.27 (dt, J=6.4, 1.4 Hz, 2H), 7.25-7.12 (m, 7H), 6.87-6.74 (m, 4H), 6.59-6.43 (m, 1H), 6.07-5.90 (m, 1H), 5.83-5.75 (m, 2H), 4.37 (dt, J=8.0, 4.1 Hz, 1H), 3.71 (dd, J=3.7, 2.8 Hz, 7H), 3.34-3.28 (m, 2H), 2.61 (td, J=6.3, 3.0 Hz, 2H), 2.51 (d, J=6.7 Hz, 2H) ppm. ¹³C NMR (151 MHz, DMSO-d₆) δ 173.24, 171.39, 158.08, 158.05, 151.83, 151.01, 149.61, 146.71, 144.65, 140.27, 135.30, 135.17, 131.68, 129.69, 129.61, 128.96, 127.79, 127.69, 127.56, 127.47, 126.71, 123.99, 113.12, 113.08, 112.80, 91.48, 90.22, 87.43, 87.20, 85.68, 79.85, 70.24, 70.14, 62.05, 55.03, 55.01, 54.98, 28.54, 28.37 ppm.

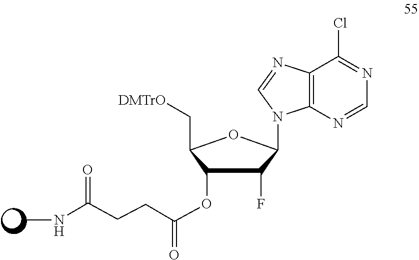

CPG 55: Added 54 (0.6 g, 868.18 μmol) and diisopropylethylamine (448.81 mg, 3.47 mmol, 604.87 μL) into rb flask. Then added dry acetonitrile (50 mL). Stirred well to dissolve and then HBTU (345.71 mg, 911.59 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜ 5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 0% H₂O/THF, then MeOH, then 10% H₂O/THF, then MeOH, then ACN, the ether (˜250 mL each solvent for washing). Transferred to rb flask and dried CPG in high vacuum overnight.

Loading: Weighted out 39 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 6.31 g, Loading: 94 μmol/g.

### Synthesis of Controlled Pore Glass (CPG) Support for N6-Me-A

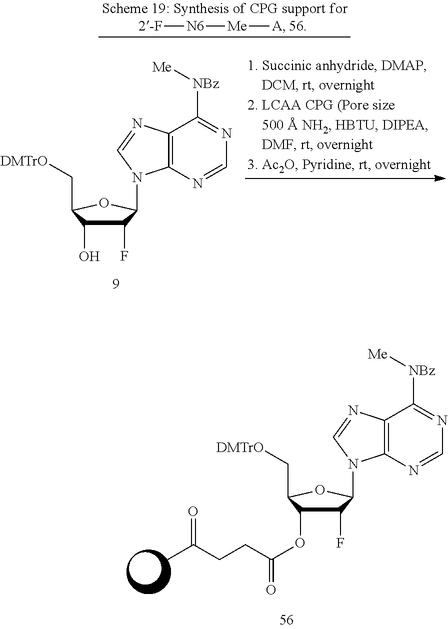

To a solution of compound 9 (100 mg, 0.145 mmol) in DCM (1 mL) were added succinic anhydride (29 mg, 0.29 mmol) and DMAP (54 mg, 0.45 mmol), and the mixture was stirred at room temperature overnight. The reaction mixture was evaporated to dryness and purified by silica gel column chromatography (DCM/MeOH/TEA, 95:4:1, v/v/v). To a solution of the obtained succinate in DMF (4 mL) were added to CPG functionalized with long chain amino alkyl (LCAA) (pore size 500 Å NH₂, loading of 171 μmol/g, 760 mg), DIPEA (0.09 mL, 0.5 mmol), and HBTU (57 mg, 0.15 mmol), and the mixture was agitated on a wrist-action shaker at room temperature overnight. The obtained CPG was filtered, washed with DCM-MeOH (9:1, v/v), and dried. To the suspension of the obtained CPG in pyridine (3 mL) was added acetic anhydride (1 mL), and the reaction mixture was agitated on a wrist-action shaker at room temperature overnight. The CPG was filtered, washed with DCM/MeOH (9:1, v/v), and dried to give CPG support 56 (88 μmol/g).

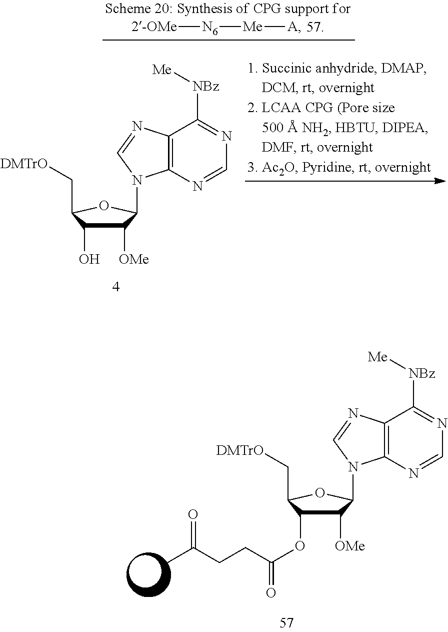

To a clear solution of 4 (0.77 g, 1.10 mmol) in dichloromethane (15 mL) was added DMAP (536.20 mg, 4.39 mmol) and succinic anhydride (329.40 mg, 3.29 mmol) sequentially. The resulting mixture was stirred for 3 hr at 22° C. Reaction mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash chromatography (gradient: 0-5% MOH in DCM) for afford 4-[(2R,5R)-5-[6-[benzoyl(methyl)amino]purin-9-yl]-2-[[bis(4-methoxyphenyl)-phenyl-methoxy]methyl]-4-methoxy-tetrahydrofuran-3-yl]oxy-4-oxo-butanoic acid (0.63 g, 72% yield) as white foam. ¹H NMR (600 MHz, DMSO-d₆) δ 12.30 (s, 1H), 8.64 (s, 1H), 8.47 (s, 1H), 7.37-7.26 (m, 5H), 7.25-7.15 (m, 9H), 6.87-6.79 (m, 4H), 6.10 (d, J=6.4 Hz, 1H), 5.46 (dd, J=5.3, 3.3 Hz, 1H), 4.91 (dd, J=6.4, 5.3 Hz, 1H), 4.24 (dt, J=5.8, 3.7 Hz, 1H), 3.72 (d, J=1.0 Hz, 7H), 3.67 (s, 3H), 3.35-3.29 (m, 1H), 3.25 (dd, J=10.6, 4.0 Hz, 1H), 3.20 (s, 3H), 2.60 (dd, J=7.7, 5.4 Hz, 2H), 2.53-2.50 (m, 3H) ppm. ¹³C NMR (151 MHz, DMSO-d₆) δ 173.35, 171.57, 171.37, 158.12, 154.32, 152.22, 151.57, 144.69, 144.50, 135.90, 135.41, 135.35, 130.82, 129.73, 128.31, 127.99, 127.84, 127.63, 126.74, 126.11, 113.19, 85.89, 85.70, 81.75, 79.09, 70.71, 63.28, 59.82, 58.14, 55.05, 54.98, 35.41, 28.72, 28.68 ppm. HRMS calc. for C₄₄H₄₄N₅O₁₀ [M+H]⁺ 802.3088, found 802.3085.

Added 4-[(2R,5R)-5-[6-[benzoyl(methyl)amino]purin-9-yl]-2-[[bis(4-methoxy phenyl)-phenyl-methoxy]methyl]-4-methoxy-tetrahydrofuran-3-yl]oxy-4-oxo-butanoic acid (0.62 g, 773.22 μmol) and diisopropylethylamine (399.73 mg, 3.09 mmol, 538.71 μL) into rb flask. Then added dry acetonitrile (50 mL). Stirred well to dissolve and then HBTU (307.90 mg, 811.88 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜ 5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight to afford 57.

Checking the Loading: Weighted out 52.9 mg and loaded into 250 ml volumetric flask. Then added 0.1M toluene-p-sulfonic acid in ACN up to measure line. Sonicated and settled for 1 hour. Checked loading by spectrophotometer and Beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 6.0 g, Loading: 93 μmol/g.

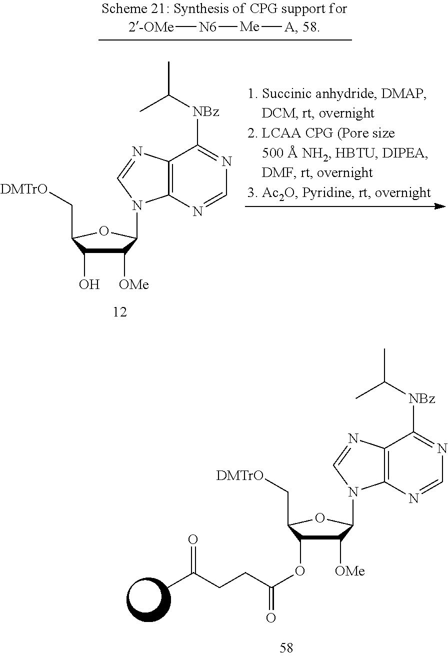

To a clear solution of 12 (0.52 g, 712.51 μmol) in dichloromethane (10 mL) was added DMAP (348.19 mg, 2.85 mmol) and succinic anhydride (213.90 mg, 2.14 mmol) sequentially. The resulting mixture was stirred for 3 hr at 22° C. Reaction mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash chromatography (gradient: 0-5% Methanol in DCM) for afford 4-[(2R,5R)-5-[6-[benzoyl(isopropyl)amino]purin-9-yl]-2-[[bis(4-methoxyphenyl)-phenyl-methoxy]methyl]-4-methoxy-tetrahydrofuran-3-yl]oxy-4-oxo-butanoic acid (0.46 μg, 78% yield) as white foam. ¹H NMR (600 MHz, DMSO-d₆) δ 12.29 (s, 1H), 8.62 (d, J=11.8 Hz, 2H), 7.32 (dd, J=6.5, 3.2 Hz, 2H), 7.27-7.23 (m, 2H), 7.23-7.14 (m, 8H), 7.07 (t, J=7.6 Hz, 2H), 6.86-6.80 (m, 4H), 6.04 (d, J=6.5 Hz, 1H), 5.44 (dd, J=5.3, 3.3 Hz, 1H), 5.04 (hept, J=6.8 Hz, 1H), 4.90-4.85 (m, 1H), 4.20 (dt, J=5.6, 3.7 Hz, 1H), 3.73 (d, J=4.2 Hz, 6H), 3.31-3.20 (m, 2H), 3.15 (s, 3H), 2.59 (dd, J=7.7, 5.4 Hz, 2H), 2.52-2.49 (m, 2H), 1.39 (dd, J=6.8, 2.9 Hz, 6H) ppm. ¹³C NMR (151 MHz, DMSO-d₆) δ 173.34, 171.56, 170.50, 158.13, 153.55, 152.25, 151.84, 145.10, 144.65, 136.98, 135.43, 135.35, 130.38, 129.74, 129.72, 128.85, 127.99, 127.83, 127.74, 127.64, 126.73, 113.22, 113.19, 85.70, 85.69, 81.64, 79.00, 70.63, 63.18, 59.82, 58.09, 55.06, 55.04, 50.60, 28.72, 28.67, 20.83, 20.64, 20.60 ppm. HRMS calc. for C₄₆H₄₈N₅O₁₀ [M+H]⁺ 830.3401, found 830.3406.

Added 4-[(2R,5R)-5-[6-[benzoyl(isopropyl)amino]purin-9-yl]-2-[[bis(4-methoxy phenyl)-phenyl-methoxy]methyl]-4-methoxy-tetrahydrofuran-3-yl]oxy-4-oxo-butanoic acid (0.48 g, 578.39 μmol) and diisopropylethylamine (299.00 mg, 2.31 mmol, 402.97 μL) into rb flask. Then added dry acetonitrile (50 mL). Stirred well to dissolve and then HBTU (230.32 mg, 607.31 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜ 5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight to afford 58.

Checking the Loading: Weighted out 33 mg and loaded into 250 mL volumetric flask. Then added 0.1M toluene-p-sulfonic acid in ACN up to measure line. Sonicated and settled for 1 hour. Checked loading by spectrophotometer and Beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 4.9 g, Loading: 93 μmol/g.

### Triphosphate Synthesis

Triphosphate synthesis is shown in the following Scheme 22:

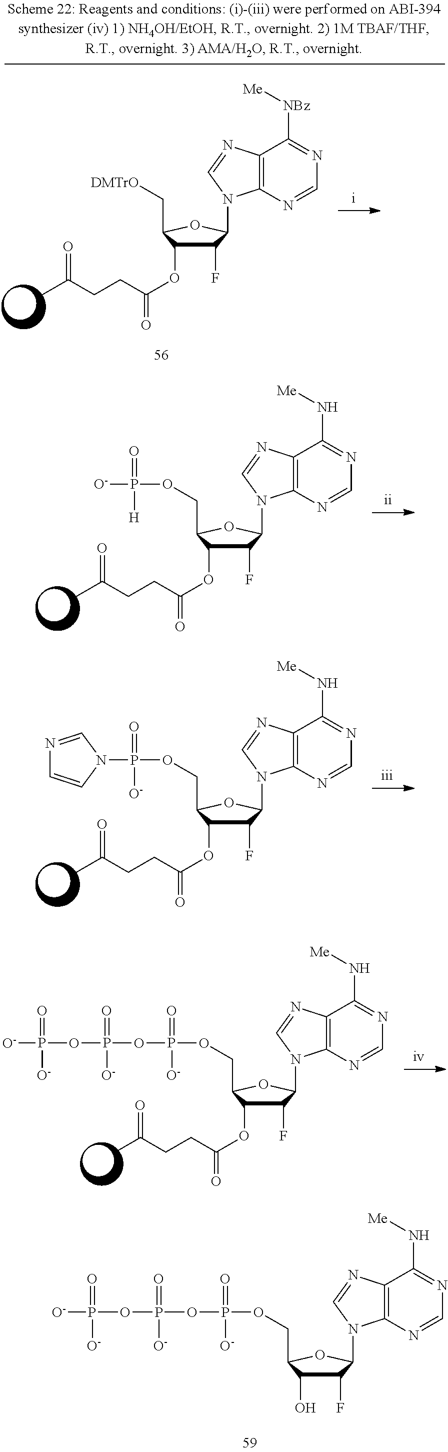

((2R,3R,4R,5R)-4-fluoro-3-hydroxy-5-(6-(methylamino)-9H-purin-9-yl)tetrahydrofuran-2-yl)methyl triphosphate: Triphosphate 59 was synthesized following previously described protocols¹². ¹H NMR (500 MHz, D₂O) δ 8.38 (s, 1H), 8.20 (s, 1H), 6.38 (dd, J=16.5, 2.1 Hz, 1H), 5.61-5.19 (m, 1H), 4.87-4.67 (m, 1H), 4.48-4.21 (m, 3H), 3.08 (s, 3H) ppm. ³¹P NMR (202 MHz, D₂O) δ −9.30 (d, J=19.5 Hz), −10.58 (d, J=19.6 Hz), −22.05 (t, J=19.5 Hz) ppm.

Monomer Synthesis for Evaluating Adenosine Deaminase Activity:

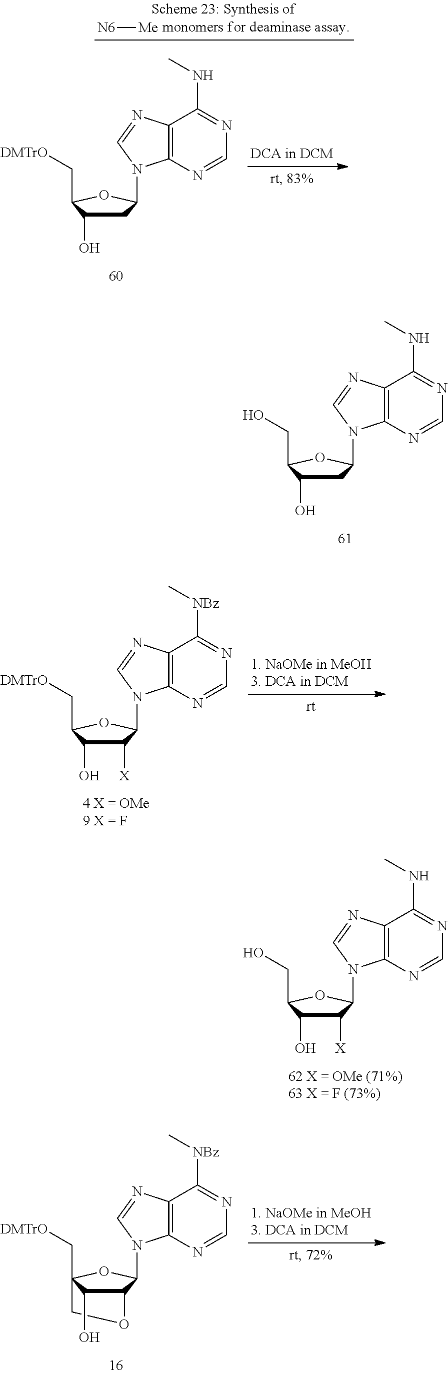

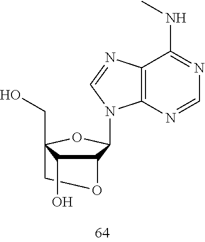 [SMILES: CNc1ncnc2c1ncn2[C@@H]1O[C@@]2(CO)CO[C@H]1C2O]

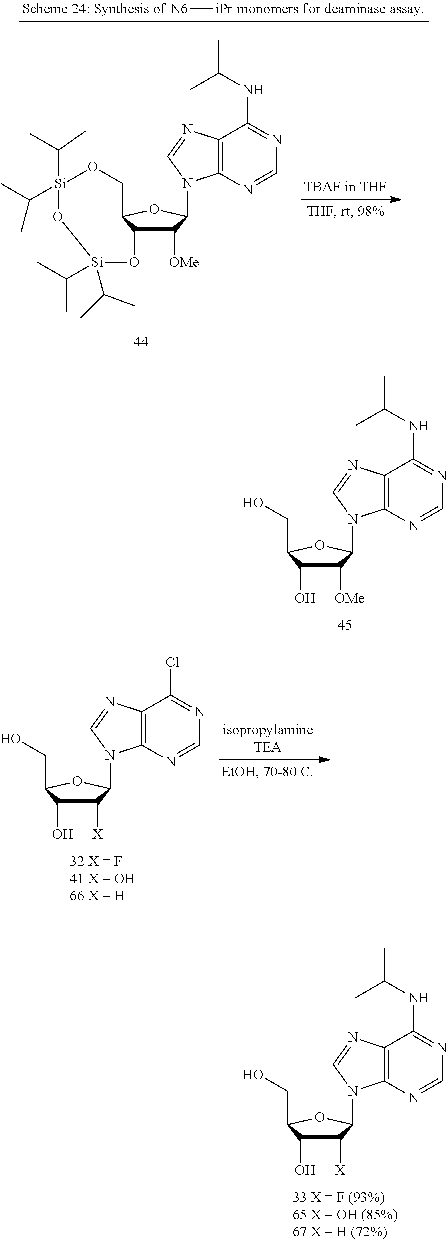

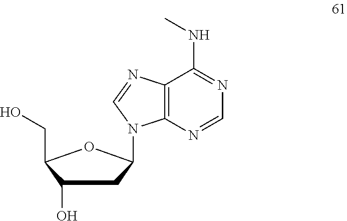 [SMILES: CNc1ncnc2c1ncn2[C@H]1C[C@@H](O)[C@@H](CO)O1]

(2R,5R)-2-(hydroxymethyl)-5-[6-(methylamino)purin-9-yl]tetrahydrofuran-3-ol [Compound 61]: To a clear solution of commercially available 60 (0.567 g, 998.88 μmol) in dichloromethane (10 mL) was added dichloroacetic acid (128.80 mg, 998.88 μmol, 82.56 μL) and stirred for 2 hrs at 15° C. All the volatile matters were removed, and crude mass was purified by column chromatography (gradient: 0-15% MeOH in DCM) to afford 61 (0.22 g, 829.35 μmol, 83.03% yield) as white solid. ¹H NMR (500 MHz, DMSO-d₆) δ 8.32 (s, 1H), 8.21 (d, J=13.4 Hz, 1H), 7.76 (s, 1H), 7.54 (s, 1H), 6.35 (dd, J=7.9, 6.1 Hz, 1H), 5.29 (s, 2H), 4.41 (dt, J=5.7, 2.7 Hz, 1H), 3.88 (td, J=4.2, 2.5 Hz, 1H), 3.62 (dd, J=11.9, 4.3 Hz, 1H), 3.52 (dd, J=11.8, 4.2 Hz, 1H), 2.95 (s, 4H), 2.72 (ddd, J=13.4, 7.9, 5.7 Hz, 1H), 2.26 (ddd, J=13.2, 6.1, 2.8 Hz, 1H) ppm. ¹³C NMR (101 MHz, DMSO-d₆) δ 155.05, 152.38, 147.90, 139.27, 138.82, 119.79, 88.03, 83.97, 70.99, 61.92, 48.60, 26.97 ppm.

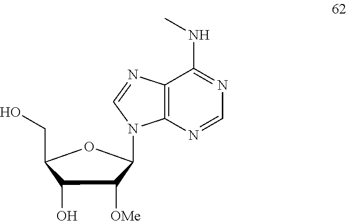

(2R,5R)-2-(hydroxymethyl)-4-methoxy-5-[6-(methylamino)purin-9-yl]tetrahydrofuran-3-ol [Compound 62]: To a clear solution of 4 (0.7 g, 997.48 μmol) in methanol (10 mL) was added sodium methoxide (25% in methanol) (431.07 mg, 1.99 mmol, 444.86 μL) and stirred for 8 hrs. Volatile matters were removed and residue was purified by column chromatography (gradient: 0-5% MeOH in DCM). Solid compound thus obtained was added in dichloromethane (10 mL) and to the clear solution was added dichloroacetic acid (128.62 mg, 997.48 μmol, 82.45 μL). Reaction mixture stirred for 2 hrs at 15° C. and then all the volatile matters were removed under high vacuum pump. Crude material thus obtained was purified by column chromatography (gradient: 0-15% MeOH in DCM) to afford 62 (0.21 g, 711.16 μmol, 71.29% yield) as white solid. ¹H NMR (500 MHz, DMSO-d₆) δ 8.38 (s, 1H), 8.24 (s, 1H), 7.82 (s, 1H), 6.02 (d, J=6.0 Hz, 1H), 5.44 (s, 1H), 5.28-5.24 (m, 1H), 4.36 (ddd, J=12.2, 6.9, 4.6 Hz, 2H), 3.99 (q, J=3.4 Hz, 1H), 3.68 (dd, J=12.1, 3.7 Hz, 1H), 3.57 (dd, J=12.1, 3.6 Hz, 1H), 2.96 (s, 3H) ppm. ¹³C NMR (101 MHz, DMSO-d₆) δ 155.11, 152.58, 148.02, 139.45, 119.83, 86.49, 85.88, 82.52, 68.84, 61.51, 57.48, 54.91, 48.62, 26.99 ppm.

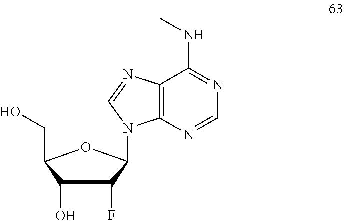

(2R,5R)-4-fluoro-2-(hydroxymethyl)-5-[6-(methylamino)purin-9-yl]tetrahydrofuran-3-ol [Compound 63]: To a clear solution of 9 (700.00 mg, 1.01 mmol) in methanol (10 mL) was added sodium methoxide (25% in methanol) (438.59 mg, 2.03 mmol, 452.63 μL) and stirred for 6 hrs at 22° C. Volatile matters were removed under high vacuum pump, and crude mass was purified by column chromatography (gradient: 0-5% MeOH in DCM). Pure white foam was dissolved in dichloromethane (10 mL) and to the clear solution was added dichloroacetic acid (261.72 mg, 2.03 mmol, 167.77 μL) in single portion. Reaction mixture was stirred for 2 hrs at 15° C. TLC checked and all the volatile matters were again removed after adding 3 mL MeOH. Crude compound was purified by column chromatography to afford 63 (0.21 g, 741.37 μmol, 73.05% yield) as white solid. ¹H NMR (500 MHz, DMSO-d₆) δ 8.36 (s, 1H), 8.25 (s, 1H), 7.82 (s, 1H), 6.24 (dd, J=16.9, 3.0 Hz, 1H), 5.76-5.69 (m, 1H), 5.58-5.36 (m, 1H), 5.26 (s, 1H), 4.54-4.44 (m, 1H), 4.09 (s, 1H), 3.99 (qd, J=3.9, 1.4 Hz, 1H), 3.75 (dd, J=12.4, 2.7 Hz, 1H), 3.59 (dd, J=12.3, 4.0 Hz, 1H), 2.96 (s, 3H) ppm. ¹³C NMR (101 MHz, DMSO-d₆) δ 155.07, 152.74, 147.80, 139.11, 119.67, 94.32, 92.46, 85.99, 85.67, 84.23, 68.47, 68.31, 60.50, 48.61, 27.00 ppm. ¹⁹F NMR (471 MHz, DMSO-d₆) δ −204.73 (dt, J=53.0, 17.0 Hz) ppm.

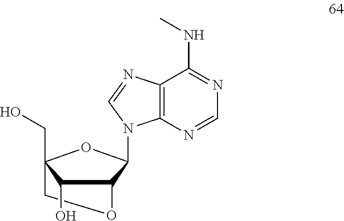 [SMILES: CNc1ncnc2c1ncn2[C@@H]1O[C@@]2(CO)COC1[C@H]2O]

(4S,6R)-4-(hydroxymethyl)-6-[6-(methylamino)purin-9-yl]-2,5-dioxabicyclo[2.2.1]heptan-7-ol [Compound 64]: To a clear solution of 16 (0.7 g, 1.00 mmol) in methanol (10 mL) was added sodium methoxide (25% in methanol) (432.31 mg, 2.00 mmol, 446.15 μL) and stirred for 6 hrs at 22° C. Volatile matters were removed under high vacuum pump, and crude mass was purified by column chromatography (gradient: 0-5% MeOH in DCM). Pure white foam was dissolved in dichloromethane (10 mL) and to the clear solution was added dichloroacetic acid (257.97 mg, 2.00 mmol, 165.37 μL) in single portion. Reaction mixture was stirred for 2 hrs at 15° C. TLC checked and all the volatile matters were again removed after adding 3 mL MeOH. Crude compound was purified by column chromatography to afford 64 (0.21 g, 716.04 μmol, 71.58% yield) as white solid. ¹H NMR (500 MHz, DMSO-d₆) δ 8.21 (s, 2H), 7.80-7.76 (m, 1H), 5.91 (s, 1H), 5.67 (d, J=4.3 Hz, 1H), 5.04 (s, 1H), 4.41 (s, 1H), 4.25 (d, J=3.8 Hz, 1H), 3.93 (d, J=7.8 Hz, 1H), 3.84-3.74 (m, 3H), 2.95 (s, 3H) ppm. ¹³C NMR (101 MHz, DMSO-d₆) δ 154.95, 152.74, 147.46, 137.61, 119.57, 88.51, 85.31, 79.27, 71.43, 69.96, 56.72, 26.97 ppm.

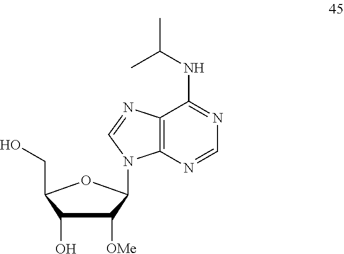

(2R,5R)-2-(hydroxymethyl)-5-[6-(isopropylamino)purin-9-yl]-4-methoxy-tetrahydrofuran-3-ol [Compound 45]: To a clear solution of 44 (1.11 g, 1.96 mmol) in THF (30 mL) was added tetrabutylammonium fluoride (1.23 g, 4.71 mmol, 1.36 mL) in single portion. Reaction mixture was stirred for 2 hr at 22° C. and TLC was checked which showed complete consumption of starting material. All the volatile matters were removed under high vacuum pump and the crude residue was purified by flash column chromatography (gradient: 0-5% MeOH in DCM) to afford 45 (0.62 g, 98% yield) as white foam. ¹H NMR (600 MHz, DMSO-d₆) δ 8.38 (s, 1H), 8.21 (s, 1H), 7.67 (s, 1H), 6.00 (d, J=6.1 Hz, 1H), 5.75 (s, 1H), 5.44 (d, J=5.9 Hz, 1H), 5.27 (d, J=5.1 Hz, 1H), 4.44 (s, 1H), 4.39-4.31 (m, 2H), 3.98 (q, J=3.4 Hz, 1H), 3.67 (dt, J=12.1, 3.9 Hz, 1H), 3.56 (ddd, J=12.1, 6.9, 3.6 Hz, 1H), 3.30 (s, 3H), 1.21 (dd, J=6.5, 2.0 Hz, 6H) ppm. ¹³C NMR (151 MHz, DMSO-d₆) δ 153.93, 152.51, 148.23, 139.36, 119.58, 86.47, 85.88, 82.47, 68.80, 61.50, 57.47, 54.93, 41.33, 22.24, 17.32, 12.63 ppm.

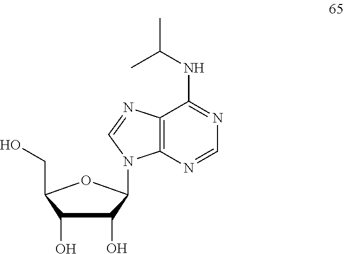

(2R,5R)-2-(hydroxymethyl)-5-[6-(isopropylamino)purin-9-yl]tetrahydrofuran-3,4-diol [Compound 65]: To a clear solution of 41 (5.6 g, 19.53 mmol) in ethanol was added triethylamine (1.98 g, 19.53 mmol, 2.72 mL) and isopropylamine (1.15 g, 19.53 mmol, 1.67 mL). The resulting mixture was heated at 70° C. for 12 hr and TLC was checked. All the volatile matters were removed under high vacuum pump and crude compound was purified by flash column chromatography (gradient: 3-10% MeOH in DCM) to afford 65 (5.16 g, 16.68 mmol, 85.40% yield) as white solid. 1H NMR (600 MHz, DMSO-d₆) δ 8.34 (s, 1H), 8.20 (s, 1H), 7.64-7.60 (m, 1H), 5.89 (d, J=6.2 Hz, 1H), 5.44 (d, J=6.2 Hz, 2H), 5.19 (d, J=4.6 Hz, 1H), 4.62 (td, J=6.2, 4.8 Hz, 1H), 4.45 (s, 1H), 4.16 (td, J=4.7, 2.9 Hz, 1H), 3.98 (q, J=3.4 Hz, 1H), 3.68 (dt, J=12.3, 3.2 Hz, 1H), 3.60-3.53 (m, 1H), 1.22 (d, J=6.6 Hz, 6H). 13C NMR (151 MHz, DMSO-d₆) δ 154.00, 152.38, 148.34, 139.63, 119.71, 88.01, 85.97, 73.51, 70.72, 61.73, 48.64, 41.37, 22.28 ppm.

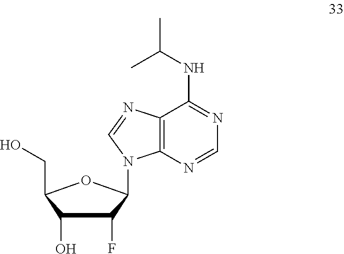

(2R,5R)-4-fluoro-2-(hydroxymethyl)-5-[6-(isopropylamino)purin-9-yl]tetrahydrofuran-3-ol [Compound 33]: To a suspension of commercially available 32 (1.5 g, 5.20 mmol) in ethanol (10 mL) was added triethylamine (1.58 g, 15.59 mmol, 2.17 mL) and isopropylamine (921.47 mg, 15.59 mmol, 1.33 mL). The resulting mixture was heated at 80° C. for 10 hr and TLC was checked. All the volatile matters were removed under high vacuum pump and crude compound was purified by flash column chromatography (gradient: 0-7% MeOH in DCM) to afford 33 (1.51 g, 93% yield) as white foam. ¹H NMR (600 MHz, DMSO-d₆) δ 8.36 (s, 1H), 8.21 (s, 1H), 7.66 (d, J=8.1 Hz, 1H), 6.23 (dd, J=16.9, 3.0 Hz, 1H), 5.71 (d, J=6.0 Hz, 1H), 5.51-5.35 (m, 1H), 5.26 (dd, J=6.3, 4.9 Hz, 1H), 4.49 (dtd, J=17.2, 6.2, 4.5 Hz, 2H), 3.98 (qd, J=3.8, 1.4 Hz, 1H), 3.75 (ddd, J=12.3, 5.0, 2.8 Hz, 1H), 3.58 (ddd, J=12.4, 6.3, 4.0 Hz, 1H), 1.21 (dd, J=6.5, 2.1 Hz, 6H). ¹⁹F NMR (565 MHz, DMSO-d₆) δ −204.44. ¹³C NMR (151 MHz, DMSO-d₆) δ 153.90, 152.67, 138.99, 119.42, 93.98, 92.74, 85.92, 85.70, 84.17, 68.40, 68.29, 60.47, 54.91, 41.32, 22.23 ppm.

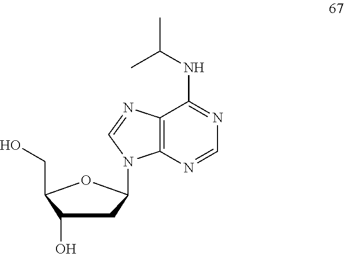 [SMILES: CC(C)Nc1ncnc2c1ncn2[C@H]1C[C@@H](O)[C@@H](CO)O1]

(2R,5R)-2-(hydroxymethyl)-5-[6-(isopropylamino)purin-9-yl]tetrahydrofuran-3-ol [Compound 67]: To a clear solution of commercially available 66 (0.271 g, 1.00 mmol) in ethanol (10 mL) was added triethylamine (303.94 mg, 3.00 mmol, 418.65 μL) and isopropylamine (177.54 mg, 3.00 mmol, 256.94 μL). The resulting mixture was heated at 80° C. for 2 hr and TLC was checked. All the volatile matters were removed under high vacuum pump and crude compound was purified by flash column chromatography (gradient: 0-7% MeOH in DCM) to afford 67 (0.21 g, 715.94 μmol, 71.51% yield) as white solid. ¹H NMR (600 MHz, DMSO-d₆) δ 8.32 (d, J=1.5 Hz, 1H), 8.18 (s, 1H), 7.58 (s, 1H), 6.34 (t, J=7.0 Hz, 1H), 5.31-5.27 (m, 1H), 5.24 (t, J=5.9 Hz, 1H), 4.41 (s, 2H), 3.90-3.85 (m, 1H), 3.62 (dt, J=10.8, 4.6 Hz, 1H), 3.52 (dt, J=11.6, 5.6 Hz, 1H), 3.17 (dd, J=5.3, 1.5 Hz, 1H), 2.72 (dt, J=13.5, 6.8 Hz, 1H), 2.24 (dt, J=13.6, 4.1 Hz, 1H), 1.21 (d, J=6.5 Hz, 6H) ppm.

### Oligonucleotide Synthesis

Oligonucleotides used for the exonuclease assay were synthesized on an ABI-394 and those used for in vitro efficacy assays were synthesized on a MerMade 192 synthesizer on 1-pmol scale using universal or custom supports. A solution of 0.25 M 5-(S-ethylthio)-1H-tetrazole in acetonitrile (CH₃CN) was used as the activator. The solutions of commercially available phosphoramidites and synthesized 5′-(R)—C-methyl-guanosine phosphoramidities were 0.15 M in anhydrous CH₃CN or ACN/DMF (9:1, v/v). The 5′-(S)—C-methyl-guanosine phosphoramidities were 0.15 M in anhydrous 15% DCM in CH₃CN. The oxidizing reagent was 0.02 M I₂ in THF/pyridine/H₂O. The detritylation reagent was 3% dichloroacetic acid in CH₂Cl₂. After completion of the automated synthesis, the oligonucleotide was manually released from support and deprotected using a mixture of aqueous MeNH₃ (40% wt) at 60° C. for 12 min.

Post synthesis substitution using the 6-chloro-adenosine phosphoramidites: N⁶-Me was obtained after deprotection using a mixture of aqueous MeNH₃ (40% wt) at 60° C. for 1 h, and N⁶-iPr using a mixture of aqueous iPrNH₃ (40% wt) at 60° C. for 1 h followed by aqueous MeNH₃ (40% wt) at 60° C. for 1 h.

After filtration through a 0.45-μm nylon filter, oligonucleotides were either purified or, for oligonucleotides containing ribose sugars, the 2′ hydroxyl was deprotected by treatment with Et₃N·3HF at 60° C. for 30 min. Oligonucleotides were purified using IEX-HPLC using an appropriate gradient of mobile phase (buffer A: 0.15 M NaCl, 10% CH₃CN; buffer B 1.0 M NaBr, 10% MeCN) and desalted using size-exclusion chromatography with water as an eluent. Oligonucleotides were then quantified by measuring the absorbance at 260 nm. Extinction coefficients were calculated using the following extinction coefficients for each residue: A, 13.86; T/U, 7.92; C, 6.57; and G, 10.53 M⁻¹ cm⁻¹. The purity and identity of modified ONs were verified by analytical anion exchange chromatography and mass spectrometry, respectively.

After the trityl-off synthesis using the MerMade 192, columns were incubated with 150 μL of 40% aqueous methylamine for 30 min at room temperature, and solutions were drained via vacuum into a 96-well plate. After repeating the incubation and draining with a fresh portion of aqueous methylamine, the plate containing the crude oligonucleotides was sealed and shaken at room temperature for 60 min to completely remove all protecting groups. In the case of RNA, the 2′ hydroxyl was deprotected by treating with Et₃N·3HF at 60° C. for 60 min. Precipitation of the crude oligonucleotides was accomplished via the addition of 1.2 mL of ACN/EtOH (9:1, v/v) to each well, followed by centrifugation at 3000 rpm for 45 min at 4° C. The supernatant was removed from each well, and the pellets were resuspended in 950 NL of 20 mM aqueous NaOAc. Oligonucleotides were desalted over a GE Hi-Trap desalting column (Sephadex G25 Superfine) using water as an eluant. The identities and purities of all oligonucleotides were confirmed using ESI-MS and IEX-HPLC, respectively.

For oligonucleotides synthesized using the ABI 394, the manufacturer's standard protocols were used for cleavage and deprotection. Crude oligonucleotides were purified using strong anion exchange with phosphate buffers (pH 8.5) containing NaBr. The identities and purities of all oligonucleotides were confirmed using ESI-LC/MS and IEX-HPLC, respectively.

### Evaluation of Use of Modified Nucleotides as Polymerase Substrates

Purified exonuclease activity-deficient human mitochondrial DNA polymerase POLG was obtained from the lab of Prof. William Copeland (National Institute of Environmental Health Science, Durham, NC, USA). Human mitochondrial POLRMT was purchased from Indigo Biosciences (Cat #MV100-40). Atto-425-labelled DNA and RNA primers were synthesized in house; DNA templates were obtained from IDT.

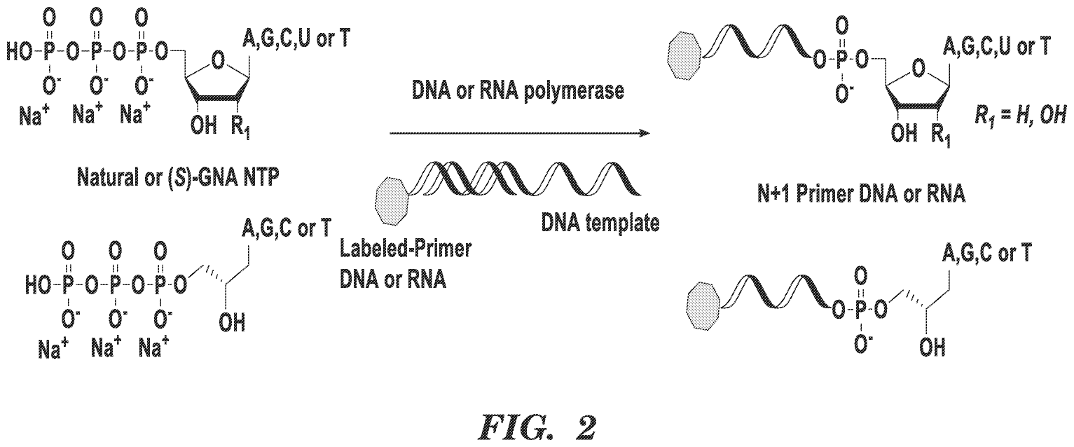

Polymerase incorporation assays are schematically shown in FIG. 2. The assays are designed for single NT addition on appropriate template/primer sets:
Atto-425 labeled primers allow for high sensitivity fluorescence detection with very low reaction volumes
Analytical IEX-HPLC with fluorescence detection for optimal resolution of primer-extended products—especially important in competition mixtures
Assays can be applied for the assessment of incorporation efficiency of novel chemistries.

Reaction conditions for the POLG incorporation assays were as follows: 100 nM DNA template, 100 nM 5′-Atto-425-labelled DNA primer, 40 units POLG, 100 μM or 1 mM NTP substrate in 20 mM Tris-HCl, pH 8.0, 2 mM β-mercaptoethanol, 0.1 mg/mL bovine serum albumin, 10 mM MgCl₂. Reactions were incubated at 37° C. for 30 min. Reactions were quenched by heating the reaction mixture to 85° C. for 5 min. The reaction mixtures were diluted with water to a final primer concentration of approximately 2.5 nM and analysed by IEX-HPLC with an attached fluorescence detector (excitation wavelength: 436 nm, emission wavelength: 485 nm) and a Dionex BioLCDNAPac PA200 4×250 mm (8 μm particle size) column. Buffer A was 20 mM sodium phosphate, 15% ACN, pH 11; and buffer B was 20 mM sodium phosphate, 15% ACN, 1 M NaBr, pH 11. The flow rate was 1 mL/min, and the gradient was 25% to 40% buffer B in 16 min. Reaction conditions for the POLRMT incorporation assays were as follows: 200 nM DNA template, 50 nM 5′-Atto-425-labelled RNA primer, 300 nM POLRMT, 100 μM or 1 mM NTP substrate in 20 mM Tris-HCl, pH 8, 10 mM MgCl2, 10 mM DTT, 0.05% Tween-20. Reactions were incubated at 37° C. for 30 min and were quenched by heating the reaction mixture to 85° C. for 5 min. The reaction mixtures were diluted with water to a final primer concentration of approximately 2.5 nM and analysed by IEX-HPLC as described above, except that the gradient was 35% to 40% buffer B in 16 min.

Results of Mito RNA pol (POLRMT), DNA pol-γ assays are shown in FIGS. 3A and 3B and summarized in Table 1.

TABLE 1
Summary of polymerase incorporation assays

| | | Pol-γ | |
|---|---|---|---|
| PoIRMT | | | Inhibition/ |
| Incorporation | Competition | Incorporation | Competition |
| Yes | Inhibits > 100:1 | Yes | No Data |
| | Competes > 10:1 | | |

As can be seen from the data summarized in Table 1, both PolRMT and Pol-γ are able to incorporate 2′Fluoro-N6Methyl-Atp triphosphate. Further, 2′Fluoro-N6-MethylAtp was able to compete for incorporation by PolRMT.

Recognition of 2′F—N6MeA NTPs by POLRMT and PolGamma: Incorporation of 2′F—N6MeA, RNA, and 2′-F-RNA NTP monomers were evaluated in a previously reported in vitro primer extension assays catalyzed by the human mitochondrial RNA polymerase POLRMT and human mitochondrial DNA polymerase PolGamma. Incorporation of 2′F—N6MeA nucleotide triphosphate (NTP) was tested. Briefly, a 5′-fluorescently labeled primer and template were incubated in the presence of a single NTP and POLRMT for 30 minutes or for 35 minutes with PolGamma, and the primer extension reaction was monitored via fluorescence detection ion-exchange high-performance liquid chromatography. As expected, native NTPs (rATP, and, dATP) were efficiently incorporated by the polymerases (FIGS. 3A and 3B). 2′-F-dNTPs were also used as substrates, albeit to a lower extent relative to canonical NTPs. Interestingly, the addition of a methyl group the N6 position of 2′F—N6MeA analogue 59, did not significantly alter the incorporation of the triphosphate compared to the 2′-F-dNTPs by POLRMT or PolGamma. This data confirms that compared to 2′-F-dNTPs, 2′F—N6MeA NTP is also a good substrate for POLRMT and PolGamma for incorporation into RNA and DNA respectively.

### Nuclease Resistance Assays

Oligonucleotides were prepared at final concentrations of 0.1 mg/mL in 50 mM Tris (pH 7.2), 10 mM MgCl2 for assays in the presence of 3′-specific SVPD or in 50 mM sodium acetate (pH 6.5), 10 mM MgCl₂ for assays in the presence of 5′-specific PDE-II. The exonuclease (75 mU/mL SVPD or 500 mU/mL PDE-II) was added to oligonucleotide solution immediately prior to the first injection onto the HPLC column, and enzymatic degradation kinetics were monitored for 24 h at 25° C. Samples collected over 24 h were immediately injected directly onto a Dionex DNAPac PA200 analytical column at 30° C. column temperature. The gradient was from 37% to 52% 1 M NaBr, 10% CH₃CN, 20 mM sodium phosphate buffer at pH 11 over 10 min with a flow rate of 1 mL/min. The full-length oligonucleotide amount was determined as the area under the curve of the peak detected at A260. Percent full-length ON was calculated by dividing the area under the curve at a given time point by that at the first time point and multiplying by 100. Activity of enzyme was verified for each experiment by including a 20-mer oligodeoxythymidylate with a terminal PS linkage in each experiment. Each aliquot of enzyme was thawed just prior to the experiment. The half-life was determined by fitting to first-order kinetics. Each degradation experiment was performed in duplicate.

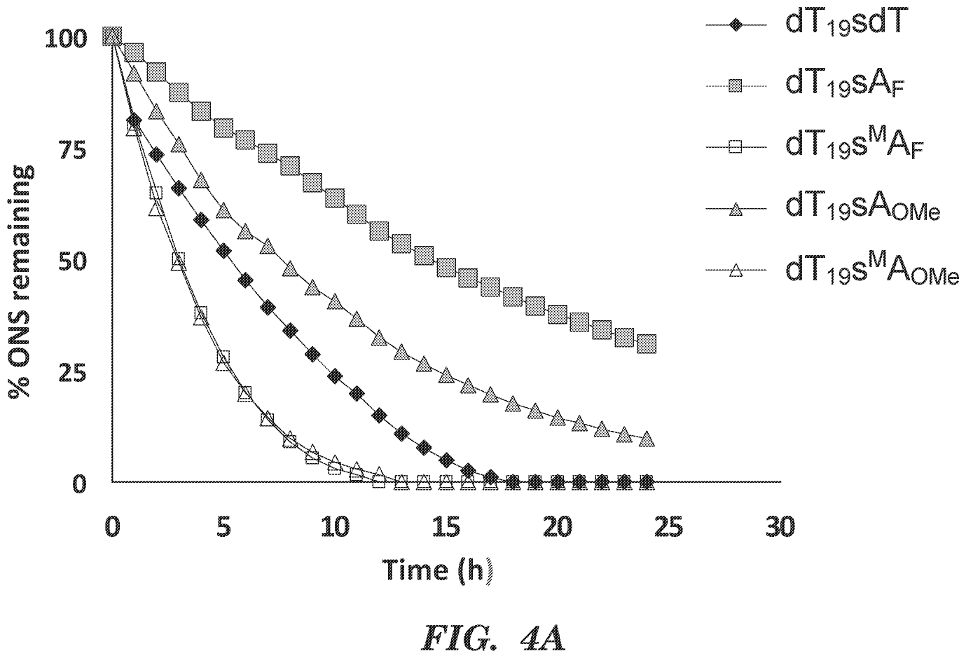

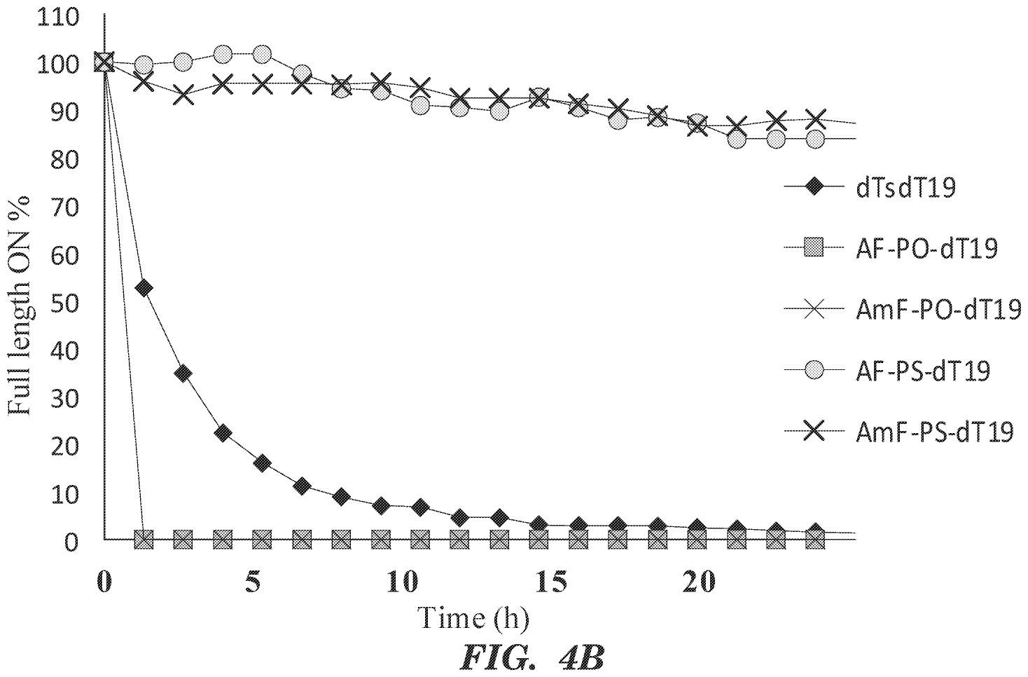

Results are summarized in Tables 2 and 3 and shown in FIGS. 4A and 4B.

TABLE 2
Results of 3′-exonuclease assay

| | | | Half- | | | | |
|---|---|---|---|---|---|---|---|
| | | | life(h) | Half-life(h) | | | |
| | Sequence | Q= | dT | 2′-FA | 2′-F^{m}A | 2′-OMeA | 2′-OMe^{m}A |
| 1 | dT19-PO-Q | | nd | <1 | <<1 | <1 | <<1 |
| 2 | dT19-Q-PO-Q | | nd | <1 | <<1 | <1 | 0.5 |
| 3 | dT19-PS-Q | | 3.0 | 13.9 | 1.9 | 7.1 | 2.1 |
| 4 | dT19-Q-PS-Q | | 3.0 | 25.7 | 10.6 | 31.2 | 11.1 |

TABLE 3
Results of 5′-exonuclease assay

| Sr. No | Code | Half-Life |
|---|---|---|
| 1 | dTsdT₁₉ | 4.5 |
| 2 | A_{F}OdT₁₉ | <1 |
| 3 | ^{M}A_{F}OdT₁₉ | <1 |
| 4 | A_{F}SdT₁₉ | >24 |
| 5 | ^{M}A_{F}SdT₁₉ | >24 |

As can be seen from Table 2 and FIG. 4A, m6A at the 3′-end of a DNA oligonucleotide is more susceptible to cleavage by 3′-exonuclease than adenine analogue in both 2′-F and 2′-OMe contexts.

As can be seen from Table 3 and FIG. 4B, a DNA oligonucleotide with 2′-F m6A at the 5′ end has comparable stability to that with 2′-F A in the presence of 5′-exonuclease.

### In Vitro Gene Silencing Assay

Primary mouse hepatocytes were obtained from Life Technologies and cultured in Williams E Medium with 10% foetal bovine serum. Transfection was carried out by adding 4.9 μL of Opti-MEM plus 0.1 μL of Lipofectamine RNAiMax (Invitrogen) per well to 5 μL of each siRNA duplex at the desired concentration to an individual well in a 384-well plate. The mixture was incubated at room temperature for 20 min, and 40 μL of complete growth media containing 5,000 cells was added to the siRNA mixture. Samples were incubated for 24 h, and then RNA was isolated. A similar procedure was followed for the transfection of 10,000,000 cells and scaled accordingly. Dose response experiments were done using eight 6-fold serial dilutions over the range of 20 nM to 75 μM or 50 nM to 187.5 μM.

RNA was isolated using a Dynabeads mRNA Isolation Kit (Invitrogen). Cells were lysed in 75 μL of Lysis/Binding Buffer containing 3 μL of beads per well and mixed for 10 min on an electrostatic shaker. Buffers were prepared according to the manufacturer's protocol. The washing steps were automated on a Biotek EL406 using a magnetic plate support. Beads were washed once in buffer A, once in buffer B, and twice in buffer E, with 90 μL volume per wash and with aspiration steps between washes.

cDNA synthesis was accomplished with the ABI High-capacity cDNA Reverse Transcription kit (Applied Biosystems). A mixture of 1 μL of 10× buffer, 0.4 μL of 25×dNTPs, 1 μL of random primers, 0.5 μL of reverse transcriptase, 0.5 μL of RNase inhibitor, and 6.6 μL of water per reaction were added per well. Plates were sealed, agitated for 10 min on an electrostatic shaker, and then incubated at 37° C. for 2 h. Following this, the plates were agitated at 80° C. for 8 min. cDNA (2 μL) was added to a master mix containing 0.5 μL mouse GAPDH TaqMan Probe (Applied Biosystems, Cat. #4308313), 0.5 μL of mouse TTR or F12 TaqMan probes (Applied Biosystems), and 5 μL of Lightcycler 480 probe master mix (Roche) per well in a 384-well plate (Roche). Real-time PCR was performed in an ABI 7900HT RT-PCR system (Applied Biosystems) using the ΔΔCt (RQ) assay. Each siRNA concentration was tested in four biological replicates. To calculate relative fold change, real-time data were analysed using the ΔΔCt method and normalised to assays performed with cells transfected with 10 nM control siRNA. IC50 values were calculated using a four-parameter fit model using XLFit.

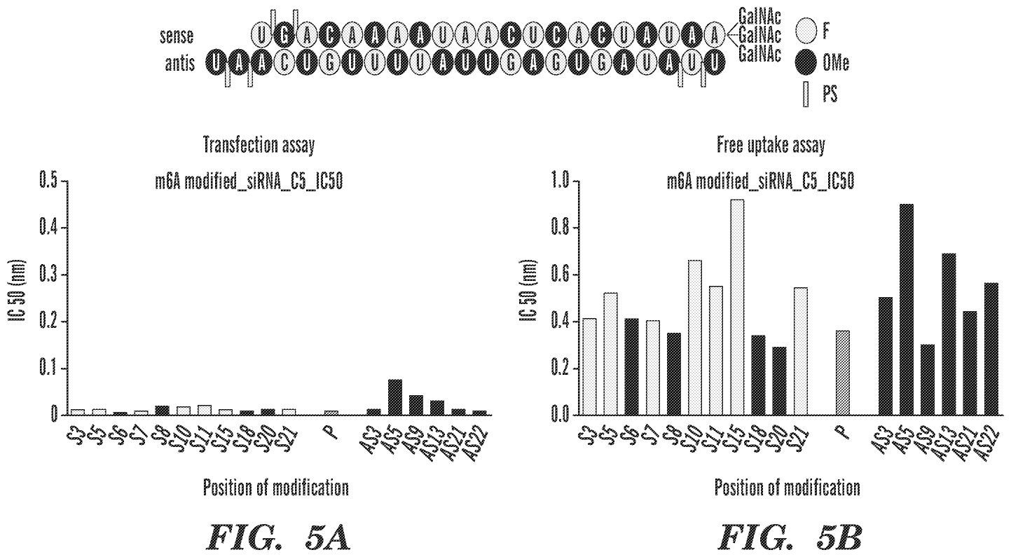

Results are summarized in Tables 4-6 and shown in FIGS. 5A-7B.

TABLE 4
IC50 values for transfection and free uptake of N6-Me A modified
siRNA duplexes targeting C5

| Position | Duplex | Sense(5′-3′)(top)/Antisense (5′-3′) (bottom) | IC50_TX_nM | IC50_FU_nM |
|---|---|---|---|---|
| P | AD-58641.44 | UfsgsAfcAfaAfaUfAfAfcUfcAfcUfaUfaAfL96 A-119322.133 | 0.01 | 0.36 |
| | | usUfsaUfaGfuGfaGfuuaUfuUfuGfuCfasasu A-119323.51 | | |
| S3 | AD-86588.1 | UfsgsQ226cAfaAfaUfAfAfcUfcAfcUfaUfaAfL96 A-171163.1 | 0.012 | 0.41 |
| | | usUfsaUfaGfuGfaGfuuaUfuUfuGfuCfasasu A-119323.62 | | |
| S5 | AD-86213.2 | UfsgsAfcQ226aAfaUfAfAfcUfcAfcUfaUfaAfL96 A-171162.1 | 0.012 | 0.52 |
| | | usUfsaUfaGfuGfaGfuuaUfuUfuGfuCfasasu A-119323.61 | | |
| S6 | AD-86212.2 | UfsgsAfcAfQ225AfaUfAfAfcUfcAfcUfaUfaAfL96 A-171161.1 | 0.007 | 0.41 |
| | | usUfsaUfaGfuGfaGfuuaUfuUfuGfuCfasasu A-119323.60 | | |
| S7 | AD-86211.2 | UfsgsAfcAfaQ226aUfAfAfcUfcAfcUfaUfaAfL96 A-171160.1 | 0.01 | 0.4 |
| | | usUfsaUfaGfuGfaGfuuaUfuUfuGfuCfasasu A-119323.59 | | |
| S8 | AD-86210.2 | UfsgsAfcAfaAfQ225UfAfAfcUfcAfcUfaUfaAfL96 A-171159.1 | 0.019 | 0.35 |
| | | usUfsaUfaGfuGfaGfuuaUfuUfuGfuCfasasu A-119323.58 | | |
| S10 | AD-86209.2 | UfsgsAfcAfaAfaUfQ226AfcUfcAfcUfaUfaAfL96 A-171158.1 | 0.017 | 0.66 |
| | | usUfsaUfaGfuGfaGfuuaUfuUfuGfuCfasasu A-119323.57 | | |
| S11 | AD-86208.2 | UfsgsAfcAfaAfaUfAfQ226cUfcAfcUfaUfaAfL96 A-171157.1 | 0.021 | 0.55 |
| | | usUfsaUfaGfuGfaGfuuaUfuUfuGfuCfasasu A-119323.56 | | |
| S15 | AD-86207.2 | UfsgsAfcAfaAfaUfAfAfcUfcQ226cUfaUfaAfL96 A-171156.1 | 0.011 | 0.92 |
| | | usUfsaUfaGfuGfaGfuuaUfuUfuGfuCfasasu A-119323.55 | | |
| S18 | AD-86206.2 | UfsgsAfcAfaAfaUfAfAfcUfcAfcUfQ225UfaAfL96 A-171155.1 | 0.01 | 0.34 |
| | | usUfsaUfaGfuGfaGfuuaUfuUfuGfuCfasasu A-119323.54 | | |
| S20 | AD-86205.2 | UfsgsAfcAfaAfaUfAfAfcUfcAfcUfaUfQ225AfL96 A-171154.1 | 0.012 | 0.29 |
| | | usUfsaUfaGfuGfaGfuuaUfuUfuGfuCfasasu A-119323.53 | | |
| S21 | AD-86204.2 | UfsgsAfcAfaAfaUfAfAfcUfcAfcUfaUfaQ226L96 A-171153.1 | 0.013 | 0.54 |
| | | usUfsaUfaGfuGfaGfuuaUfuUfuGfuCfasasu A-119323.52 | | |
| AS3 | AD-86218.2 | UfsgsAfcAfaAfaUfAfAfcUfcAfcUfaUfaAfL96 A-119322.139 | 0.013 | 0.5 |
| | | usUfsQ225UfaGfuGfaGfuuaUfuUfuGfuCfasasu A-171169.1 | | |
| AS5 | AD-86589.1 | UfsgsAfcAfaAfaUfAfAfcUfcAfcUfaUfaAfL96 A-119322.138 | 0.076 | 0.9 |
| | | usUfsaUfQ225GfuGfaGfuuaUfuUfuGfuCfasasu A-171168.1 | | |
| AS9 | AD-86217.2 | UfsgsAfcAfaAfaUfAfAfcUfcAfcUfaUfaAfL96 A-119322.137 | 0.042 | 0.3 |
| | | usUfsaUfaGfuGfQ225GfuuaUfuUfuGfuCfasasu A-171167.1 | | |
| AS13 | AD-86216.2 | UfsgsAfcAfaAfaUfAfAfcUfcAfcUfaUfaAfL96 A-119322.136 | 0.031 | 0.69 |
| | | usUfsaUfaGfuGfaGfuuQ225UfuUfuGfuCfasasu A-171166.1 | | |
| AS21 | AD-86215.2 | UfsgsAfcAfaAfaUfAfAfcUfcAfcUfaUfaAfL96 A-119322.135 | 0.012 | 0.44 |
| | | usUfsaUfaGfuGfaGfuuaUfuUfuGfuCfQ225sasu A-171165.1 | | |
| AS22 | AD-86214.2 | UfsgsAfcAfaAfaUfAfAfcUfcAfcUfaUfaAfL96 A-119322.134 | 0.008 | 0.56 |
| | | usUfsaUfaGfuGfaGfuuaUfuUfuGfuCfasQ225su A-171164.1 | | |

TABLE 5
IC50 values for transfection and free uptake of N6-Me A modified siRNA
duplexes targeting β-catenin

| Position | Duplex | Sense (5′-3′)/Antisense (5′-3′) | IC50_TX_nM | IC50_FU_nM |
|---|---|---|---|---|
| Parent | AD-64919.13 | UfsasCfuGfuUfgGfAfUfuGfaUfuCfgAfaAfL10 A-129065.14 | 1.317 | 54.711 |
| | | VPusUfsuCfgAfaUfcAfaucCfaAfcAfgUfasgsc A-129066.32 | | |
| S2 | AD-86223.2 | UfsQ225sCfuGfuUfgGfAfUfuGfaUfuCfgAfaAfL10 A-171174.1 | 0.993 | 44.497 |
| | | VPusUfsuCfgAfaUfcAfaucCfaAfcAfgUfasgsc A-129066.37 | | |
| S10 | AD-86222.2 | UfsasCfuGfuUfgGfQ226UfuGfaUfuCfgAfaAfL10 A-171173.1 | 0.9 | 44.579 |
| | | VPusUfsuCfgAfaUfcAfaucCfaAfcAfgUfasgsc A-129066.36 | | |
| S19 | AD-86221.2 | UfsasCfuGfuUfgGfAfUfuGfaUfuCfgQ226aAfL10 A-171172.1 | 1.744 | 112.297 |
| | | VPusUfsuCfgAfaUfcAfaucCfaAfcAfgUfasgsc A-129066.35 | | |
| S20 | AD-86220.2 | UfsasCfuGfuUfgGfAfUfuGfaUfuCfgAfQ225AfL10 A-171171.1 | 1.484 | 34.136 |
| | | VPusUfsuCfgAfaUfcAfaucCfaAfcAfgUfasgsc A-129066.34 | | |
| S21 | AD-86219.2 | UfsasCfuGfuUfgGfAfUfuGfaUfuCfgAfaQ226L10 A-171170.1 | 1.25 | 86.277 |
| | | VPusUfsuCfgAfaUfcAfaucCfaAfcAfgUfasgsc A-129066.33 | | |
| AS5 | AD-86590.1 | UfsasCfuGfuUfgGfAfUfuGfaUfuCfgAfaAfL10 A-129065.21 | 2.61 | 90 |
| | | VPusUfsuCfgQ226aUfcAfaucCfaAfcAfgUfasgsc A-171181.1 | | |
| AS9 | AD-86229.2 | UfsasCfuGfuUfgGfAfUfuGfaUfuCfgAfaAfL10 A-129065.20 | 2.41 | 80 |
| | | VPusUfsuCfgAfaUfcQ226aucCfaAfcAfgUfasgsc A-171180.1 | | |
| AS10 | AD-86228.2 | UfsasCfuGfuUfgGfAfUfuGfaUfuCfgAfaAfL10 A-129065.19 | 2.19 | 84.313 |
| | | VPusUfsuCfgAfaUfcAfQ225ucCfaAfcAfgUfasgsc A-171179.1 | | |
| AS14 | AD-86227.2 | UfsasCfuGfuUfgGfAfUfuGfaUfuCfgAfaAfL10 A-129065.18 | 1.3 | 30.838 |
| | | VPusUfsuCfgAfaUfcAfaucCfQ225AfcAfgUfasgsc A-171178.1 | | |
| AS15 | AD-86226.2 | UfsasCfuGfuUfgGfAfUfuGfaUfuCfgAfaAfL10 A-129065.17 | 1.7 | 49.302 |
| | | VPusUfsuCfgAfaUfcAfaucCfaQ226cAfgUfasgsc A-171177.1 | | |
| AS16 | AD-86225.2 | UfsasCfuGfuUfgGfAfUfuGfaUfuCfgAfaAfL10 A-129065.16 | 0.45 | 15.761 |
| | | VPusUfsuCfgAfaUfcAfaucCfaAfcQ226gUfasgsc A-171176.1 | | |
| AS18 | AD-86224.2 | UfsasCfuGfuUfgGfAfUfuGfaUfuCfgAfaAfL10 A-129065.15 | 0.19 | 14.267 |
| | | VPusUfsuCfgAfaUfcAfaucCfaAfcAfgUfQ225sgsc A-171175.1 | | |

TABLE 6
IC50 values for transfection and free uptake of N6-Me A modified
siRNA duplexes targeting mTTR

| Duplex | Sense(5′-3′)/Antisense(5′-3′) | IC50 TX_nM | IC50_FU_nM |
|---|---|---|---|
| AD-86196.2 | Q226sasCfaGfuGfuUfCfUfuGfcUfcUfaUfaAfL96 A-171144.1 | 0.037 | 0.130 |
| | usUfsaUfaGfaGfcAfagaAfcAfcUfgUfususu A-117800.246 | | |
| AD-86195.2 | AfsQ225sCfaGfuGfuUfCfUfuGfcUfcUfaUfaAfL96 A-171143.1 | 0.018 | 0.650 |
| | usUfsaUfaGfaGfcAfagaAfcAfcUfgUfususu A-117800.245 | | |
| AD-86194.2 | AfsasCfQ225GfuGfuUfCfUfuGfcUfcUfaUfaAfL96 A-171142.1 | 0.032 | 0.260 |
| | usUfsaUfaGfaGfcAfagaAfcAfcUfgUfususu A-117800.244 | | |
| AD-86193.2 | AfsasCfaGfuGfuUfCfUfuGfcUfcUfQ225UfaAfL96 A-171141.1 | 0.036 | 0.510 |
| | usUfsaUfaGfaGfcAfagaAfcAfcUfgUfususu A-117800.243 | | |
| AD-86192.2 | AfsasCfaGfuGfuUfCfUfuGfcUfcUfaUfQ225AfL96 A-171140.1 | 0.048 | 0.450 |
| | usUfsaUfaGfaGfcAfagaAfcAfcUfgUfususu A-117800.242 | | |
| AD-86191.2 | AfsasCfaGfuGfuUfCfUfuGfcUfcUfaUfaQ226L96 A-171139.1 | 0.082 | 0.670 |
| | usUfsaUfaGfaGfcAfagaAfcAfcUfgUfususu A-117800.241 | | |
| AD-57727.125 | AfsasCfaGfuGfuUfCfUfuGfcUfcUfaUfaAfL96 A-117799.327 | 0.05 | 0.454 |
| | usUfsaUfaGfaGfcAfagaAfcAfcUfgUfususu A-117800.240 | | |
| AD-86203.2 | AfsasCfaGfuGfuUfCfUfuGfcUfcUfaUfaAfL96 A-117799.335 | 0.032 | 0.15 |
| | usUfsQ225UfaGfaGfcAfagaAfcAfcUfgUfususu A-171152.1 | | |
| AD-86587.1 | AfsasCfaGfuGfuUfCfUfuGfcUfcUfaUfaAfL96 A-117799.334 | 0.098 | 0.75 |
| | usUfsaUfQ225GfaGfcAfagaAfcAfcUfgUfususu A-171151.1 | | |
| AD-86202.2 | AfsasCfaGfuGfuUfCfUfuGfcUfcUfaUfaAfL96 A-117799.333 | 0.13 | 0.7 |
| | usUfsaUfaGfQ225GfcAfagaAfcAfcUfgUfususu A-171150.1 | | |
| AD-86201.2 | AfsasCfaGfuGfuUfCfUfuGfcUfcUfaUfaAfL96 A-117799.332 | 0.11 | 0.320 |
| | usUfsaUfaGfaGfcQ226agaAfcAfcUfgUfususu A-171149.1 | | |
| AD-86200.2 | AfsasCfaGfuGfuUfCfUfuGfcUfcUfaUfaAfL96 A-117799.331 | 0.29 | 0.800 |
| | usUfsaUfaGfaGfcAfQ225gaAfcAfcUfgUfususu A-171148.1 | | |
| AD-86199.2 | AfsasCfaGfuGfuUfCfUfuGfcUfcUfaUfaAfL96 A-117799.330 | 0.26 | 0.760 |
| | usUfsaUfaGfaGfcAfagQ225AfcAfcUfgUfususu A-171147.1 | | |
| AD-86198.2 | AfsasCfaGfuGfuUfCfUfuGfcUfcUfaUfaAfL96 A-117799.329 | 0.032 | 0.450 |
| | usUfsaUfaGfaGfcAfagaQ226cAfcUfgUfususu A-171146.1 | | |
| AD-86197.2 | AfsasCfaGfuGfuUfCfUfuGfcUfcUfaUfaAfL96 A-117799.328 | 0.19 | 0.66 |
| | usUfsaUfaGfaGfcAfagaAfcQ226cUfgUfususu A-171145.1 | | |

As can be seen from the data in Table 4 and FIGS. 5A and 5B, show that m6A influences RNAi activity in a position-dependent fashion in C5-targeted GalNAc-conjugated siRNA showing similar IC₅₀ as control. As can be seen, m6A is better tolerated in sense strand than antisense strand when transfected into cells. In free uptake assay, activities of m6A-modified siRNAs are similar to the parent. The data also show that terminal modifications in antisense strands are better tolerated than internal modifications

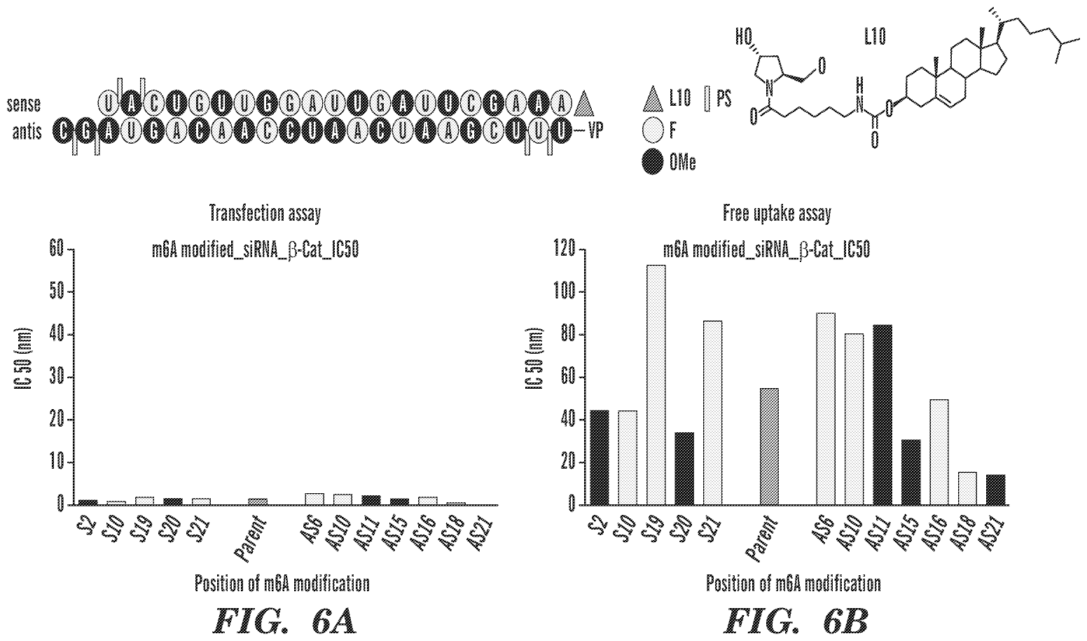

The data in Table 5 and FIGS. 6A and 6B show that m6A influences RNAi activity in a position-dependent fashion in β-catenin-targeted GalNAc-conjugated siRNA. As can be seen, m6A is better tolerated in sense strand than antisense strand when transfected into cells. In free uptake assays, the m6A enhances activity at certain positions. The data in Table 6 and FIGS. 7A and 7B show that m6A influences RNAi activity in a position-dependent fashion in TTR-targeted GalNAc-conjugated siRNA. As can be seen, m6A is better tolerated in sense strand than antisense strand when transfected into cells. In free uptake assays, the m6A enhances activity at certain positions.

### Conclusion

Both 2′-F- and 2′-OMe-modified m6A destabilize DNA:DNA duplexes but not RNA:RNA duplexes. While both m6A-2′-F and m6A-2′-OMe have comparable stability in the presence of 5′-exonuclease to 2′-modified adenine analogs, both m6A-2′-F and m6A-2′-OMe are less stable in the presence of 3′-exonuclease than 2′-modified adenine analogs. Further, both m6A-2′-F and m6A-2′-OMe are well tolerated at tested positions in sense strands in vitro but only terminal modification with m6A is well tolerated in antisense strands in vitro.

TABLE 7A
Oligonucleotides synthesized for ADA assay as
single strands

| | | Single | |
|---|---|---|---|
| Entry | Target | Strand | Sequence 5′-3′ |
| 1 | mTTR | A-173480 | ususcuugCfuCfUfAfuaaaccguguL96 |
| 2 | mTTR | A-140893 | asascaguGfuUfCfUfugcucuauauL96 |
| 3 | mTTR | A-3018917 | Q225sascaguGfuUfCfUfugcucuauauL96 |
| 4 | mTTR | A-3018918 | Q418sascaguGfuUfCfUfugcucuauauL96 |
| 5 | mTTR | A-3196488 | Q226sascaguGfuUfCfUfugcucuauauL96 |
| 6 | mTTR | A-3196489 | Q419sascaguGfuUfCfUfugcucuauauL96 |
| 7 | mTTR | A-3205689 | (aas)ascaguGfuUfCfUfugcucuauauL96 |
| 8 | mTTR | A-173481 | asCfsacgguuuauagAfgCfaagaascsa |
| 9 | mTTR | A-1036679 | Q225sCfsacgguuuauagAfgCfaagaascsa |
| 10 | mTTR | A-1036678 | asCfsacgguuuauagAfgCfaagaascsQ225 |
| 11 | mTTR | A-3018913 | Q225sCfsacgguuuauagAfgCfaagaascsQ225 |
| 12 | mTTR | A-3018914 | Q418SCfsacgguuuauagAfgCfaagaascsa |
| 13 | mTTR | A-3018915 | asCfsacgguuuauagAfgCfaagaascsQ418 |
| 14 | mTTR | A-3018916 | Q418sCfsacgguuuauagAfgCfaagaascsQ418 |
| 15 | mTTR | A-3205691 | (aas)CfsacgguuuauagAfgCfaagaascsa |
| 16 | mTTR | A-3205692 | asCfsacgguuuauagAfgCfaagaascs(aa) |
| 17 | mTTR | A-3205693 | (aas)CfsacgguuuauagAfgCfaagaascs(aa) |
| 18 | mTTR | A-168563 | asUfsauaGfagcaagaAfcAfcuguususu |
| 19 | mTTR | A-1700751 | Q225sUfsauaGfagcaagaAfcAfcuguususu |
| 20 | mTTR | A-3018919 | Q418sUfsauaGfagcaagaAfcAfcuguususu |
| 21 | mTTR | A-3196490 | Q226sUfsauaGfagcaagaAfcAfcuguususu |
| 22 | mTTR | A-3196491 | Q419sUfsauaGfagcaagaAfcAfcuguususu |
| 23 | mTTR | A-3205690 | (aas)UfsauaGfagcaagaAfcAfcuguususu |

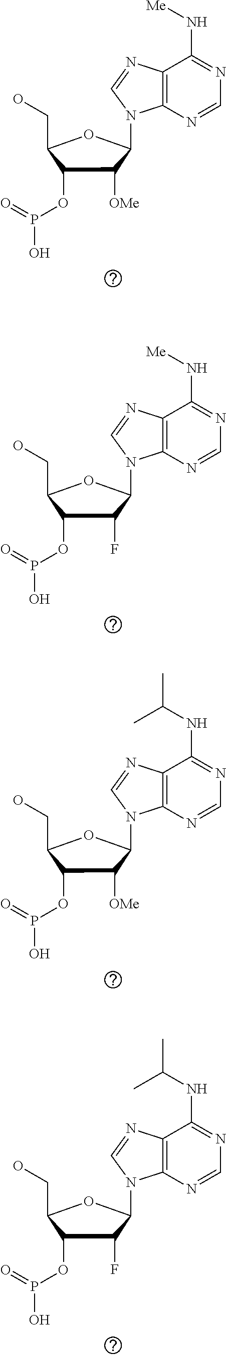

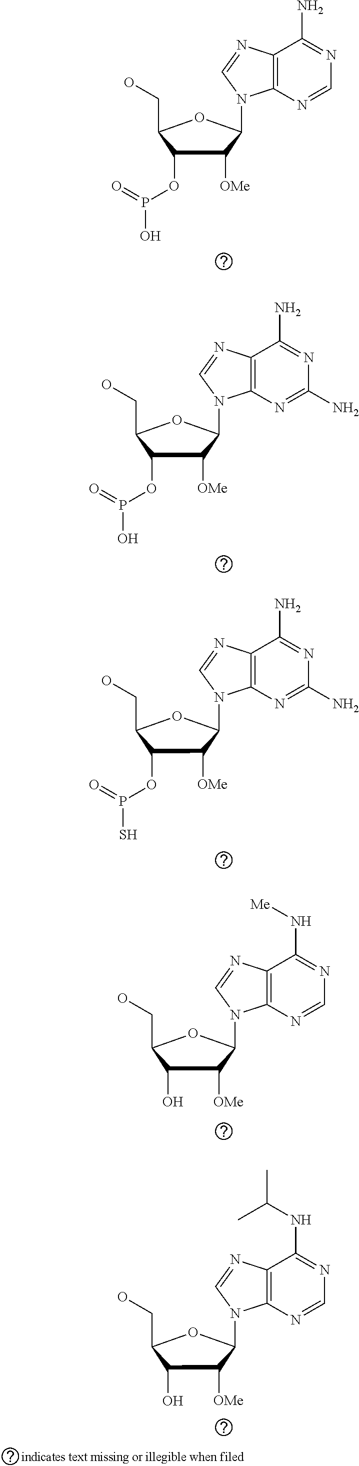

TABLE 7B
siRNAs synthesized for ADA assay

| | | | sense (S) | |
|---|---|---|---|---|
| | | | and | |
| | | | antisense | |
| Entry | Target | Duplex | (AS) | Sequence 5′-3′ |
| 1 | mTTR | AD-125773 | S | ususcuugCfuCfUfAfuaaaccguguL96 |
| | | | AS | asCfsacgguuuauagAfgCfaagaascsa |
| 2 | mTTR | AD-538705 | S | ususcuugCfuCfUfAfuaaaccguguL96 |
| | | | AS | asCfsacgguuuauagAfgCfaagaascsQ225 |
| 3 | mTTR | AD-538706 | S | ususcuugCfuCfUfAfuaaaccguguL96 |
| | | | AS | Q225sCfsacgguuuauagAfgCfaagaascsa |
| 4 | mTTR | AD-1637607 | S | ususcuugCfuCfUfAfuaaaccguguL96 |
| | | | AS | Q225sCfsacgguuuauagAfgCfaagaascsQ225 |
| 5 | mTTR | AD-1637609 | S | ususcuugCfuCfUfAfuaaaccguguL96 |
| | | | AS | asCfsacgguuuauagAfgCfaagaascsQ418 |
| 6 | mTTR | AD-1637610 | S | ususcuugCfuCfUfAfuaaaccguguL96 |
| | | | AS | Q418sCfsacgguuuauagAfgCfaagaascsQ418 |
| 7 | mTTR | | S | ususcuugCfuCfUfAfuaaaccguguL96 |
| | | | AS | Q418sCfsacgguuuauagAfgCfaagaascsa |
| 8 | mTTR | | S | ususcuugCfuCfUfAfuaaaccguguL96 |
| | | | AS | (aas)CfsacgguuuauagAfgCfaagaascsa |
| 9 | mTTR | | S | ususcuugCfuCfUfAfuaaaccguguL96 |
| | | | AS | asCfsacgguuuauagAfgCfaagaascs(aa) |
| 10 | mTTR | | S | ususcuugCfuCfUfAfuaaaccguguL96 |
| | | | AS | (aas)CfsacgguuuauagAfgCfaagaascs(aa) |
| 11 | mTTR | AD-84850 | S | asascaguGfuUfCfUfugcucuauauL96 |
| | | | AS | asUfsauaGfagcaagaAfcAfcuguususu |
| 12 | mTTR | AD-1637611 | | Q225sascaguGfuUfCfUfugcucuauauL96 |
| | | | | Q225sUfsauaGfagcaagaAfcAfcuguususu |
| 13 | mTTR | AD-1637612 | | Q418sascaguGfuUfCfUfugcucuauauL96 |
| | | | | Q418sUfsauaGfagcaagaAfcAfcuguususu |
| 14 | mTTR | AD-1789920 | | Q226sascaguGfuUfCfUfugcucuauauL96 |
| | | | | Q226sUfsauaGfagcaagaAfcAfcuguususu |
| 15 | mTTR | AD-1789921 | | Q419sascaguGfuUfCfUfugcucuauauL96 |
| | | | | Q419sUfsauaGfagcaagaAfcAfcuguususu |
| 16 | mTTR | | | (aas)ascaguGfuUfCfUfugcucuauauL96 |
| | | | | (aas)UfsauaGfagcaagaAfcAfcuguususu |

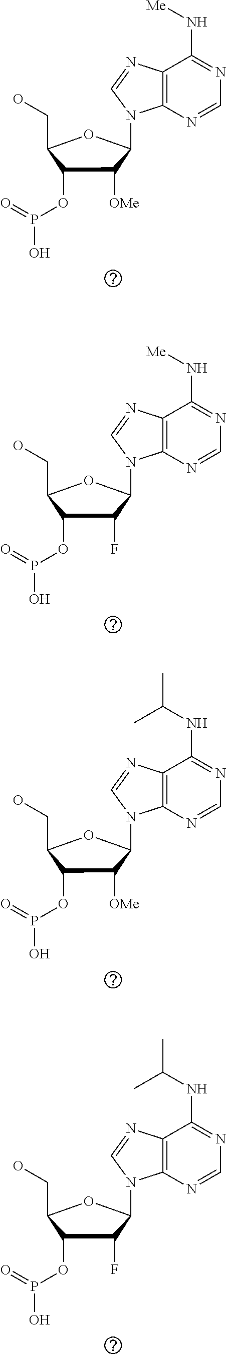

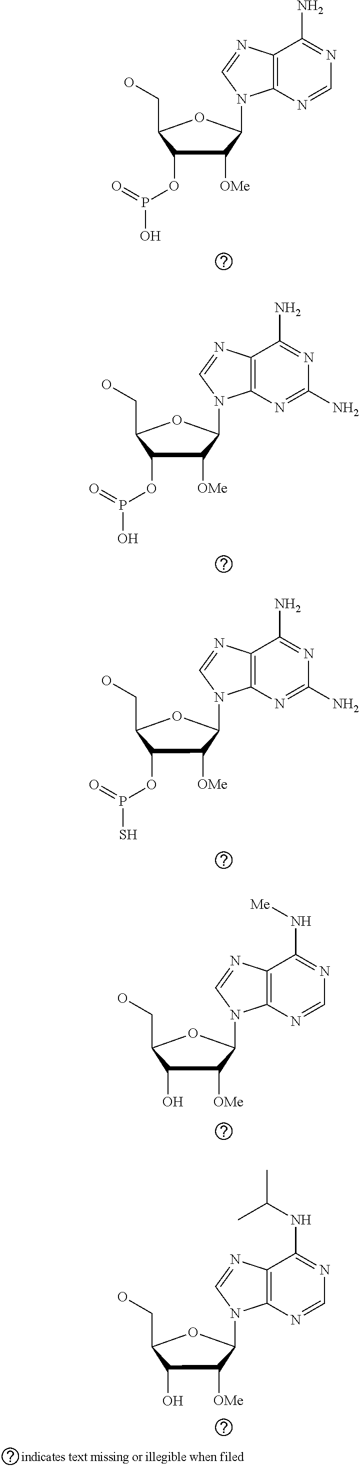

TABLE 7C
N6-alkyl modified siRNA duplexes for in vitro and in vivo assays

| | | | sense (S) | |
|---|---|---|---|---|
| | | | and | |
| | | | antisense | |
| Entry | Target | Duplex | (AS) | Sequence 5′-3′ |
| 1 | mTTR | AD-125773 | S | ususcuugCfuCfUfAfuaaaccguguL96 |
| | | | AS | asCfsacgguuuauagAfgCfaagaascsa |
| 2 | mTTR | AD-538705 | S | ususcuugCfuCfUfAfuaaaccguguL96 |
| | | | AS | asCfsacgguuuauagAfgCfaagaascsQ225 |
| 3 | mTTR | AD-538706 | S | ususcuugCfuCfUfAfuaaaccguguL96 |
| | | | AS | Q225sCfsacgguuuauagAfgCfaagaascsa |
| 4 | mTTR | AD-1637607 | S | ususcuugCfuCfUfAfuaaaccguguL96 |
| | | | AS | Q225sCfsacgguuuauagAfgCfaagaascsQ225 |
| 5 | mTTR | AD-1637609 | S | ususcuugCfuCfUfAfuaaaccguguL96 |
| | | | AS | asCfsacgguuuauagAfgCfaagaascsQ418 |
| 6 | mTTR | AD-1637610 | S | ususcuugCfuCfUfAfuaaaccguguL96 |
| | | | AS | Q418sCfsacgguuuauagAfgCfaagaascsQ418 |
| 7 | mTTR | | S | ususcuugCfuCfUfAfuaaaccguguL96 |
| | | | AS | Q418sCfsacgguuuauagAfgCfaagaascsa |
| 8 | mTTR | | S | ususcuugCfuCfUfAfuaaaccguguL96 |
| | | | AS | (aas)CfsacgguuuauagAfgCfaagaascsa |
| 9 | mTTR | | S | ususcuugCfuCfUfAfuaaaccguguL96 |
| | | | AS | asCfsacgguuuauagAfgCfaagaascs(aa) |
| 10 | mTTR | | S | ususcuugCfuCfUfAfuaaaccguguL96 |
| | | | AS | (aas)CfsacgguuuauagAfgCfaagaascs(aa) |
| 11 | mTTR | AD-84850 | S | asascaguGfuUfCfUfugcucuauauL96 |
| | | | AS | asUfsauaGfagcaagaAfcAfcuguususu |
| 12 | mTTR | AD-1637611 | | Q225sascaguGfuUfCfUfugcucuauauL96 |
| | | | | Q225sUfsauaGfagcaagaAfcAfcuguususu |
| 13 | mTTR | AD-1637612 | | Q418sascaguGfuUfCfUfugcucuauauL96 |
| | | | | Q418sUfsauaGfagcaagaAfcAfcuguususu |
| 14 | mTTR | AD-1789920 | | Q226sascaguGfuUfCfUfugcucuauauL96 |
| | | | | Q226sUfsauaGfagcaagaAfcAfcuguususu |
| 15 | mTTR | AD-1789921 | | Q419sascaguGfuUfCfUfugcucuauauL96 |
| | | | | Q419sUfsauaGfagcaagaAfcAfcuguususu |
| 16 | mTTR | | | (aas)ascaguGfuUfCfUfugcucuauauL96 |
| | | | | (aas)UfsauaGfagcaagaAfcAfcuguususu |

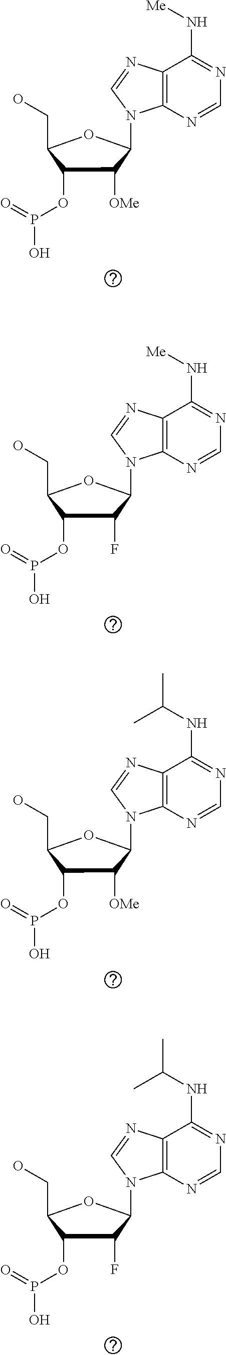

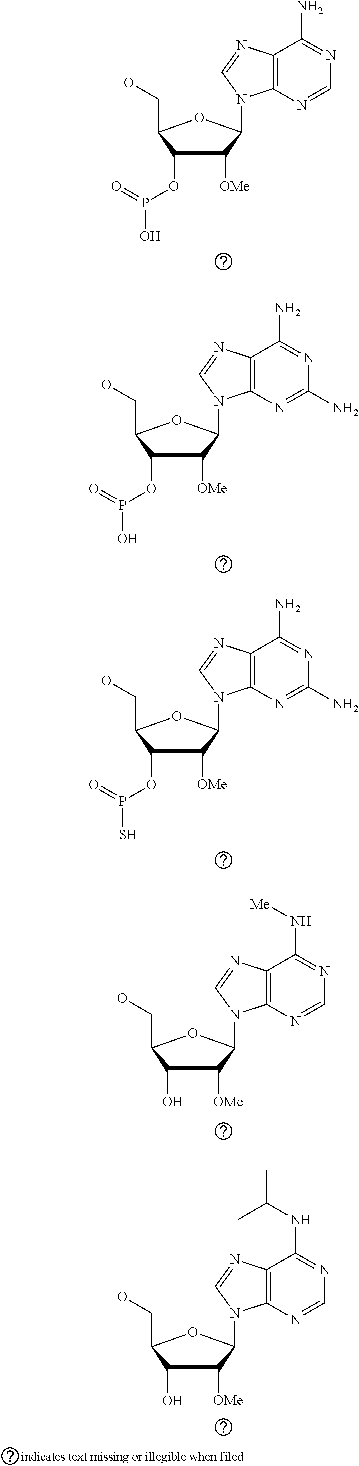

TABLE 8
Characterization of oligonucleotides used for ADA assay

| | | | Mass data | |
|---|---|---|---|---|
| Entry | Strand | Sequence 5′-3′ | Calc. | Obs. |
| 1 | A-173480 | ususcuugCfuCfUfAfuaaaccguguL96 | 8639.391 | 8638.92 |
| 2 | A-140893 | asascaguGfuUfCfUfugcucuauauL96 | 8663.416 | 8663.06 |
| 3 | A-3018917 | Q225sascaguGfuUfCfUfugcucuauauL96 | 8677.441 | 8677.08 |
| 4 | A-3018918 | Q415sascaguGfuUfCfUfugcucuauauL96 | 8705.495 | 8704.9 |
| 5 | A-3196488 | Q226sascaguGfuUfCfUfugcucuauauL96 | 8665.405 | 8665.04 |
| 6 | A-3196489 | Q419sascaguGfuUfCfUfugcucuauauL96 | 8693.459 | 8693.06 |
| 7 | A-3205689 | (aas)ascaguGfuUfCfUfugcucuauauL96 | 8678.429 | 8677.83 |
| 8 | A-173481 | asCfsacgguuuauagAfgCfaagaascsa | 7752.287 | 7751.88 |
| 9 | A-1036679 | Q225sCfsacgguuuauagAfgCfaagaascsa | 7766.313 | 7766.06 |
| 10 | A-1036678 | asCfsacgguuuauagAfgCfaagaascsQ225 | 7766.317 | 7765.87 |
| 11 | A-3018913 | Q225sCfsacgguuuauagAfgCfaagaascsQ225 | 7780.34 | 7779.9 |
| 12 | A-3018914 | Q418sCfsacgguuuauagAfgCfaagaascsa | 7794.364 | 7794.12 |
| 13 | A-3018915 | asCfsacgguuuauagAfgCfaagaascsQ418 | 7794.367 | 7794.1 |
| 14 | A-3018916 | Q418sCfsacgguuuauagAfgCfaagaascsQ418 | 7836.448 | 7836.04 |
| 15 | A-3205691 | (aas)CfsacgguuuauagAfgCfaagaascsa | 7767.298 | 7766.92 |
| 16 | A-3205692 | asCfsacgguuuauagAfgCfaagaascs(aa) | 7767.297 | 7766.61 |
| 17 | A-3205693 | (aas)CfsacgguuuauagAfgCfaagaascs(aa) | 7782.312 | 7781.8 |
| 18 | A-168563 | asUfsauaGfagcaagaAfcAfcuguususu | 7679.157 | 7678.76 |
| 19 | A-1700751 | Q225sUfsauaGfagcaagaAfcAfcuguususu | 7693.183 | 7692.86 |
| 20 | A-3018919 | Q418sUfsauaGfagcaagaAfcAfcuguususu | 7721.237 | 7720.85 |
| 21 | A-3196490 | Q226sUfsauaGfagcaagaAfcAfcuguususu | 7681.147 | 7680.78 |
| 22 | A-3196491 | Q419sUfsauaGfagcaagaAfcAfcuguususu | 7709.201 | 7708.81 |
| 23 | A-3205690 | (aas)UfsauaGfagcaagaAfcAfcuguususu | 7694.171 | 7692.58 |

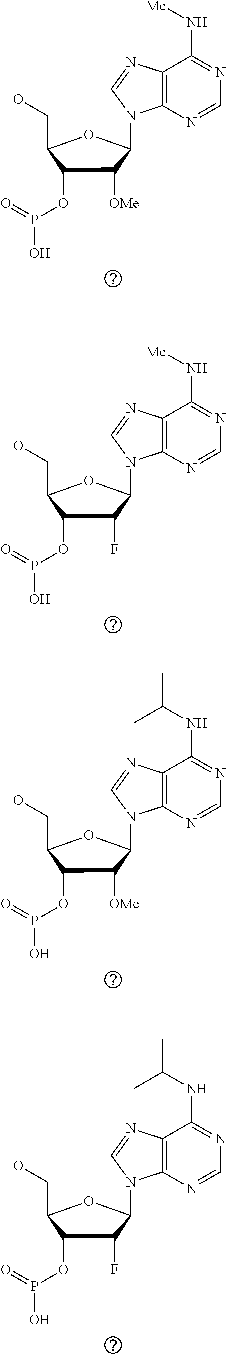

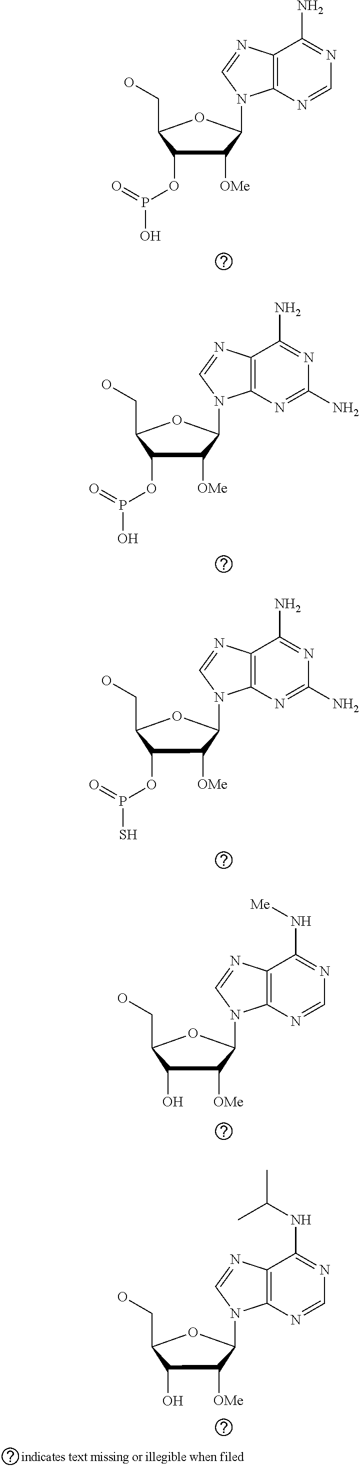

### Thermodynamic Stability

Effect of incorporating 2′-F m6A (2′-F^{m}A) and 2′-OMe m6A (2′-OMeF^{m}A) into DNA/DNA, DNA/RNA, RNA/RNA and RNA/DNA was studied.

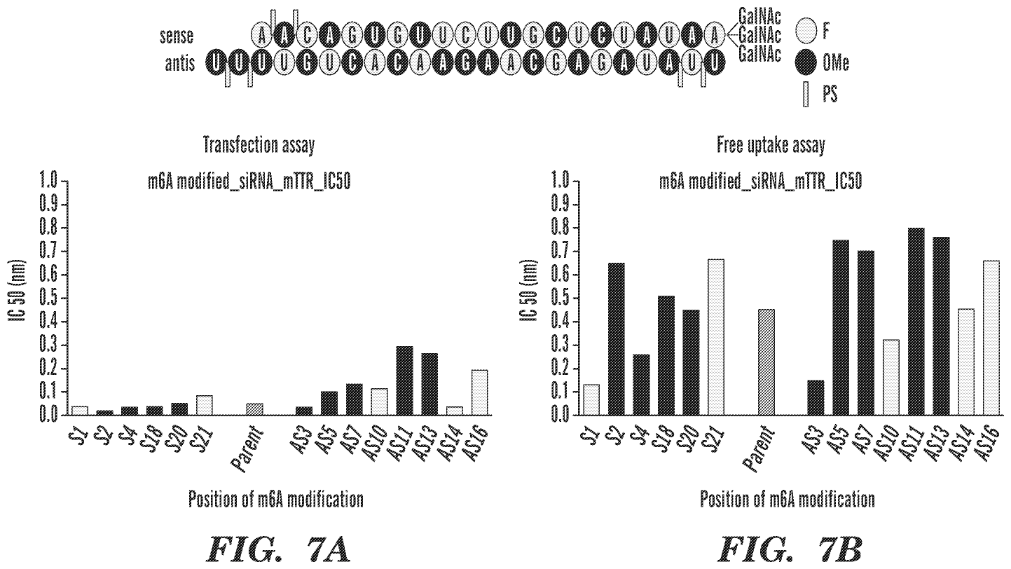

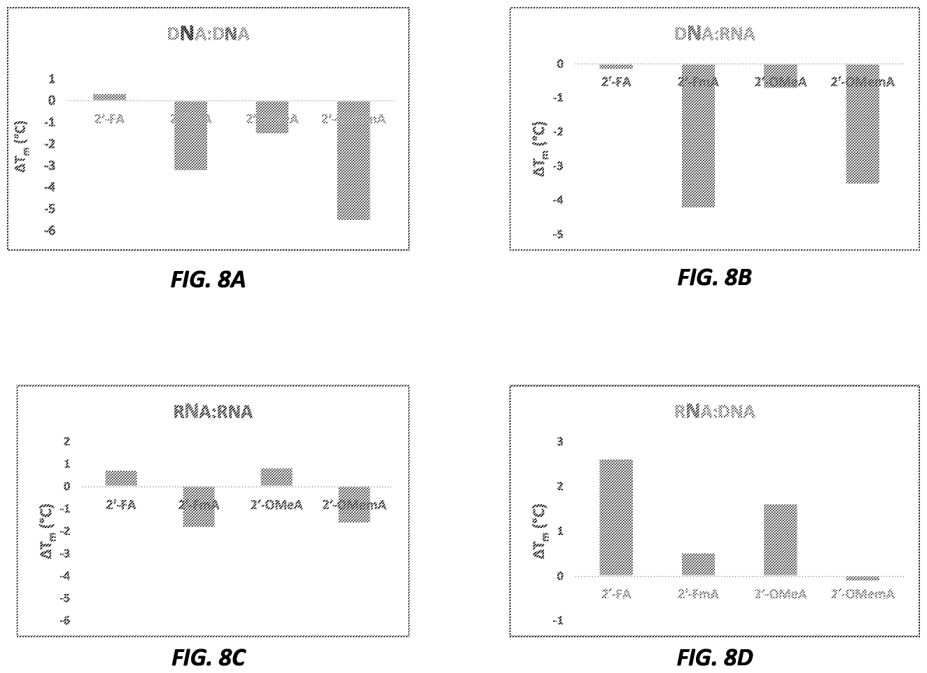

Results are summarized in Table 9 and shown in FIGS. 8A-8D. As can be seen, 2′-F- and 2′-OMe-modified m6A destabilizes DNA:DNA duplexes and enhances stability of RNA:RNA and RNA:DNA duplexes.

TABLE 9
Thermodynamic stability of m6A

| | | | | Tₘ (° C.) | ΔTₘ (° C.) | | |
|---|---|---|---|---|---|---|---|
| Duplexes | Sequence | X = | A | 2′-FA | 2′-F^{m}A | 2′-OMeA | 2′-OMe^{m}A |

| | | | | | | |
|---|---|---|---|---|---|---|
| DNA:DNA | 5′-d(TACAGTCTATGT) | 44.0 | +0.3 | −3.2 | −1.5 | −5.5 |
| | 3′-d(ATGTCXGATACA) | | | | | |
| DNA:RNA | 5′-r(UACAGUCUAUGU) | 43.3 | −0.15 | −4.2 | −0.7 | −3.5 |
| | 3′-d(ATGTCXGATACA) | | | | | |
| RNA:RNA | 5′-r(UACAGUCUAUGU) | 53.7 | +0.7 | −1.8 | +0.8 | −1.6 |
| | 3′-r(AUGUCXGAUACA) | | | | | |
| RNA:DNA | 5′-d(TACAGTCTATGT) | 40.9 | +2.6 | +0.5 | +1.6 | −0.1 |
| | 3′-r(AUGUCXGAUACA) | | | | | |

### REFERENCES

1. O'Brown, Z. K.; Greer, E. L., N6-Methyladenine: A Conserved and Dynamic DNA Mark. Adv. Exp. Med. Biol. 2016, 945 (DNA Methyltransferases—Role and Function), 213-246.
2. Niu, Y.; Zhao, X.; Wu, Y.-s.; Li, M.-m.; Wang, X.-j.; Yang, Y.-g., N6-methyl-adenosine (m6A) in RNA: an old modification with a novel epigenetic function. Genomics, Proteomics Bioinf 2013, 11 (1), 8-17.
3. Roost, C.; Lynch, S. R.; Batista, P. J.; Qu, K.; Chang, H. Y.; Kool, E. T., Structure and thermodynamics of N6-methyladenosine in RNA: A spring-loaded base modification. J. Am. Chem. Soc. 2015, 137 (5), 2107-2115.
4. Schirle, N. T.; Sheu-Gruttadauria, J.; Chandradoss, S. D.; Joo, C.; MacRae, I. J., Water-mediated recognition of t1-adenosine anchors Argonaute2 to microRNA targets. eLife 2015, 4, 07646/1-07646/16.
5. Shelton, S. B.; Reinsborough, C.; Xhemalce, B., Who watches the watchmen: roles of RNA modifications in the RNA interference pathway. PLoS Genet. 2016, 12 (7), e1006139/1-e1006139/10.
6. Bae, S.; Chaturvedi, S.; Lakshman, M. K., O6-(benzotriazol-1-yl)inosine derivatives for C6 modification of purine nucleosides. Curr Protoc Nucleic Acid Chem 2009, Chapter 1, Unit 1.22.
7. Shelton, J.; Lu, X.; Hollenbaugh, J. A.; Cho, J. H.; Amblard, F.; Schinazi, R. F., Metabolism, Biochemical Actions, and Chemical Synthesis of Anticancer Nucleosides, Nucleotides, and Base Analogs. Chem. Rev. (Washington, DC, U.S.) 2016, 116 (23), 14379-14455.
8. Inoue, H.; Hayase, Y.; Imura, A.; Iwai, S.; Miura, K.; Ohtsuka, E., Synthesis and hybridization studies on two complementary nona(2′-O-methyl)ribonucleotides. Nucleic Acids Research 1987, 15 (15), 6131-6148.
9. Koshkin, A. A.; Singh, S. K.; Nielsen, P.; Rajwanshi, V. K.; Kumar, R.; Meldgaard, M.; Olsen, C. E Wengel, J, LNA (locked nucleic acids): synthesis ofthe adenine, cytosine, guanine, 5-methylcytosine, thymine and uracil bicyclonucleoside monomers, oligomerization, and unprecedented nucleic acid recognition. Tetrahedron 1998, 54 (14), 3607-3630.
10. Kierzek, R., The synthesis of 5′-O-(dimethoxytrityl)-N-acyl-2′-deoxynucleosides. Improved “transient protection” approach. NucleotidesNucleotides 1985, 4 (5), 641-9.
11. Perrino, F. W.; Harvey, S.; Blans, P.; Gelhaus, S.; LaCourse, W. R.; Fishbein, J. C., Polymerization PasttheN2-sopropylguanine andtheN6-sopropyladenineDNALesions with the Translesion Synthesis DNA Polymerases hr and the Replicative DNA Polymerase ot. Chem. Res. Toxicol. 2005, 18 (9), 1451-1461.
12. Janas, M. M.; Zlatev, I.; Liu, J.; Jiang, Y, Barros, S A Sutherland, J. E.; Davis, W. P.; Liu, J.; Brown, C. R.; Liu, X.; Schlegel, M. K.; Blair, L.; Zhang, X.; Das, B.; Tran, C.; Aluri, K.; Li, J.; Agarwal, S.; Indrakanti, R.; Charisse, K.; Nair, J.; Matsuda, S.; Raj eev, K. G.; Zimmermann, T.; Sepp-Lorenzino, L.; Xu, Y.; Akinc, A.; Fitzgerald, K.; Vaishnaw, A. K.; Smith, P. F.; Manoharan, M.; Jadhav, V.; Wu, J. T.; Maier, M. A., Safety evaluation of 2′-deoxy-2′-fluoro nucleotides in GalNAc-siRNA conjugates. Nucleic Acids Res 2019, 47 (7), 3306-3320.

TABLE 10
Abbreviations of nucleotide monomers used in nucleic acid sequences

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| dT | 2′-deoxythymidine |
| dTs | 2′-deoxythymidine-3′-phosphorothioate |
| dU | 2′-deoxyuridine |
| dUs | 2′-deoxyuridine-3′-phosphorothioate |
| (m5dC) | 2′-deoxy-5-methylcytidine-3′-phosphate |
| VPus | 5′-vinylphosphonate-uridine-3′-phosphorothioate |
| s | phosphorothioate linkage |
| L10 | 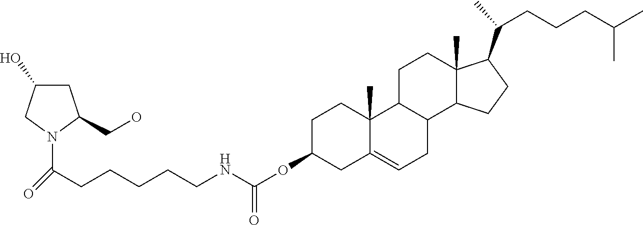 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| L96 | N-[tris(GalNAc-alkyl)-amidodecanoyl)]-4-hydroxyprolinol Hyp-(GalNAc-alkyl)3 |
| Q225 | 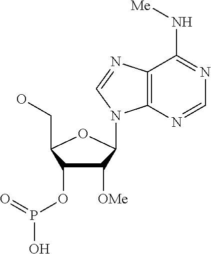 |
| Q226 | 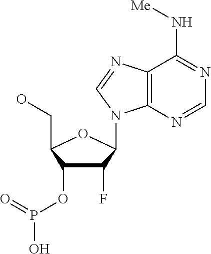 |
| Q418 | 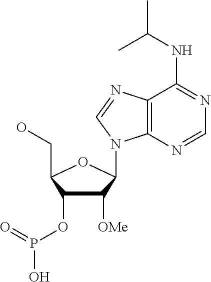 |
| Q419 | 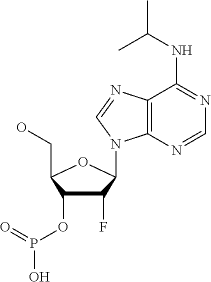 |
| (aa) | 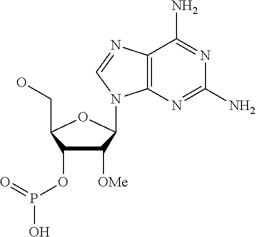 |
| (aas) | 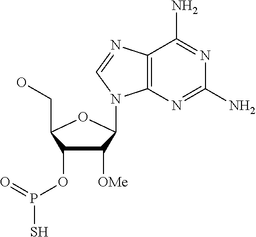 |
| Y239 | 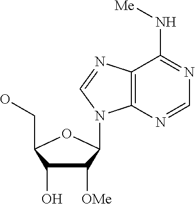 |
| Y238 | 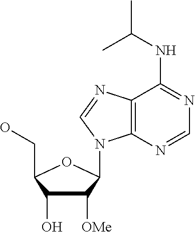 |

### Example 2: Adenosine Deaminase Assays

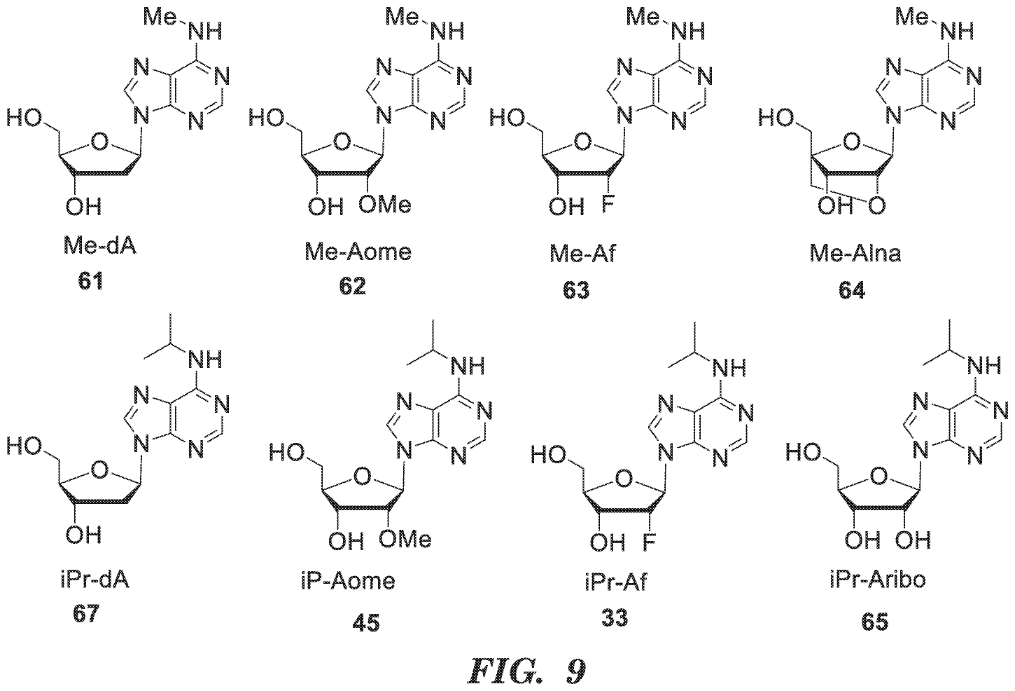

Experimental conditions for evaluating adenosine deaminase activity using human liver S9 fractions:
4 different N6-alkyl (methyl and isopropyl) derivatives of adenosine (with ribose, deoxyribose, 2′-fluoro, 2′-OMe, and LNA sugar moieties) shown in FIG. 9 were incubated with human liver S9 fractions with/without adding 1 mM NADPH.
Incubations were performed in potassium phosphate buffer, pH 7.4, with 3.3 mM MgCl₂ and 1 mg/mL human liver S9.
Nucleosides were added at 1 μM concentration and incubated for up to 2 hours (samples were taken at 0, 10, 20, 30, 40, 60, 90, and 120 minutes).
Verapamil (at 5 μM) and 2′-fluoro Adenosine (2′F-A) were used as positive controls.
Enzymatic stop reaction/sample preparation involved protein precipitation by adding 3:1 ice-cold acetonitrile, followed by solvent evaporation and sample reconstitution in aqueous solution.
LC-MS analysis was performed with Phenomenex Luna Omega Polar column on Thermo Q-Exactive mass spectrometer.

In the adenosine deaminase assays, 2′-fluoroadenosie was used as a positive control for the deaminase activity:

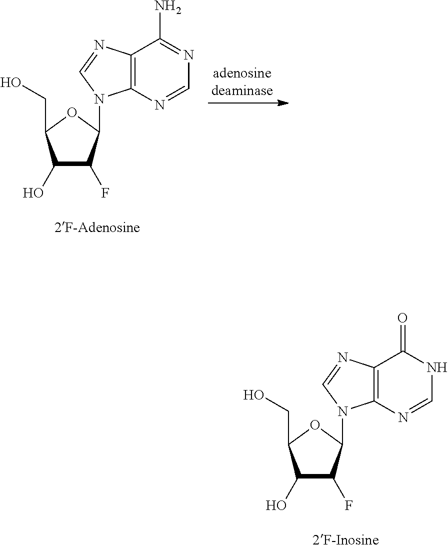

In the adenosine deaminase assays, verapamil, structure shown below, was used as a positive control for CYP enzymes:

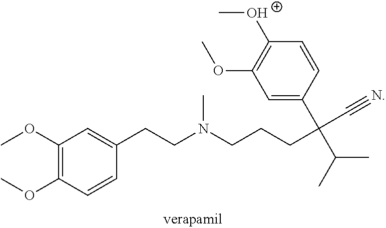 [SMILES: COc1ccc(CCN(C)CCCC(C#N)(c2ccc([OH+]C)c(OC)c2)C(C)C)cc1OC]

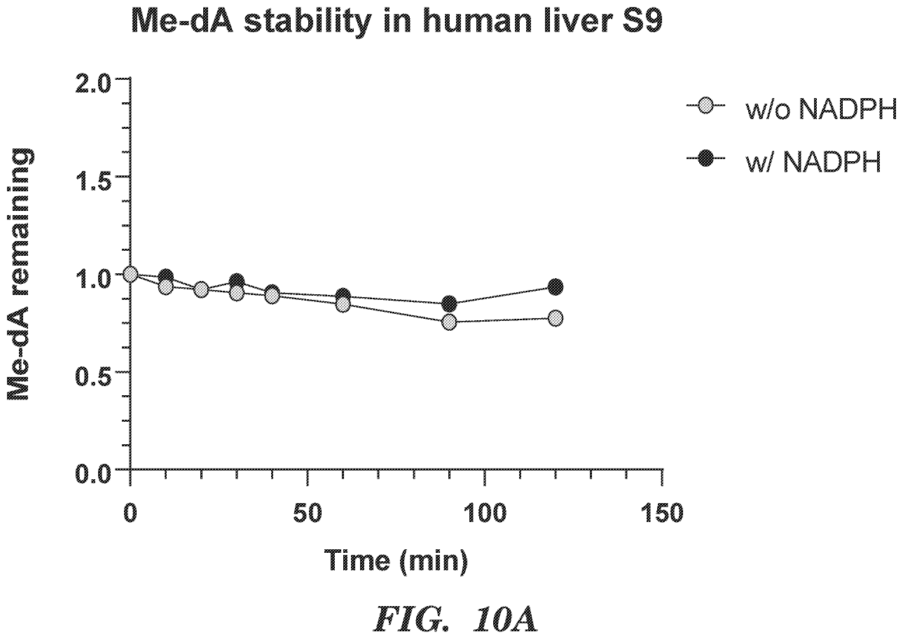

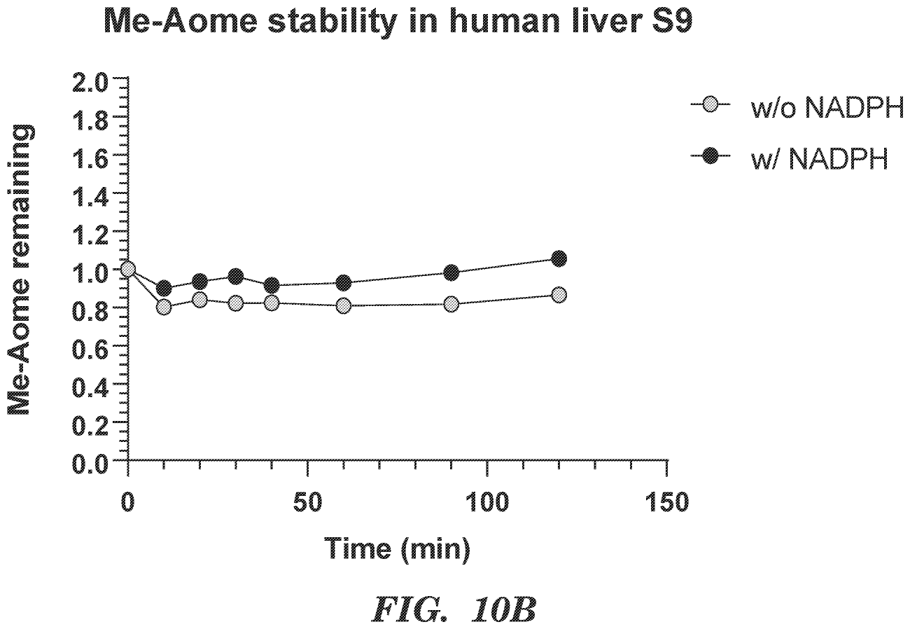

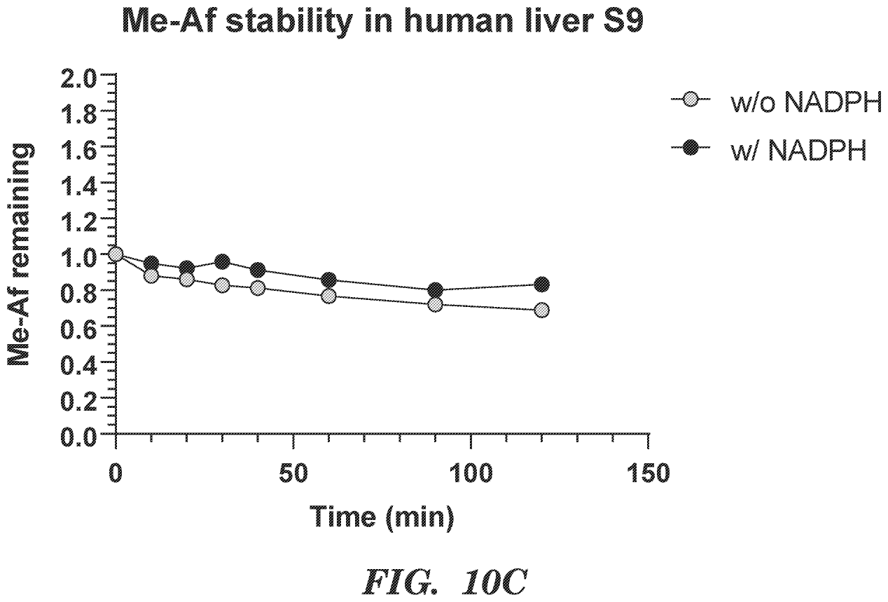

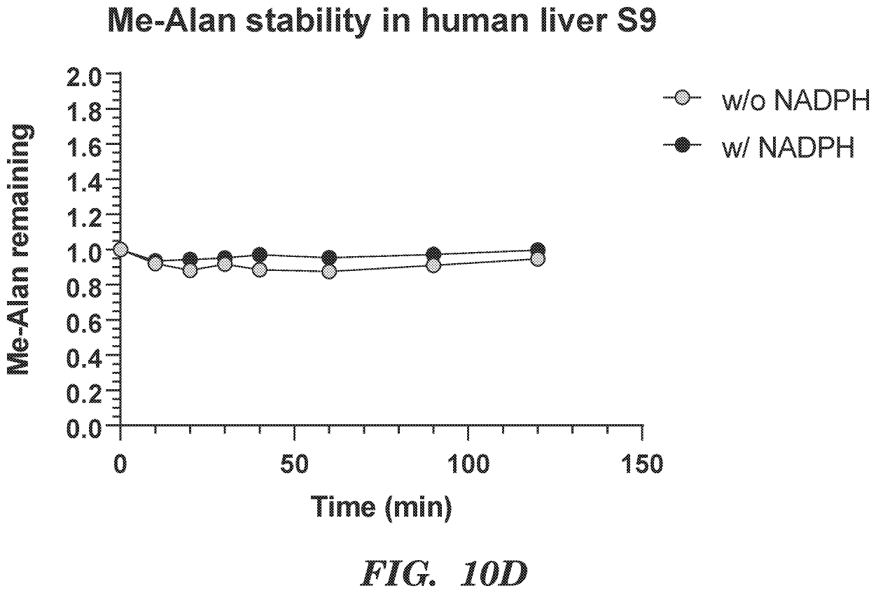

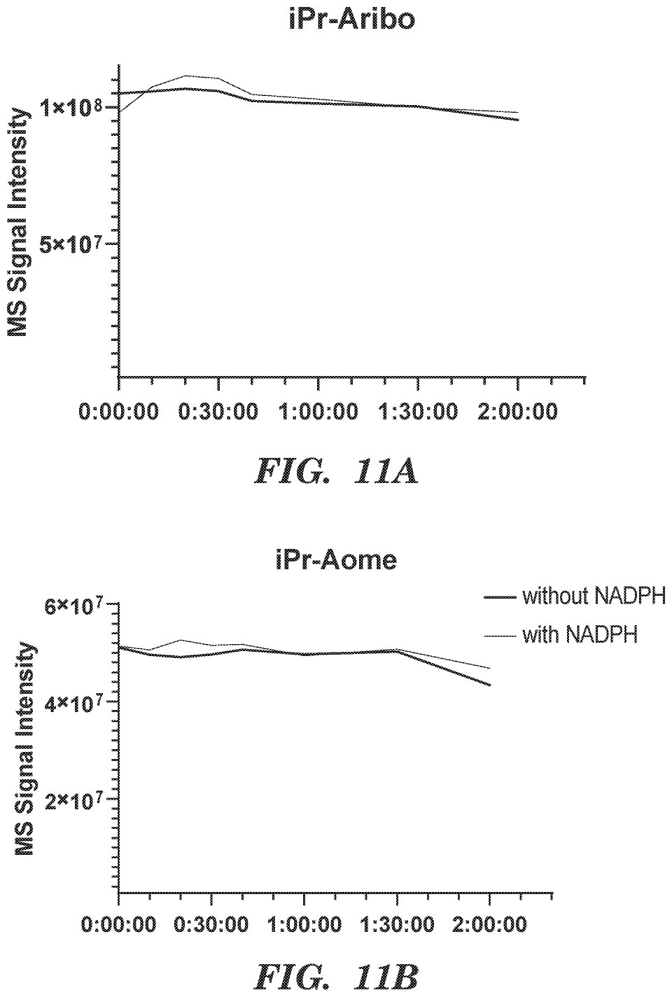

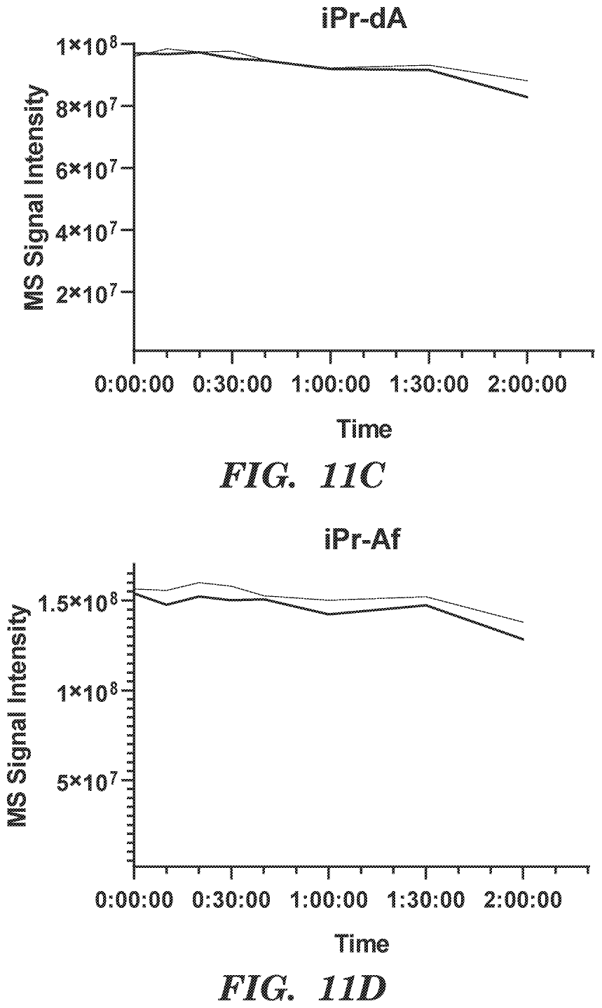

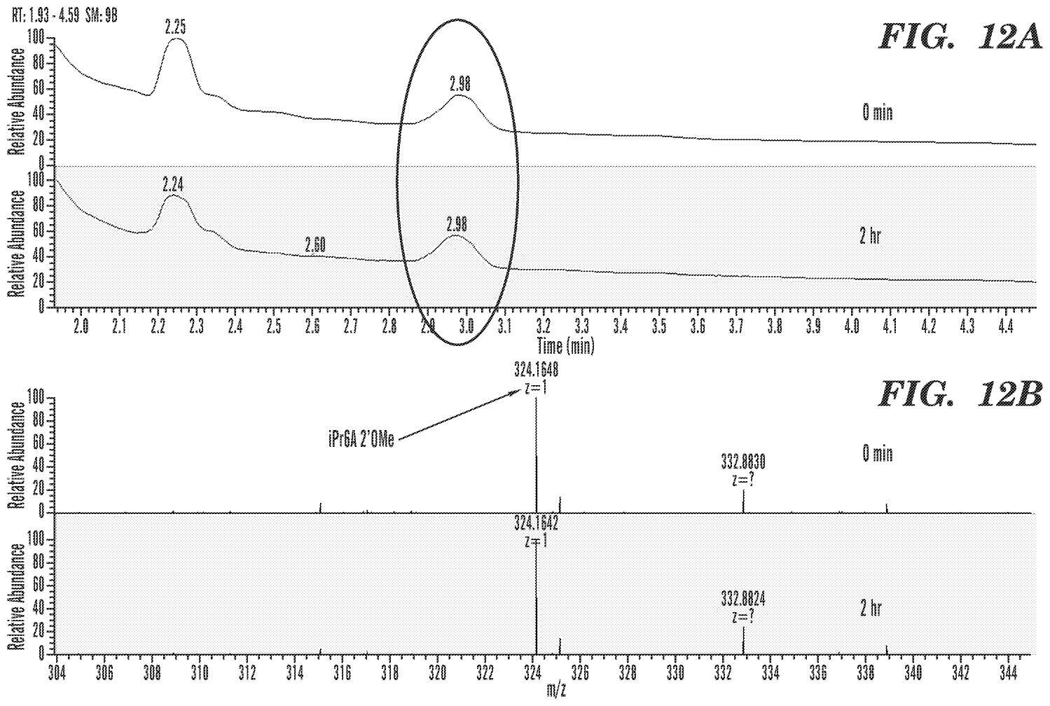

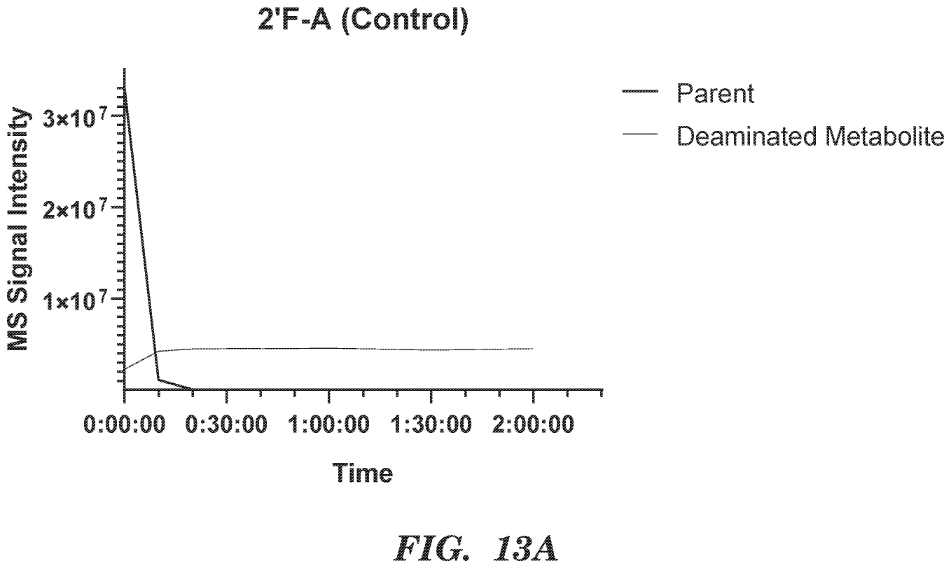

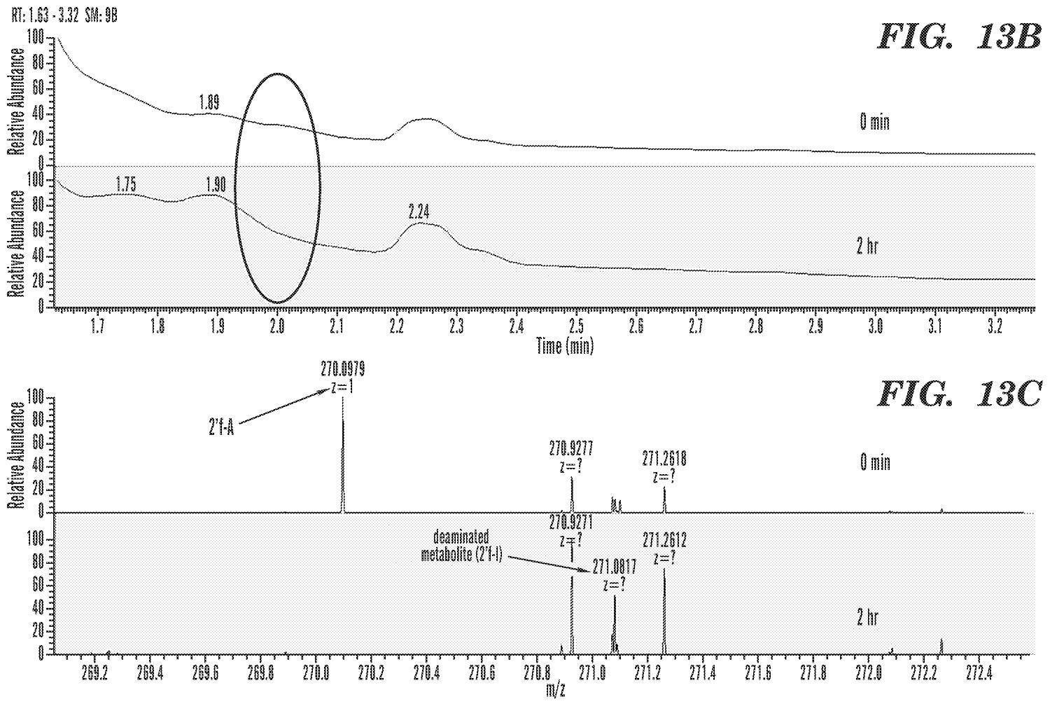

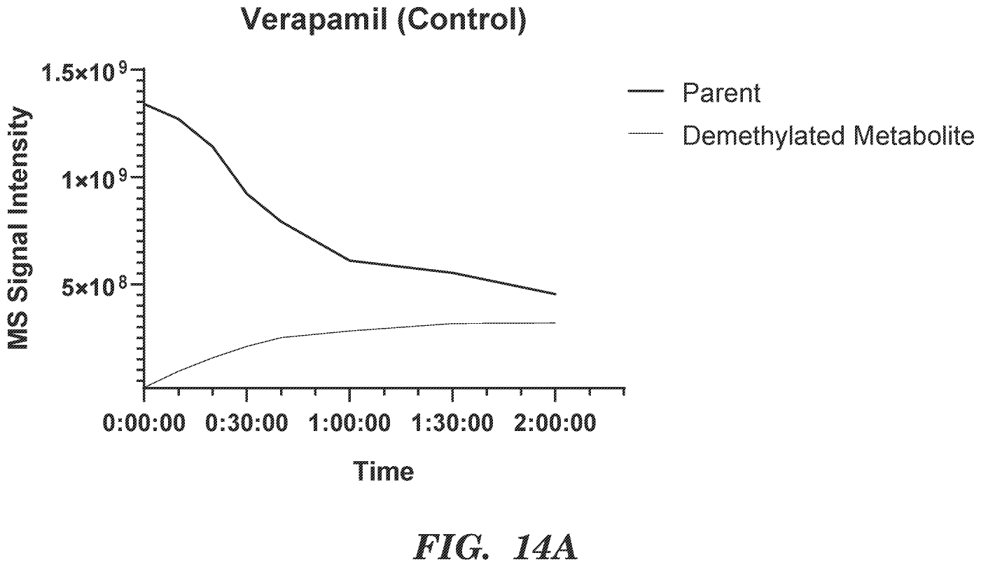

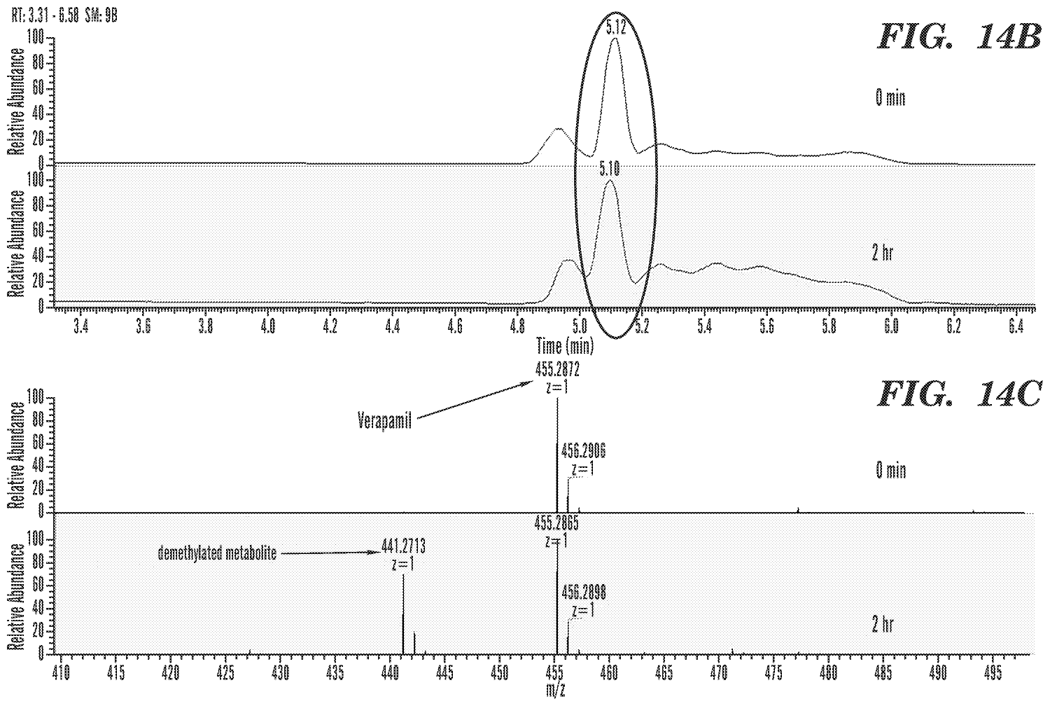

Results are shown in FIGS. 10A-14C.

All of the U.S. patents, U.S. patent application publications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An oligonucleotide comprising at least one nucleoside of Formula (I): 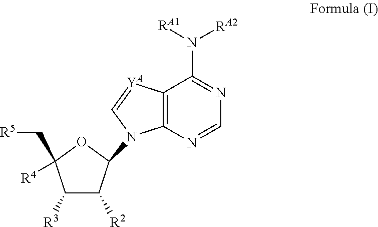
wherein:
Y^{A} is N or CH;
R^{A1} is optionally substituted C₁₋₃₀ alkyl, optionally substituted C₂₋₃₀alkenyl, optionally substituted C₂₋₃₀alkynyl, alkylester, optionally substituted aryl, optionally substituted heteroaryl, optionally substituted heterocyclyl, optionally substituted cycloalkyl, or optionally substituted benzyl, a ligand, or a linker covalently bonded to one or more ligands;
R^{A2} is H or nitrogen protecting group;
R² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a bond to an internucleotide linkage to a subsequent nucleotide, a 3′-oligonucleotide capping group, a ligand, a linker covalently bonded to one or more ligands, a solid support, a linker or a linker covalently bonded a solid support;
R³ is a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—N-methylacetamido, —O—C₄₋₃₀alkyl-ON(CH₂R⁸)(CH₂R⁹), —O—C₄₋₃₀alkyl-ON(CH₂R⁸)(CH₂R⁹), a 3′-oligonucleotide capping group, a ligand, a linker covalently bonded to one or more ligands, a solid support, a linker or a linker covalently bonded to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R² taken together are 4′-C(R¹⁰R¹¹)ᵥ—Y-2′ or 4′-Y—C(R¹° R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
v is 1, 2 or 3;
or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl;
R⁵ represents a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—XP, XP is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is O or S;
a and b are each independently 1-10;
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl, and
provided that,
(i) no more than one of R² and R³ is a bond to an internucleotide linkage to a subsequent nucleotide; and
(ii) when both of R² and R³ are not a bond to an internucleotide linkage, then R⁵ is a bond to an internucleotide linkage to a preceding nucleotide;
provided that the nucleoside of Formula (I) is not where Y^{A} is N; R^{A1} is methyl; R^{A2} is H or nitrogen protecting group; R²² is hydroxyl or protected hydroxyl; R²³ is a bond to an internucleotide linkage to a subsequent nucleotide, hydroxyl or protected hydroxyl; R⁴ is H; and R²⁵ is a bond to an internucleotide linkage to a preceding nucleotide, hydroxyl or protected hydroxyl, and both of R²³ and R²⁵ are not hydroxyl or protected hydroxyl at the same time.

2. The oligonucleotide of claim 1, wherein Y^{A} is N.

3. (canceled)

4. The oligonucleotide of claim 1, wherein R^{A1} is optionally substituted C₁₋₃₀ alkyl, optionally substituted C₂₋₃₀alkenyl, optionally substituted C₂₋₃₀alkynyl, optionally substituted aryl, optionally substituted cycloalkyl, or optionally substituted benzyl group.

5. The oligonucleotide of claim 1, wherein R^{A1} is optionally substituted C₁₋₃₀ alkyl, or 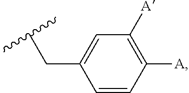
where A and A′ independently are hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl, alkoxyalkylamine, alkoxyoxycarboxylate, amino, alkylamino, dialkylamino, protected amino, a ligand, or a linker covalently bonded to one or more ligands.

6. (canceled)

7. (canceled)

8. The oligonucleotide of claim 1, wherein R^{A2} is hydrogen.

9. (canceled)

10. The oligonucleotide of claim 1, wherein R² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support; or R² and R⁴ taken together are 4′-C(R¹⁰R¹¹)ᵥ—Y-2′ or 4′-Y—C(R¹⁰R¹¹)ᵥ-2′.

11. (canceled)

12. The oligonucleotide of claim 1, wherein R² is hydrogen, hydroxyl, protected hydroxyl, fluoro, methoxy, ethoxy, 2-methoxyethoxy, —O—N-methylacetamido, C₆₋₂₄ alkyl (e.g., n-C₆₋₂₄ alkyl) or C₆₋₂₄alkoxy (e.g., n-C₆₋₂₄ alkoxy); or R² and R⁴ taken together are 4′-C(R¹⁰R¹¹)ᵥ—Y-2′ or 4′-Y—C(R¹⁰R¹¹)ᵥ-2′.

13. (canceled)

14. The oligonucleotide of claim 1, wherein R⁴ is H.

15. The oligonucleotide of claim 1, wherein R³ is a bond to an internucleotide linkage to a subsequent nucleotide, hydroxyl, optionally substituted C₁₋₃₀ alkoxy, a 3′-oligonucleotide capping group, a solid support, a linker or a linker covalently bonded to a solid support.

16. The oligonucleotide of claim 1, wherein R³ is a bond to an internucleotide linkage to a subsequent nucleotide, a hydroxyl or protected hydroxyl.

17. (canceled)

18. The oligonucleotide of claim 1, wherein R⁵ is a bond to an internucleotide linkage to a preceding nucleotide, hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate, phosphate mimic, or a bond to an internucleotide linkage to a preceding nucleotide.

19. The oligonucleotide of claim 1, wherein R⁵ is hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate.

20. The oligonucleotide of claim 1, wherein R⁵ is a bond to an internucleotide linkage to a preceding nucleotide.

21. The oligonucleotide of claim 1, wherein the oligonucleotide comprises from 3 to 50 nucleotides.

22. The oligonucleotide of claim 1, wherein the oligonucleotide comprises at least one ribonucleotide, or comprises at least one 2′-deoxyribonucleotide, or at least one nucleotide with a modified or non-natural nucleobase in addition to the nucleotide of Formula (I), or comprises at least one nucleotide with a modified ribose sugar in addition to the nucleotide of Formula (I), or comprises at least one nucleotide comprising a group other than H or OH at the 2′-position of the ribose sugar in addition to the nucleotide of Formula (I), or comprises at least one nucleotide comprising a moiety other than a ribose sugar in addition to the nucleotide of Formula (I).

23.-26. (canceled)

27. The oligonucleotide of claim 1, wherein the oligonucleotide comprises at least one nucleotide with a 2′-F ribose in addition to the nucleotide of Formula (I), or comprises at least one nucleotide with a 2′-OMe ribose in addition to the nucleotide of Formula (I).

28. (canceled)

29. (canceled)

30. The oligonucleotide of claim 1, wherein the oligonucleotide comprises at least one modified internucleotide linkage.

31. (canceled)

32. The oligonucleotide of claim 1, wherein oligonucleotide comprises at least one ligand.

33. (canceled)

34. A double-stranded nucleic acid comprising a first oligonucleotide strand and a second oligonucleotide strand substantially complementary to the first strand, wherein the first or second strand is an oligonucleotide of claim 1.

35.-39. (canceled)

40. A method of reducing the expression of a target gene in a subject, comprising administering to the subject either:
(i) a double-stranded RNA according to claim 34, wherein the first strand or the second strand is complementary to a target gene; or
(ii) an oligonucleotide according to claim 1, wherein the oligonucleotide is complementary to a target gene.

41. A compound of Formula (II): 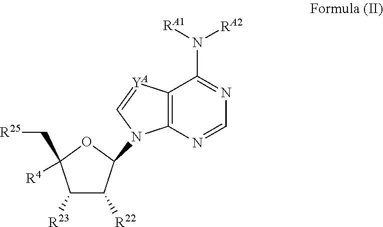
wherein:
Y^{A} is N or CH;
R^{A1} is optionally substituted C₁₋₃₀ alkyl, optionally substituted C₂₋₃₀alkenyl, optionally substituted C₂₋₃₀alkynyl, alkylester, optionally substituted aryl, optionally substituted heteroaryl, optionally substituted heterocyclyl, optionally substituted cycloalkyl, or optionally substituted benzyl, a ligand, or a linker covalently bonded to one or more ligands; and
R^{A2} is H or nitrogen protecting group;
R²² is hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a ligand, a linker covalently bonded to one or more ligands, a solid support, a linker or a linker covalently bonded a solid support;
R²³ is hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a ligand, a linker covalently bonded to one or more ligands, a solid support, a linker or a linker covalently bonded to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R²² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
v is 1, 2 or 3;
or R⁴ and R²³ taken together with the atoms to which they are attached form an optionally substituted C₃₋₈cycloalkyl, optionally substituted C₃₋₈cycloalkenyl, or optionally substituted 3-8 membered heterocyclyl;
R²⁵ is hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄-30alkyl-ON(CH₂R⁸)(CH₂R⁹), —O—C₄₋₃₀alkyl-ON(CH₂R⁸)(CH₂R⁹), vinylphosphonate (VP) group (e.g., ═CH—XP, XP is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R¹)(OH)(O)P—O-5′, R¹ is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is O or S;
a and b are each independently 1-10;
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl, and
provided that
only one of R²² and R²³ is a reactive phosphorous group; and
provided that the compound is not where Y^{A} is N; R^{A1} is methyl; R^{A2} is H or nitrogen protecting group; R²² is hydroxyl, protected hydroxyl, reactive phosphorous, a linker or a linker attached to a solid-support; R²³ is hydroxyl, protected hydroxyl, reactive phosphorous, a linker or a linker attached to a solid-support; R⁴ is H; and R²⁵ is hydroxyl or protected hydroxyl.

42.-86. (canceled)

87. A compound of Formula (III): 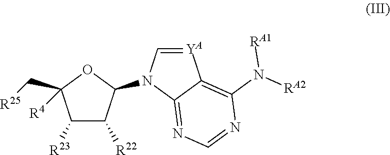
wherein:
Y^{A} is N or CH;
R^{A1} is optionally substituted C₁₋₃₀ alkyl, optionally substituted C₂₋₃₀alkenyl, optionally substituted C₂₋₃₀alkynyl, alkylester, optionally substituted aryl, optionally substituted heteroaryl, optionally substituted heterocyclyl, optionally substituted cycloalkyl, or optionally substituted benzyl, a ligand, or a linker covalently bonded to one or more ligands; and
R^{A2} is hydrogen or a nitrogen protecting group;
one of R²² and R²³ is protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a ligand, or a linker covalently bonded to one or more ligands;
the other of R²² and R²³ is a reactive phosphorous group, a protected hydroxyl, or a hydroxyl;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R²² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
v is 1, 2 or 3;
or R⁴ and R²³ taken together with the atoms to which they are attached form an optionally substituted C₃₋₈cycloalkyl, optionally substituted C₃₋₈cycloalkenyl, or optionally substituted 3-8 membered heterocyclyl;
and R²⁵ is protected hydroxyl.

88.-103. (canceled)
